(19) United States
(12) Patent Application Publication
(10) Pub. No.: US 2026/0176254 A1
(43) Pub. Date: Jun. 25, 2026

(54) INHIBITORS OF PROSTATE SPECIFIC MEMBRANE ANTIGEN AND USE THEREOF

(71) Applicant: SHANGHAI SINOTAU BIOTECH. CO., LTD, Shanghai (CN)

(72) Inventors: Yun JIN, Shanghai (CN); Yang LIU, Shanghai (CN)

(21) Appl. No.: 19/124,242

(22) PCT Filed: Sep. 11, 2024
(86) PCT No.: PCT/CN2024/118273

§ 371 Date: Apr. 24, 2025

(30) Foreign Application Priority Data
Sep. 12, 2023 (WO) PCT/CN2023/118176
May 10, 2024 (WO) PCT/CN2024/092402

(51) Int. Cl. C07D 401/14

(52) U.S. Cl. CPC C07D 401/14

(57) ABSTRACT

Provided herein are a compound of Formula (I), a pharmaceutical composition comprising said compound, and method of use of the compound or pharmaceutical composition in the diagnosis or treatment of a disease or disorder, e.g., a disease or disorder characterized by overexpression of prostate-specific membrane antigen (PSMA).

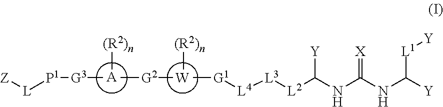

### CROSS-REFERENCE TO RELATED APPLICATIONS

This application claims priority to International Patent Application No. PCT/CN2023/118176 filed on Sep. 12, 2023 and International Patent Application No. PCT/CN2024/092402 filed on May 10, 2024, the entirety of each of which is incorporated herein by reference.

### FIELD

Provided herein are certain compounds that inhibits prostate-specific membrane antigen (PSMA), pharmaceutical compositions comprising said compounds, and method of use of the compounds or pharmaceutical compositions in the diagnosis or treatment of a disease or disorder, e.g., a disease or disorder characterized by overexpression of PSMA.

### BACKGROUND

Prostate cancer is the second most common male malignancy and the most prevalent cancer in the male reproductive system (Rawla 2019). It accounted for 3.8% all death caused by cancer in man in 2018. Worldwide, an estimated 1.4 million people were diagnosed with prostate cancer in 2020 as per GLOBOCAN data and there will be over one million new cases in 2040 (Deo et al 2022). Despite a high 5-year survival rate thanks to early detection and intervention, around 62 per 100,000 men develop metastatic castration-resistant prostate cancer (mCRPC), an advanced stage of prostate cancer (Thurin et al 2020). CRPC is highly mortal and the median overall survival (OS) is less than 2 years (Khoshkar et al 2022). Thus, there is a huge unmet medical need.

Over 80% patients of prostate cancer highly express prostate specific membrane antigen (PSMA), a type-II, 750 amnio acid transmembrane protein, also known as folate hydrolase I or glutamate carboxypeptidase II (O'Keefe et al 2018). It cleaves terminal carboxy glutamates from both and gamma-linked folate polyglutamate and the neuronal dipeptide N-acetylaspartylglutamate (NAAG). PSMA, albeit has low expression in brain, salivary, kidney and small intestines, has an increased expression level of 100 to 1000-fold in prostatic cancer (Heston 1997). Nevertheless, PSMA is positively correlated with Gleason score and cancer aggressiveness and remains highly expressed in CRPC, which makes it an ideal target for diagnosis and therapy.

Many modalities are being developed to treat prostate cancer based on PSMA targeting, including but not limited to prodrug, antibody-drug conjugates, cellular immunotherapy, photodynamic therapy, imaging-guided surgery, ultrasound-mediated nanobubble destruction (Wang et al 2022). Among them, PSMA-targeted radiotheranostics is proved feasible. Pluvicto (lutetium Lu 177 vipivotide tetraxetan) and Locametz (gallium Ga-68 gozetotide) were approved for PSMA-positive mCRPC therapy and diagnostic agent for positron emission tomography (PET) of PSMA-positive lesions respectively in 2022. The median OS treated with Pluvicto was 15.3 months, 4 months longer than standard of care (SOC). About a third (30%) of patients with evaluable disease at baseline demonstrated an overall response per RECIST 1.1 criteria with Pluvicto plus SOC, compared to 2% in the SOC alone (Sartor et al 2021). The modest improvement on median OS and response rate suggests that there is a call for ligands of a higher safety margin and directing more tumor lesion absorption and longer retention, eventually driving a better clinical outcome.

PSMA targeted therapy might also be clinically beneficial for cancers other than prostate. PSMA is found to have elevated expression level in adenoid cystic carcinoma (²Wang et al 2022), salivary duct carcinoma (Terroir et al 2023), sarcomas (Kleiburg et al 2022) and et al. A few PSMA radioligand therapies have already been explored in the clinical setting or used for compassionate treatment (Wang et al 2022, Terroir et al 2023), though the real medical benefit remains to be validated in larger cohorts and better designed studies.

In summary, PSMA targeted therapy, stand alone or in combination with other therapeutic options, is of great value for the treatment and diagnosis of PSMA-positive cancer.

### SUMMARY

In one embodiment, provided herein is a compound of Formula (I):

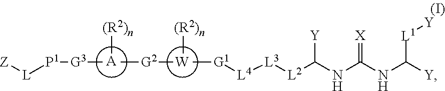

or a stereoisomer, a mixture of stereoisomers, a tautomer, or a pharmaceutically acceptable salt thereof, wherein L¹ to L⁴, X, Y, G¹, G², G³, Ring W, Ring A, R², n, P¹, L, and Z are as defined herein or elsewhere.

Also provided herein is a complex formed by a compound provided herein and a divalent or trivalent metal cation.

Also provided herein is a pharmaceutical composition comprising a compound provided herein or a complex of provided herein, and a pharmaceutically acceptable excipient.

Also provided herein is a method of treating or diagnosing a prostate-specific membrane antigen (PSMA) positive cancer, comprising administering a therapeutically effective amount of a compound provided herein or a complex provided herein to a subject in need thereof.

Also provided herein is a method of detecting cells or tissues expressing prostate-specific membrane antigen (PSMA) comprising (i) contacting the PSMA-expressing cells or tissues with a compound provided herein or a complex provided herein and (ii) applying one or more imaging method to detect the cells or tissues.

### BRIEF DESCRIPTION OF THE DRAWINGS

FIG. 1 shows in vivo tumor uptake of [¹⁷⁷Lu]Lu-E8 in LnCAP xenograft mouse model, compared with reference compound [¹⁷⁷Lu]Lu-PSMA-617.

FIG. 2 shows in vivo tumor uptake of [¹⁷⁷Lu]Lu-E7 and [¹⁷⁷Lu]Lu-E8 in 22Rv1 mouse model, compared with reference compound [¹⁷⁷Lu]Lu-PSMA-617.

FIG. 3 shows the inhibition of tumor growth (tumor volume) by [¹⁷⁷Lu]Lu-E8 at ascending doses (103 μCi, 198 μCi, or 516 μCi) in LnCAP CDX xenograft mouse model, compared with saline and reference compound [¹⁷⁷Lu]Lu-PSMA-617 (201 μCi or 512 μCi).

FIG. 4 shows the inhibition of tumor growth (tumor weight) by [¹⁷⁷Lu]Lu-E8 at ascending doses (103 μCi, 198 μCi, or 516 μCi) in LnCAP CDX xenograft mouse model, compared with saline and reference compound [¹⁷⁷Lu]Lu-PSMA-617 (201 μCi or 512 μCi).

### DETAILED DESCRIPTION

It is to be understood that the application provided herein is not limited to the particular methodology, protocols and reagents described herein as these may vary. It is also to be understood that the terminology used herein is for the purpose of describing particular embodiments only, and is not intended to limit the scope of the invention provided herein. Unless defined otherwise, all technical and scientific terms used herein have the same meanings as commonly understood by one of ordinary skill in the art.

Several documents are cited throughout the text of this specification. Each of the documents cited herein (including all patents, patent applications, scientific publications, manufacturer's specifications, instructions etc.), whether supra or infra, is hereby incorporated by reference in its entirety. In the event of a conflict between the definitions or teachings of such incorporated references and definitions or teachings recited in the present specification, the text of the present specification takes precedence.

### Definitions

Unless defined otherwise, all technical and scientific terms used herein have the same meaning as is commonly understood by one of ordinary skill in the art. In one embodiment, unless otherwise specified, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, and unless otherwise specified, the term “alkyl” refers to a straight or branched hydrocarbon chain radical consisting solely of carbon and hydrogen atoms, which is saturated. In one embodiment, the alkyl group has, for example, from one to twenty-four carbon atoms (C₁-C₂₄ alkyl), four to twenty carbon atoms (C₄-C₂₀ alkyl), six to sixteen carbon atoms (C₆-C₁₆ alkyl), six to nine carbon atoms (C₆-C₉ alkyl), one to fifteen carbon atoms (C₁-C₁₅ alkyl), one to twelve carbon atoms (C₁-C₁₂ alkyl), one to eight carbon atoms (C₁-C₈ alkyl) or one to six carbon atoms (C₁-C₆ alkyl) and which is attached to the rest of the molecule by a single bond. Examples of alkyl groups include, but are not limited to, methyl, ethyl, n-propyl, 1-methylethyl (isopropyl), n-butyl, n-pentyl, 1,1-dimethylethyl (t-butyl), 3-methylhexyl, 2-methylhexyl, and the like. Unless otherwise specified, an alkyl group is optionally substituted.

As used herein, and unless otherwise specified, the term “alkenyl” refers to a straight or branched hydrocarbon chain radical consisting solely of carbon and hydrogen atoms, which contains one or more carbon-carbon double bonds. The term “alkenyl” also embraces radicals having “cis” and “trans” configurations, or alternatively, “E” and “Z” configurations, as appreciated by those of ordinary skill in the art. In one embodiment, the alkenyl group has, for example, from two to twenty-four carbon atoms (C₂-C₂₄ alkenyl), four to twenty carbon atoms (C₄-C₂₀ alkenyl), six to sixteen carbon atoms (C₆-C₁₆ alkenyl), six to nine carbon atoms (C₆-C₉ alkenyl), two to fifteen carbon atoms (C₂-C₁₅ alkenyl), two to twelve carbon atoms (C₂-C₁₂ alkenyl), two to eight carbon atoms (C₂-C₈ alkenyl) or two to six carbon atoms (C₂-C₆ alkenyl) and which is attached to the rest of the molecule by a single bond. Examples of alkenyl groups include, but are not limited to, ethenyl, prop-1-enyl, but-1-enyl, pent-1-enyl, penta-1,4-dienyl, and the like. Unless otherwise specified, an alkenyl group is optionally substituted.

As used herein, and unless otherwise specified, the term “alkynyl” refers to a straight or branched hydrocarbon chain radical consisting solely of carbon and hydrogen atoms, which contains one or more carbon-carbon triple bonds. In one embodiment, the alkynyl group has, for example, from two to twenty-four carbon atoms (C₂-C₂₄ alkynyl), four to twenty carbon atoms (C₄-C₂₀ alkynyl), six to sixteen carbon atoms (C₆-C₁₆ alkynyl), six to nine carbon atoms (C₆-C₉ alkynyl), two to fifteen carbon atoms (C₂-C₁₅ alkynyl), two to twelve carbon atoms (C₂-C₁₂ alkynyl), two to eight carbon atoms (C₂-C₈ alkynyl) or two to six carbon atoms (C₂-C₆ alkynyl) and which is attached to the rest of the molecule by a single bond. Examples of alkynyl groups include, but are not limited to, ethynyl, propynyl, butynyl, pentynyl, and the like. Unless otherwise specified, an alkynyl group is optionally substituted.

As used herein, and unless otherwise specified, the term “cycloalkyl” refers to a non-aromatic monocyclic or polycyclic hydrocarbon radical consisting solely of carbon and hydrogen atoms, and which is saturated. Cycloalkyl group may include fused, bridged, or spiro ring systems. In one embodiment, the cycloalkyl has, for example, from 3 to 15 ring carbon atoms (C₃-C₁₅ cycloalkyl), from 3 to 10 ring carbon atoms (C₃-C₁₀ cycloalkyl), or from 3 to 8 ring carbon atoms (C₃-C₈ cycloalkyl). The cycloalkyl is attached to the rest of the molecule by a single bond. Examples of monocyclic cycloalkyl radicals include, but are not limited to, cyclopropyl, cyclobutyl, cyclopentyl, cyclohexyl, cycloheptyl, and cyclooctyl. Examples of polycyclic cycloalkyl radicals include, but are not limited to, adamantyl, norbornyl, decalinyl, 7,7-dimethyl-bicyclo[2.2.1]heptanyl, spiro[3,3]heptyl, spiro[3,4]octyl, spiro[4,3]octyl, spiro[3,5]nonyl, spiro[5,3]nonyl, spiro[3,6]decyl, spiro[6,3]decyl, spiro[4,5]decyl, spiro[5,4]decyl, bicyclo[2.2.1]heptyl, bicyclo[2.2.2]octyl, and the like. Unless otherwise specified, a cycloalkyl group is optionally substituted.

As used herein, and unless otherwise specified, the term “heteroalkyl” refers to a saturated straight or branched carbon chain that is interrupted one or more times with the same or different heteroatoms independently selected from nitrogen, oxygen, phosphorous, and sulfur. Examples of heteroalkyl include, but are not limited to, —O—CH₃, —S—CH₃, —CH₂—O—CH₃, —CH₂—O—C₂H₅, —CH₂—S—CH₃, —CH₂—S—C₂H₅, —C₂H₄—O—CH₃, —C₂H₄—O—C₂H₅, —C₂H₄—S—CH₃, —C₂H₄—S—C₂H₅, and the like. Unless otherwise specified, a heteroalkyl group is optionally substituted.

As used herein, and unless otherwise specified, the term “heterocyclyl” refers to a non-aromatic radical monocyclic or polycyclic moiety that contains one or more (e.g., one, one or two, one to three, or one to four) heteroatoms independently selected from nitrogen, oxygen, phosphorous, and sulfur. The heterocyclyl may be attached to the main structure at any heteroatom or carbon atom. A heterocyclyl group can be a monocyclic, bicyclic, tricyclic, tetracyclic, or other polycyclic ring system, wherein the polycyclic ring systems can be a fused, bridged or spiro ring system. Heterocyclyl polycyclic ring systems can include one or more heteroatoms in one or more rings. A heterocyclyl group can be saturated or partially unsaturated. Saturated heterocycloalkyl groups can be termed “heterocycloalkyl”. Partially unsaturated heterocycloalkyl groups can be termed “heterocycloalkenyl” if the heterocyclyl contains at least one double bond, or “heterocycloalkynyl” if the heterocyclyl contains at least one triple bond. In one embodiment, the heterocyclyl has, for example, 3 to 18 ring atoms (3- to 18-membered heterocyclyl), 4 to 18 ring atoms (4- to 18-membered heterocyclyl), 5 to 14 ring atoms (5- to 14-membered heterocyclyl), 5 to 18 ring atoms (5- to 18-membered heterocyclyl), 4 to 8 ring atoms (4- to 8-membered heterocyclyl), or 5 to 8 ring atoms (5- to 8-membered heterocyclyl). Whenever it appears herein, a numerical range such as “3 to 18” refers to each integer in the given range; e.g., “3 to 18 ring atoms” means that the heterocyclyl group can consist of 3 ring atoms, 4 ring atoms, 5 ring atoms, 6 ring atoms, 7 ring atoms, 8 ring atoms, 9 ring atoms, 10 ring atoms, etc., up to and including 18 ring atoms. Examples of heterocyclyl groups include, but are not limited to, imidazolidinyl, oxazolidinyl, thiazolidinyl, pyrazolidinyl, isoxazolidinyl, isothiazolidinyl, morpholinyl, pyrrolidinyl, tetrahydrofuryl, and piperidinyl. Examples of heterocyclyl groups also include, but are not limited to, 1-(1,2,5,6-tetrahydropyridyl), 1-piperidinyl, 2-piperidinyl, 3-piperidinyl, 4-morpholinyl, 3-morpholinyl, 1,8 diazo-spiro-[4,5]decyl, 1,7 diazo-spiro-[4,5]decyl, 1,6 diazo-spiro-[4,5]decyl, 2,8 diazo-spiro[4,5]decyl, 2,7 diazo-spiro[4,5]decyl, 2,6 diazo-spiro[4,5]decyl, 1,8 diazo-spiro-[5,4]decyl, 1,7 diazo-spiro-[5,4]decyl, 2,8 diazo-spiro-[5,4]decyl, 2,7 diazo-spiro[5,4]decyl, 3,8 diazo-spiro[5,4]decyl, 3,7 diazo-spiro[5,4]decyl, 1-azo-7,11-dioxo-spiro[5,5]undecyl, 1,4-diazabicyclo[2.2.2]oct-2-yl, tetrahydrofuran-2-yl, tetrahydrofuran-3-yl, tetrahydrothien-2-yl, tetrahydrothien-3-yl, 1-piperazinyl, 2-piperazinyl, and the like. Unless otherwise specified, a heterocyclyl group is optionally substituted.

As used herein, and unless otherwise specified, the term “aryl” refers to a monocyclic aromatic group and/or multicyclic monovalent aromatic group that contain at least one aromatic hydrocarbon ring. In certain embodiments, the aryl has from 6 to 20 ring carbon atoms (C₆-C₂₀ aryl), from 6 to 18 ring carbon atoms (C₆-C₁₈ aryl), from 6 to 14 ring carbon atoms (C₆-C₁₄ aryl), or from 6 to 10 ring carbon atoms (C₆-C₁₀ aryl). Examples of aryl groups include, but are not limited to, phenyl, naphthyl, fluorenyl, azulenyl, anthryl, phenanthryl, pyrenyl, biphenyl, and terphenyl. The term “aryl” also refers to bicyclic, tricyclic, or other multicyclic hydrocarbon rings, where at least one of the rings is aromatic and the others of which may be saturated, partially unsaturated, or aromatic, for example, dihydronaphthyl, indenyl, indanyl, or tetrahydronaphthyl (tetralinyl). Unless otherwise specified, an aryl group is optionally substituted.

As used herein, and unless otherwise specified, the term “heteroaryl” refers to a monocyclic aromatic group and/or multicyclic aromatic group that contains at least one aromatic ring, wherein at least one aromatic ring contains one or more (e.g., one, one or two, one to three, or one to four) heteroatoms independently selected from O, S, and N. The heteroaryl may be attached to the main structure at any heteroatom or carbon atom. In certain embodiments, the heteroaryl has from 5 to 20, from 5 to 15, or from 5 to 10 ring atoms. The term “heteroaryl” also refers to bicyclic, tricyclic, or other multicyclic rings, where at least one of the rings is aromatic and the others of which may be saturated, partially unsaturated, or aromatic, wherein at least one aromatic ring contains one or more heteroatoms independently selected from O, S, and N. Examples of monocyclic heteroaryl groups include, but are not limited to, pyrrolyl, pyrazolyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to, indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to, carbazolyl, benzindolyl, phenanthrollinyl, acridinyl, phenanthridinyl, and xanthenyl. Unless otherwise specified, a heteroaryl group is optionally substituted.

As used herein, and unless otherwise specified, the term “alkylene” or “alkylene chain” refers to a straight or branched multivalent (e.g., divalent or trivalent) hydrocarbon chain linking the rest of the molecule to a radical group (or groups), consisting solely of carbon and hydrogen, which is saturated. In one embodiment, the alkylene has, for example, from one to twenty-four carbon atoms (C₁-C₂₄ alkylene), one to fifteen carbon atoms (C₁-C₁₅ alkylene), one to twelve carbon atoms (C₁-C₁₂ alkylene), one to eight carbon atoms (C₁-C₁₅ alkylene), one to six carbon atoms (C₁-C₆ alkylene), two to four carbon atoms (C₂-C₄ alkylene), one to two carbon atoms (C₁-C₂ alkylene). Examples of alkylene groups include, but are not limited to, methylene, ethylene, propylene, n-butylene, and the like. The alkylene chain is attached to the rest of the molecule through a single bond and to the radical group through a single bond. The points of attachment of the alkylene chain to the rest of the molecule and to the radical group(s) can be through one carbon or any two (or more) carbons within the chain. Unless otherwise specified, an alkylene chain is optionally substituted.

As used herein, and unless otherwise specified, the term “alkynylene” is a multivalent (e.g., divalent or trivalent) alkynyl group; the term “cycloalkylene” is a multivalent (e.g., divalent or trivalent) cycloalkyl group; the term “heterocyclylene” is a multivalent (e.g., divalent or trivalent) heterocyclyl group; the term “arylene” is a multivalent (e.g., divalent or trivalent) aryl group; and the term “heteroarylene” is a multivalent (e.g., divalent or trivalent) heteroaryl group. Other “ylene” terms can be constructed similarly from the corresponding “yl” terms.

It is to be understood that a “yl” term as used herein includes and can be replaced by the corresponding “ylene” term, if proper based on the valence of the group. For example, when a ring moiety of a compound provided herein is described as heterocyclyl, the ring moiety is also heterocyclylene if it is multivalent (e.g., divalent or trivalent), i.e., it is connected to multiple parts of the compound.

As used herein, and unless otherwise specified, the term “aralkyl refers to an alkyl moiety, which is substituted by aryl. An example is the benzyl radical. As used herein, and unless otherwise specified, the term “heteroaralkyl” refers to an alkyl moiety, which is substituted by heteroaryl. Unless otherwise specified, the terms for other similar composite moieties can be constructed similarly.

When the groups described herein are said to be “substituted,” they may be substituted with any appropriate substituent or substituents. Illustrative examples of substituents include, but are not limited to, those found in the exemplary compounds and embodiments provided herein, as well as: a halogen atom such as F, Cl, Br, or I; cyano; oxo (═O); hydroxyl (—OH); alkyl; alkenyl; alkynyl; cycloalkyl; aryl; —(C═O)OR′; —O(C═O)R′; —C(═O)R′; —OR′; —S(O)ₓR′; —S—SR′; —C(═O)SR′; —SC(═O)R′; —NR′R′; —NR′C(═O)R′; —C(═O)NR′R′; —NR′C(═O)NR′R′; —OC(═O)NR′R′; —NR′C(═O)OR′; —NR′S(O)ₓNR′R′; —NR′S(O)ₓR′; and —S(O)ₓNR′R′, wherein: R′ is, at each occurrence, independently H, C₁-C₁₅ alkyl or cycloalkyl, and x is 0, 1 or 2. In some embodiments the substituent is a C₁-C₁₂ alkyl group. In other embodiments, the substituent is a cycloalkyl group. In other embodiments, the substituent is a halo group, such as fluoro. In other embodiments, the substituent is an oxo group. In other embodiments, the substituent is a hydroxyl group. In other embodiments, the substituent is an alkoxy group (—OR′). In other embodiments, the substituent is a carboxyl group. In other embodiments, the substituent is an amino group (—NR′R′).

As used herein, and unless otherwise specified, the term “optional” or “optionally” (e.g., optionally substituted) means that the subsequently described event of circumstances may or may not occur, and that the description includes instances where said event or circumstance occurs and instances in which it does not. For example, “optionally substituted alkyl” means that the alkyl radical may or may not be substituted and that the description includes both substituted alkyl radicals and alkyl radicals having no substitution.

As used herein, and unless otherwise specified, the term “halo” or “halogen” refers to a halogen residue selected from the group consisting of F, Cl, Br, and I.

As used herein, and unless otherwise specified, the term “linker” refers to any chemically suitable linker. In one embodiment, a linker is not or only slowly cleaved under physiological conditions. In one embodiment, the linker does not comprise recognition sequences for proteases or recognition structures for other degrading enzymes. In one embodiment, when the compounds provided herein are administered systemically to allow broad access to all compartments of the body and subsequently enrichment of the compounds provided herein wherever in the body the tumor is located, the linker is chosen in such that it is not or only slowly cleaved in blood. In one embodiment, the cleavage is considered slowly, if less than 50% of the linkers are cleaved 2 h after administration of the compound to a human patient. Suitable linkers include, but are not limited to, optionally substituted alkyl, heteroalkyl, cycloalkyl, cycloheteroalkyl, aryl, heteroaryl, aralkyl, heteroaralyl, alkenyl, heteroalkenyl, cycloalkenyl, cycloheteroalkenyl, alkynyl, sulfonyl, amines, ethers, thioethers phosphines, phosphoramidates, carboxamides, esters, imidoesters, amidines, thioesters, sulfonamides, 3-thiopyrrolidine-2,5-dion, carbamates, ureas, guanidines, thioureas, disulfides, oximes, hydrazines, hydrazides, hydrazones, diaza bonds, triazoles, triazolines, tetrazines, platinum complexes and amino acids, or combinations thereof. In one embodiment, the linker comprises 1,4-piperazine, 1,3-propane and a phenolic ether or combinations thereof.

The linker can also be a cleavable linker such as a peptide motif that is cleaved by cathepsin. Any suitable linker that is cleavable by cathepsin can be used. Certain suitable cleavable peptide linkers are described in Peterson et al., Bioconjugate Chem., 1998. Suitable cleavable linkers, for example, comprises optionally substituted NO₂Tyr-Gln-Gly-Val-Gln-Phe-Lys(Aminobenzoyl), NO₂Tyr-Asn-Gly-Thr-Gly-Phe-Lys(Aminobenzoyl), NO₂Tyr-Ser-Val-Val-Phe-Phe-Lys(Aminobenzoyl), NO₂Tyr-Val-Gln-Ser-Ala-Phe, Multiple-Val-Gln-Phe-Val, NO₂Tyr-Gly-Val-Phe-Gln-Phe, NO₂Tyr-Gly-Thr-Val-Ala-Phe-Lys(Aminobenzoyl), NO₂Tyr-Ala-Thr-Ala-Phe-Phe-Lys(Aminobenzoyl), NO₂Tyr-Gly-Ser-Val-Gln-Phe-Lys(Aminobenzoyl), NO₂Tyr-Gly-Gly-Gln-Phe-Phe-Lys(Aminobenzoyl), NO₂Tyr-Gln-Ser-Val-Gly-Phe-Lys(Aminobenzoyl), NO₂Tyr-Gly-Ser-Thr-Phe-Phe-Lys(Aminobenzoyl), NO₂Tyr-Gly-Thr-Val-Gln-Phe-Lys(Aminobenzoyl), NO₂Tyr-Gly-Ser-Thr-Phe-Phe-Lys(Aminobenzoyl), NO₂Tyr-Gly-Val-Ala-Gly-Phe-Lys(Aminobenzoyl), NO₂Tyr-Gly-Ser-Thr-Phe-Phe-Lys(Aminobenzoyl), NO₂Tyr-Ala-Ala-Gly-Thr-Phe-Lys(Aminobenzoyl), NO₂Tyr-Val-Ala-Gln-Phe, NO₂Tyr-Gln-Gly-Val-Gly-Phe-Lys(Aminobenzoyl), NO₂Tyr-Val-Asn-Asn-Asn-Phe-Lys(Aminobenzoyl), NO₂Tyr-Ala-Ser-Ala-Asn-Phe-Lys(Aminobenzoyl), NO₂Tyr-Phe-Gln-Thr-Gln-Phe-Lys(Aminobenzoyl), NO₂Tyr-Ala-Ala-Ala-Ser-Phe-Lys(Aminobenzoyl), NO₂Tyr-Gln-Tyr-Ser-Gly-Phe-Lys(Aminobenzoyl), NO₂Tyr-Ala-Ala-Thr-Ala-Phe-Lys(Aminobenzoyl), NO₂Tyr-Ala-Thr-Gln-Phe-Phe-Lys(Aminobenzoyl), NO₂Tyr-Gln-Ser-Ala-Ser-Phe-Lys(Aminobenzoyl), NO₂Tyr-Gly-Thr-Ser-Phe-Phe-Lys(Aminobenzoyl), NO₂Tyr-Thr-Ala-Gly-Ala-Phe-Lys(Aminobenzoyl), NO₂Tyr-Ala-Thr-Thr-Phe-Phe-Lys(Aminobenzoyl), NO₂Tyr-Ala-Ser-Gly-Ser-Phe-Lys(Aminobenzoyl), NO₂Tyr-Gly-Thr-Thr-Phe-Phe-Lys(Aminobenzoyl), NO₂Tyr-Gly-Ala-Ala-Gly-Phe-Lys(Aminobenzoyl), NO₂Tyr-Gly-Thr-Gln-Phe-Phe-Lys(Aminobenzoyl), NO₂Tyr-Ala-Ala-Thr-Gly-Phe-Lys(Aminobenzoyl), NO₂Tyr-Gly-Thr-Gln-Phe-Phe-Lys(Aminobenzoyl), NO₂Tyr-Gln-Thr-Val-Gly-Phe-Lys(Aminobenzoyl), NO₂Tyr-Gly-Thr-Gln-Phe-Phe-Lys(Aminobenzoyl), NO₂Tyr-Ala-Ser-Ala-Gly-Phe-Lys(Aminobenzoyl), NO₂Tyr-Gly-Gln-Ser-Phe-Phe-Lys(Aminobenzoyl), NO₂Tyr-Thr-Ser-Ala-Thr-Phe-Lys(Aminobenzoyl), NO₂Tyr-Gly-Thr-Val-Ala-Phe-Lys(Aminobenzoyl), NO₂Tyr-Thr-Ala-Gln-Ala-Phe-Lys(Aminobenzoyl), NO₂Tyr-Gly-Val-Ala-Ala-Phe-Lys(Aminobenzoyl), NO₂Tyr-Val-Ala-Ser-Ala-Phe-Lys(Aminobenzoyl), NO₂Tyr-Gln-Gly-Ser-Phe-Phe-Lys(Aminobenzoyl), NO₂Tyr-Thr-Ala-Thr-Asn-Phe-Lys(Aminobenzoyl), NO₂Tyr-Ala-Thr-Ser-Phe-Phe-Lys(Aminobenzoyl), NO₂Tyr-Thr-Gly-Val-Gly-Phe-Lys(Aminobenzoyl), NO₂Tyr-Gly-Thr-Ala-Phe-Phe-Lys(Aminobenzoyl), NO₂Tyr-Gln-Val-Ala-Gly-Phe-Lys(Aminobenzoyl), NO₂Tyr-Gly-Ser-Ala-Gln-Phe-Lys(Aminobenzoyl), NO₂Tyr-Val-Ala-Ala-Gln-Phe-Lys(Aminobenzoyl), NO₂Tyr-Gln-Thr-Ala-Thr-Phe-Lys(Aminobenzoyl), NO₂Tyr-Thr-Gly-Tyr-Thr-Phe-Lys(Aminobenzoyl), NO₂Tyr-Ser-Ala-Gly-Thr-Phe-Lys(Aminobenzoyl), NO₂Tyr-Val-Tyr-Tyr-Val-Phe, NO₂Tyr-Ala-Ser-Tyr-Gly-Phe, Z-Phe-Lys-PABC, Z-Phe-Lys, Z-Val-Lys-PABC, Z-Ala-Lys-PABC, Phe-Phe-Lys-PABC, D-Phe-Phe-Lys-PABC, D-Ala-Phe-Lys-PABC, Gly-Phe-Lys-PABC, Ac-Phe-Lys-PABC, HCO-Phe-Lys-PABC, Phe-Lys-PABC, Z-Lys-PABC, Z-Val-Cit-PABC, Z-Val-Cit, Z-Phe-Cit-PABC, Z-Leu-Cit-PABC, Z-Ile-Cit-PABC, Z-Trp-Cit-PABC, Z-Phe-Arg(NO₂)-PABC, and Z-Phe-Arg(Ts)-PABC.

As used herein, and unless otherwise specified, the term “amino acid” refers to any organic acid containing one or more amino substituents, e.g., α-, β- or γ-amino, derivatives of aliphatic carboxylic acids. In the polypeptide notation used herein, e.g., Xaa1Xaa2Xaa3Xaa4Xaa5, wherein Xaa1 to Xaa5 are each and independently selected from amino acids as defined, the left hand direction is the amino terminal direction and the right hand direction is the carboxy terminal direction, in accordance with standard usage and convention.

As used herein, and unless otherwise specified, the term “conventional amino acid” refers to the twenty naturally occurring amino acids, and encompasses all stereometric isoforms, i.e., D, L-, D- and L-amino acids thereof. These conventional amino acids can herein also be referred to by their conventional three-letter or one-letter abbreviations and their abbreviations follow conventional usage (see, for example, Immunology—A Synthesis, 2nd Edition, E. S. Golub and D. R. Gren, Eds., Sinauer Associates, Sunderland Mass. (1991)).

As used herein, and unless otherwise specified, the term “non-conventional amino acid” refers to unnatural amino acids or chemical amino acid analogues, e.g. α,α-disubstituted amino acids, N-alkyl amino acids, homo-amino acids, dehydroamino acids, aromatic amino acids (other than phenylalanine, tyrosine and tryptophan), and ortho-, meta- or para-aminobenzoic acid. Non-conventional amino acids also include compounds which have an amine and carboxyl functional group separated in a 1,3 or larger substitution pattern, such as β-alanine, γ-amino butyric acid, Freidinger lactam, the bicyclic dipeptide (BTD), amino-methyl benzoic acid and others known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art may also be used. The use of analogues or non-conventional amino acids may improve the stability and biological half-life of the added peptide since they are more resistant to breakdown under physiological conditions. The person skilled in the art will be aware of similar types of substitution which may be made. A non-limiting list of non-conventional amino acids which may be used as suitable building blocks for a peptide and their standard abbreviations (in brackets) is as follows: α-aminobutyric acid (Abu), L-N-methylalanine (Nmala), α-amino-α-methylbutyrate (Mgabu), L-N-methylarginine (Nmarg), aminocyclopropane (Cpro), L-N-methylasparagine (Nmasn), carboxylate L-N-methylaspartic acid (Nmasp), aniinoisobutyric acid (Aib), L-N-methylcysteine (Nmcys), aminonorbornyl (Norb), L-N-methylglutamine (Nmgln), carboxylate L-N-methylglutamic acid (Nmglu), cyclohexylalanine (Chexa), L-N-methylhistidine (Nmhis), cyclopentylalanine (Cpen), L-N-methylisolleucine (Nmile), L-N-methylleucine (Nmleu), L-N-methyllysine (Nmlys), L-N-methylmethionine (Nmmet), L-N-methylnorleucine (Nmnle), L-N-methylnorvaline (Nmnva), L-N-methylornithine (Nmorn), L-N-methylphenylalanine (Nmphe), L-N-methylproline (Nmpro), L-N-methylserine (Nmser), L-N-methylthreonine (Nmthr), L-N-methyltryptophan (Nmtrp), D-ornithine (Dorn), L-N-methyltyrosine (Nmtyr), L-N-methylvaline (Nmval), L-N-methylethylglycine (Nmetg), L-N-methyl-t-butylglycine (Nmtbug), L-norleucine (NIe), L-norvaline (Nva), α-methyl-aminoisobutyrate (Maib), α-methyl-γ-aminobutyrate (Mgabu), D-α-methylalanine (Dmala), α-methylcyclohexylalanine (Mchexa), D-α-methylarginine (Dmarg), α-methylcylcopentylalanine (Mcpen), D-α-methylasparagine (Dmasn), α-methyl-α-napthylalanine (Manap), D-α-methylaspartate (Dmasp), α-methylpenicillamine (Mpen), D-α-methylcysteine (Dmcys), N-(4-aminobutyl)glycine (NgIu), D-α-methylglutamine (Dmgln), N-(2-aminoethyl)glycine (Naeg), D-α-methylhistidine (Dmhis), N-(3-aminopropyl)glycine (Norn), D-α-methylisoleucine (Dmile), N-amino-α-methylbutyrate (Nmaabu), D-α-methylleucine (Dmleu), α-napthylalanine (Anap), D-α-methyllysine (Dmlys), N-benzylglycine (Nphe), D-α-methylmethionine (Dmmet), N-(2-carbamylethyl)glycine (NgIn), D-α-methylornithine (Dmorn), N-(carbamylmethyl)glycine (Nasn), D-α-methylphenylalanine (Dmphe), N-(2-carboxyethyl)glycine (NgIu), D-α-methylproline (Dmpro), N-(carboxymethyl)glycine (Nasp), D-α-methylserine (Dmser), N-cyclobutylglycine (Nebut), D-α-methylthreonine (Dmthr), N-cycloheptylglycine (Nchep), D-α-methyltryptophan (Dmtrp), N-cyclohexylglycine (Nchex), D-α-methyltyrosine (Dmty), N-cyclodecylglycine (Nedec), D-α-methylvaline (Dmval), N-cylcododecylglycine (Nedod), D-N-methylalanine (Dnmala), N-cyclooctylglycine (Neoct), D-N-methylarginine (Dnmarg), N-cyclopropylglycine (Nepro), D-N-methylasparagine (Dnmasn), N-cycloundecylglycine (Neund), D-N-methylaspartate (Dnmasp), N-(2,2-diphenylethyl)glycine (Nbhm), D-N-methyleysteine (Dnmcys), N-(3,3-diphenylpropyl)glycine (Nbhe), D-N-methylglutamine (Dnmgln), N-(3-guanidinopropyl)glycine (Narg), D-N-methylglutamate (Dnmglu), N-(1-hydroxyethyl)glycine (Ntbx), D-N-methylhistidine (Dnmhis), N-(hydroxyethyl))glycine (Nser), D-N-methylisoleucine (Dnmile), N-(imidazolylethyl))glycine (Nhis), D-N-methylleucine (Dnmleu), N-(3-indolylyethyl)glycine (Nhtrp), D-N-methyllysine (Dnnilys), N-methyl-γ-aminobutyrate (Nmgabu), N-methylcyclohexylalanine (Nmchexa), D-N-methylmethionine (Dnmmet), D-N-methylornithine (Dnmom), N-methylcyclopentylalanine (Nmcpen), N-methylglycine (Nala), D-N-methylphenylalanine (Dnmphe), N-methylaminoisobutyrate (Nmaib), D-N-methylproline (Dnmpro), N-(1-methylpropyl)glycine (Nile), D-N-methylserine (Dnmser), N-(2-methylpropyl)glycine (Nleu), D-N-methylthreonine (Dnmthr), D-N-methyltryptophan (Dnmtrp), N-(1-methylethyl)glycine (Nval), D-N-methyltyrosine (Dnmtyr), N-methyla-napthylalanine (Nmanap), D-N-methylvaline (Dnmval), N-methylpenicillamine (Nmpen), γ-aminobutyric acid (Gabu), N-(p-hydroxyphenyl)glycine (Nhtyr), L-/-butylglycine (Tbug), N-(thiomethyl)glycine (Ncys), L-ethylglycine (Etg), penicillamine (Pen), L-homophenylalanine (Hphe), L-α-methylalanine (Mala), L-α-methylarginine (Marg), L-α-methylasparagine (Masn), L-α-methylaspartate (Masp), L-α-methyl-t-butylglycine (Mtbug), L-α-methylcysteine (Meys), L-methylethylglycine (Metg), L-α-methylglutamine (MgIn), L-α-methylglutamate (MgIu), L-α-methylhistidine (Mhis), L-α-methylhomophenylalanine (Mhphe), L-α-methylisoleucine (Mile), N-(2-methylthioethyl)glycine (Nmet), L-α-methylleucine (Mleu), L-α-methyllysine (Mlys), L-α-methylmethionine (Mmet), L-α-methylnorleucine (MnIe), L-α-methylnorvaline (Mnva), L-α-methylomithine (Mom), L-α-methylphenylalanine (Mphe), L-α-methylproline (Mpro), L-α-methylserine (Mser), L-α-methylthreonine (Mthr), L-α-methyltryptophan (Mtrp), L-α-methyltyrosine (Mtyr), L-α-methylvaline (Mval), L-N-methylhomophenylalanine (Nmhphe), N—(N-(2,2-diphenylethyl)carbamylmethyl)glycine (Nnbhm), N—(N-(3,3-diphenylpropyl)-carbamylmethyl)glycine (Nnbhe), 1-carboxy-1-(2,2-diphenyl-ethylamino)cyclopropane (Nmbc), L-O-methyl serine (Omser), L-O-methyl homoserine (Omhser).

As used herein, and unless otherwise specified, the term “radioactive moiety” refers to a molecular assembly which carries a radioactive nuclide. The nuclide is bound either by covalent or coordinate bonds which remain stable under physiological conditions.

As used herein, and unless otherwise specified, the term “fluorescent isotope” refers to an isotope that emits electromagnetic radiation after excitation by electromagnetic radiation of a shorter wavelength.

As used herein, and unless otherwise specified, the term “radioisotope” is a radioactive isotope of an element (included by the term “radionuclide”) emitting α-, β-, and/or γ-radiation.

As used herein, and unless otherwise specified, the term “radioactive drug” refers to a biologic active compound which is modified by a radioisotope. Especially, intercalating substances can be used to deliver the radioactivity to direct proximity of DNA (e.g. a ¹³¹I-carrying derivative of Hoechst-33258).

As used herein, and unless otherwise specified, the terms “chelating agent” or “chelator” are used interchangeably and refer to a molecule, often an organic one, and often a Lewis base, having two or more unshared electron pairs available for donation to a metal ion. The metal ion is usually coordinated by two or more electron pairs to the chelating agent. The term “chelating atom” refers to an atom that provides unshared electron pair available for donation to a metal ion. The terms “bidentate chelating agent”, “tridentate chelating agent”, “tetradentate chelating agent”, “hexadentate chelating agent”, and “octadentate chelating agent” refer to chelating agents having, respectively, two, three, four, six and eight electron pairs readily available for simultaneous donation to a metal ion coordinated by the chelating agent. In one embodiment, the chelating agent has 3 or 4 nitrogen chelating atoms. In one embodiment, the chelating agent further has 3 or 4 oxygen-containing groups that contain oxygen chelating atoms. In one embodiment, the oxygen-containing group is a carboxylic acid (—COOH) or a phosphonic acid (—PO₃H₂), or a derivative thereof. Usually, the electron pairs of a chelating agent forms coordinate bonds with a single metal ion; however, in certain examples, a chelating agent may form coordinate bonds with more than one metal ion, with a variety of binding modes being possible.

As used herein and unless otherwise specified, when a “chelating agent” is part of a compound provided herein (e.g., chelating agent Z in a compound of Formula (I) provided herein), it refers to a chelating moiety of a chelating agent complete molecule (even if the chemical name or abbreviation of a complete molecule is used). As used herein and unless otherwise specified, a “chelating moiety” of a chelating agent complete molecule refers to a partial structure of the chelating agent complete molecule, and the partial structure has the same or substantially the same chelating atoms as the complete molecule chelating agent does. For example, DOTA normally refers to a complete molecule of 1,4,7,10-tetraazacyclododecane-N,N′,N,N′-tetra acetic acid. As used herein and unless otherwise specified, a “chelating moiety” of DOTA refers to a partial structure of DOTA that has the same or substantially the same chelating atoms as DOTA, such as the following partial structures:

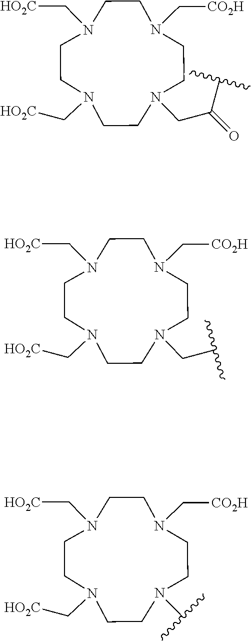 [SMILES: *C(=O)CN1CCN(CC(=O)O)CCN(CC(=O)O)CCN(CC(=O)O)CC1 ; *CN1CCN(CC(=O)O)CCN(CC(=O)O)CCN(CC(=O)O)CC1 ; *N1CCN(CC(=O)O)CCN(CC(=O)O)CCN(CC(=O)O)CC1]

A person of ordinary skill in the art, when looking at the structure of a compound provided herein, would be able to tell the point of attachment of the chelating moiety to the remainder of the compound. In one embodiment, the point of attachment for a chelating agent provided herein is on a chelating atom (e.g., a nitrogen atom). In one embodiment, the point of attachment for a chelating agent provided herein is on a carbon atom from an alkylene group attached to a chelating atom. In one embodiment, the point of attachment for a chelating agent provided herein is on a ring carbon atom from a ring containing a chelating atom (e.g., a ring carbon atom of a pyridine ring). In one embodiment, a chelating moiety provided herein is resulted from removal of an —OH group from an acid group or a derivative thereof. For example, in one embodiment, the point of attachment is on the carbon atom of a carboxylic acid (—COOH) after removal of an —OH group; in another embodiment, the point of attachment is on the phosphorus atom of a phosphonic acid (—PO₃H₂) after removal of an —OH group. Non-limiting point of attachments are also illustrated in the chelating agent structures in Table 1.

As used herein, and unless otherwise specified, the term “fluorescent dye” refers to a compound that emits visible or infrared light after excitation by electromagnetic radiation of a shorter and suitable wavelength. It is understood by the skilled person that each fluorescent dye has a predetermined excitation wavelength.

As used herein, and unless otherwise specified, the term “contrast agent” refers to a compound which increases the contrast of structures or fluids in medical imaging. The enhancement is achieved by absorbing electromagnetic radiation or altering electromagnetic fields.

As used herein, and unless otherwise specified, the term “paramagnetic” refers to paramagnetism induced by unpaired electrons in a medium. A paramagnetic substance induces a magnetic field if an external magnetic field is applied. Unlike diamagnetism the direction of the induced field is the same as the external field and unlike ferromagnetism the field is not maintained in absence of an external field.

As used herein, and unless otherwise specified, the term “nanoparticle” as used herein refers to particles, such as particles of spheric shape, with diameters of sizes between 1 and 100 nanometers. Depending on the composition, nanoparticles can possess magnetical, optical or physico-chemical qualities that can be assessed. Additionally surface modification is achievable for many types of nanoparticles.

As used herein, and unless otherwise specified, a “pharmaceutically acceptable salt” includes both acid and base addition salts. Suitable pharmaceutically acceptable salts of the compound provided herein include acid addition salts which may, for example, be formed by mixing a solution of choline or derivative thereof with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compound provided herein carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counter anions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphor sulfonate, camsylate, carbonate, chloride, citrate, clavulanate, cyclopentane propionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds provided herein contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes provided herein.

In addition to salt forms, also provided herein are compounds that are in a prodrug form. Prodrugs of a compound readily undergoes chemical changes under physiological conditions to provide the compound. A prodrug is an active or inactive compound that is modified chemically through in vivo physiological action, such as hydrolysis, metabolism and the like, into a compound provided herein following administration of the prodrug to a patient. Additionally, prodrugs can be converted to the compounds provided herein by chemical or biochemical methods in an ex vivo environment. For example, prodrugs can be slowly converted to the compounds provided herein when placed in a transdermal patch reservoir with a suitable enzyme. The suitability and techniques involved in making and using prodrugs are known by those skilled in the art. For a general discussion of prodrugs involving esters see Svensson and Tunek Drug Metabolism Reviews 16.5 (1988) and Bundgaard Design of Prodrugs, Elsevier (1985). Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxyl groups have been masked as esters and ethers. EP 0 039 051 (Sloan and Little, Apr. 11, 1981) discloses Mannich-base hydroxamic acid prodrugs, their preparation and use.

As used herein, and unless otherwise specified, the term “isomer” refers to different compounds that have the same molecular formula. “Stereoisomers” are isomers that differ only in the way the atoms are arranged in space. “Atropisomers” are stereoisomers from hindered rotation about single bonds. “Enantiomers” are a pair of stereoisomers that are non-superimposable mirror images of each other. A mixture of a pair of enantiomers in any proportion can be known as a “racemic” mixture. “Diastereoisomers” are stereoisomers that have at least two asymmetric atoms, but which are not mirror-images of each other. The absolute stereochemistry can be specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. However, the sign of optical rotation, (+) and (−), is not related to the absolute configuration of the molecule, R and S. Certain compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques.

As used herein, and unless otherwise specified, the term “enantiomeric purity” or “enantiomer purity” refers to a qualitative or quantitative measure of a purified enantiomer. The enantiomeric purity of compounds described herein may be described in terms of enantiomeric excess (ee), which indicates the degree to which a sample contains one enantiomer in greater amounts than the other. A racemic mixture has an ee of 0%, while a single completely pure enantiomer has an ee of 100%. Examples of the enantiomeric purity include an ee of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about or at least about 99%. Similarly, “diastereomeric purity” may be described in terms of diasteriomeric excess (de), which indicates the degree to which a sample contains one diastereoisomers in greater amounts than the other(s).

As used herein, and unless otherwise specified, the term “substantially purified enantiomer” refers to a compound wherein one enantiomer has been enriched over the other. In one embodiment, the other enantiomer represents less than about 20%, less than about 10%, less than about 5%, or less than about 2% of the enantiomer.

“Stereoisomers” can also include E and Z isomers, or a mixture thereof, and cis and trans isomers or a mixture thereof. In certain embodiments, a compound described herein is isolated as either the E or Z isomer. In other embodiments, a compound described herein is a mixture of the E and Z isomers.

As used herein, and unless otherwise specified, the term “tautomer” refers to one of two or more structural isomers which exist in equilibrium and which are readily converted from one isomeric form to another. If tautomers possibly exist (such as in solution), the chemical equilibrium of tautomers can be reached. For example, proton tautomer (also called prototropic tautomer) includes interconversion through proton migration, such as keto-enol isomerization, imine-enamine isomerization. Valence tautomer includes some recombination of bonding electrons for mutual transformation. A specific example of keto-enol tautomerization is the tautomerism between two tautomers of pentane-2,4-dione and 4-hydroxypent-3-en-2-one. Another example is 2-pyridone and 2-hydroxypyridine tautomerization.

Compounds provided herein can be synthesized according to one or more of the methods/examples provided herein. It should be noted that the general procedures may be shown as it relates to preparation of compounds having unspecified stereochemistry. However, such procedures are generally applicable to those compounds of a specific stereochemistry, e.g., where the stereochemistry about a group is (S) or (R). In addition, the compounds having one stereochemistry (e.g., (R)) can often be utilized to produce those having opposite stereochemistry (i.e., (S)) using well-known methods, for example, by inversion.

Certain compounds provided herein possess asymmetric carbon atoms (optical centers) or double bonds; the racemates, diastereomers, geometric isomers and individual isomers are intended to be encompassed within the scope of this application.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are intended to be encompassed within the scope of this application.

As used herein, and unless otherwise specified, the term “pharmaceutical composition” refers to a substance and/or a combination of substances being used for the identification, prevention or treatment of a tissue status or disease. The pharmaceutical composition is formulated to be suitable for administration to a patient in order to prevent and/or treat disease. Further a pharmaceutical composition refers to the combination of an active agent with a carrier, inert or active, making the composition suitable for therapeutic use. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems provided herein. Semisolid compositions provided herein comprise emulsion, suspension, creams, lotions, gels, globules, buccal tablets and suppositories.

As used herein, and unless otherwise specified, the term “pharmaceutically acceptable” means approved by a regulatory agency of the Federal or a state government or listed in the U.S. Pharmacopeia or other generally recognized pharmacopeia for use in animals, and more particularly in humans.

As used herein, and unless otherwise specified, the term “carrier” or “excipient”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

As used herein, and unless otherwise specified, the term “cytotoxic effect” refers to the depletion, elimination and/or the killing of a target cell(s). As used herein, and unless otherwise specified, the term “cytotoxic agent” refers to an agent that has a cytotoxic and/or cytostatic effect on a cell. The term is intended to include chemotherapeutic agents, and toxins such as enzymatically active toxins of bacterial, fungal, plant, or animal origin, and fragments thereof. As used herein, and unless otherwise specified, the term “cytostatic effect” refers to the inhibition of cell proliferation. As used herein, and unless otherwise specified, the term “cytostatic agent” refers to an agent that has a cytostatic effect on a cell, thereby inhibiting the growth and/or expansion of a specific subset of cells.

As used herein, and unless otherwise specified, the term “cytokine” refers to small proteins (˜5-20 kDa) that are involved in autocrine signaling, paracrine signaling and endocrine signaling as immunomodulating agents. Cytokines include chemokines, interferons, interleukins, lymphokines, and tumor necrosis factors but generally not hormones or growth factors.

As used herein, and unless otherwise specified, the term “immunomodulatory molecule” refers to substance that stimulates or suppresses the immune system and may help the body fight cancer, infection, or other diseases. Specific immunomodulating molecules can be monoclonal antibodies, cytokines, and vaccines, which affect specific parts of the immune system.

As used herein, and unless otherwise specified, the term “amphiphilic substance” refers to compounds with both hydrophilic and lipophilic properties. Common amphiphilic substances are phospholipids, cholesterol, glycolipids, fatty acids, bile acids, saponins, pediocins, local anesthetics, Ab proteins and antimicrobial peptides.

As used herein, and unless otherwise specified, the term “protein” and “polypeptide” are used interchangeably herein and refer to any peptide-bond-linked chain of amino acids, regardless of length or post-translational modification. In one embodiment, the amino acid is any of the amino acids provided herein. Proteins provided herein (including protein derivatives, protein variants, protein fragments, protein segments, protein epitopes and protein domains) can be further modified by chemical modification. This means such a chemically modified polypeptide comprises other chemical groups than the 20 naturally occurring amino acids. Examples of such other chemical groups include without limitation glycosylated amino acids and phosphorylated amino acids. Chemical modifications of a polypeptide may provide advantageous properties as compared to the parent polypeptide, e.g., one or more of enhanced stability, increased biological half-life, or increased water solubility.

As used herein, and unless otherwise specified, the terms “nucleic acid” and “polynucleotide” are used interchangeably herein and refer to polymeric or oligomeric macromolecules, or large biological molecules, essential for all known forms of life. Nucleic acids, which include DNA (deoxyribonucleic acid) and RNA (ribonucleic acid), are made from monomers known as nucleotides. Most naturally occurring DNA molecules consist of two complementary biopolymer strands coiled around each other to form a double helix. The DNA strand is also known as polynucleotides consisting of nucleotides. Each nucleotide is composed of a nitrogen-containing nucleobase as well as a monosaccharide sugar called deoxyribose or ribose and a phosphate group. Naturally occurring nucleobases comprise guanine (G), adenine (A), thymine (T), uracil (U) or cytosine (C). The nucleotides are joined to one another in a chain by covalent bonds between the sugar of one nucleotide and the phosphate of the next, resulting in an alternating sugar-phosphate backbone. If the sugar is desoxyribose, the polymer is DNA. If the sugar is ribose, the polymer is RNA. Typically, a polynucleotide is formed through phosphodiester bonds between the individual nucleotide monomers. As used herein, and unless otherwise specified, the term “nucleic acid” includes but is not limited to ribonucleic acid (RNA), deoxyribonucleic acid (DNA), and mixtures thereof such as RNA-DNA hybrids (within one strand), as well as cDNA, genomic DNA, recombinant DNA, cRNA and mRNA. A nucleic acid may consist of an entire gene, or a portion thereof, the nucleic acid may also be a miRNA, siRNA, piRNA or shRNA. miRNAs are short ribonucleic acid (RNA) molecules, which are on average 22 nucleotides long but may be longer and which are found in all eukaryotic cells, i.e., in plants, animals, and some viruses, which functions in transcriptional and post-transcriptional regulation of gene expression. miRNAs are post-transcriptional regulators that bind to complementary sequences on target messenger RNA transcripts (mRNAs), usually resulting in translational repression and gene silencing. Small interfering RNAs (siRNAs), sometimes known as short interfering RNA or silencing RNA, are short ribonucleic acid (RNA molecules), between 20-25 nucleotides in length. They are involved in the RNA interference (RNAi) pathway, where they interfere with the expression of specific genes. A short hairpin RNA (shRNA) or small hairpin RNA (shRNA) is an artificial RNA molecule with a tight hairpin turn that can be used to silence target gene expression via RNA interference (RNAi). Expression of shRNA in cells is typically accomplished by delivery of plasmids or through viral or bacterial vectors. piRNAs are also short RNAs which usually comprise 26-31 nucleotides and derive their name from so-called piwi proteins they are binding to. The nucleic acid can also be an artificial nucleic acid. Artificial nucleic acids include polyamide or peptide nucleic acid (PNA), morpholino and locked nucleic acid (LNA), as well as glycol nucleic acid (GNA) and threose nucleic acid (TNA). Each of these is distinguished from naturally-occurring DNA or RNA by changes to the backbone of the molecule. The nucleic acids, can, e.g., be synthesized chemically, e.g., in accordance with the phosphotriester method (see, for example, Uhlmann, E. & Peyman, A. (1990) Chemical Reviews, 90, 543-584).

As used herein, and unless otherwise specified, the term “viral structural protein” (VSP) refers to viral coat proteins (VCP) or viral envelope glycoproteins (VEG). As used herein, and unless otherwise specified, the term “viral coat protein” (VCP) refers to a structural virus capsid protein of a virus. In one embodiment, the virus is a double-stranded DNA virus, single-stranded DNA virus, double-stranded RNA virus, single-stranded RNA virus, negative-sense single-stranded RNA virus, single-stranded RNA reverse transcribing virus, double-stranded RNA reverse transcribing virus. The VCP can comprise major capsid proteins of adeno-associated virus (AAV).

As used herein, and unless otherwise specified, the term “viral envelope glycoproteins” (VEG) refers to viral proteins that are part of the viral envelope. The viral envelope is typically derived from portions of the host cell membrane, e.g., comprises phospholipids, and additionally comprise viral glycoproteins that, e.g., help the virus to avoid the immune system. Enveloped viruses comprise DNA viruses, such as Herpesviruses, Poxviruses, and Hepadnaviruses; RNA viruses, such as Flavivirus, Togavirus, Coronavirus, Hepatitis D, Orthomyxovirus, Paramyxovirus, Rhabdovirus, Bunyavirus, Filovirus and Retroviruses. In one embodiment, the viral envelop glycoprotein is derived from any of these viruses.

As used herein, and unless otherwise specified, the term “liposome” refers to uni- or multilamellar (e.g., 2, 3, 4, 5, 6, 7, 8, 9, and 10 lamellar) lipid structures enclosing an aqueous interior, depending on the number of lipid membranes formed. Lipids, which are capable of forming a liposomes include all substances having fatty or fat-like properties. Such lipids comprise an extended apolar residue (X) and usually a water soluble, polar, hydrophilic residue (Y), which can be characterized by the basic formula

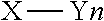

wherein n equals or is greater than zero. Lipids with n=0 are termed “apolar lipids”, while lipids with n>1 are referred to as “polar lipids”. In one embodiment, lipids, which can make up the lipids in the liposomes provided herein are selected from the group consisting of glycerides, glycerophospholipids, sulfolipids, sphingolipids, phospholipids, isoprenolides, steroids, stearines, steroles and carbohydrate containing lipids.

A virus like particle (VLP) is a multimer of VSP, such as VCPs and/or VEPs that does not comprise polynucleotides but which otherwise has properties of a virus, e.g., binds to cell surface receptors, is internalized with the receptor, is stable in blood, and/or comprises glycoproteins etc. VLPs are typically assembled of multimers of VCPs and/or VEPs, in particular of VCPs. VLPs are known in the art and have been produced from a number of viruses including Parvoviridae (e.g., adeno-associated virus), Retroviridae (e.g., HIV), Flaviviridae (e.g., Hepatitis C virus) and bacteriophages (e.g., QP, AP205).

It should be noted that if there is a discrepancy between a depicted structure and a name for that structure, the depicted structure is to be accorded more weight.

### Compounds

In one embodiment, provided herein is a compound of Formula (I):

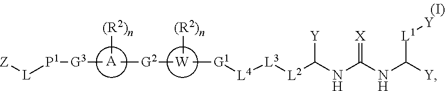

or a stereoisomer, a mixture of stereoisomers, a tautomer, or a pharmaceutically acceptable salt thereof, wherein:
X is O, S, or NH;
each Y is independently —CO₂H, —SO₂H, —SO₃H, —OSO₃H, —PO₂H, —PO₃H₂, —OPO₃H₂, or

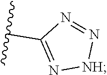 [SMILES: *c1nn[nH]n1]

L¹ is optionally substituted C₁-C₆ alkylene, wherein one or two —CH₂— in the alkylene is independently optionally replaced by a —NHC(═O)—*, —C(═O)NH—*, —O—, —S—, —S(═O)₂—, —S(═O)—, —C(═O)—, —C(═O)O—*, —OC(═O)—*, or —NH—, wherein * refers to the direction toward the Y adjacent to L¹;
L² is optionally substituted C₁-C₆ alkylene, wherein one or two —CH₂— in the alkylene is independently optionally replaced by —O—, —S—, C₃-C₆ cycloalkylene, C₃-C₆ cycloalkenylene, or 3 to 6-membered heterocyclylene;
L³ is —(C═O)—NR′—*, —NR′—(C═O)—*, —C(═O)—, —O—, —S—, —S—S—, —S—CH₂—S—, —S(═O)—, —S(O)₂—, NR′, —NR′—(C═O)—NR′—, —C(═O)—NR′—C(═O)—, —OC(═O)—NR′—*, —NR′C(═O)O—*, —OC(═S)—NR′—*, —NR′C(═S)O—*,

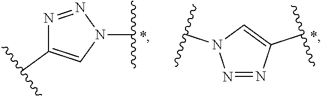 [SMILES: *c1cn(*)nn1 ; *c1cn(*)nn1]

—(C═O)-(3- to 10-membered optionally substituted N-containing ring)-*, or -(3- to 10-membered optionally substituted N-containing ring)-(C═O)—*, wherein * refers to the direction toward L²;
L⁴ is optionally substituted C₁-C₆ alkylene;
G¹ is absent, —O—, —S—, —NR⁴—, —NR⁴—(C═O)—*, —(C═O)—NR⁴—*, or optionally substituted C₂-C₆ alkylene, wherein one or more —CH₂— in the alkylene is independently replaced by a —O—, —S—, —S(═O)₂—, —S(═O)—, —C(═O)—, —C(═O)O—*, —OC(═O)—*, NR⁴, —(C═O)—NR⁴—*, or —NR⁴—(C═O)—*, wherein * refers to the direction toward L⁴;
G² and G³ are each independently absent, —O—, —S—, —NR′—, —NR′—(C═O)—*, —(C═O)—NR′—*, —NR′—(C═S)—*, or optionally substituted C₁-C₆ alkylene, wherein one or more —CH₂-in the alkylene is independently optionally replaced by a —O—, —S—, —S(═O)₂—, —S(═O)—, —C(═O)—, —C(═O)O—*, —OC(═O)—*, NR′, —(C═O)—NR′—*, —NR′—(C═O)—*, or —NR′—(C═S)—*, wherein * refers to the direction toward G¹;
R⁴ is H or optionally substituted C₁-C₆ alkyl; or R⁴ and NR¹ of G² together with the intervening atoms form a 5 to 12-membered heterocyclyl ring;
P¹ is absent, NR⁸, C₆-C₁₀ aryl, 5 to 10-membered heteroaryl, C₃-C₁₄ cycloalkyl, or 5 to 14-membered heterocyclyl; wherein the aryl, heteroaryl, cycloalkyl, and heterocyclyl are independently optionally substituted;
R⁸ is independently H or optionally substituted C₁-C₆ alkyl;
Ring W is a 5 to 10-membered heteroaryl, C₃-C₈ cycloalkyl, C₃-C₈ cycloalkenyl, or 5 to 14-membered heterocyclyl;
Ring A is C₆-C₁₀ aryl, 5 to 10-membered heteroaryl, C₃-C₁₄ cycloalkyl, or 5 to 14-membered heterocyclyl;
each R¹ is independently H or optionally substituted C₁-C₆ alkyl;
each R² is independently OH, halogen, oxo, C₁-C₆ alkyl, —O—(C₁-C₆ alkyl), —S—(C₁-C₆ alkyl), —NH₂, —NH—(C₁-C₆ alkyl), or —N(C₁-C₆ alkyl)₂, wherein each alkyl is independently optionally substituted with one or more OH, oxo, or halogen;
n is an integer from 0 to 6 as valency permits;
L is absent or a linker; and
Z is a radioactive moiety, a chelating agent, a fluorescent dye, a contrast agent, a cytostatic or cytotoxic agent, a cytokine, an immunomodulatory molecule, an amphiphilic substance, a nucleic acid, a viral structural protein, a protein, or biotin.

As described herein, one or more —CH₂— in an alkylene group (e.g., L¹, L², G¹, G², and G³) can be optionally replaced by a ring moiety provided herein. When such group is a C₁ alkylene and the —CH₂— (i.e., C₁ alkylene) is replaced by a ring moiety, such group becomes the ring moiety itself.

In one embodiment, P¹ is absent. In one embodiment, P¹ is NR⁸. In one embodiment, P¹ is NH. In one embodiment, P¹ is optionally substituted C₆-C₁₀ aryl. In one embodiment, P¹ is optionally substituted 5 to 10-membered heteroaryl. In one embodiment, P¹ is optionally substituted C₃-C₁₄ cycloalkyl. In one embodiment, P¹ is optionally substituted 5 to 14-membered heterocyclyl. In one embodiment, the aryl, heteroaryl, cycloalkyl, and heterocyclyl, and their optional substituents are as described in Ring B and R², i.e., P¹ corresponds to Ring W optionally substituted with n instances of R² as described herein.

In one embodiment, G¹ is absent. In one embodiment, G¹ is —O—. In one embodiment, G¹ is —S—. In one embodiment, G¹ is —NR⁴—. In one embodiment, G¹ is —NH—. In one embodiment, G¹ is optionally substituted C₂-C₆ alkylene, wherein one or more —CH₂— in the alkylene is independently replaced by a —O—, —S—, —S(═O)₂—, —S(═O)—, —C(═O)—, —C(═O)O—*, —OC(═O)—*, NR⁴, —(C═O)—NR⁴—*, or —NR⁴—(C═O)—*, wherein * refers to the direction toward L⁴. In one embodiment, the C₂-C₆ alkylene of G¹ is unsubstituted.

In one embodiment, the compound is a compound of Formula (II-A), (II-B), (II-C), or (II-D):

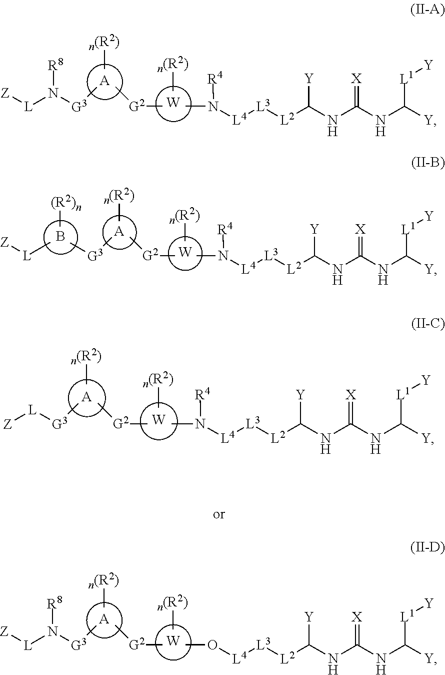

or a stereoisomer, a mixture of stereoisomers, a tautomer, or a pharmaceutically acceptable salt thereof, wherein:
Ring B is C₆-C₁₀ aryl, 5 to 10-membered heteroaryl, C₃-C₁₄ cycloalkyl, or 5 to 14-membered heterocyclyl;
each instance of R² is independently OH, halogen, oxo, C₁-C₆ alkyl, —O—(C₁-C₆ alkyl), —S—(C₁-C₆ alkyl), —NH₂, —NH—(C₁-C₆ alkyl), or —N(C₁-C₆ alkyl)₂, wherein each alkyl is independently optionally substituted with one or more OH, oxo, or halogen; and
each instance of n is an integer from 0 to 6 as valency permits.

In one embodiment, R⁸ is H. In one embodiment, R⁸ is C₁-C₆ alkyl. In one embodiment, R⁸ is C₁-C₃ alkyl. In one embodiment, R⁸ is methyl. In one embodiment, R⁸ is ethyl. In one embodiment, the alkyl in R⁸ is unsubstituted. In one embodiment, the alkyl in R⁸ is substituted. In one embodiment, the alkyl in R⁸ is substituted with one or more halogen, oxo, hydroxyl, or C₁-C₆ alkoxy.

In one embodiment, L⁴ is —(CH₂)₀₋₃CH(R³)(CH₂)₀₋₃—, or R⁴ is —(CH₂)₁₋₃—R^{3a}, wherein R³ is C₁-C₂₀ alkyl, C₂-C₂₀ alkenyl, C₂-C₂₀ alkynyl, C₃-C₈ cycloalkyl, or —(CH₂)₀₋₃—R^{3a}, wherein each R^{3a} independently is C₆-C₂₀ aryl, C₃-C₁₄ cycloalkyl, 3 to 14-membered heterocyclyl, or 5 to 20-membered heteroaryl, and wherein the alkyl, alkenyl, alkynyl, aryl, cycloalkyl, heterocyclyl, or heteroaryl in R³ or R^{3a} is optionally substituted with one or more halogen, OH, C₁-C₆ alkyl, C₆-C₁₀ aryl, C₆-C₁₀ cycloalkyl, 5- to 10-membered heteroaryl, 3 to 8-membered heterocyclyl, C₆-C₁₀ aryloxy, C₆-C₁₀ cycloalkyloxy, or 5- to 10-membered heteroaryloxy.

In one embodiment, Ring A is C₆-C₁₀ aryl. In one embodiment, Ring A is C₆-C₈ aryl. In one embodiment, Ring A is phenyl. In one embodiment, Ring A is naphthyl.

In one embodiment, Ring A is 5 to 10-membered heteroaryl. In one embodiment, Ring A is 5 to 8-membered heteroaryl. In one embodiment, Ring A is 5-membered heteroaryl. In one embodiment, Ring A is 6-membered heteroaryl. In one embodiment, Ring A is a 5 or 6-membered heteroaryl containing one or more nitrogen, oxygen, or sulfur ring atoms.

In one embodiment, Ring A is C₃-C₁₄ cycloalkyl. In one embodiment, Ring A is C₃-C₁₂ cycloalkyl. In one embodiment, Ring A is C₅-C₁₂ cycloalkyl. In one embodiment, Ring A is C₃-C₈ cycloalkyl. In one embodiment, Ring A is C₃ cycloalkyl. In one embodiment, Ring A is C₄ cycloalkyl. In one embodiment, Ring A is C₅ cycloalkyl. In one embodiment, Ring A is C₆ cycloalkyl. In one embodiment, Ring A is C₇ cycloalkyl. In one embodiment, Ring A is C₈ cycloalkyl. In one embodiment, Ring A is C₉ cycloalkyl. In one embodiment, Ring A is C₁₀ cycloalkyl. In one embodiment, Ring A is C₁₁ cycloalkyl. In one embodiment, Ring A is C₁₂ cycloalkyl. In one embodiment, Ring A is C₁₃ cycloalkyl. In one embodiment, Ring A is C₁₄ cycloalkyl. In one embodiment, the cycloalkyl is a fused, bridged, or spiro cycloalkyl. In one embodiment, the cycloalkyl is a monocyclic cycloalkyl.

In one embodiment, Ring A is a fused C₆-C₁₄ cycloalkyl. In one embodiment, Ring A is a fused C₆-C₁₂ cycloalkyl. In one embodiment, Ring A is a fused C₆-C₁₀ cycloalkyl. In one embodiment, Ring A is a bridged C₈ cycloalkyl. In one embodiment, Ring A is a bridged C₅-C₁₄ cycloalkyl. In one embodiment, Ring A is a bridged C₅-C₁₂ cycloalkyl. In one embodiment, Ring A is a bridged C₅-C₁₀ cycloalkyl. In one embodiment, Ring A is a spiro C₆-C₁₄ cycloalkyl. In one embodiment, Ring A is a spiro C₆-C₁₂ cycloalkyl. In one embodiment, Ring A is a spiro C₆-C₁₀ cycloalkyl.

In one embodiment, Ring A is a monocyclic C₃-C₈ cycloalkyl. In one embodiment, Ring A is cyclopropyl. In one embodiment, Ring A is cyclobutyl. In one embodiment, Ring A is cyclopentyl. In one embodiment, Ring A is cyclohexyl.

In one embodiment, Ring A is 5 to 14-membered heterocyclyl. In one embodiment, Ring A is 5 to 12-membered heterocyclyl. In one embodiment, Ring A is 5 to 10-membered heterocyclyl. In one embodiment, Ring A is 5-membered heterocyclyl. In one embodiment, Ring A is 6-membered heterocyclyl. In one embodiment, Ring A is 7-membered heterocyclyl. In one embodiment, Ring A is 8-membered heterocyclyl. In one embodiment, Ring A is 9-membered heterocyclyl. In one embodiment, Ring A is 10-membered heterocyclyl. In one embodiment, Ring A is 11-membered heterocyclyl. In one embodiment, Ring A is 12-membered heterocyclyl. In one embodiment, Ring A is 13-membered heterocyclyl. In one embodiment, Ring A is 14-membered heterocyclyl. In one embodiment, Ring A is 5 to 14-membered N-containing heterocyclyl. In one embodiment, Ring A is 5 to 10-membered N-containing heterocyclyl. In one embodiment, the heterocyclyl is a fused, bridged, or spiro heterocyclyl. In one embodiment, the heterocyclyl is monocyclic heterocyclyl.

In one embodiment, Ring A is a fused, bridged or spiro C₅-C₁₂ cycloalkyl. In one embodiment, Ring A is a fused, bridged or spiro 5 to 12-membered heterocyclyl. In one embodiment, Ring A is a fused Cia aryl. In one embodiment, Ring A is a fused 9 or 10-membered heteroaryl.

In one embodiment, Ring A is a fused 6 to 14-membered heterocyclyl. In one embodiment, Ring A is a fused 6 to 12-membered heterocyclyl. In one embodiment, Ring A is a fused 6 to 10-membered heterocyclyl. In one embodiment, Ring A is a bridged 5 to 14-membered heterocyclyl. In one embodiment, Ring A is a bridged 5 to 12-membered heterocyclyl. In one embodiment, Ring A is a bridged 5 to 10-membered heterocyclyl. In one embodiment, Ring A is a spiro 6 to 14-membered heterocyclyl. In one embodiment, Ring A is a spiro 6 to 12-membered heterocyclyl. In one embodiment, Ring A is a spiro 6 to 10-membered heterocyclyl.

In one embodiment, Ring A is a monocyclic 3 to 8-membered heterocyclyl. In one embodiment, Ring A is a monocyclic 3 to 8-membered nitrogen-containing heterocyclyl. In one embodiment, Ring A is a monocyclic 3 to 6-membered heterocyclyl. In one embodiment, Ring A is a monocyclic 5 or 6-membered nitrogen-containing heterocyclyl.

In one embodiment, Ring A is

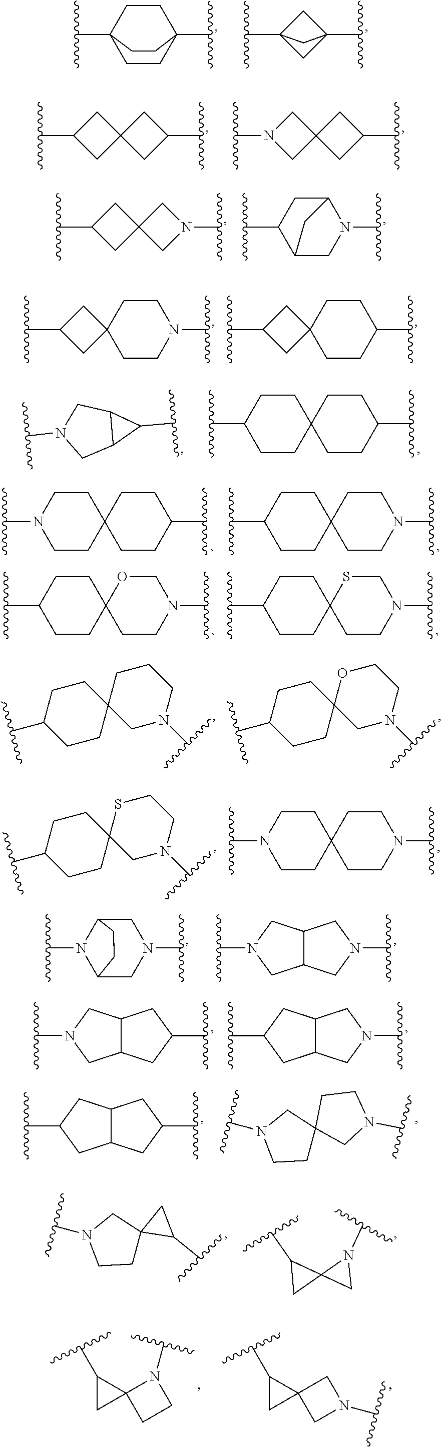

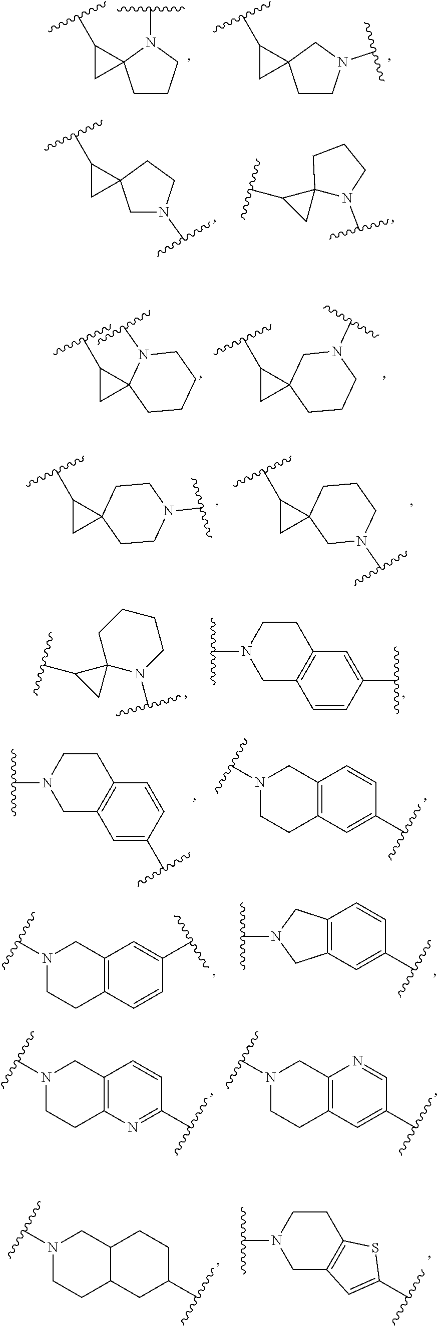

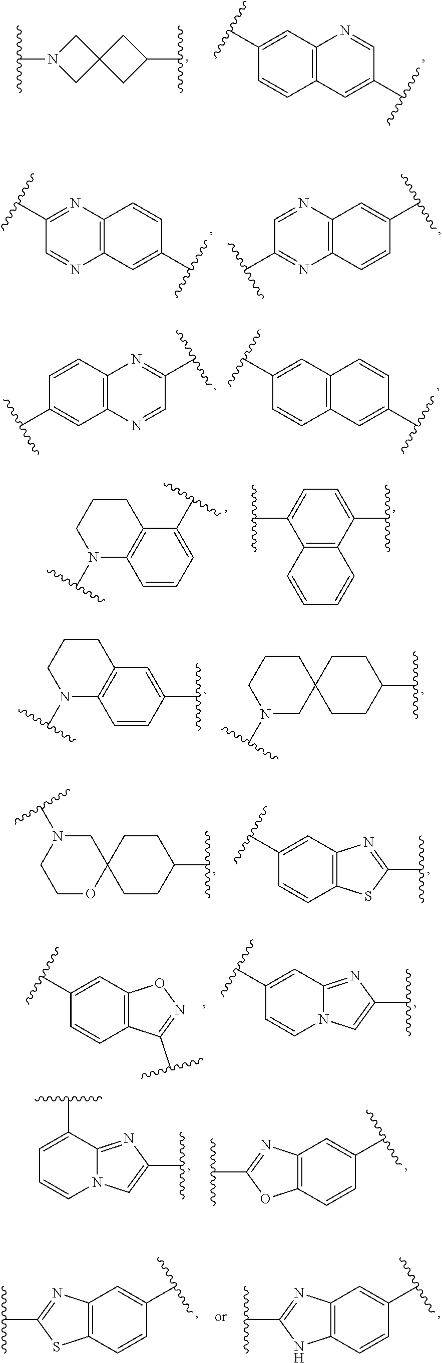

wherein the attachment to the left is to the direction of Z.

In one embodiment, the Ring A is cyclohexyl. In one embodiment, the Ring A is azetidinyl. In one embodiment, Ring A is pyrrolidinyl. In one embodiment, the Ring A is piperidinyl. In one embodiment, Ring A is azepanyl. In one embodiment, Ring A is azocanyl. In one embodiment, Ring A is piperazinyl. In one embodiment, Ring A is

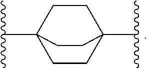 [SMILES: *C12CCC(*)(CC1)CC2]

In one embodiment, Ring A is

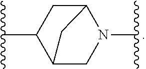 [SMILES: *C1CC2CC1CN2*]

In one embodiment, Ring A is

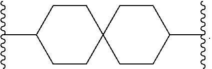 [SMILES: *C1CCC2(CC1)CCC(*)CC2]

In one embodiment, Ring A is

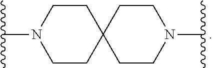 [SMILES: *N1CCC2(CC1)CCN(*)CC2]

In one embodiment, Ring A is

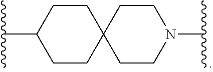 [SMILES: *C1CCC2(CC1)CCN(*)CC2]

In one embodiment, Ring A is

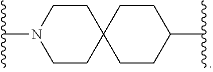 [SMILES: *C1CCC2(CC1)CCN(*)CC2]

In one embodiment, Ring A is

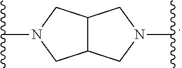 [SMILES: *N1CC2CN(*)CC2C1]

In one embodiment, Ring A is

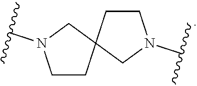 [SMILES: *N1CCC2(CCN(*)C2)C1]

In these embodiments, the attachment to the left is to the direction of Z.

In one embodiment, L¹ is C₁-C₆ alkylene. In one embodiment, L¹ is methylene. In one embodiment, L¹ is ethylene. In one embodiment, L¹ is C₃ alkylene. In one embodiment, L¹ is C₄ alkylene. In one embodiment, L¹ is C₅ alkylene. In one embodiment, L¹ is C₆ alkylene. In one embodiment, L¹ is unsubstituted C₁-C₆ alkylene. In one embodiment, L¹ is C₁-C₆ alkylene substituted with one or more halogen, oxo, hydroxyl, or C₁-C₆ alkoxy. In one embodiment, L¹ is C₁-C₆ alkylene substituted with one or more halogen (e.g., substituted with one or more F).

In one embodiment, L¹ is C₁-C₆ alkylene, wherein one or two —CH₂— in the alkylene is independently optionally replaced by a —NHC(═O)—*, —C(═O)NH—*, —O—, —S—, —S(═O)₂—, —S(═O)—, —C(═O)—, —C(═O)O—*, —OC(═O)—*, or —NH—, wherein * refers to the direction toward the Y adjacent to L. In one embodiment, L¹ is C₁-C₆ alkylene, and wherein one —CH₂— in L¹ is replaced by a —NHC(═O)—*, —C(═O)NH—*, —O—, —S—, —S(═O)₂—, or —S(═O)—, wherein * refers to the direction toward the Y adjacent to L¹. In one embodiment, L¹ is C₁-C₆ alkylene, and wherein one —CH₂— in L¹ is replaced by a —NHC(═O)—*, wherein * refers to the direction toward the Y adjacent to L.

In one embodiment, L¹ is —CH₂—, —CH₂CH₂—, —CH₂CH₂CH₂—, —CH₂—NHC(═O)—, —CH₂CH₂—NHC(═O)—, —CH₂CH₂CH₂—NHC(═O)—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH₂CH(OH)CH₂—, —CH₂CHFCH₂—, —(CH₂)₂CH(OH)—, —(CH₂)₂CHF—, —CH₂OCH₂—, —CH₂SCH₂—, —CH(OH)CH₂CH₂—, —CH(CH₃)—O—CH₂—, —C(CH₃)₂—O—CH₂—, —CH₂O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(═O)NH—CH₂—, —NHC(═O)—CH₂CH₂—, —C(═O)NH—CH(CH₃)—, —NHC(═O)—CH₂CH₂CH₂—, or —C(═O)NH—C(CH₃)₂—.

In one embodiment, L¹ is —CH₂CH₂—. In one embodiment, L¹ is —CH₂CH₂CH₂—. In one embodiment, L¹ is —CH₂CH₂—NHC(═O)—. In one embodiment, L¹ is —CH₂—NHC(═O)—.

In one embodiment, L² is C₁-C₆ alkylene. In one embodiment, L² is a straight C₁-C₆ alkylene. In one embodiment, L² is methylene. In one embodiment, L² is ethylene. In one embodiment, L² is C₃ alkylene. In one embodiment, L² is C₄ alkylene. In one embodiment, L² is —CH₂CH₂CH₂CH₂—. In one embodiment, L² is C₅ alkylene. In one embodiment, L² is C₆ alkylene. In one embodiment, L² is straight C₁-C₆ alkylene. In one embodiment, L² is unsubstituted C₁-C₆ alkylene. In one embodiment, L² is C₁-C₆ alkylene substituted with one or more halogen, oxo, hydroxyl, or C₁-C₆ alkoxy.

In one embodiment, L² is C₁-C₆ alkylene, wherein one or two —CH₂— in the alkylene is replaced by —O—. In one embodiment, one or two —CH₂— in the alkylene (in L²) is replaced by —S—. In one embodiment, one —CH₂— in the alkylene (in L²) is replaced by C₃-C₆ cycloalkylene. In one embodiment, one —CH₂— in the alkylene (in L²) is replaced by C₃-C₆ cycloalkenylene. In one embodiment, one —CH₂— in the alkylene (in L²) is replaced by 3 to 6-membered heterocyclylene.

In one embodiment, L³ is —(C═O)—NR¹—*. In one embodiment, L³ is —(C═O)—NH—*. In one embodiment, L³ is —NR¹—(C═O)—*. In one embodiment, L³ is —NH—(C═O)—*. In one embodiment, L³ is —C(═O)—. In one embodiment, L³ is —O—. In one embodiment, L³ is —S—. In one embodiment, L³ is —S—S—. In one embodiment, L³ is —S—CH₂—S—. In one embodiment, L³ is —S(═O)—. In one embodiment, L³ is —S(O)₂—. In one embodiment, L³ is NR¹. In one embodiment, L³ is NH. In one embodiment, L³ is —NR¹—(C═O)—NR¹—. In one embodiment, L³ is —C(═O)—NR¹—C(═O)—. In one embodiment, L³ is —OC(═O)—NR′—*. In one embodiment, L³ is —NR¹C(═O)O—*. In one embodiment, L³ is —OC(═S)—NR¹—*. In one embodiment, L³ is —NR¹C(═S)O—*. In one embodiment, L³ is

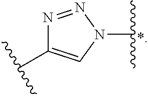 [SMILES: *c1cn(*)nn1]

In one embodiment, L³ is

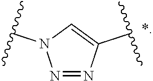 [SMILES: *c1cn(*)nn1]

In one embodiment, L³ is —(C═O)-(3- to 10-membered optionally substituted N-containing ring)-*. In one embodiment, L³ is —(C═O)-(3- to 8-membered optionally substituted N-containing ring)-*. In one embodiment, L³ is —(C═O)-(5 or 6-membered optionally substituted N-containing ring)-*. In one embodiment, L³ is -(3- to 10-membered optionally substituted N-containing ring)-(C═O)—*. In one embodiment, L³ is -(3- to 8-membered optionally substituted N-containing ring)-(C═O)—*. In one embodiment, L³ is -(5 or 6-membered optionally substituted N-containing ring)-(C═O)—*. In these embodiment, * refers to the direction toward L².

In one embodiment, L⁴ is —(CH₂)₀₋₃CH(R³)(CH₂)₀₋₃—. In one embodiment, L⁴ is —(CH₂)₀₋₃CH(R³)(CH₂)₀₋₃—, wherein R³ is C₁-C₂₀ alkyl, C₂-C₂₀ alkenyl, C₂-C₂₀ alkynyl, C₃-C₈cycloalkyl, or —(CH₂)₀₋₃—R^{3a}, wherein R^{3a} is C₆-C₂₀ aryl, C₃-C₁₄ cycloalkyl, 3 to 14-membered heterocyclyl, or 5 to 20-membered heteroaryl, and wherein the alkyl, alkenyl, alkynyl, aryl, cycloalkyl, heterocyclyl, or heteroaryl in R³ or R^{3a} is optionally substituted with one or more halogen, OH, C₁-C₆ alkyl, C₆-C₁₀ aryl, C₆-C₁₀ cycloalkyl, 5- to 10-membered heteroaryl, 3 to 8-membered heterocyclyl, C₆-C₁₀ aryloxy, C₆-C₁₀ cycloalkyloxy, or 5- to 10-membered heteroaryloxy. In one embodiment, L⁴ is —(CH₂)₀₋₃CH(CH₂R^{3a})(CH₂)₀₋₃—. In one embodiment, L⁴ is —CH(R³)—. In one embodiment, L⁴ is —CH(R³)—, and R³ is —(CH₂)₀₋₃—R^{3a}. In one embodiment, L⁴ is —CH(R³)—, and R³ is —(CH₂)₁₋₃—R^{3a}. In one embodiment, L⁴ is —CH(R³)—, and R³ is —CH₂—R^{3a}.

In one embodiment, L⁴ is C₁-C₆ alkylene. In one embodiment, L⁴ is methylene. In one embodiment, L⁴ is ethylene. In one embodiment, L⁴ is C₃ alkylene. In one embodiment, L⁴ is C₄ alkylene. In one embodiment, L⁴ is C₅ alkylene. In one embodiment, L⁴ is C₆ alkylene. In one embodiment, L⁴ is unsubstituted C₁-C₆ alkylene.

In one embodiment, R¹ is H. In one embodiment, R¹ is C₁-C₆ alkyl. In one embodiment, R¹ is C₁-C₃ alkyl. In one embodiment, R¹ is methyl. In one embodiment, R¹ is ethyl. In one embodiment, the alkyl in R¹ is unsubstituted. In one embodiment, the alkyl in R¹ is substituted. In one embodiment, R¹ is C₁-C₆ alkyl optionally substituted with one or more OH, halogen, oxo,

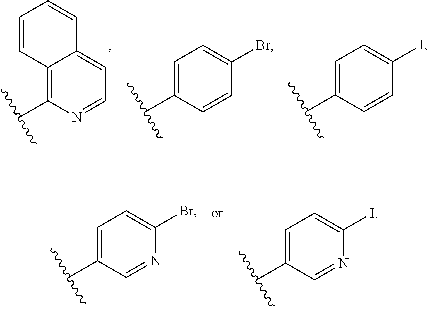

In one embodiment, R¹ is

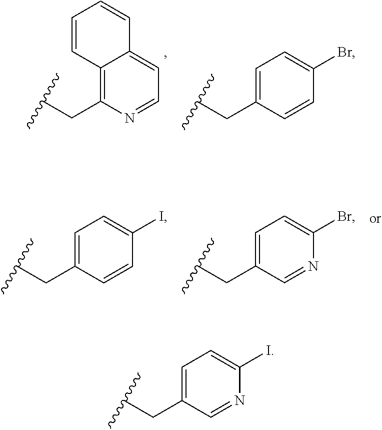

In one embodiment, each instance of R² is independently OH, halogen, oxo, C₁-C₆ alkyl, —O—(C₁-C₆ alkyl), —S—(C₁-C₆ alkyl), —NH₂, —NH—(C₁-C₆ alkyl), or —N(C₁-C₆ alkyl)₂, each of said C₁-C₆ alkyl being independently and optionally substituted with one or more OH, oxo, or halo. In one embodiment, each instance of R² is independently OH, oxo, halogen, —NH₂, or C₁-C₆ alkyl.

In one embodiment, n is 0 (i.e., R² is absent). In one embodiment, n is 1. In one embodiment, n is 2. In one embodiment, n is 3. In one embodiment, n is 4. In one embodiment, n is 5. In one embodiment, n is 6. In one embodiment, when there are more than one n, each n is independent, i.e., each of them can be the same or different. In one embodiment, each n is independently 0, 1, or 2.

In one embodiment, R³ is C₁-C₂₀ alkyl. In one embodiment, R³ is C₁-C₁₆ alkyl. In one embodiment, R³ is C₁-C₁₂ alkyl. In one embodiment, R³ is C₁-C₆ alkyl.

In one embodiment, R³ is C₂-C₂₀ alkenyl. In one embodiment, R³ is C₂-C₁₆ alkenyl. In one embodiment, R³ is C₂-C₁₂ alkenyl. In one embodiment, R³ is C₂-C₆ alkenyl.

In one embodiment, R³ is C₂-C₂₀ alkynyl. In one embodiment, R³ is C₂-C₁₆ alkynyl. In one embodiment, R³ is C₂-C₁₂ alkynyl. In one embodiment, R³ is C₂-C₆alkynyl.

In one embodiment, R³ is C₃-C₈ cycloalkyl. In one embodiment, R³ is C₃-C₆cycloalkyl. In one embodiment, R³ is cyclopropyl. In one embodiment, R³ is cyclobutyl. In one embodiment, R³ is cyclopentyl. In one embodiment, R³ is cyclohexyl.

In one embodiment, R³ is —(CH₂)₀₋₃—R^{3a}. In one embodiment, R³ is —(CH₂)₁₋₃—R^{3a}. In one embodiment, R³ is —CH₂R^{3a}. In one embodiment, R³ is —CH₂CH₂R^{3a}. In one embodiment, R³ is —CH₂CH₂CH₂R^{3a}. In one embodiment, R³ is —(C₂-C₆ alkenyl)-R^{3a}. In one embodiment, R³ is (Z)—CH₂—CH═CH—R^{3a}. In one embodiment, R³ is (E)-CH₂—CH═CH—R^{3a}.

In one embodiment, R^{3a} is C₆-C₂₀ aryl. In one embodiment, R^{3a} is C₆-C₁₈ aryl. In one embodiment, R^{3a} is C₆ aryl. In one embodiment, R^{3a} is C₁₀ aryl. In one embodiment, R^{3a} is C₄ aryl. In one embodiment, R^{3a} is C₁₈ aryl. In one embodiment, R^{3a} comprises one or more phenyl. In one embodiment, R^{3a} is phenyl. In one embodiment, R^{3a} is pyridyl. In one embodiment, R^{3a} is biphenyl. In one embodiment, R^{3a} is bipyridyl. In one embodiment, R^{3a} is anthracenyl. In one embodiment, R^{3a} is acridinyl. In one embodiment, R^{3a} is acenaphthylenyl. In one embodiment, R^{3a} is indenyl. In one embodiment, R^{3a} is phenanthrenyl. In one embodiment, R^{3a} is phenalenyl. In one embodiment, R^{3a} is triphenylenyl. In one embodiment, R^{3a} is naphthalenyl. In one embodiment, R^{3a} is tetracenyl. In one embodiment, R^{3a} is chrysenyl. In one embodiment, R^{3a} is pyrenyl.

In one embodiment, R^{3a} is C₃-C₁₄ cycloalkyl. In one embodiment, R^{3a} is C₃-C₁₀ cycloalkyl. In one embodiment, R^{3a} is C₃-C₆ cycloalkyl. In one embodiment, R^{3a} is C₃ cycloalkyl. In one embodiment, R^{3a} is C₄ cycloalkyl. In one embodiment, R^{3a} is C₅ cycloalkyl. In one embodiment, R^{3a} is C₆ cycloalkyl. In one embodiment, R^{3a} is C₇ cycloalkyl. In one embodiment, R^{3a} is C₈ cycloalkyl. In one embodiment, the cycloalkyl is a fused, bridged, or spiro cycloalkyl. In one embodiment, the cycloalkyl is a monocyclic cycloalkyl.

In one embodiment, R^{3a} is 3 to 14-membered heterocyclyl. In one embodiment, R^{3a} is 5 to 14-membered heterocyclyl. In one embodiment, R^{3a} is 5 to 12-membered heterocyclyl. In one embodiment, R^{3a} is 5 to 10-membered heterocyclyl. In one embodiment, R^{3a} is 5-membered heterocyclyl. In one embodiment, R^{3a} is 6-membered heterocyclyl. In one embodiment, R^{3a} is 7-membered heterocyclyl. In one embodiment, R^{3a} is 8-membered heterocyclyl. In one embodiment, R^{3a} is 9-membered heterocyclyl. In one embodiment, R^{3a} is 10-membered heterocyclyl. In one embodiment, R^{3a} is 11-membered heterocyclyl. In one embodiment, R^{3a} is 12-membered heterocyclyl. In one embodiment, R^{3a} is 13-membered heterocyclyl. In one embodiment, R^{3a} is 14-membered heterocyclyl. In one embodiment, R^{3a} is 5 to 14-membered N-containing heterocyclyl. In one embodiment, R^{3a} is 5 to 10-membered N-containing heterocyclyl. In one embodiment, the heterocyclyl is a fused, bridged, or spiro heterocyclyl. In one embodiment, the heterocyclyl is monocyclic heterocyclyl.

In one embodiment, R^{3a} is 5 to 20-membered heteroaryl. In one embodiment, R^{3a} is 5 to 18-membered heteroaryl. In one embodiment, R^{3a} is 5 to 12-membered heteroaryl. In one embodiment, R^{3a} is 5 to 8-membered heteroaryl. In one embodiment, R^{3a} is 5-membered heteroaryl. In one embodiment, R^{3a} is 6-membered heteroaryl. In one embodiment, R^{3a} is 9-membered heteroaryl. In one embodiment, R^{3a} is 10-membered heteroaryl. In one embodiment, R^{3a} is 14-membered heteroaryl. In one embodiment, R^{3a} is 16-membered heteroaryl. In one embodiment, R^{3a} is 18-membered heteroaryl. In one embodiment, the heteroaryl comprises one or more nitrogen, oxygen, or sulfur ring atoms. In one embodiment, the heteroaryl is a fused heteroaryl. In one embodiment, the heteroaryl is monocyclic heteroaryl. In one embodiment, the heteroaryl comprises one or more pyridine rings.

In one embodiment, R^{3a} is unsubstituted. In one embodiment, R^{3a} is substituted. In one embodiment, R^{3a} is substituted with one or more halogen, OH, C₁-C₆ alkyl, C₆-C₁₀ aryl, C₆-C₁₀ cycloalkyl, C₆-C₁₀ aryloxy, or 3 to 8-membered heterocyclyl. In one embodiment, R^{3a} is substituted with one or more C₆-C₁₀ aryl, C₆-C₁₀ cycloalkyl, or 3 to 8-membered heterocyclyl, and wherein each aryl, cycloalkyl, and heterocyclyl is independently optionally substituted with one or more halogen, OH, or C₁-C₆ alkyl. In one embodiment, R^{3a} is substituted with one or more phenyl, naphthyl, cyclopropyl, cyclobutyl, cyclopentyl, cyclohexyl, pyridyl, or imidazolyl. In one embodiment, R^{3a} is substituted with one or more halogen, OH, or C₁-C₆ alkyl.

In one embodiment, R^{3a} is a C₆-C₂₀ aryl, 5 to 20-membered heteroaryl, or 3 to 14-membered heterocyclyl, wherein the aryl, heteroaryl and heterocyclyl are optionally substituted with one or more halogen, OH, C₁-C₆ alkyl, C₆-C₁₀ aryl, C₆-C₁₀ cycloalkyl, C₆-C₁₀ aryloxy, or 3 to 8-membered heterocyclyl. In one embodiment, R^{3a} is phenyl, pyridyl, biphenyl, bipyridyl, anthracenyl, acridinyl, acenaphthylenyl, indenyl, phenanthrenyl, phenalenyl, triphenylenyl, naphthalenyl, tetracenyl, chrysenyl, or pyrenyl, wherein R^{3a} is optionally substituted with one or more halogen, OH, or C₁-C₆ alkyl.

In one embodiment, R^{3a} is

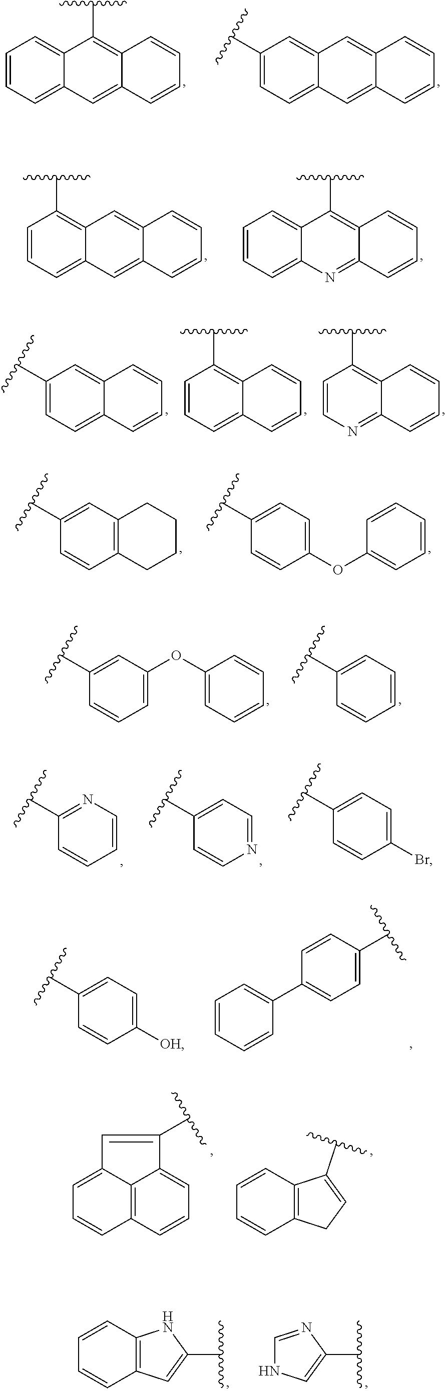

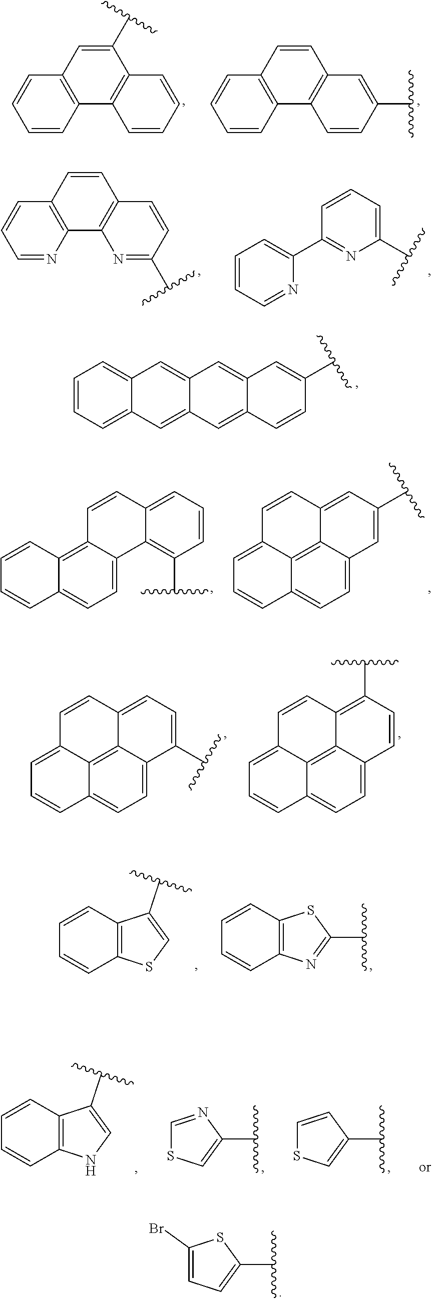

In one embodiment, R^{3a} is

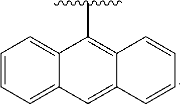 [SMILES: *c1c2ccccc2cc2ccccc12]

In one embodiment, R^{3a} is

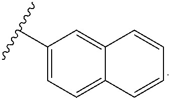 [SMILES: *c1ccc2ccccc2c1]

In one embodiment, R⁴ is H. In one embodiment, R⁴ is C₁-C₆ alkyl. In one embodiment, R⁴ is methyl. In one embodiment, R⁴ is ethyl. In one embodiment, R⁴ is C₃ alkyl. In one embodiment, R⁴ is C₄ alkyl. In one embodiment, R⁴ is C₅ alkyl. In one embodiment, R⁴ is C₆ alkyl. In one embodiment, the alkyl in R⁴ is unsubstituted. In one embodiment, the alkyl in R⁴ is substituted. In one embodiment, the alkyl in R⁴ is substituted with R^{3a}. In one embodiment, R⁴ is —(CH₂)₁₋₃—R^{3a}. In one embodiment, R⁴ is —CH₂—R^{3a}. In one embodiment, R⁴ is —CH₂CH₂—R^{3a}.

In one embodiment, R⁴ and NR¹ of G² together with the intervening atoms form a 5 to 12-membered heterocyclyl ring. In one embodiment, R⁴ and NR¹ of G² together with the intervening atoms form a 5 to 8-membered heterocyclyl ring. In one embodiment, the

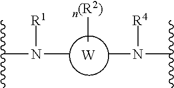

moiety is

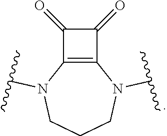 [SMILES: *N1CCCN(*)c2c1c(=O)c2=O]

In one embodiment, the compound is a compound of Formula (III-A), (III-B), (III-C), (III-D), (III-E), (II-F), or (II-G):

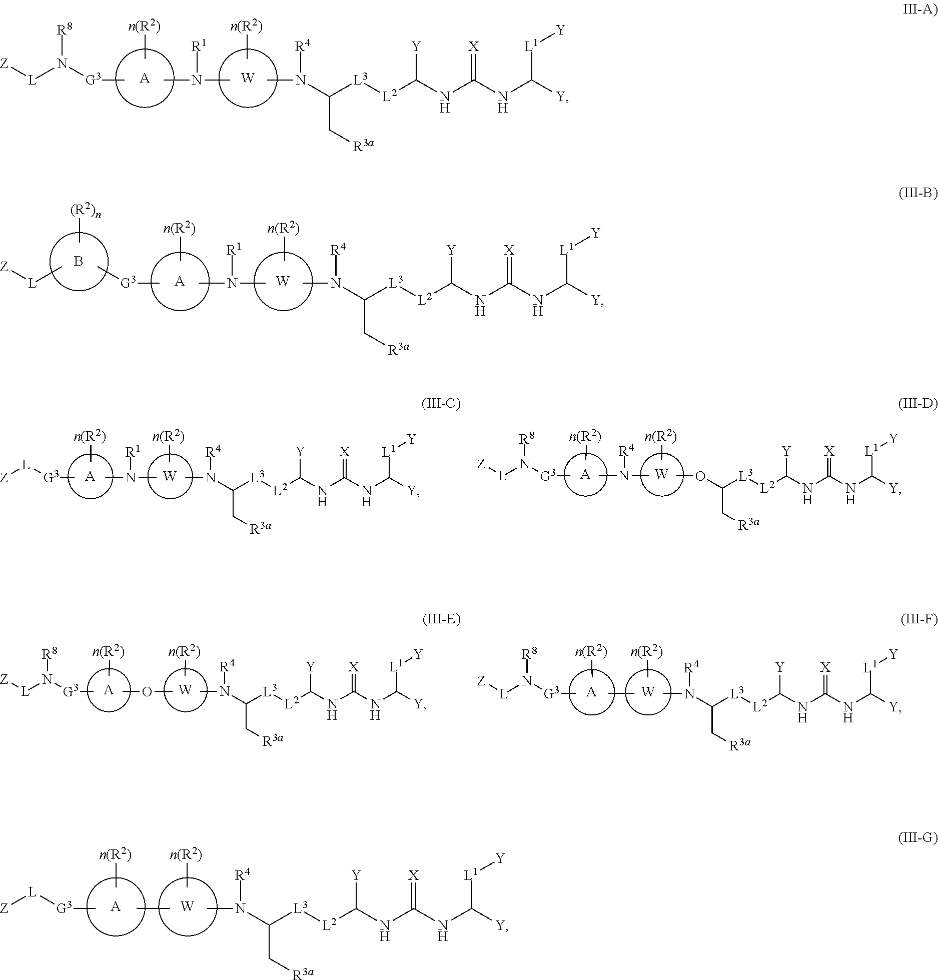

or a stereoisomer, a mixture of stereoisomers, a tautomer, or a pharmaceutically acceptable salt thereof, wherein:
R^{3a} is C₆-C₂₀ aryl, C₃-C₁₄ cycloalkyl, 3 to 14-membered heterocyclyl, or 5 to 20-membered heteroaryl, and wherein the alkyl, alkenyl, alkynyl, aryl, cycloalkyl, heterocyclyl, or heteroaryl in R³ or R^{3a} is optionally substituted with one or more halogen, OH, C₁-C₆ alkyl, C₆-C₁₀ aryl, C₆-C₁₀ cycloalkyl, 5 to 10-membered heteroaryl, 3 to 8-membered heterocyclyl, C₆-C₁₀ aryloxy, C₆-C₁₀ cycloalkyloxy, or 5- to 10-membered heteroaryloxy.

In one embodiment, the carbon connected to —CH₂R^{3a} has an S-configuration. In one embodiment, the carbon connected to —CH₂R^{3a} has an R-configuration.

In one embodiment, the compound is a compound of Formula (III-A′), (III-B′), (III-C′), (III-D′), (III-E′), (III-F′), or (III-G′):

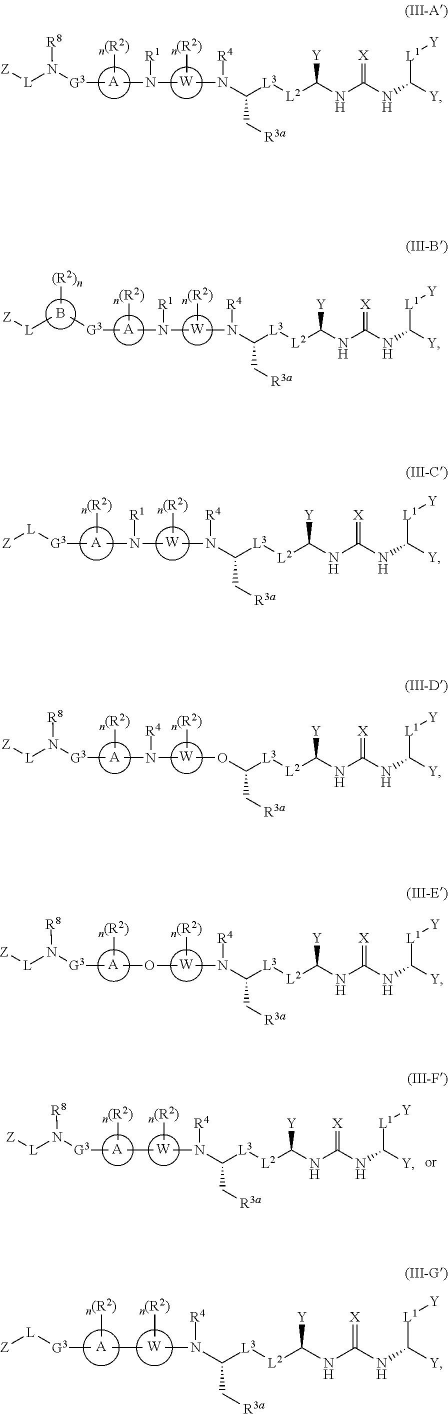

or a tautomer, or a pharmaceutically acceptable salt thereof.

In one embodiment, X is O. In one embodiment, X is S. In one embodiment, X is NH.

In one embodiment, Y is —CO₂H. In one embodiment, Y is —SO₂H. In one embodiment, Y is —SO₃H. In one embodiment, Y is —OSO₃H. In one embodiment, Y is —PO₂H. In one embodiment, Y is —PO₃H₂. In one embodiment, Y is —OPO₃H₂. In one embodiment, Y is

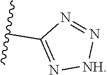 [SMILES: *c1nn[nH]n1]

In one embodiment, all Y are COOH. In one embodiment, only one of the Y is COOH. In one embodiment, only two of the Y is COOH. In one embodiment, each of the Y is different.

In one embodiment, when a carbon connected to Y is a chiral center, it has S-configuration. In one embodiment, when a carbon connected to Y is a chiral center, it has R-configuration. In one embodiment, the carbon connected to —Y and -L² has S-configuration. In one embodiment, the carbon connected to —Y and -L¹-Y has S-configuration.

In one embodiment, the compound is a compound of Formula (IV-A1), (IV-A2), (IV-B1), (IV-B2), (IV-C1), or (IV-C2):

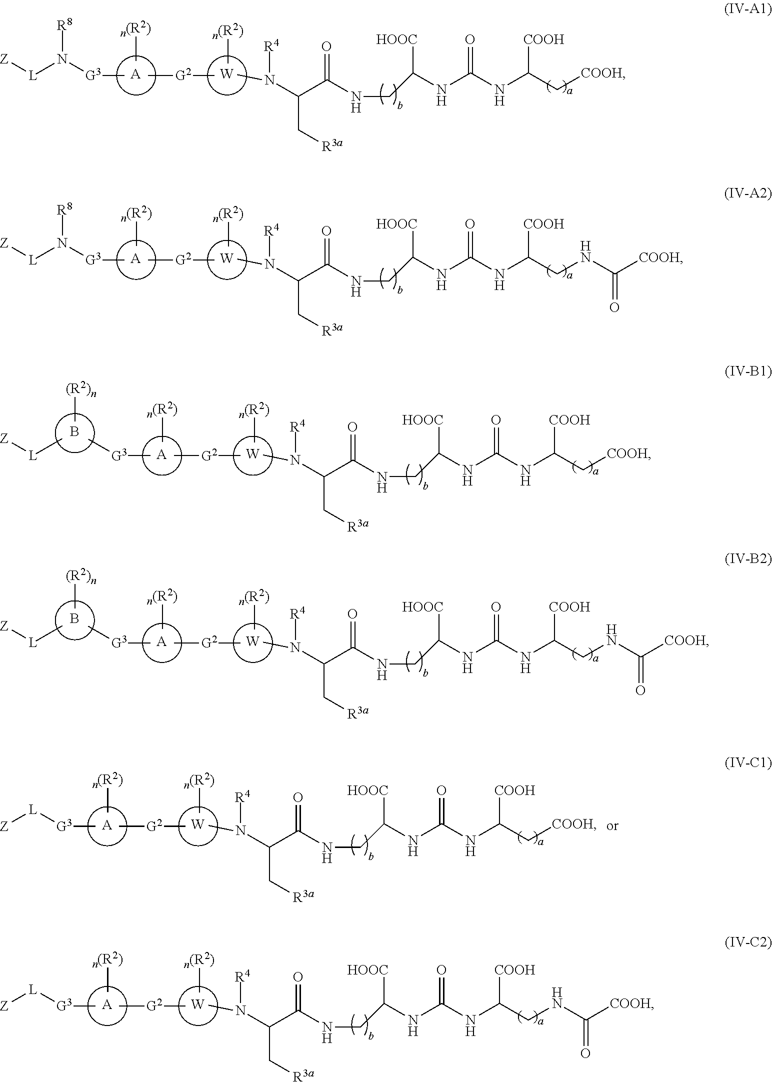

or a stereoisomer, a mixture of stereoisomers, a tautomer, or a pharmaceutically acceptable salt thereof, wherein a and b are each independently an integer from 1 to 5.

In one embodiment, the compound is a compound of Formula (IV-A1′), (IV-A2′) (IV-B1′), (IV-B2′), (IV-C1′), or IV-C2′):

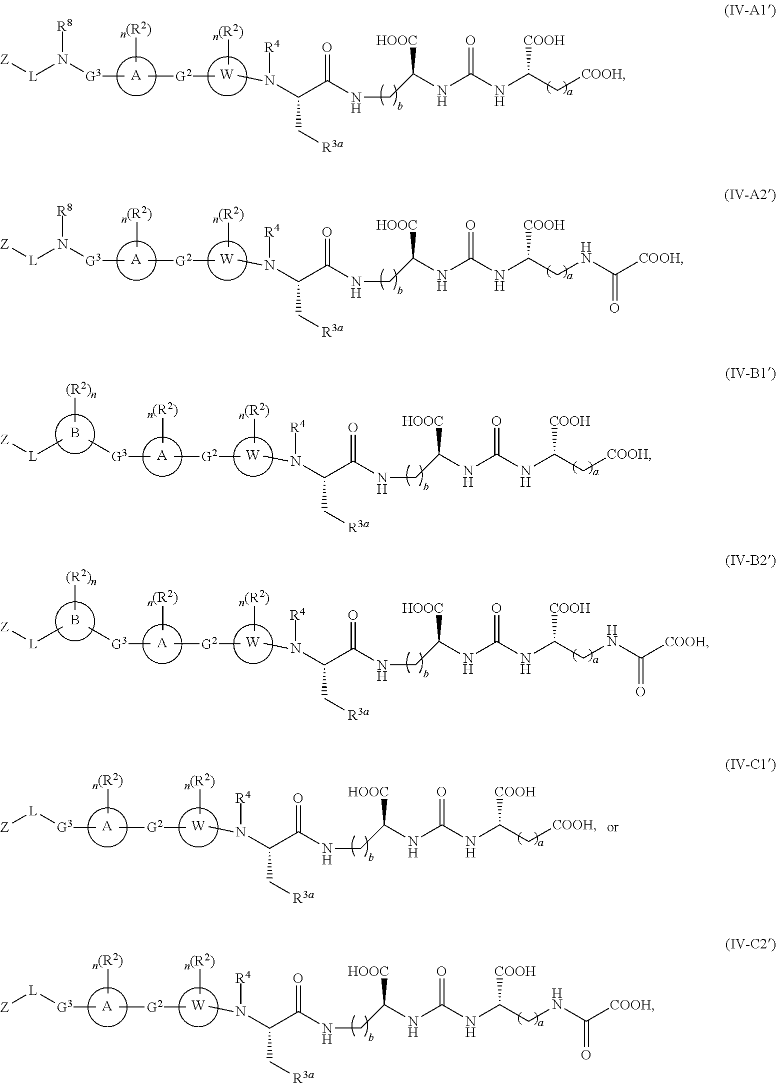

or a tautomer, or a pharmaceutically acceptable salt thereof, wherein a and b are each independently an integer from 1 to 5.

In one embodiment, a is 1. In one embodiment, a is 2. In one embodiment, a is 3. In one embodiment, a is 4. In one embodiment, a is 5. In one embodiment, b is 1. In one embodiment, b is 2. In one embodiment, b is 3. In one embodiment, b is 4. In one embodiment, b is 5.

In one embodiment, Ring B is a 5 to 10-membered heteroaryl. In one embodiment, Ring B is a 5 to 8-membered heteroaryl. In one embodiment, Ring B is a 5 or 6-membered heteroaryl. In one embodiment, Ring B is a 5 or 6-membered heteroaryl comprising one or more N, O, or S atoms on the ring. In one embodiment, Ring B is a 5-membered nitrogen-containing heteroaryl. In one embodiment, Ring B is a 5-membered sulfur-containing heteroaryl. In one embodiment, Ring B is a 5-membered oxygen-containing heteroaryl. In one embodiment, Ring B is a 6-membered nitrogen-containing heteroaryl. In one embodiment, Ring B is a 6-membered oxygen-containing heteroaryl. In one embodiment, Ring B is a 6-membered sulfur-containing heteroaryl.

In one embodiment, Ring B is oxadiazole ring. In one embodiment, Ring B is oxazole ring. In one embodiment, Ring B is isoxazole ring. In one embodiment, Ring B is triazole ring. In one embodiment, Ring B is imidazole ring. In one embodiment, Ring B is pyrazole ring. In one embodiment, Ring B is pyridine ring. In one embodiment, Ring B is pyrimidine ring. In one embodiment, Ring B is pyrazine ring. In one embodiment, Ring B is pyridazine ring. In one embodiment, Ring B is thiadiazole ring. In one embodiment, Ring B is thiazole ring. In one embodiment, Ring B is isothiazole ring. In one embodiment, Ring B is pyridone ring. In one embodiment, Ring B is pyrazinone ring.

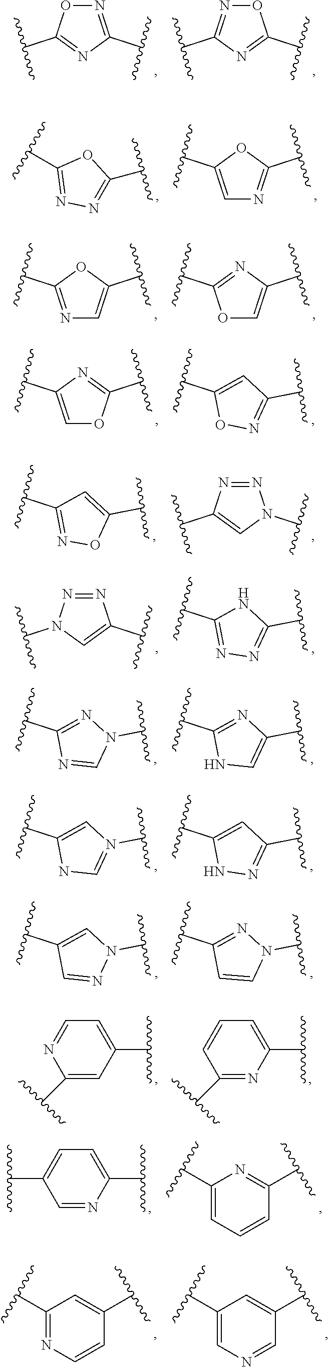

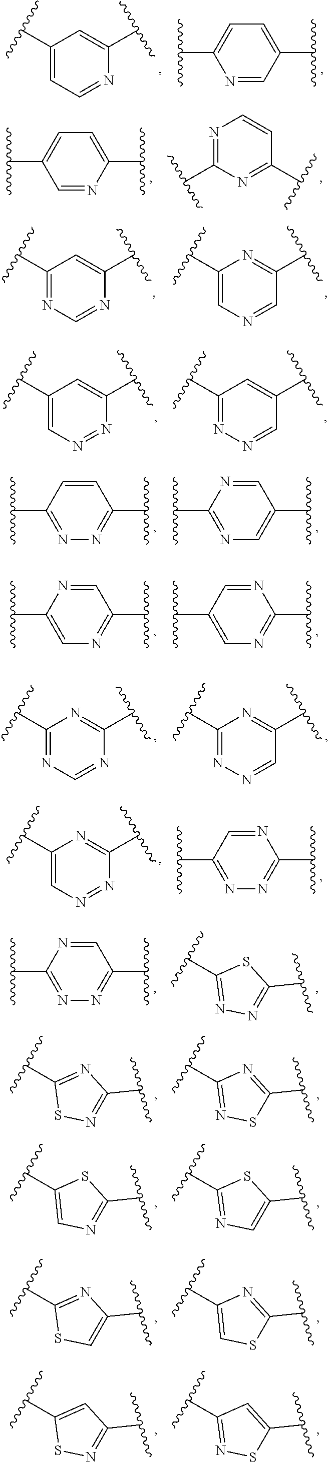

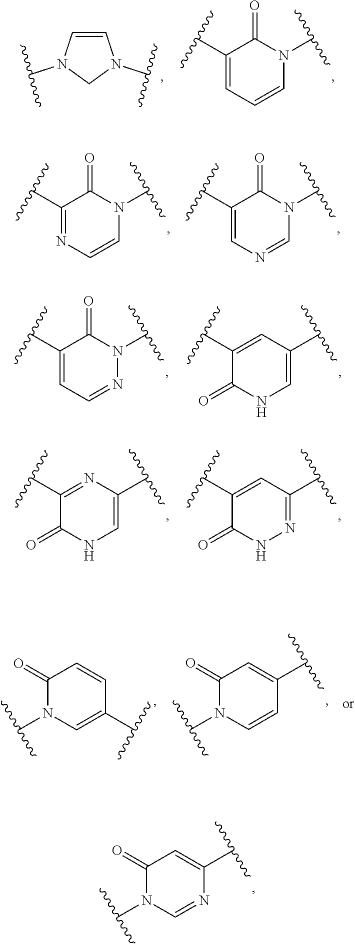

wherein the attachment to the left is to the direction of Z.

In one embodiment, Ring B is

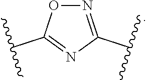 [SMILES: *c1noc(*)n1]

In one embodiment, Ring B is

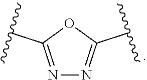 [SMILES: *c1nnc(*)o1]

In one embodiment, Ring B is

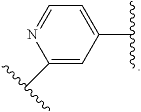 [SMILES: *c1ccnc(*)c1]

In one embodiment, Ring B is

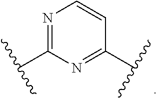 [SMILES: *c1ccnc(*)n1]

In one embodiment, Ring B is

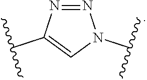 [SMILES: *c1cn(*)nn1]

In one embodiment, Ring B is

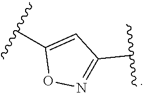 [SMILES: *c1cc(*)on1]

In these embodiments, the attachment to the left is to the direction of Z.

In one embodiment, Ring B is C₆-C₁₀ aryl. In one embodiment, Ring B is phenyl.

In one embodiment, Ring B is a C₃-C₁₄ cycloalkyl. In one embodiment, Ring B is C₃-C₁₀ cycloalkyl. In one embodiment, Ring B is C₃-C₆ cycloalkyl. In one embodiment, Ring B is cyclopropyl. In one embodiment, Ring B is cyclobutyl. In one embodiment, Ring B is cyclopentyl. In one embodiment, Ring B is cyclohexyl. In one embodiment, the cycloalkyl is a fused, bridged, or spiro cycloalkyl. In one embodiment, the cycloalkyl is a monocyclic cycloalkyl.

In one embodiment, Ring B is 3 to 14-membered heterocyclyl. In one embodiment, Ring B is 5 to 14-membered heterocyclyl. In one embodiment, Ring B is 5 to 12-membered heterocyclyl. In one embodiment, Ring B is 5 to 10-membered heterocyclyl. In one embodiment, Ring B is 3-membered heterocyclyl. In one embodiment, Ring B is 4-membered heterocyclyl (e.g., azetidinyl). In one embodiment, Ring B is 5-membered heterocyclyl (e.g., pyrrolidinyl). In one embodiment, Ring B is 6-membered heterocyclyl (e.g., piperidinyl). In one embodiment, Ring B is 7-membered heterocyclyl. In one embodiment, Ring B is 8-membered heterocyclyl. In one embodiment, the heterocyclyl is N-containing heterocyclyl. In one embodiment, the heterocyclyl is a fused, bridged, or spiro heterocyclyl. In one embodiment, the heterocyclyl is monocyclic heterocyclyl.

In one embodiment, G² is absent. In one embodiment, G² is —O—. In one embodiment, G² is —S—. In one embodiment, G² is NR¹. In one embodiment, G² is —NR¹—(C═O)—*. In one embodiment, G² is —(C═O)—NR¹—*. In one embodiment, G² is NH. In one embodiment, G² is —NHC(═O)—*. In one embodiment, G² is —(C═O)—NH—*. In one embodiment, G² is —NR¹—(C═S)—*. In these embodiments, * refers to the direction toward NR⁴.

In one embodiment, G² is C₁-C₆ alkylene. In one embodiment, G² is methylene. In one embodiment, G² is ethylene. In one embodiment, G² is C₃ alkylene. In one embodiment, G² is C₄ alkylene. In one embodiment, G² is C₅ alkylene. In one embodiment, G² is C₆ alkylene. In one embodiment, G² is unsubstituted C₁-C₆ alkylene. In one embodiment, G² is C₁-C₆ alkylene substituted with one or more halogen, oxo, hydroxyl, or C₁-C₆ alkoxy.

In one embodiment, G² is C₁-C₆ alkylene, wherein one or two —CH₂— in the alkylene is independently replaced by a —O—, —S—, —S(═O)₂—, —S(═O)—, —C(═O)—, —C(═O)O—*, —OC(═O)—*, NR¹, —(C═O)—NR¹—*, —NR¹—(C═O)—*, or —NR¹—(C═S)—*, wherein * refers to the direction toward NR⁴. In one embodiment, G² is C₁-C₃ alkylene, wherein one of the —CH₂— in the alkylene is independently replaced by a —O—, —S—, NR′, —(C═O)—NR¹—*, —NR¹—(C═O)—*, or —NR¹—(C═S)—*, wherein * refers to the direction toward NR⁴. In one embodiment, G² is C₁-C₃ alkylene, wherein one of the —CH₂— in the alkylene is replaced by a NH, —C(═O)NH—*, —NHC(═O)—*, or —NR¹—(C═S)—*, wherein * refers to the direction toward NR⁴.

In one embodiment, G² is absent, —O—, —NH—, —NHC(═O)—*, —NR¹—(C═S)—*, or unsubstituted C₁-C₆ alkylene, wherein * refers to the direction toward NR⁴. In one embodiment, G² is C₁-C₆ alkylene, wherein one or two —CH₂— in G² is replaced by a —O—, —S—, —S(═O)₂—, —S(═O)—, —C(═O)—, —C(═O)O—*, —OC(═O)—*, —NH—, —(C═O)—NH—*, —NH—(C═O)—*, or —NR′—(C═S)—*, wherein * refers to the direction toward NR⁴.

In one embodiment, G³ is absent. In one embodiment, G³ is —O—. In one embodiment, G³ is —S—. In one embodiment, G³ is NR¹. In one embodiment, G³ is —NR¹—(C═O)—*. In one embodiment, G³ is —(C═O)—NR¹—*. In one embodiment, G³ is NH. In one embodiment, G³ is —NHC(═O)—*. In one embodiment, G³ is —(C═O)—NH—*. In one embodiment, G³ is —NR¹—(C═S)—*. In these embodiments, * refers to the direction toward NR⁴.

In one embodiment, G³ is C₁-C₆ alkylene. In one embodiment, G³ is methylene. In one embodiment, G³ is ethylene. In one embodiment, G³ is C₃ alkylene. In one embodiment, G³ is C₄ alkylene. In one embodiment, G³ is C₅ alkylene. In one embodiment, G³ is C₆ alkylene. In one embodiment, G³ is unsubstituted C₁-C₆ alkylene. In one embodiment, G³ is C₁-C₆ alkylene substituted with one or more halogen, oxo, hydroxyl, or C₁-C₆ alkoxy. In one embodiment, G³ is —CH₂—. In one embodiment, G³ is —CH₂CH₂—. In one embodiment, G³ is —CH₂CH₂CH₂—.

In one embodiment, G³ is C₁-C₆ alkylene, wherein one or two —CH₂— in the alkylene is independently replaced by a —O—, —S—, —S(═O)₂—, —S(═O)—, —C(═O)—, —C(═O)O—*, —OC(═O)—*, NR¹, —(C═O)—NR¹—*, —NR¹—(C═O)—*, or —NR¹—(C═S)—*, wherein * refers to the direction toward NR⁴. In one embodiment, G³ is C₁-C₃ alkylene, wherein one of the —CH₂— in the alkylene is independently replaced by a —O—, —S—, NR¹, —(C═O)—NR¹—*, —NR¹—(C═O)—*, or —NR¹—(C═S)—*, wherein * refers to the direction toward NR⁴. In one embodiment, G³ is C₁-C₃ alkylene, wherein one of the —CH₂— in the alkylene is replaced by a NH, —C(═O)NH—*, —NHC(═O)—*, or —NR′—(C═S)—*, wherein * refers to the direction toward NR⁴.

In one embodiment, G³ is absent, —O—, —NH—, —NHC(═O)—*, —NR¹—(C═S)—*, or unsubstituted C₁-C₆ alkylene, wherein * refers to the direction toward NR⁴. In one embodiment, G³ is C₁-C₆ alkylene, wherein one or two —CH₂— in G³ is replaced by a —O—, —S—, —S(═O)₂—, —S(═O)—, —C(═O)—, —C(═O)O—*, —OC(═O)—*, —NH—, —(C═O)—NH—*, —NH—(C═O)—*, or —NR¹—(C═S)—*, wherein * refers to the direction toward NR⁴.

In one embodiment, Ring B is phenyl, and G³ is —NR¹—(C═S)—*. In one embodiment, (Ring B)-G³ is

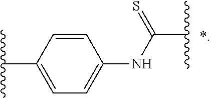 [SMILES: *C(=S)Nc1ccc(*)cc1]

In one embodiment, G² and G³ are both absent.

In one embodiment, Ring W is a 5 to 10-membered heteroaryl. In one embodiment, Ring W is a 5 to 8-membered heteroaryl. In one embodiment, Ring W is a 5 or 6-membered heteroaryl. In one embodiment, Ring W is a 5 or 6-membered heteroaryl comprising one or more N, O, or S atoms on the ring. In one embodiment, Ring W is a 5-membered nitrogen-containing heteroaryl. In one embodiment, Ring W is a 5-membered sulfur-containing heteroaryl. In one embodiment, Ring W is a 5-membered oxygen-containing heteroaryl. In one embodiment, Ring W is a 6-membered nitrogen-containing heteroaryl. In one embodiment, Ring W is a 6-membered oxygen-containing heteroaryl. In one embodiment, Ring W is a 6-membered sulfur-containing heteroaryl.

In one embodiment, Ring W is oxadiazole ring. In one embodiment, Ring W is oxazole ring. In one embodiment, Ring W is isoxazole ring. In one embodiment, Ring W is triazole ring. In one embodiment, Ring W is imidazole ring. In one embodiment, Ring W is pyrazole ring. In one embodiment, Ring W is pyridine ring. In one embodiment, Ring W is pyrimidine ring. In one embodiment, Ring W is pyrazine ring. In one embodiment, Ring W is pyridazine ring. In one embodiment, Ring W is thiadiazole ring. In one embodiment, Ring W is thiazole ring. In one embodiment, Ring W is isothiazole ring. In one embodiment, Ring W is pyridone ring. In one embodiment, Ring W is pyrazinone ring.

In one embodiment, Ring W is

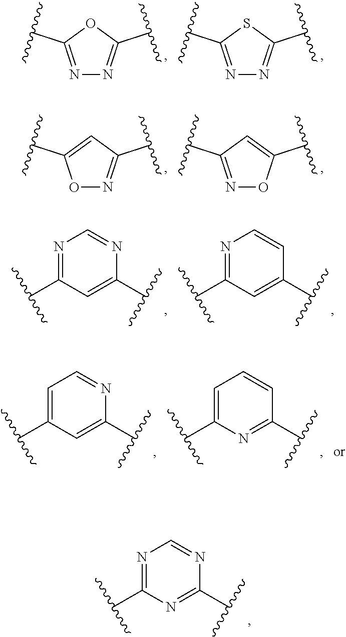

wherein the attachment to the left is to the direction of Z.

In one embodiment, Ring W is C₃-C₈ cycloalkyl. In one embodiment, Ring W is C₃-C₆ cycloalkyl. In one embodiment, Ring W is cyclopropyl. In one embodiment, Ring W is cyclobutyl. In one embodiment, Ring W is cyclopentyl. In one embodiment, Ring W is cyclohexyl.

In one embodiment, Ring W is C₃-C₈ cycloalkenyl. In one embodiment, Ring W is C₃-C₆ cycloalkenyl. In one embodiment, Ring W is cyclopropane. In one embodiment, Ring W is cyclobutene. In one embodiment, Ring W is cyclopentene. In one embodiment, Ring W is cyclohexene. In one embodiment, Ring W is

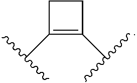 [SMILES: *C1=C(*)CC1]

In one embodiment, Ring W is 5 to 14-membered heterocyclyl. In one embodiment, Ring W is 5 to 12-membered heterocyclyl. In one embodiment, Ring W is 5 to 10-membered heterocyclyl. In one embodiment, Ring W is 5-membered heterocyclyl. In one embodiment, Ring W is 6-membered heterocyclyl. In one embodiment, Ring W is 7-membered heterocyclyl. In one embodiment, Ring W is 8-membered heterocyclyl. In one embodiment, the heterocyclyl is N-containing heterocyclyl. In one embodiment, the heterocyclyl is a fused, bridged, or spiro heterocyclyl. In one embodiment, the heterocyclyl is monocyclic heterocyclyl. In one embodiment, Ring W is

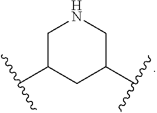 [SMILES: *C1CNCC(*)C1]

In one embodiment, Ring W is

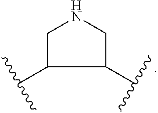 [SMILES: *C1CNCC1*]

In one embodiment,

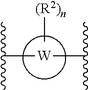

is

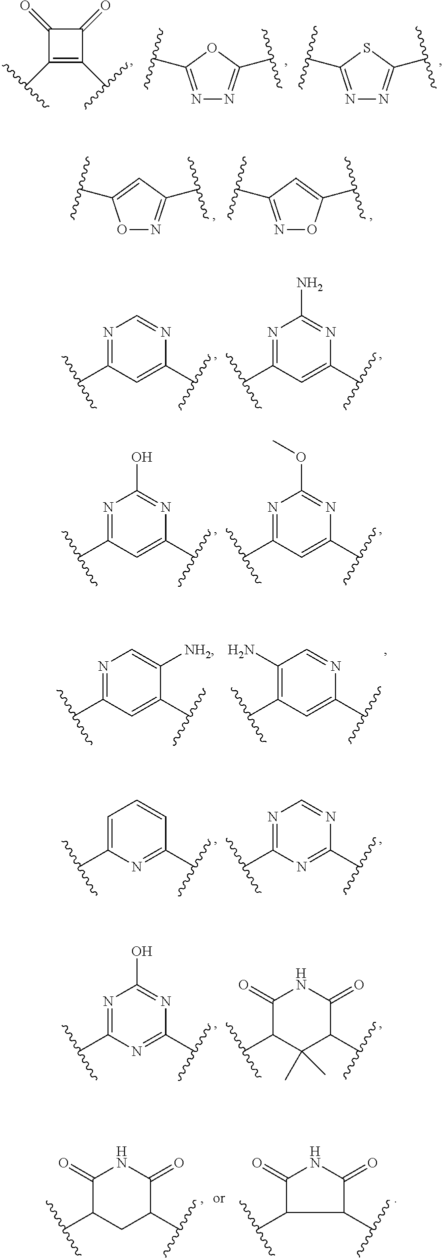

In one embodiment,

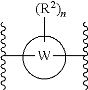

is

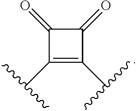 [SMILES: *c1c(*)c(=O)c1=O]

In one embodiment

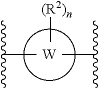

is

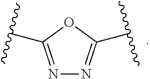 [SMILES: *c1nnc(*)o1]

In these embodiments, the attachment to the left is to the direction of Z.

In one embodiment, Ring W is a C₃-C₈ cycloalkyl, and Ring A is a C₃-C₁₄ cycloalkyl. In one embodiment, Ring W is a C₃-C₆ cycloalkyl, and Ring A is a C₃-C₁₀ cycloalkyl. In one embodiment, Ring W is a C₃-C₆ cycloalkyl, and Ring A is a bridged C₅-C₁₀ cycloalkyl. In one embodiment, Ring W is a C₃-C₆ cycloalkyl, and Ring A is

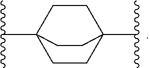 [SMILES: *C12CCC(*)(CC1)CC2]

In one embodiment, Ring W is a C₃-C₈ cycloalkenyl, and Ring A is a C₃-C₁₄ cycloalkyl. In one embodiment, Ring W is a C₃-C₆ cycloalkenyl, and Ring A is a C₃-C₁₀ cycloalkyl. In one embodiment, Ring W is a C₃-C₆ cycloalkenyl, and Ring A is a bridged C₅-C₁₀ cycloalkyl. In one embodiment, Rino W is a C₃-C₆ cycloalkenyl, and Ring A is

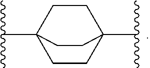 [SMILES: *C12CCC(*)(CC1)CC2]

In one embodiment,

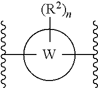

is

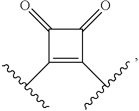 [SMILES: *c1c(*)c(=O)c1=O]

### and Ring A is

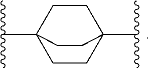 [SMILES: *C12CCC(*)(CC1)CC2]

In one embodiment, Ring W is a 5 to 10-membered heteroaryl, and Ring A is a C₃-C₁₄ cycloalkyl. In one embodiment, Ring W is a 5 to 6-membered heteroaryl, and Ring A is a C₃-C₁₀ cycloalkyl. In one embodiment, Ring W is a 5 to 6-membered heteroaryl, and Ring A is a bridged C₅-C₁₀ cycloalkyl. In one embodiment, Ring W is a 5 to 6-membered heteroaryl, and Ring A is

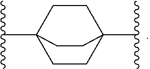 [SMILES: *C12CCC(*)(CC1)CC2]

In one embodiment, Ring W is a 5 to 14-membered heterocyclyl, and Ring A is a C₃-C₁₄ cycloalkyl. In one embodiment, Ring W is a 5 to 6-membered heterocyclyl, and Ring A is a C₃-C₁₀ cycloalkyl. In one embodiment, Ring W is a 5 to 6-membered heterocyclyl, and Ring A is a bridged C₅-C₁₀ cycloalkyl. In one embodiment, Ring W is a 5 to 6-membered heterocyclyl, and Ring A is

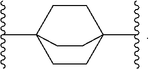 [SMILES: *C12CCC(*)(CC1)CC2]

In one embodiment (in any of the formula provided herein where both Ring A and Ring B are present), Ring B is a 5 to 10-membered heteroaryl, and Ring A is a C₃-C₁₄ cycloalkyl. In one embodiment, Ring B is a 5 to 6-membered heteroaryl, and Ring A is a C₃-C₁₀ cycloalkyl. In one embodiment, Ring B is a 5 to 6-membered heteroaryl, and Ring A is a bridged C₅-C₁₀ cycloalkyl. In one embodiment, Ring B is a 5 to 6-membered heteroaryl, and Ring A is

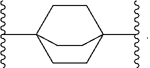 [SMILES: *C12CCC(*)(CC1)CC2]

In one embodiment, at least one of Ring A and Ring B is not phenyl. In one embodiment, Ring B is phenyl, and Ring A is

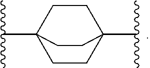 [SMILES: *C12CCC(*)(CC1)CC2]

In one embodiment, Ring B is

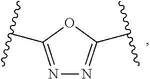 [SMILES: *c1nnc(*)o1]

### and Ring A is

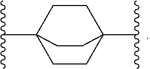 [SMILES: *C12CCC(*)(CC1)CC2]

In one embodiment, P¹ (or Ring B as applicable) is 5 to 6-membered heteroaryl, Ring A is a bridged C₅-C₁₀ cycloalkyl, and Ring W is a C₃-C₆ cycloalkenyl. In one embodiment, P¹ (or Ring B as applicable) is

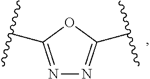 [SMILES: *c1nnc(*)o1]

### Ring A is

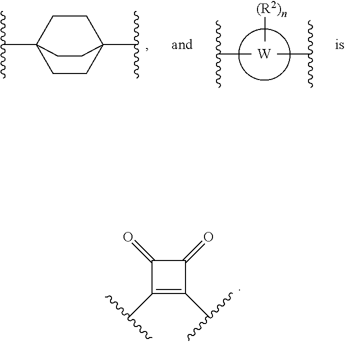

In one embodiment, P¹ (or NR⁸ as applicable) is NH, Ring A is a bridged C₅-C₁₀ cycloalkyl, and Ring W is a C₃-C₆ cycloalkenyl. In one embodiment, P¹ (or NR⁸ as applicable) is NH, Ring A

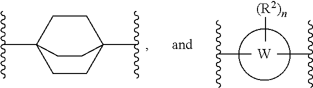

is

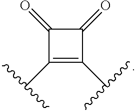 [SMILES: *c1c(*)c(=O)c1=O]

In one embodiment, L is absent. In one embodiment, L is a linker.

In one embodiment, L is C₁-C₁₂ alkylene. In one embodiment, L is C₁-C₆ alkylene. In one embodiment, L is methylene. In one embodiment, L is ethylene. In one embodiment, L is C₃ alkylene. In one embodiment, L is C₄ alkylene. In one embodiment, L is C₅ alkylene. In one embodiment, L is C₆ alkylene. In one embodiment, L is unsubstituted C₁-C₁₂ alkylene. In one embodiment, L is C₁-C₁₂ alkylene substituted with one or more halogen, oxo, hydroxyl, or C₁-C₆ alkoxy. In one embodiment, L is —(CH₂)₁₋₆—. In one embodiment, L is —CH₂—. In one embodiment, L is —CH₂CH₂—. In one embodiment, L is —CH₂CH₂CH₂—.

In one embodiment, L comprises or is C₁-C₁₂ alkylene, wherein one or more —CH₂— in the alkylene is independently replaced by a —NHC(═O)—*, —C(═O)NH—*, —NHC(═S)—*, —C(═S)NH—*, —O—, —S—, —S(═O)₂—, —S(═O)—, —C(═O)—, —C(═O)O—*, —OC(═O)—*, or —NH—, wherein * refers to the direction toward P¹. In one embodiment, L comprises an optionally C₁-C₆ alkylene, wherein one or two —CH₂— in the alkylene is independently replaced by a —NHC(═O)—*, —C(═O)NH—*, —NHC(═S)—*, —C(═S)NH—*, —O—, —S, or —NH—, wherein * refers to the direction toward P¹. In one embodiment, one or more non-terminal —CH₂— in the alkylene of L is independently replaced by a group provided herein. In one embodiment, in addition to or independently from the one or more non-terminal —CH₂—, the terminal —CH₂— connecting Z and/or the terminal —CH₂— connecting P¹ is independently replaced by a group provided herein.

In one embodiment, L comprises or is —(CH₂)₀₋₅—NHC(═O)—(CH₂)₀₋₅—*. In one embodiment, L comprises or is —(CH₂)₀₋₅—NHC(═O)—(CH₂)₁₋₅—*. In one embodiment, L comprises or is —(CH₂)₀₋₅—C(═O)NH—(CH₂)₀₋₅—*. In one embodiment, L comprises or is —(CH₂)₀₋₅—C(═O)NH—(CH₂)₁₋₅—*. In one embodiment, L comprises or is —(CH₂)₀₋₅—O—(CH₂)₀₋₅—*. In one embodiment, L comprises or is —(CH₂)₀₋₅—O—(CH₂)₁₋₅—*. In one embodiment, L comprises or is —(CH₂)₀₋₅—NHC(═S)—(CH₂)₀₋₅—*. In one embodiment, L comprises or is —(CH₂)₀₋₅—NHC(═S)—(CH₂)₁₋₅—*. In one embodiment, L comprises or is —(CH₂)₁₋₅—NHC(═O)—(CH₂)₁₋₅—*. In one embodiment, L comprises or is —(CH₂)₁₋₅—C(═O)NH—(CH₂)₁₋₅—*. In one embodiment, L comprises or is —(CH₂)₁₋₅—O—(CH₂)₁₋₅—*. In one embodiment, L comprises or is —(CH₂)₁₋₅—NHC(═S)—(CH₂)₁₋₅—*. In these embodiment, * refers to the direction toward P¹.

In one embodiment, L comprises or is a peptide comprising 1 to 5 amino acids. In one embodiment, L comprises or is a peptide comprising 2 to 4 amino acids. In one embodiment, the amino acid(s) are conventional amino acids. In one embodiment, L is a cleavable peptide (e.g., by cathepsin).

In one embodiment, L is —(CH₂)₀₋₆-(Xaa¹)₁₋₅-(CH₂)₀₋₆—*. In one embodiment, L (L) is —(CH₂)₀₋₆-(Xaa¹)₁-(CH₂)₀₋₆—*. In one embodiment, L (L) is —(CH₂)₀₋₆-(Xaa¹)₂-(CH₂)₀₋₆—*. In one embodiment, L (L) is —(CH₂)₀₋₆-(Xaa¹)₃-(CH₂)₀₋₆—*. In one embodiment, L (L) is —(CH₂)₀₋₆-(Xaa¹)₄-(CH₂)₀₋₆—*. In one embodiment, L (L) is —(CH₂)₀₋₆-(Xaa¹)₅-(CH₂)₀₋₆—*. In one embodiment, L (L) is -(Xaa¹)₁₋₅-*. Xaa¹ is an amino acid of formula —N(R⁸)R⁹C(═O)—*; each R⁸ is independently H or optionally substituted C₁-C₆ alkyl; and each R⁹ is independently C₁-C₂₀ alkylene, wherein one or more —CH₂— in the alkylene is independently optionally replaced by C₃-C₁₄ cycloalkylene, C₃-C₁₄ cycloalkenylene, C₆-C₁₀ arylene, 5 to 14-membered heterocyclylene, or 5 to 10-membered heteroarylene; wherein the alkylene, cycloalkylene, cycloalkenylene, arylene, and heteroarylene are independently optionally substituted; or R⁸ and R⁹, or R⁸ and part of R⁹, together with the nitrogen they are attached to form a 5 to 12-membered optionally substituted heterocyclyl. In these embodiments, * refers to the direction toward P¹.

In one embodiment, P¹ is NR⁸, and L is absent. In one embodiment, P¹ is NRx, L is absent, and Z is a chelating moiety resulted from removal of an —OH group from an acid group or a derivative thereof. In one embodiment, P¹ is Ring B, and L is —(CH₂)₁₋₆—.

In one embodiment, the compound is a compound of Formula (V-A1), (V-A2), (V-A3), (V-B1), (V-B2), (V-B3), (V-C1), (V-C2), or (V-C3):

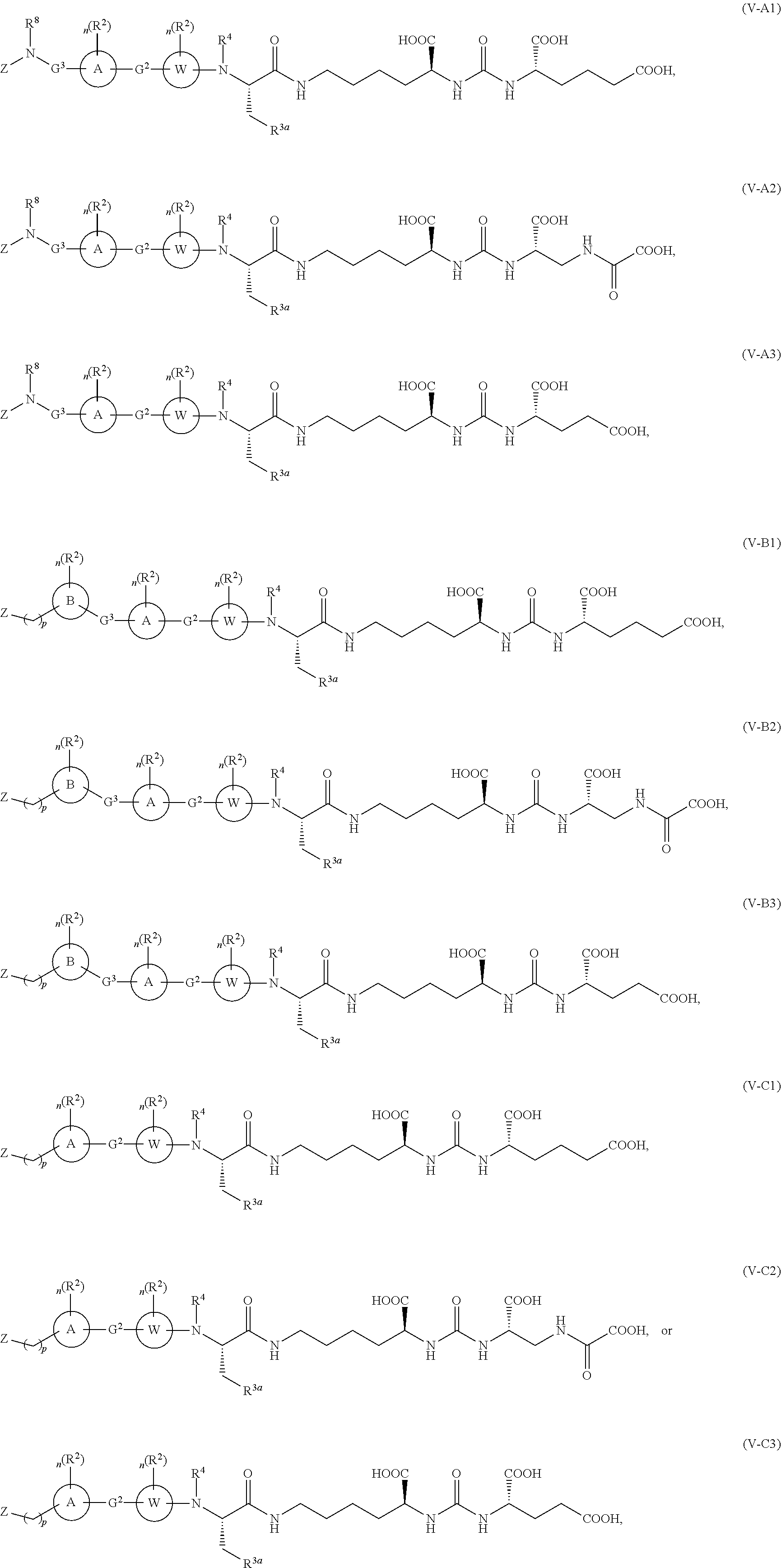

or a stereoisomer, a mixture of stereoisomers, a tautomer, or a pharmaceutically acceptable salt thereof, wherein p is an integer from 0 to 6.

In one embodiment, the compound is a compound of Formula (V-A1′) or Formula (V-B1′):

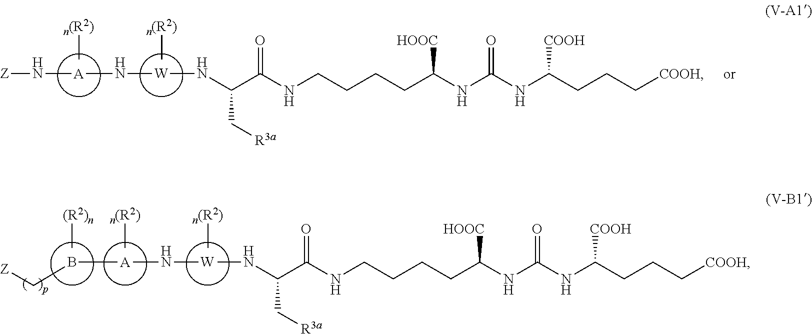

or a stereoisomer, a mixture of stereoisomers, a tautomer, or a pharmaceutically acceptable salt thereof.

In one embodiment, p is 0. In one embodiment, p is 1. In one embodiment, p is 2. In one embodiment, p is 3. In one embodiment, p is 4. In one embodiment, p is 5. In one embodiment, p is 6.

In one embodiment, Z is a radioactive moiety. In one embodiment, the radioactive moiety is a fluorescent isotope, a radioisotope, or a radioactive drug. In one embodiment, the radioactive moiety is a radioactive isotope suitable for diagnostic use. In one embodiment, the radioactive moiety is a radioactive isotope suitable for therapeutic use. In one embodiment, the radioactive moiety is a radioactive isotope suitable for medical imaging or radiotherapy. In one embodiment, the radioactive moiety is selected from the group consisting of alpha radiation emitting isotopes, beta radiation emitting isotopes, gamma radiation emitting isotopes, Auger electron emitting isotopes, X-ray emitting isotopes, and fluorescence emitting isotopes.

In one embodiment, the radioactive moiety is a complex formed by a radioisotope of a metal cation and a chelating agent. In one embodiment, the radioactive moiety is a complex formed by a cation of ¹⁷⁷Lu, Al¹⁸F, ²⁰³Pb, ²¹²Pb, ⁵¹Cr, ⁶⁷Ga, ⁶⁸Ga, ⁸⁹Zr, ¹¹¹In, ^{99m}Tc, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ⁴⁷Sc, ¹⁴²Pr, ¹⁵⁹Gd, ²¹²Bi, ²¹³Bi, ⁹⁷Ru, ¹⁰⁹Pd, ¹⁰⁵Rh, ^{101m}Rh, ¹¹⁹Sb, ¹²⁸Ba, ¹⁹⁷Hg, ¹⁵¹Eu, ¹⁵³Eu, ¹⁶⁹Eu, ²⁰¹Tl, ⁶⁴Cu, ⁶⁷Cu, ¹⁸⁸Re, ¹⁸⁶Re, ¹⁹⁸Au, ²²⁵Ac, ²²⁷Th, or ¹⁹⁹Ag and a chelating agent provided herein. In one embodiment, the radioactive moiety is a complex formed by a cation of ¹⁷⁷Lu, ⁶⁸Ga, ⁹⁰Y, Al¹⁸F, ²⁰³Pb, ²¹²Pb, ⁶⁴Cu, or ²²⁵Ac and a chelating agent provided herein. In one embodiment, radioactive moiety is a complex formed by a cation of ¹⁷⁷Lu and a chelating agent provided herein. In one embodiment, radioactive moiety is a complex formed by a cation of ⁶⁸Ga and a chelating agent provided herein. In one embodiment, the chelating agent is a chelating agent provided in Table 1.

As used herein and unless otherwise specified, where the chemical name or abbreviation refers to a complete molecule, the chelating agent is a chelating moiety of the complete molecule. In one embodiment, the point of attachment of the chelating moiety is on a chelating atom (e.g., a nitrogen atom). In one embodiment, the point of attachment of the chelating moiety is on a carbon atom from an alkylene group attached to a chelating atom. In one embodiment, the point of attachment of the chelating moiety is on a ring carbon atom from a ring containing a chelating atom (e.g., a ring carbon atom of a pyridine ring).

In one embodiment, the radioactive moiety is (a metal-chelating moiety of) ¹⁷⁷Lu-DOTA, ¹⁷⁷Lu-DOTAGA, ⁶⁸Ga-DOTA, ⁹⁰Y-DOTA, Al¹⁸F-NOTA, ²⁰³Pb-TCMC, ²¹²Pb—PSC, ²⁰³Pb—PSC, ²¹²Pb-TCMC, ⁶⁴Cu-DOTA, or ²²⁵Ac-DOTA. In one embodiment, the radioactive moiety is ¹⁷⁷Lu-DOTA. In one embodiment, the radioactive moiety is ¹⁷⁷Lu-DOTAGA. In one embodiment, the radioactive moiety is ⁶⁸Ga-DOTA.

In one embodiment, the radioactive moiety comprises ¹¹C, ¹⁸F, ⁷²As, ⁷²Se, ¹²³I, ¹²⁴I, ¹³¹I, or ²¹¹At.

In one embodiment, Z is a fluorescent dye. In one embodiment, the fluorescent dye is an Xanthene, an Acridine, an Oxazine, an Cyanine, a Styryl dye, a Coumarin, a Porphine, a Metal-Ligand-Complex, a Fluorescent protein, a Nanocrystals, a Perylene, a Boron-dipyrromethene, or a Phthalocyanine, or a conjugate or combination thereof.

In one embodiment, Z is a chelating agent. A wide variety of chelating agents have been reported, e.g., by Banerjee et al. (Banerjee, et al., Dalton Trans, 2005, 24: 3886), by Price, et al. (Chem Soc Rev, 2014, 43: 260), by Wadas, et al. (Chem Rev, 2010, 110: 2858), as well as in U.S. Pat. Nos. 5,367,080, 5,367,080, 5,364,613, 5,021,556, 5,075,099, and 5,886,142, the entirety of each of which is incorporated herein by reference. In one embodiment, the chelating agent is a linear chelating agent. In one embodiment, the chelating agent is a cyclic agent. In one embodiment, the chelating agent is a macrocyclic chelating agent. In one embodiment, the chelating agent is a nitrogen-containing macrocyclic chelating agent. In one embodiment, the chelating agent is a tetrapyridine chelating agent, N3S chelating agent, N2S2 chelating agent, or N4 chelating agent.

In one embodiment, the chelating agent is capable of binding with a radioactive moiety. In one embodiment, the binding is through ionic, covalent, dipolar, or ion-dipole interactions. In one embodiment, the chelating agent binds directly to the radioactive moiety. In one embodiment, the chelating agent binds indirectly to the radioactive moiety (e.g., through a linker).

In one embodiment, the chelating agent comprises one or more amines (e.g., primary amine, secondary amine, or tertiary amine). In one embodiment, the chelating agent comprises one or more ring oxygen atoms. In one embodiment, the chelating agent comprises one or more ring nitrogen atoms. In one embodiment, the chelating agent comprises two ring nitrogen atoms. In one embodiment, the chelating agent comprises three ring nitrogen atoms. In one embodiment, the chelating agent comprises four ring nitrogen atoms. In one embodiment, the chelating agent comprises one or more carboxylic acids. In one embodiment, the chelating agent comprises two carboxylic acids. In one embodiment, the chelating agent comprises three carboxylic acids. In one embodiment, the chelating agent comprises four carboxylic acids. In one embodiment, the chelating agent comprises two or more ring nitrogen atoms, and two or more carboxylic acids.

In one embodiment, the chelating agent is a tetradentate chelating agent. In one embodiment, the chelating agent is a hexadentate chelating agent. In one embodiment, the chelating agent is an octadentate chelating agent. In one embodiment, the chelating agent comprises an optionally substituted 8 to 20-membered nitrogen-containing heterocyclyl.

In one embodiment, the chelating agent is a chelating agent that forms a complex with a divalent or trivalent metal cation. In one embodiment, the chelating agent is (a chelating moiety of) 1,4,7,10-tetraazacyclododecane-N,N′,N,N′-tetra acetic acid (DOTA), ethylenediaminetetraacetic acid (EDTA), 1,4,7-triazacyclononane-1,4,7-triacetic acid (NOTA), 1,4,7,10-tetraazacyclododecane-1-(glutaric acid)-4,7,10-triacetic acid (DOTAGA), 2-[4,7,10-tris(2-amino-2-oxoethyl)-1,4,7,10-tetrazacyclododec-1-yl]acetamide (TCMC), triethylenetetramine (TETA), iminodiacetic acid, diethylenetriamine-N,N,N′,N′,N″-penta acetic acid (DTPA), bis-(carboxymethyl imidazole)glycine, or 6-hydrazinopyridine-3-carboxylic acid (HYNIC). In one embodiment, the chelating agent is DOTA, NOTA, EDTA, DTPA, TETA, DO3A, PCTA, or desferrioxamine.

In one embodiment, the chelating agent is (a chelating moiety of) AAZTA, BAT, CDTA, DTA, CyEDTA, EDTMP, DTPMP, CyDTPA, Cy2DTPA, DTPA-MA, DTPA-BA, BOPA, NTA, NOC, NOTP, CY-DTA, DTCBP, CTA, cyclam, CB-Cyclam, cyclen, TETA, sarcophagine, CPTA, TEAMA, Cyclen, DATA, DFO, DATA(M), DATA(P), DATA(Ph), DATA(PPh), DEDPA, H₄octapa, H₂dedpa, H₅decapa, H₂azapa, H2CHX-DEDPA, DFO, DFO-Chx-MAL, DFO-p-SCN, DFO-1AC, DFO-BAC, p-SCN-Bn-DFO, DFO-pPhe-NCS, DFO-HOPO, DFC, diphosphine, DOTAGA, DOTA-MFCO, DOTAM, DOTAM-mono-acid, DOTA-MA, DOTA-pNB, DOTA-4AMP, nitro-DOTA, nitro-PA-DOTA, p-NCS-Bz-DOTA, PA-DOTA, DOTA-NCS, DOTA-NHS, CB-DO2A, PCTA, p-NH₂-Bn-PCTA, p-SCN-Bn-PCTA, p-SCN-Bn-DOTA, DOTMA, NB-DOTA, H4NB-DOTA, H4TCE-DOTA, HOPO, 3,4,3-(Li-1,2-HOPO), TREN(Me-3,2-HOPO), TCE-DOTA, DOTP, DOTMP, DOTEP, DOTMPE, F-DOTPME, DOTPP, DOTBzP, DOTA-monoamide, DOXP, p-NCS-DOTA, p-NCS-PADOTA, p-NCS-TRITA, TRITA, TETA, 3p-C-DEPA, 3p-C-DEPA-NCS, p-NH₂—BN—OXO-DO3A, p-SCN—BN-TCMC, TCMC, 4-aminobutyl-DOTA, azido-mono-amide-DOTA, BCN-DOTA, butyne-DOTA, BCN-DOTA-GA, DOA3P, DO2a2p, DO2A(trans-H2do2a), DO3A, DO3A-thiol, DO3AtBu-N-(2-aminoethyl)ethanamide, DO3TMP-monoamide, DO2AP, CB-DO2A, C3B-DO2A, HP-DO3A, DOTA-NHS-ester, maleimide-DOTA-GA, maleimido-mono-aminde-DOTA, maleimide-DOTA, NH₂-DOTA-GA, NH₂-PEG4-DOTA-GA, GA, p-NH₂-Bn-DOTA, p-NO₂-Bn-DOTA, p-SCN-Bn-DOTA, p-SCN-Bz-DOTA, TA-DOTA, TA-DOTA-GA, OTTA, DOXP, TSC, FSC, DTC, DTCBP, PTSM, ATSM, H2ATSM, H2PTSM, Dp44mT, DpC, Bp44mT, QT, hybrid thiosemicarbazone-benzothiazole, thiosemicarbazone-styrylpyridine tetradentate ligands H₂L2-4, HBED, HBED-CC, dmHBED, dmEHPG, HBED-nn, SHBED, Br-Me2HBED, BPCA, HEHA, BF-HEHA, deferiprone, THP, HYNIC (2-hydrazino nicotinamide), NHS—HYNIC, HYNIC-Kp-DPPB, HYNIC-Ko-DPPB, (HYNIC)(tricine)2, (HYNIC)(EDDA)Cl, p-EDDHA, AIM, AIMA, IAMB, MAMA, MAMA-DGal, MAMA-MGal, MAMA-DA, MAMA-HAD, PSC, macropa, macropaquin, macroquin-SO3, Crown, MAG3B, NODAGA, SCN-Bz-NOTA-R, NOT-P (NOTMP), NOTAM, p-NCS-NOTA, TACN, TACN-TM, NETA, NETA-monoamine, p-SCN-PhPr-NE3TA, C-NE3TA-NCS, C-NETA-NCS, 3p-C-NETA, NODASA, NOPO, NODA, NODA-MPAA, NO₂A, N-benzyl-NODA, C-NOTA, BCNOT-monoamine, maleimido-mono-amide-NOTA, NO₂A-azide, NO₂A-butyne, NO₂AP, NO₃AP, N-NOTA, oxo-DO3A, p-NH₂-Bn-NOTA, p-NH₂-Bn-oxo-DO3A, p-NO₂-Bn-cyclen, p-SCN-Bn-NOTA, p-SCN-Bn-oxo-DO3A, TRAP, PEPA, BF-PEPA, pycup, pycup2A, pycup1A1Bn, pycup2Bn, SarAr-R, DiAmSar, AmBaSar-R, siamSar, Sar, Tachpyr, tachpyr-(6-Me), TAM A, TAM B, TAME, TAME-Hex, THP-Ph-NCS, THP-NCS, THP-TATE, NTP, H3THP, THPN, CB-TE2A, PCB-TE1A1P, TETA-NHS, CPTA, CPTA-NHS, CB-TE1K1P, CB-TE2A, TE2A, H2CB-TE2A, TE2P, CB-TE2P, MM-TE2A, DM-TE2A, 2C-TETA, 6C-TETA, BAT, BAT-6, NHS-BAT ester, SSBAT, CHX-A″-DTPA, SCN—CHX-A-DTPA-P, SCN-TETA, TMT-amine, p-BZ-HTCPP, H4pypa, H4octox, p-NO₂-Bn-neunpa, p-SCN-Bn-H4neunpa, TTHA, tBu4pypa-C7-NHS, H4neunpa, H2macropa, BT-DO3A, DO3A-Nprop, DO3AP, DOTPMB, DOTAMAE, DOTAMAP, DO3AMBu, DEPA, p-NO₂-Bn-PCTA, symPC2APA, symPCA2PA, asymPC2APA, asymPCA2PA, ^{99m}Tc(CO)₃-Chelators, NₓS₄₋ₓ (N₄, N₂S₂, N₃S), or MeO-DOTA-NCS. In one embodiment, the chelating agent is DOTA, DOTAGA, NOPO, PCTA, NOTA, NODAGA, NODA-MPAA, HBED, TETA, CB-TE2A, DTPA, CHX-A″-DTPA, DFO, Macropa, Crown, DOTAM (also called TCMC), PSC, HOPO, HEHA, TRAP, THP, DATA, NOTP, sarcophagine, FSC, NETA, H4octapa, Pycup, NₓS₄₋ₓ (N₄, N₂S₂, N₃S), Hynic, ^{99m}Tc(CO)₃-chelators, or their analogs. In one embodiment, the chelating agent is DOTA, DOTAGA, NOPO, PCTA, DOTAM, PSC, Macropa, Crown, NOTA, NODAGA, NODA-MPAA, HBED, CB-TE2A, DFO, THP, or N₄. In one embodiment, the chelating agent is DOTA, DOTAGA, NOPO, PCTA, DOTAM, PSC, Macropa, Crown, NOTA, or NODAGA. In one embodiment, the chelating agent is DOTA, NOPO, PCTA, Macropa or Crown. The structures of the chelating agents are known in the art and have been reported, e.g., in U.S. Pat. Nos. 4,885,363, 5,720,934, 5,367,080, 5,364,613, 5,021,556, 5,075,099, and 5,886,142; Li, et al., Nucl Med Biol, 2001, 28:145; Eisenwiener, et al., Bioconjug Chem, 2002, 13:530; Brechbiel, et al., Bioconjug Chem, 1991, 2:187; Price, et al., Chem Soc Rev, 2014, 43:260; Schwartz, et al., Bioconjug Chem, 1991, 2:333; Nock, et al., J Nucl Med, 2005, 46:1727; McAuley, et al., Canadian J Chem, 1989, 67:1657; Doulias et al., Free Radic Biol Med, 2003, 35:719; Pfister, et al., EJNMMI Res, 2015, 5:74; Cusnir, et al., Int J Mol Sci, 2017, 18; Demoin, et al., Nucl Med Biol, 2016, 43:802; Thiele, et al., Angew Chem Int Ed, 2017, 56:14712; Price, et al., Chem Soc Rev, 2014, 43:260; Allott, et al., Chem Commun (Camb), 2017, 53:8529; Tomesello, et al., Molecules, 2017, 22:1282; Ma, et al., Dalton Trans, 2015, 44:4884; Babich, et al., J Nucl Med, 1993, 34:1964; Babich, et al., Nucl Med Biol, 1995, 22:25; and WO 2022/123462, the entirely of each of which is incorporated herein by reference.

In one embodiment, the chelating agent (Z) is a structure in Table 1.

TABLE 1

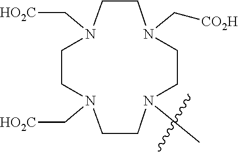 [SMILES: *N1CCN(CC(=O)O)CCN(CC(=O)O)CCN(CC(=O)O)CC1]

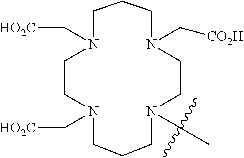 [SMILES: *N1CCCN(CC(=O)O)CCN(CC(=O)O)CCCN(CC(=O)O)CC1]

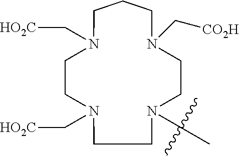 [SMILES: *N1CCN(CC(=O)O)CCCN(CC(=O)O)CCN(CC(=O)O)CC1]

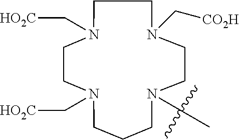 [SMILES: *N1CCCN(CC(=O)O)CCN(CC(=O)O)CCN(CC(=O)O)CC1]

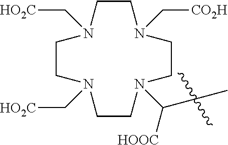 [SMILES: *C(C(=O)O)N1CCN(CC(=O)O)CCN(CC(=O)O)CCN(CC(=O)O)CC1]

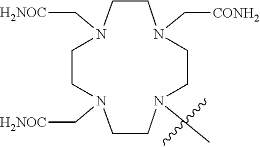 [SMILES: *N1CCN(CC(N)=O)CCN(CC(N)=O)CCN(CC(N)=O)CC1]

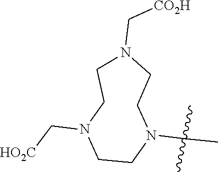 [SMILES: *N1CCN(CC(=O)O)CCN(CC(=O)O)CC1]

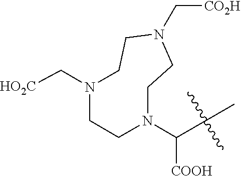 [SMILES: *C(C(=O)O)N1CCN(CC(=O)O)CCN(CC(=O)O)CC1]

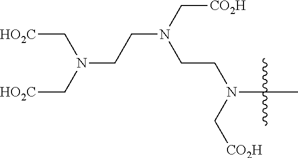 [SMILES: *N(CCN(CCN(CC(=O)O)CC(=O)O)CC(=O)O)CC(=O)O]

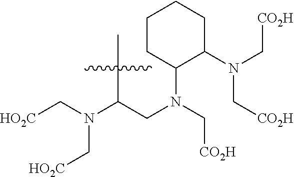 [SMILES: *C(CN(CC(=O)O)C1CCCCC1N(CC(=O)O)CC(=O)O)N(CC(=O)O)CC(=O)O]

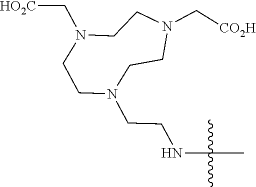 [SMILES: *NCCN1CCN(CC(=O)O)CCN(CC(=O)O)CC1]

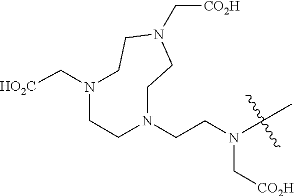 [SMILES: *N(CCN1CCN(CC(=O)O)CCN(CC(=O)O)CC1)CC(=O)O]

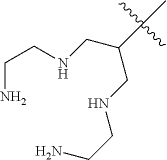 [SMILES: *C(CNCCN)CNCCN]

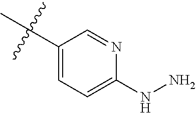 [SMILES: *c1ccc(NN)nc1]

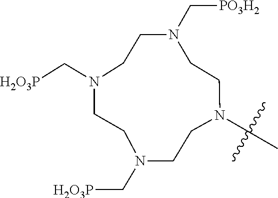

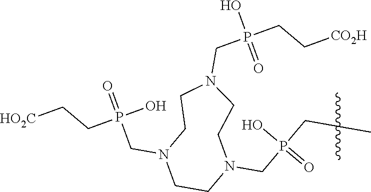 [SMILES: *CP(=O)(O)CN1CCN(CP(=O)(O)CCC(=O)O)CCN(CP(=O)(O)CCC(=O)O)CC1]

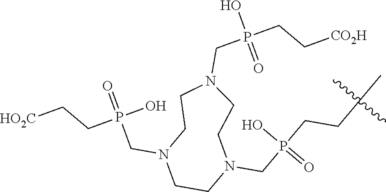 [SMILES: *CCP(=O)(O)CN1CCN(CP(=O)(O)CCC(=O)O)CCN(CP(=O)(O)CCC(=O)O)CC1]

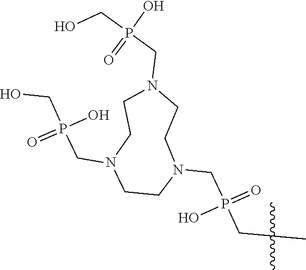 [SMILES: *CP(=O)(O)CN1CCN(CP(=O)(O)CO)CCN(CP(=O)(O)CO)CC1]

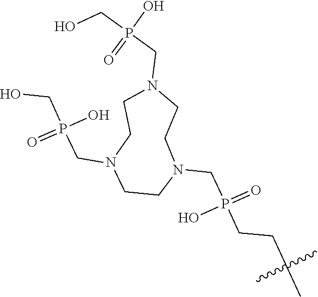 [SMILES: *CCP(=O)(O)CN1CCN(CP(=O)(O)CO)CCN(CP(=O)(O)CO)CC1]

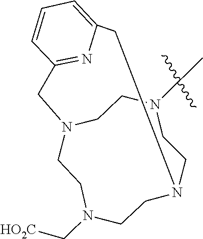 [SMILES: *N1CCN2CCN(CC(=O)O)CCN(CC1)Cc1cccc(n1)C2]

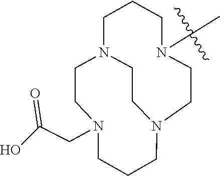 [SMILES: *N1CCCN2CCN(CCCN(CC(=O)O)CC2)CC1]

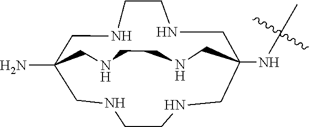

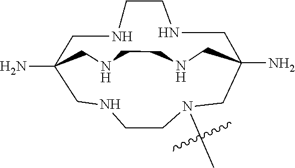

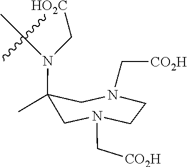 [SMILES: *N(CC(=O)O)C1(C)CN(CC(=O)O)CCN(CC(=O)O)C1]

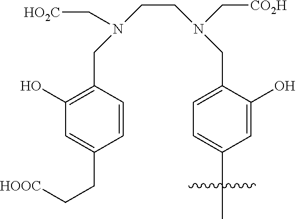 [SMILES: *c1ccc(CN(CCN(CC(=O)O)Cc2ccc(CCC(=O)O)cc2O)CC(=O)O)c(O)c1]

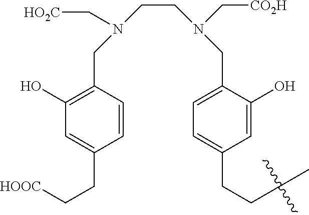 [SMILES: *CCc1ccc(CN(CCN(CC(=O)O)Cc2ccc(CCC(=O)O)cc2O)CC(=O)O)c(O)c1]

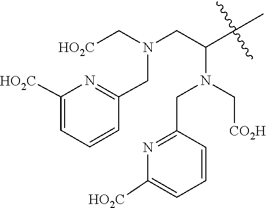 [SMILES: *C(CN(CC(=O)O)Cc1cccc(C(=O)O)n1)N(CC(=O)O)Cc1cccc(C(=O)O)n1]

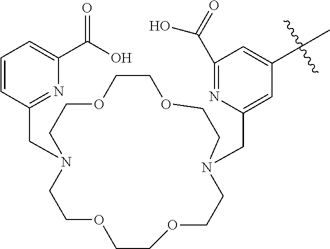 [SMILES: *c1cc(CN2CCOCCOCCN(Cc3cccc(C(=O)O)n3)CCOCCOCC2)nc(C(=O)O)c1]

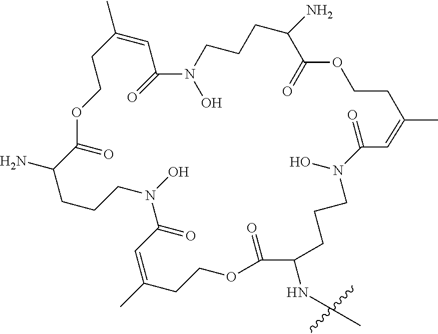 [SMILES: *NC1CCCN(O)C(=O)/C=C(/C)CCOC(=O)C(N)CCCN(O)C(=O)/C=C(/C)CCOC(=O)C(N)CCCN(O)C(=O)/C=C(/C)CCOC1=O]

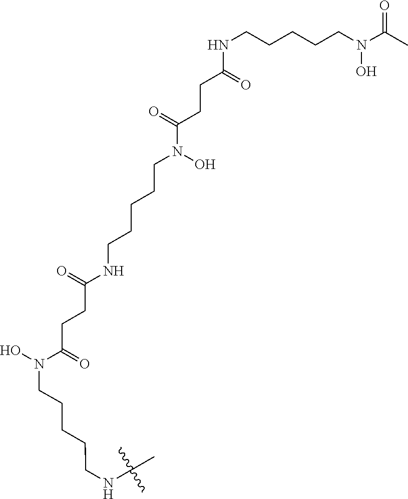 [SMILES: *NCCCCCN(O)C(=O)CCC(=O)NCCCCCN(O)C(=O)CCC(=O)NCCCCCN(O)C(C)=O]

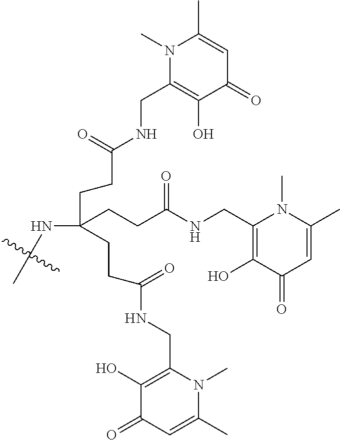 [SMILES: *NC(CCC(=O)NCc1c(O)c(=O)cc(C)n1C)(CCC(=O)NCc1c(O)c(=O)cc(C)n1C)CCC(=O)NCc1c(O)c(=O)cc(C)n1C]

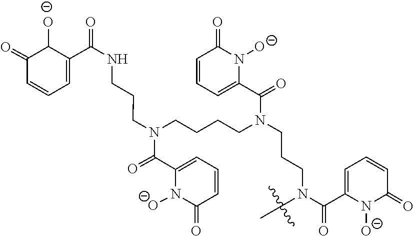 [SMILES: *N(CCCN(CCCCN(CCCNC(=O)C1=CC=CC(=O)C1[O-])C(=O)c1cccc(=O)n1[O-])C(=O)c1cccc(=O)n1[O-])C(=O)c1cccc(=O)n1[O-]]

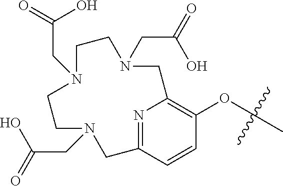 [SMILES: *Oc1ccc2nc1CN(CC(=O)O)CCN(CC(=O)O)CCN(CC(=O)O)C2]

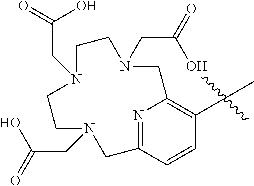 [SMILES: *c1ccc2nc1CN(CC(=O)O)CCN(CC(=O)O)CCN(CC(=O)O)C2]

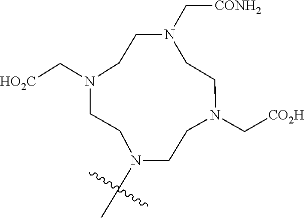 [SMILES: *N1CCN(CC(=O)O)CCN(CC(N)=O)CCN(CC(=O)O)CC1]

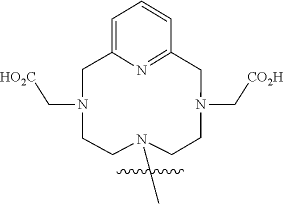 [SMILES: *N1CCN(CC(=O)O)Cc2cccc(n2)CN(CC(=O)O)CC1]

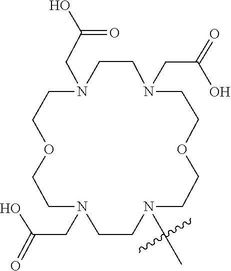 [SMILES: *N1CCOCCN(CC(=O)O)CCN(CC(=O)O)CCOCCN(CC(=O)O)CC1]

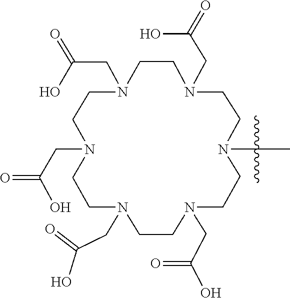 [SMILES: *N1CCN(CC(=O)O)CCN(CC(=O)O)CCN(CC(=O)O)CCN(CC(=O)O)CCN(CC(=O)O)CC1]

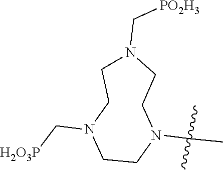

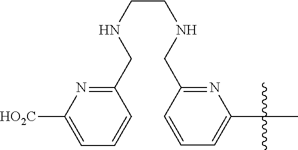 [SMILES: *c1cccc(CNCCNCc2cccc(C(=O)O)n2)n1]

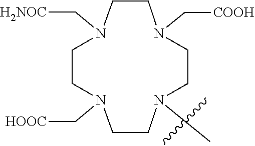 [SMILES: *N1CCN(CC(=O)O)CCN(CC(N)=O)CCN(CC(=O)O)CC1]

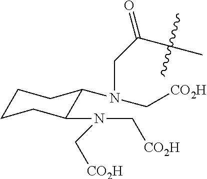 [SMILES: *C(=O)CN(CC(=O)O)C1CCCCC1N(CC(=O)O)CC(=O)O]

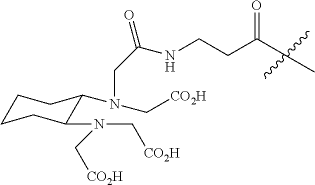 [SMILES: *C(=O)CCNC(=O)CN(CC(=O)O)C1CCCCC1N(CC(=O)O)CC(=O)O]

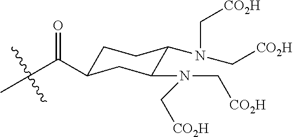 [SMILES: *C(=O)C1CCC(N(CC(=O)O)CC(=O)O)C(N(CC(=O)O)CC(=O)O)C1]

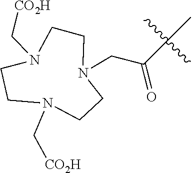 [SMILES: *C(=O)CN1CCN(CC(=O)O)CCN(CC(=O)O)CC1]

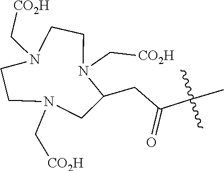 [SMILES: *C(=O)CC1CN(CC(=O)O)CCN(CC(=O)O)CCN1CC(=O)O]

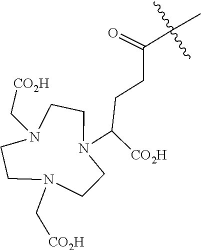 [SMILES: *C(=O)CCC(C(=O)O)N1CCN(CC(=O)O)CCN(CC(=O)O)CC1]

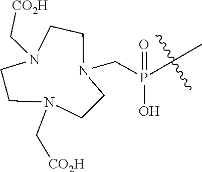 [SMILES: *P(=O)(O)CN1CCN(CC(=O)O)CCN(CC(=O)O)CC1]

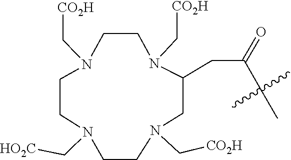 [SMILES: *C(=O)CC1CN(CC(=O)O)CCN(CC(=O)O)CCN(CC(=O)O)CCN1CC(=O)O]

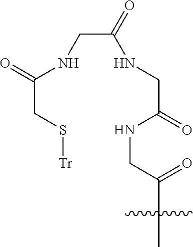

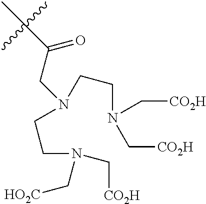 [SMILES: *C(=O)CN(CCN(CC(=O)O)CC(=O)O)CCN(CC(=O)O)CC(=O)O]

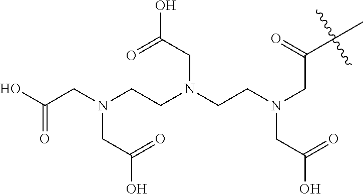 [SMILES: *C(=O)CN(CCN(CCN(CC(=O)O)CC(=O)O)CC(=O)O)CC(=O)O]

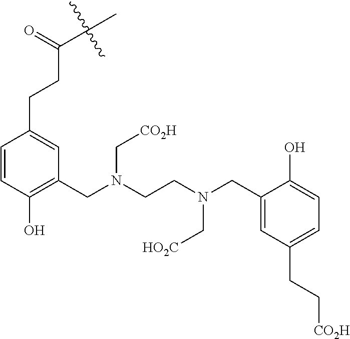 [SMILES: *C(=O)CCc1ccc(O)c(CN(CCN(CC(=O)O)Cc2cc(CCC(=O)O)ccc2O)CC(=O)O)c1]

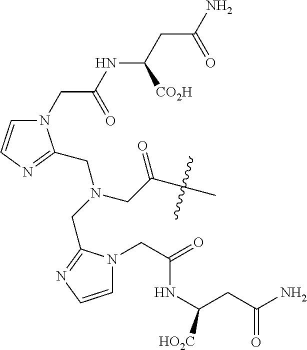 [SMILES: *C(=O)CN(Cc1nccn1CC(=O)N[C@@H](CC(N)=O)C(=O)O)Cc1nccn1CC(=O)N[C@@H](CC(N)=O)C(=O)O]

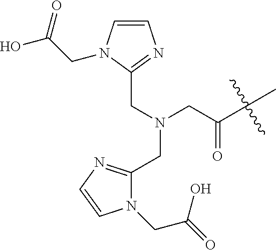 [SMILES: *C(=O)CN(Cc1nccn1CC(=O)O)Cc1nccn1CC(=O)O]

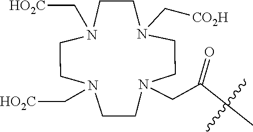 [SMILES: *C(=O)CN1CCN(CC(=O)O)CCN(CC(=O)O)CCN(CC(=O)O)CC1]

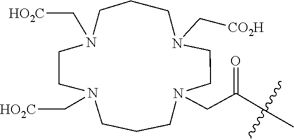 [SMILES: *C(=O)CN1CCCN(CC(=O)O)CCN(CC(=O)O)CCCN(CC(=O)O)CC1]

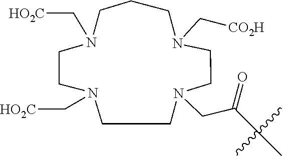 [SMILES: *C(=O)CN1CCN(CC(=O)O)CCCN(CC(=O)O)CCN(CC(=O)O)CC1]

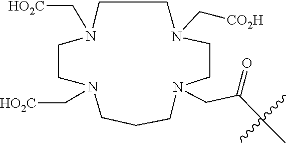 [SMILES: *C(=O)CN1CCCN(CC(=O)O)CCN(CC(=O)O)CCN(CC(=O)O)CC1]

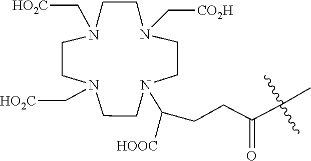 [SMILES: *C(=O)CCC(C(=O)O)N1CCN(CC(=O)O)CCN(CC(=O)O)CCN(CC(=O)O)CC1]

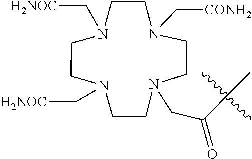 [SMILES: *C(=O)CN1CCN(CC(N)=O)CCN(CC(N)=O)CCN(CC(N)=O)CC1]

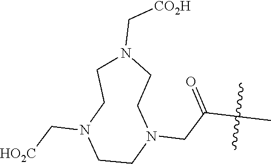 [SMILES: *C(=O)CN1CCN(CC(=O)O)CCN(CC(=O)O)CC1]

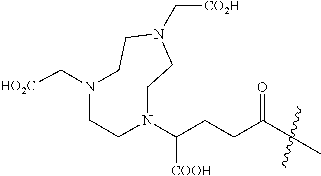 [SMILES: *C(=O)CCC(C(=O)O)N1CCN(CC(=O)O)CCN(CC(=O)O)CC1]

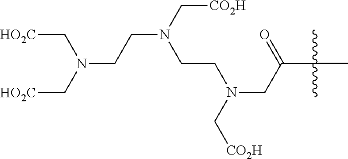 [SMILES: *C(=O)CN(CCN(CCN(CC(=O)O)CC(=O)O)CC(=O)O)CC(=O)O]

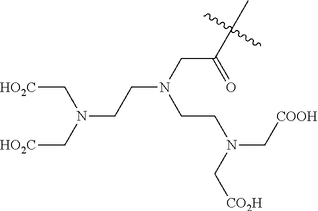 [SMILES: *C(=O)CN(CCN(CC(=O)O)CC(=O)O)CCN(CC(=O)O)CC(=O)O]

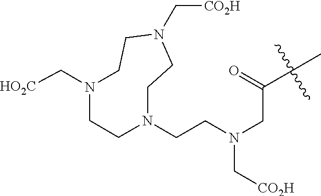 [SMILES: *C(=O)CN(CCN1CCN(CC(=O)O)CCN(CC(=O)O)CC1)CC(=O)O]

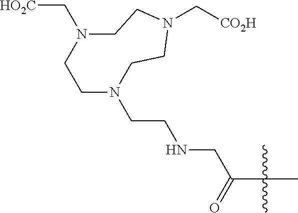 [SMILES: *C(=O)CNCCN1CCN(CC(=O)O)CCN(CC(=O)O)CC1]

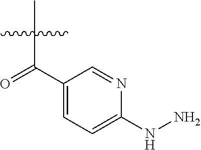 [SMILES: *C(=O)c1ccc(NN)nc1]

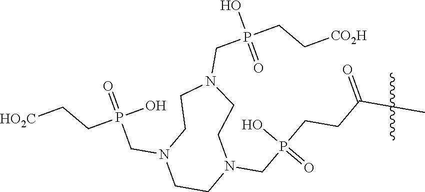 [SMILES: *C(=O)CCP(=O)(O)CN1CCN(CP(=O)(O)CCC(=O)O)CCN(CP(=O)(O)CCC(=O)O)CC1]

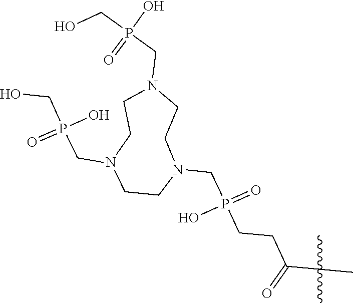 [SMILES: *C(=O)CCP(=O)(O)CN1CCN(CP(=O)(O)CO)CCN(CP(=O)(O)CO)CC1]

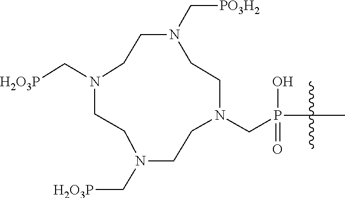

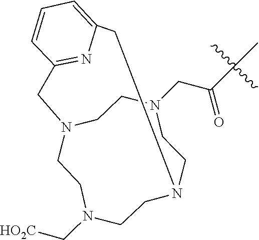 [SMILES: *C(=O)CN1CCN2CCN(CC(=O)O)CCN(CC1)Cc1cccc(n1)C2]

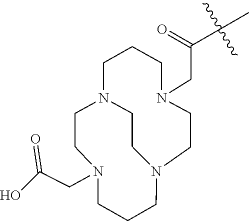 [SMILES: *C(=O)CN1CCCN2CCN(CCCN(CC(=O)O)CC2)CC1]

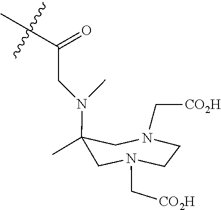 [SMILES: *C(=O)CN(C)C1(C)CN(CC(=O)O)CCN(CC(=O)O)C1]

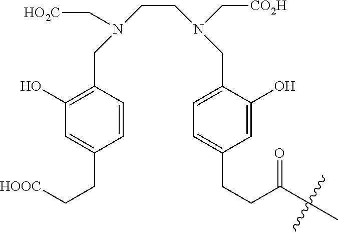 [SMILES: *C(=O)CCc1ccc(CN(CCN(CC(=O)O)Cc2ccc(CCC(=O)O)cc2O)CC(=O)O)c(O)c1]

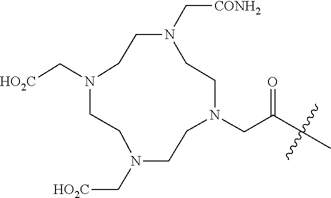 [SMILES: *C(=O)CN1CCN(CC(N)=O)CCN(CC(=O)O)CCN(CC(=O)O)CC1]

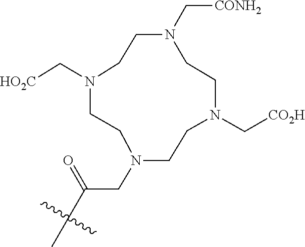 [SMILES: *C(=O)CN1CCN(CC(=O)O)CCN(CC(N)=O)CCN(CC(=O)O)CC1]

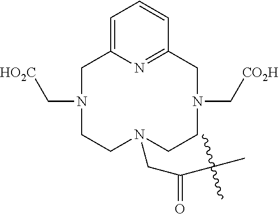 [SMILES: *C(=O)CN1CCN(CC(=O)O)Cc2cccc(n2)CN(CC(=O)O)CC1]

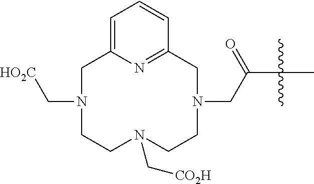 [SMILES: *C(=O)CN1CCN(CC(=O)O)CCN(CC(=O)O)Cc2cccc(n2)C1]

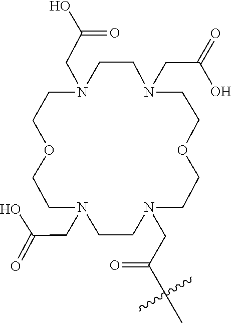 [SMILES: *C(=O)CN1CCOCCN(CC(=O)O)CCN(CC(=O)O)CCOCCN(CC(=O)O)CC1]

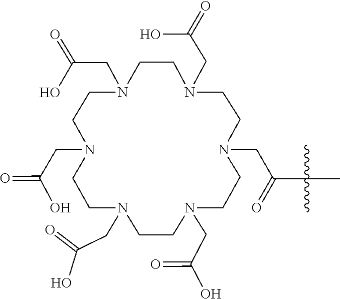 [SMILES: *C(=O)CN1CCN(CC(=O)O)CCN(CC(=O)O)CCN(CC(=O)O)CCN(CC(=O)O)CCN(CC(=O)O)CC1]

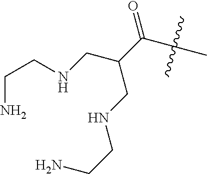 [SMILES: *C(=O)C(CNCCN)CNCCN]

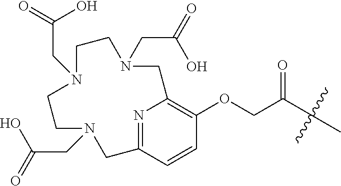 [SMILES: *C(=O)COc1ccc2nc1CN(CC(=O)O)CCN(CC(=O)O)CCN(CC(=O)O)C2]

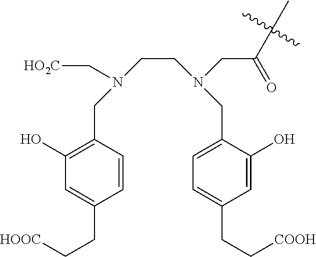 [SMILES: *C(=O)CN(CCN(CC(=O)O)Cc1ccc(CCC(=O)O)cc1O)Cc1ccc(CCC(=O)O)cc1O]

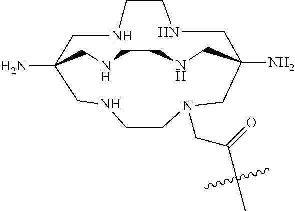

In one embodiment, Z is

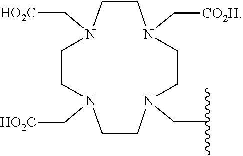 [SMILES: *CN1CCN(CC(=O)O)CCN(CC(=O)O)CCN(CC(=O)O)CC1]

In one embodiment, Z is

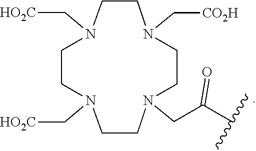 [SMILES: *C(=O)CN1CCN(CC(=O)O)CCN(CC(=O)O)CCN(CC(=O)O)CC1]

In one embodiment, Z-L-P¹-G³ (or a sub-formula thereof) is

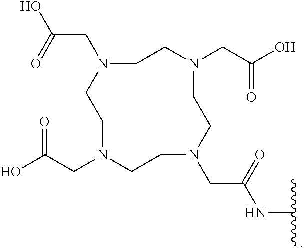 [SMILES: *NC(=O)CN1CCN(CC(=O)O)CCN(CC(=O)O)CCN(CC(=O)O)CC1]

In one embodiment, Z-L-P¹-G³ (or a sub-formula thereof) is

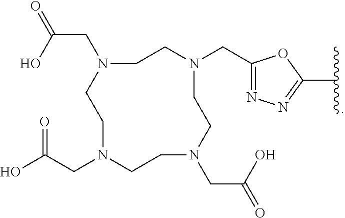 [SMILES: *c1nnc(CN2CCN(CC(=O)O)CCN(CC(=O)O)CCN(CC(=O)O)CC2)o1]

In one embodiment, Z is a contrast agent. In one embodiment, the contrast agent comprises a paramagnetic agent. In one embodiment, the paramagnetic agent comprises paramagnetic nanoparticles.

In one embodiment, Z is a cytostatic and/or cytotoxic agent. In one embodiment, the cytostatic and/or cytotoxic agent is selected from the group consisting of alkylating substances, anti-metabolites, antibiotics, epothilones, nuclear receptor agonists and antagonists, anti-androgenes, anti-estrogens, platinum compounds, hormones and antihormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneimenes, methylmelamines, trazines, vinca alkaloids, pyrimidine analogs, purine analogs, alkylsulfonates, folic acid analogs, anthracendiones, substituted urea, methylhydrazin derivatives, such as acediasulfone, aclarubicine, a-amanitin, ambazone, aminoglutethimide, L-asparaginase, monomethyl auristatin E, azathioprine, bleomycin, busulfan, calcium folinate, carboplatin, capecitabine, carmustine, celecoxib, chlorambucil, cis-platin, cladribine, cyclophosphamide, cytarabine, dacarbazine, dactinomycin dapsone, daunorubicin, dibrompropamidine, diethylstilbestrole, docetaxel, dolastatin 10 and 15, doxorubicin, enediynes, epirubicin, epothilone B, epothilone D, estramucin phosphate, estrogen, ethinylestradiole, etoposide, flavopiridol, floxuridine, fludarabine, fluorouracil, fluoxymesterone, flutamide fosfestrol, furazolidone, gemcitabine, gonadotropin releasing hormone analog, hexamethylmelamine, hydroxycarbamide, hydroxymethylnitrofurantoin, hydroxyprogesteronecaproat, hydroxyurea, idarubicin, idoxuridine, ifosfamide, interferon a, irinotecan, leuprolide, lomustine, lurtotecan, mafenide sulfate olamide, mechlorethamine, medroxyprogesterone acetate, megastrolacetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, scriflavinium chloride, semustine streptozocine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, tinidazole, topotecan, triaziquone, treosulfan, trimethoprim, trofosfamide, UCN-01, vinblastine, vincristine, vindesine, vinblastine, vinorelbine, zorubicin, or their respective derivatives or analogs thereof and combinations thereof.

In one embodiment, the cytostatic and/or cytotoxic agent is selected from the group consisting of doxorubicin, α-amanitin and monomethyl auristatin E. In one embodiment, Z is doxorubicin.

In one embodiment, Z is a cytokine. In one embodiment, the cytokine is a chemokine molecule. In one embodiment, the chemokine molecule is selected from the group consisting of CXCL9, CXCL10 and CX3CL1. In one embodiment, Z is CXCL9. In one embodiment, Z is CXCL10. In one embodiment, Z is CX3CL1.

In one embodiment, Z is an immunomodulatory molecule. In one embodiment, the immunomodulatory molecule is selected from the group consisting of CXCL1, CXCL2, CXCL3, CXCL4, CXCL5, CXCL6, CXCL7, CXCL8, CXCL11, CXCL12, CXCL13, CXCL14, CXCL15, CXCL16, CXCL17, CX3CL1, CCL1, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, interleukin-2, interferon alpha and interferon gamma. In one embodiment, the immunomodulatory molecule is selected from the group consisting of CXCL3, interleukin-2 and CCL8. In one embodiment, Z is interleukin-2.

In one embodiment, Z is an amphiphilic substance. In one embodiment, the amphiphilic substance is selected from the group consisting of a lipid, a phospholipid and other highly lipophilic moiety conjugated to a polar group such as an ammonium ion or inositol triphosphate. In one embodiment, the lipid is selected from the group consisting of saccharolipids, prenol lipids, sterol lipids, glycerolipids, polyketides and fatty acids and the phospholipid is selected from the group consisting of plasmalogens, sphingo lipids, phophatidates and phosphoinositides. In one embodiment, the amphiphilic substance is a lipid or a phospholipid. In one embodiment, the amphiphilic substance is N-PEGylated 1,2-disteaorylglycero-3-phosphoethanolamine. In one embodiment, Z is a lipid. In one embodiment, Z is a phospholipid. In one embodiment, Z is N-PEGylated 1,2-disteaorylglycero-3-phosphoethanolamine.

In one embodiment, Z is a nucleic acid. In one embodiment, the nucleic acid is selected from the group consisting of DNA, RNA, siRNA, mRNA, PNA and cDNA. In one embodiment, the nucleic acid encodes a cytokine and/or an immunomodulatory molecule provided herein. In one embodiment, the nucleic acid is a siRNA or PNA.

In one embodiment, Z is a viral structural protein. In one embodiment, the viral structural protein is of a virus selected from the group consisting of
(i) double-stranded DNA virus, such as Myoviridae, Siphoviridae, Podoviridae, Herpesviridae, Adenoviridae, Baculoviridae, Papillomaviridae, Polydnaviridae, Polyomaviridae, Poxviridae;
(ii) single-stranded DNA virus, such as Anelloviridae, Inoviridae, Parvoviridae;
(iii) double-stranded RNA virus, such as Reoviridae;
(iv) single-stranded RNA virus, such as Coronaviridae, Picomaviridae, Caliciviridae, Togaviridae, Flaviviridae, Astroviridae, Arteriviridae, Hepeviridae;
(v) negative-sense single-stranded RNA virus, such as Arenaviridae, Filoviridae, Paramyxoviridae, Rhabdoviridae, Bunyaviridae, Orthomyxoviridae, Bomaviridae;
(vi) single-stranded RNA reverse transcribing virus, such as Retroviridae;
(vii) double-stranded DNA reverse transcribing virus, such as Caulimoviridae, Hepadnaviridae.

In one embodiment, the viral structural protein, such as VCP is derived from a virus selected from the group consisting of double-stranded DNA virus, such as Myoviridae, Siphoviridae, Podoviridae, Herpesviridae, Adenoviridae, Baculoviridae, Papillomaviridae, Polydnaviridae, Polyomaviridae, Poxviridae; single-stranded DNA virus, such as Anelloviridae, Inoviridae, Parvoviridae; double-stranded RNA virus, such as Reoviridae; single-stranded RNA virus, such as Coronaviridae, Picomaviridae, Caliciviridae, Togaviridae, Flaviviridae, Astroviridae, Arteriviridae, Hepeviridae; negative-sense single-stranded RNA vims, such as Arenaviridae, Filoviridae, Paramyxoviridae, Rhabdoviridae, Bunyaviridae, Orthomyxoviridae, Bomaviridae; single-stranded RNA reverse transcribing vims, such as Retroviridae; double-stranded DNA reverse transcribing vims, such as Caulimoviridae, Hepadnaviridae. In one embodiment, the VCP is from a family of the Parvoviridae, such as from adeno-associated vims. In one embodiment, the VCP is human AAV, bovine AAV, caprine AAV, avian AAV, canine parvovirus (CPV), mouse parvovirus; minute vims of mice (MVM); parvovirus B19 (B19); parvovirus Hl (Hl); human bocavims (HBoV); feline panleukopenia vims (FPV); or goose parvovirus (GPV). In one embodiment, the VCP is from a certain AAV-serotype, such as AAV-1, AAV-2, AAV-2-AAV-3 hybrid, AAV-3a, AAV-3b, AAV-4, AAV-5, AAV-6, AAV-6.2, AAV-7, AAV-8, AAV-9, AAV-10, AAVrh.10, AAV-11, AAV-12, AAV-13 or AAVrh32.33. In one embodiment, the VCP is from AAV-2 or a variant thereof that is capable of assembling into a VLP.

In one embodiment, Z is protein. In one embodiment, the protein is selected from the group consisting of a membrane bound protein and unbound protein. Examples of the protein include but are not limited to CEA, CA19-9, Macrophage Migration Inhibition Factor (MIF), IL-8 (interleukin 8), AXL, MER and c-MET.

In one embodiment, Z is biotin. In one embodiment, provided herein is a liposome comprising a compound provided herein, wherein Z is an amphiphilic substance.

The liposomes provided herein can be various types of liposomes, for example, as described in Alavi et al., Adv Pharm Bull, 2017. In one embodiment, the liposomes provided herein is a stealth liposome. Stealth liposomes are known in the art and are for example reviewed by Immordino et al., Int J Nanonedicine, 2006.

The liposome provided herein can be positively charged, negatively charged or neutral liposomes. The charge of a liposome is determined by the lipid composition and is the average of all charges of the lipids comprised in the liposome. For example, a mixture of a negatively charged phospholipid and cholesterol will yield a negatively charged liposome.

In one embodiment, lipids/phospholipids to be used in liposomes include but are not limited to glycerides, glycerophospholipides, glycerophosphinolipids, glycerophosphonolipids, sulfolipids, sphingolipids, phospholipids, isoprenolides, steroids, stearines, steroles and carbohydrate containing lipids.

In one embodiment, the negatively charged lipid/phospholipid is selected from the group consisting of phosphatidylserine (PS), phosphatidylglycerol (PG) and phosphatidic acid (PA). PS and PG are collective terms for lipids sharing a similar phosphatidylserine and phosphatidylglycerol, respectively, head group. However, many different apolar residues can be attached to these head groups. Thus, PSs and PGs isolated from different natural sources vary substantially in the length, composition and/or chemical structure of the attached apolar residues and naturally occurring PS and PG usually is a mixture of PSs and PGs with different apolar residues.

In one embodiment, the PS employed in the liposomes provided herein is selected from the group consisting of palmitoyloleoylphosphatidylserine, palmitoyllinoeoylphosphatidylserine, palmitoylarachidonoylphosphatidylserine, palmitoyldocosahexaenoylphosphatidylserine, stearoyloleoylphosphatidylserine, stearoyllinoleoylphosphatidylserine, stearoyl-arachidonoylphosphatidylserine, stearoyldocosahexaenoylphosphatidylserine, dicaprylphosphatidylserine, dilauroylphosphatidylserine, dimyristoylphosphatidylserine, diphytanoylphosphatidylserine, diheptadecanoylphosphatidylserine, dioleoylphosphatidylserine, dipalmitoylphosphatidylserine, distearoylphosphatidylserine, dilinoleoylphosphatidylserine dierucoylphosphatidylserine, didocosahexaenoyl-phospahtidylserine, PS from brain, and PS from soy bean; in one embodiment, dioleoylphosphatidylserine.

In one embodiment, the PG employed in the liposome provided herein is selected from the group consisting of palmitoyloleoylphosphatidylglycerol, palmitoyllinoleoylphosphatidylglycerol, palmitoylarachidonoylphosphatidylglycerol, palmitoyldocosahexaenoylphosphatidylglycerol, stearoyloleoylphosphatidylglycerol, stearoyllinoleoylphosphatidylglycerol, stearoylarachidonoylphosphatidylglycerol, stearoyldocosa-hexaenoylphosphatidylglycerol, dicaprylphosphatidylglycerol dilauroylphosphatidylglycerol, diheptadecanoylphosphatidylglycerol, diphytanoyl-phosphatidylglycerol, dimyristoylphosphatidylglycerol, dipalmitoylphosphatidylglycerol, dielaidoylphosphatidylglycerol, distearoylphosphatidylglycerol, dioleoylphosphatidylglycerol, dilinoeoylphosphatidylglycerol, diarachidonoylphosphatidylglycerol, docosahexaenoylphosphatidylglycerol, and PG from egg; in one embodiment, dioleoylphosphatidylglycerol.

Similar to PS and PG, PE is also a generic term for lipids sharing a phosphatidylethanolamine head group. In one embodiment, the PE is selected from the group consisting of palmitoyloleoylphosphatidylethanolamine, palmitoyllinoleoylphosphatidylethanolamine, palmitoylarachidonoylphosphatidylethanolamine, palmitoyldocosahexaenoylphosphatidylethanolamine, stearoyloleoylphosphatidylethanolamine, stearoyllinoleoylphosphatidylethanolamine, stearoylarachidonoylphosphatidylethanolamine, stearoyldocosahexaenoylphosphatidylethanolamine, dilauroylphosphatidylethanolamine, dimyristoylphosphatidylethanolamine, diphytanoylphosphatidylethanolamine, dipalmitoylphosphatidylethanolamine, diheptadecanoylphosphatidylethanolamine, distearoylphosphatidylethanolamine, dielaidoylphosphatidylethanolamine, diarachidonoylphosphatidylethanolamine, docosahexaenoylphosphatidylethanolamine, PE from bacteria, PE from heart, PE from brain, PE from liver, PE from egg, and PE from soybean, in one embodiment, 1,2-diacyl-sn-glycero-3-PE, 1-acyl-2-acyl-sn-glycero-3-PE, 1,2-dipalmitoyl-PE and/or 1,2-dilauroyl-sn-glycero-3-PE (DLPE).

The liposome provided herein can comprise at least one further component selected from the group consisting of an adjuvant, additive, and auxiliary substance. In one embodiment, adjuvants are selected from the group consisting of unmethylated DNA, such as unmethylated DNA comprising CpG dinucleotides (CpG motif), such as CpG ODN with phosphorothioate (PTO) backbone (CpG PTO ODN) or phosphodiester (PO) backbone (CpG PO ODN); bacterial products from the outer membrane of Gram-negative bacteria, such as monophosphoryl lipid A (MPLA), lipopolysaccharides (LPS), muramyl dipeptides and derivatives thereof; synthetic lipopeptide derivatives, such as ParmCys; lipoarabinomannan; peptidoglycan; zymosan; heat shock proteins (HSP), such as HSP 70; dsRNA and synthetic derivatives thereof, such as Poly Epoly C; polycationic peptides, such as poly-L-arginine; taxol; fibronectin; flagellin; imidazoquinoline; cytokines with adjuvant activity, such as GM-CSF, interleukin-(IL-)2, IL-6, IL-7, IL-18, type I and II, interferons, such as interferon-gamma, TNF-alpha; 25-dihydroxyvitamin D3 (calcitriol); synthetic oligopeptides, such as MHCII-presented peptides; gel-like precipitates of aluminum hydroxide (alum). In one embodiment, adjuvants, which can be comprised in the liposome provided herein are selected from the group unmethylated DNA, such as unmethylated DNA comprising CpG dinucleotides (CpG motif), such as CpG ODN with phosphorothioate (PTO) backbone (CpG PTO ODN) or phosphodiester (PO) backbone (CpG PO ODN), bacterial products from the outer membrane of Gram-negative bacteria, such as monophosphoryl lipid A (MPLA) and synthetic lipopeptide derivatives, such as ParmCys.

As used herein, and unless otherwise specified, the term “additive” comprises substances, which stabilize any component of the liposome or of the liquid medium like, for example, antioxidants, radical scavengers or the like. In one embodiment, stabilizers are selected from the group consisting of a-tocopherol or carbohydrates, such as glucose, sorbitol, sucrose, maltose, trehalose, lactose, cellubiose, raffmose, maltotriose, or dextran. The stabilizers can be comprised in the lipid membranes of the liposomes, the interior of the liposomes and/or within the liquid medium surrounding the liposomes.

Liposomes provided herein can have a diameter between 10 and 1000 nm. In one embodiment, they have a diameter of between 30 and 800 nm, between 40 and 500 nm, between 50 and 300 nm, or between 100 and 200 nm. The diameter of the liposomes can be affected, for example, by extrusion of the liposomal composition through sieves or meshes with a known pore size. This and further methods of controlling the size of liposomes are known in the art and are described, for example, in Mayhew et al. (1984) Biochim. Biophys. Acta 775:169-174 or Olson et al. (1979) Biochim. Biophys. Acta 557:9-23.

In one embodiment, the liposome or the mixture of liposomes provided herein are comprised in a liquid medium. As used herein, and unless otherwise specified, the term “liquid medium” comprises all biocompatible, physiological acceptable liquids and liquid compositions such as FLO, aqueous salt solutions, and buffer solutions like, for example, PBS, Ringer solution and the like.

In one embodiment, the liposome is loaded with a substance selected from the group consisting of an agent and a nucleic acid.

In one embodiment, the agent that the liposome is loaded with is a cytostatic and/or cytotoxic agent provided herein. In one embodiment, the nucleic acid that the liposome is loaded with is a nucleic acid provided herein. A variety of methods are available in the art to “load” a liposome with a given therapeutic agent. In one embodiment, the therapeutic agent(s) is (are) admixed with the lipid components during formation of the liposomes. Other passive loading methods include dehydration-rehydration (Kirby & Gregoriadis (1984) Biotechnology 2:979), reverse-phase evaporation (Szoka & Papahadjopoulos (1978) Proc. Natl. Acad. Sci. USA 75:4194), or detergent-depletion (Milsmann et al. (1978) Biochim. Biophys. Acta 512:147-155). Other methodologies for encapsulating therapeutic agents include so called “remote loading” or “active loading” in which due to a gradient, for example, a pH or salt gradient between the exterior and the interior of a preformed liposome the therapeutic agent is transported into the liposome along the gradient (see, for example, Cheung et al. (1998) Biochim. Biophys. Acta 1414:205-216; Cullis et al. (1991) Trends Biotechnol. 9:268-272; Mayer et al. (1986) Chem. Phys. Lipids 40:333-345).

In one embodiment, the compound is a compound in Table 2 or Table 2A, or a stereoisomer, a mixture of stereoisomers, a tautomer, or a pharmaceutically acceptable salt thereof.

TABLE 2

| | |
|---|---|
| 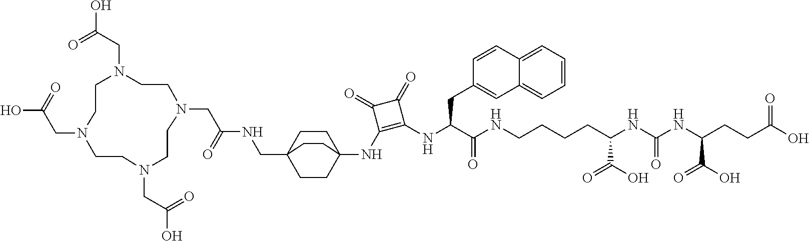 [SMILES: O=C(O)CC[C@H](NC(=O)N[C@@H](CCCCNC(=O)[C@H](Cc1ccc2ccccc2c1)Nc1c(NC23CCC(CNC(=O)CN4CCN(CC(=O)O)CCN(CC(=O)O)CCN(CC(=O)O)CC4)(CC2)CC3)c(=O)c1=O)C(=O)O)C(=O)O] | E1 |
| 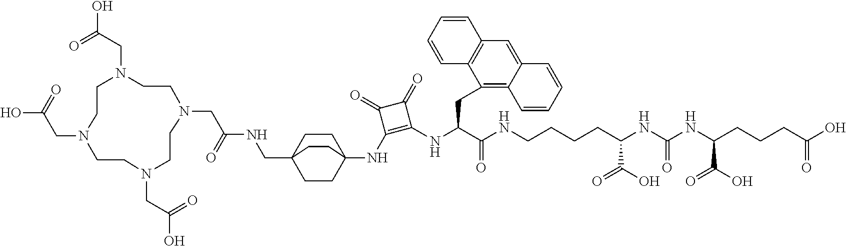 [SMILES: O=C(O)CCC[C@H](NC(=O)N[C@@H](CCCCNC(=O)[C@H](Cc1c2ccccc2cc2ccccc12)Nc1c(NC23CCC(CNC(=O)CN4CCN(CC(=O)O)CCN(CC(=O)O)CCN(CC(=O)O)CC4)(CC2)CC3)c(=O)c1=O)C(=O)O)C(=O)O] | E2 |
| 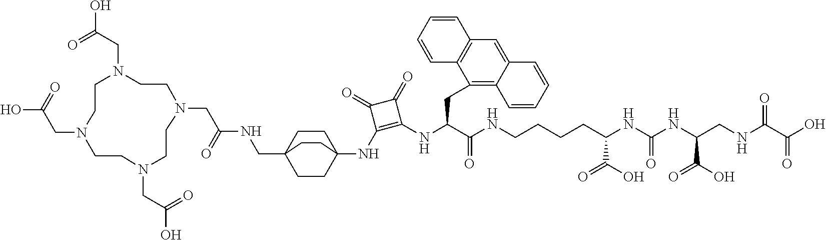 [SMILES: O=C(O)CN1CCN(CC(=O)O)CCN(CC(=O)NCC23CCC(Nc4c(N[C@@H](Cc5c6ccccc6cc6ccccc56)C(=O)NCCCC[C@H](NC(=O)N[C@@H](CNC(=O)C(=O)O)C(=O)O)C(=O)O)c(=O)c4=O)(CC2)CC3)CCN(CC(=O)O)CC1] | E3 |
| 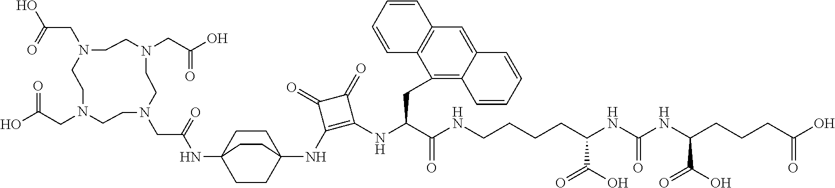 [SMILES: O=C(O)CCC[C@H](NC(=O)N[C@@H](CCCCNC(=O)[C@H](Cc1c2ccccc2cc2ccccc12)Nc1c(NC23CCC(NC(=O)CN4CCN(CC(=O)O)CCN(CC(=O)O)CCN(CC(=O)O)CC4)(CC2)CC3)c(=O)c1=O)C(=O)O)C(=O)O] | E4 |
| 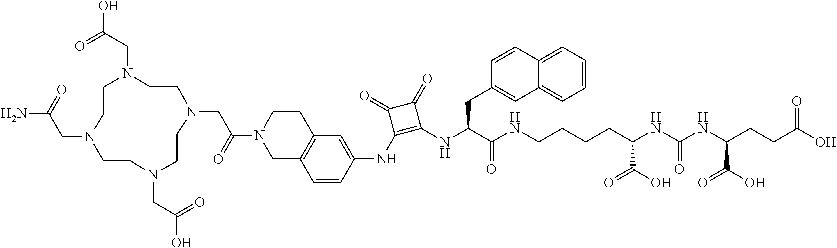 [SMILES: NC(=O)CN1CCN(CC(=O)O)CCN(CC(=O)N2CCc3cc(Nc4c(N[C@@H](Cc5ccc6ccccc6c5)C(=O)NCCCC[C@H](NC(=O)N[C@@H](CCC(=O)O)C(=O)O)C(=O)O)c(=O)c4=O)ccc3C2)CCN(CC(=O)O)CC1] | E5 |
| 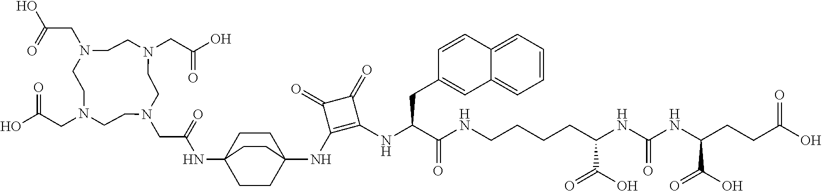 [SMILES: O=C(O)CC[C@H](NC(=O)N[C@@H](CCCCNC(=O)[C@H](Cc1ccc2ccccc2c1)Nc1c(NC23CCC(NC(=O)CN4CCN(CC(=O)O)CCN(CC(=O)O)CCN(CC(=O)O)CC4)(CC2)CC3)c(=O)c1=O)C(=O)O)C(=O)O] | E6 |
| 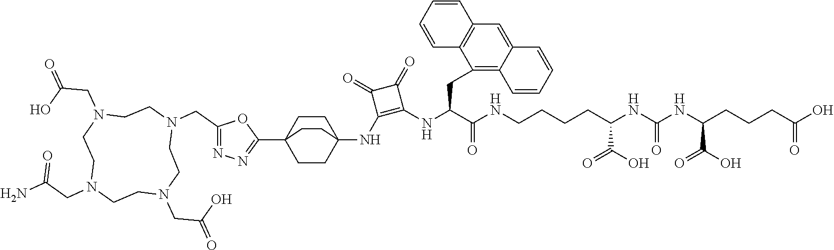 [SMILES: NC(=O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CCCC(=O)O)C(=O)O)C(=O)O)c(=O)c5=O)(CC3)CC4)o2)CCN(CC(=O)O)CC1] | E7 |
| 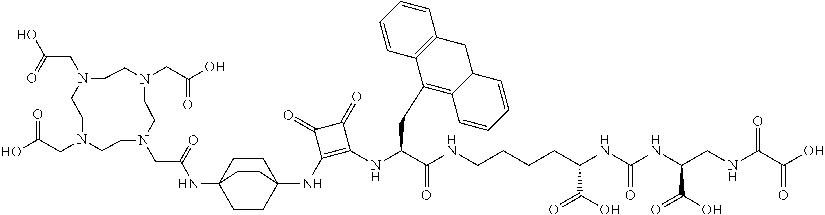 [SMILES: O=C(O)CN1CCN(CC(=O)O)CCN(CC(=O)NC23CCC(Nc4c(N[C@@H](CC5=C6C=CC=CC6Cc6ccccc65)C(=O)NCCCC[C@H](NC(=O)N[C@@H](CNC(=O)C(=O)O)C(=O)O)C(=O)O)c(=O)c4=O)(CC2)CC3)CCN(CC(=O)O)CC1] | E8 |
| 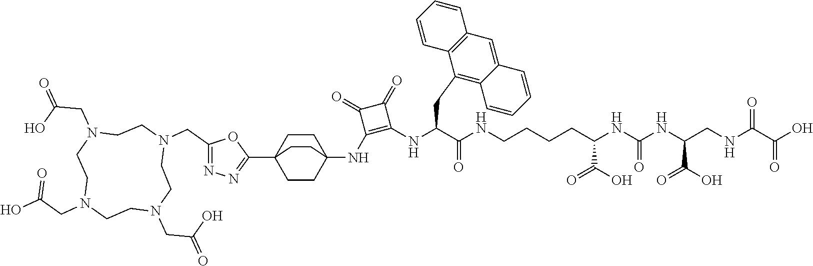 [SMILES: O=C(O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CNC(=O)C(=O)O)C(=O)O)C(=O)O)c(=O)c5=O)(CC3)CC4)o2)CCN(CC(=O)O)CC1] | E9 |
| 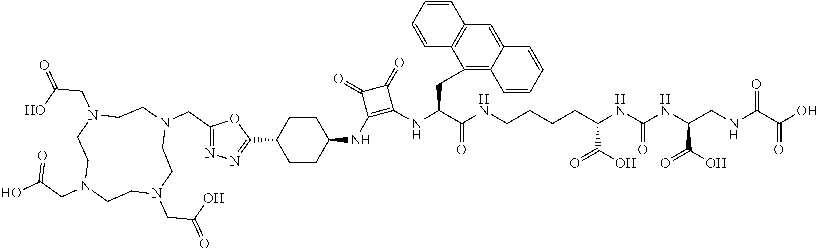 [SMILES: O=C(O)CN1CCN(CC(=O)O)CCN(Cc2nnc([C@H]3CC[C@H](Nc4c(N[C@@H](Cc5c6ccccc6cc6ccccc56)C(=O)NCCCC[C@H](NC(=O)N[C@@H](CNC(=O)C(=O)O)C(=O)O)C(=O)O)c(=O)c4=O)CC3)o2)CCN(CC(=O)O)CC1] | E10 |
| 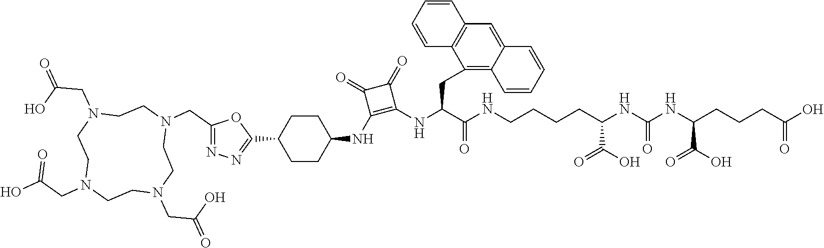 [SMILES: O=C(O)CCC[C@H](NC(=O)N[C@@H](CCCCNC(=O)[C@H](Cc1c2ccccc2cc2ccccc12)Nc1c(N[C@H]2CC[C@H](c3nnc(CN4CCN(CC(=O)O)CCN(CC(=O)O)CCN(CC(=O)O)CC4)o3)CC2)c(=O)c1=O)C(=O)O)C(=O)O] | E11 |
| 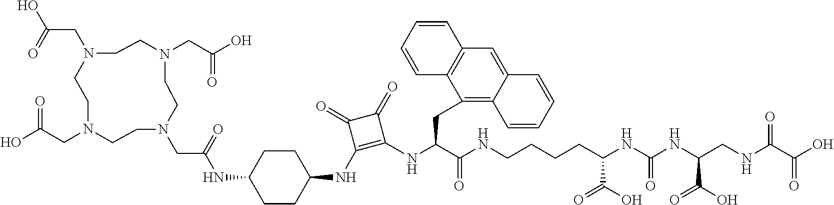 [SMILES: O=C(O)CN1CCN(CC(=O)O)CCN(CC(=O)N[C@H]2CC[C@H](Nc3c(N[C@@H](Cc4c5ccccc5cc5ccccc45)C(=O)NCCCC[C@H](NC(=O)N[C@@H](CNC(=O)C(=O)O)C(=O)O)C(=O)O)c(=O)c3=O)CC2)CCN(CC(=O)O)CC1] | E12 |
| 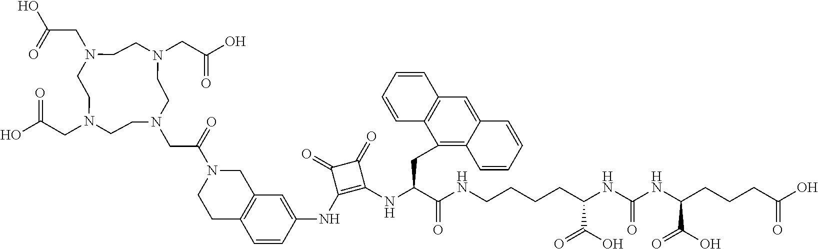 [SMILES: O=C(O)CCC[C@H](NC(=O)N[C@@H](CCCCNC(=O)[C@H](Cc1c2ccccc2cc2ccccc12)Nc1c(Nc2ccc3c(c2)CN(C(=O)CN2CCN(CC(=O)O)CCN(CC(=O)O)CCN(CC(=O)O)CC2)CC3)c(=O)c1=O)C(=O)O)C(=O)O] | E13 |
| 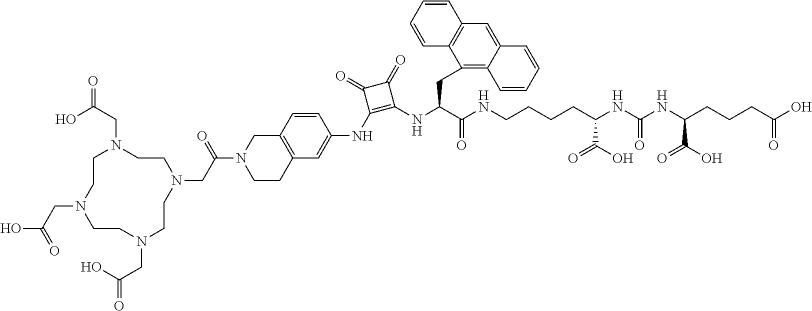 [SMILES: O=C(O)CCC[C@H](NC(=O)N[C@@H](CCCCNC(=O)[C@H](Cc1c2ccccc2cc2ccccc12)Nc1c(Nc2ccc3c(c2)CCN(C(=O)CN2CCN(CC(=O)O)CCN(CC(=O)O)CCN(CC(=O)O)CC2)C3)c(=O)c1=O)C(=O)O)C(=O)O] | E14 |
| 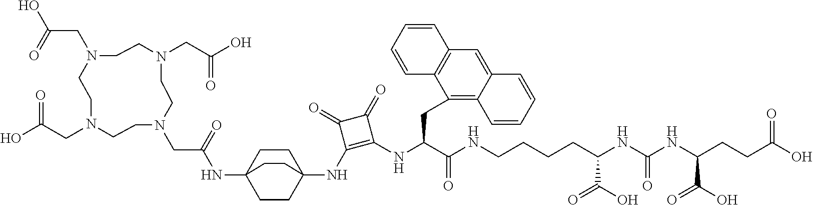 [SMILES: O=C(O)CC[C@H](NC(=O)N[C@@H](CCCCNC(=O)[C@H](Cc1c2ccccc2cc2ccccc12)Nc1c(NC23CCC(NC(=O)CN4CCN(CC(=O)O)CCN(CC(=O)O)CCN(CC(=O)O)CC4)(CC2)CC3)c(=O)c1=O)C(=O)O)C(=O)O] | E15 |
| 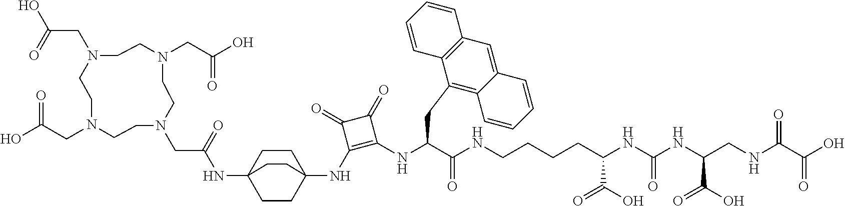 [SMILES: O=C(O)CN1CCN(CC(=O)O)CCN(CC(=O)NC23CCC(Nc4c(N[C@@H](Cc5c6ccccc6cc6ccccc56)C(=O)NCCCC[C@H](NC(=O)N[C@@H](CNC(=O)C(=O)O)C(=O)O)C(=O)O)c(=O)c4=O)(CC2)CC3)CCN(CC(=O)O)CC1] | E16 |
| 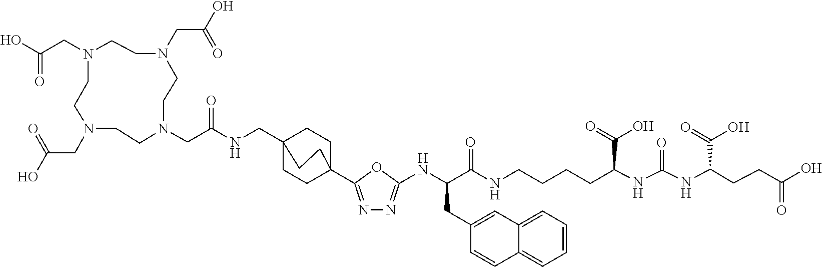 [SMILES: O=C(O)CC[C@H](NC(=O)N[C@@H](CCCCNC(=O)[C@@H](Cc1ccc2ccccc2c1)Nc1nnc(C23CCC(CNC(=O)CN4CCN(CC(=O)O)CCN(CC(=O)O)CCN(CC(=O)O)CC4)(CC2)CC3)o1)C(=O)O)C(=O)O] | E17 or E18 |
| 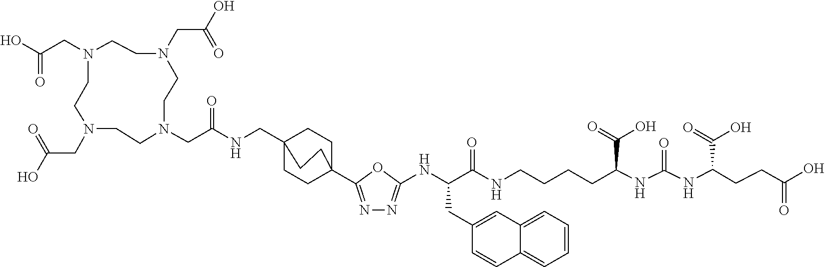 [SMILES: O=C(O)CC[C@H](NC(=O)N[C@@H](CCCCNC(=O)[C@H](Cc1ccc2ccccc2c1)Nc1nnc(C23CCC(CNC(=O)CN4CCN(CC(=O)O)CCN(CC(=O)O)CCN(CC(=O)O)CC4)(CC2)CC3)o1)C(=O)O)C(=O)O] | E18 or E17 |
| 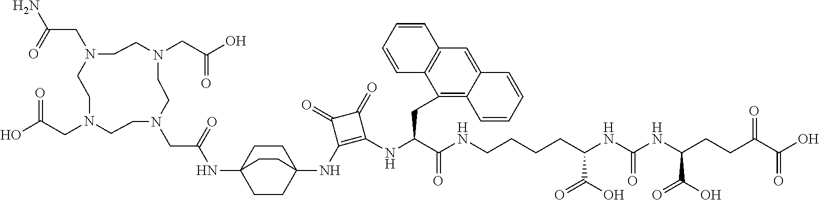 [SMILES: NC(=O)CN1CCN(CC(=O)O)CCN(CC(=O)NC23CCC(Nc4c(N[C@@H](Cc5c6ccccc6cc6ccccc56)C(=O)NCCCC[C@H](NC(=O)N[C@@H](CCC(=O)C(=O)O)C(=O)O)C(=O)O)c(=O)c4=O)(CC2)CC3)CCN(CC(=O)O)CC1] | E19 |
| 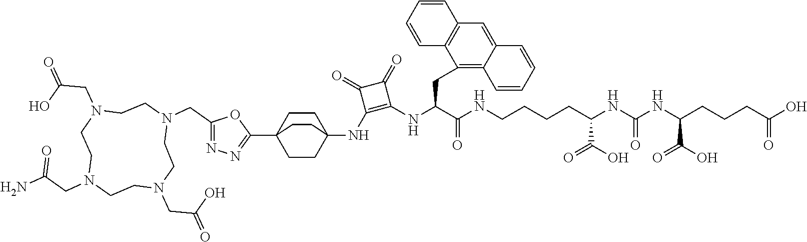 [SMILES: NC(=O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CCCC(=O)O)C(=O)O)C(=O)O)c(=O)c5=O)(CC3)CC4)o2)CCN(CC(=O)O)CC1] | E20 |
| 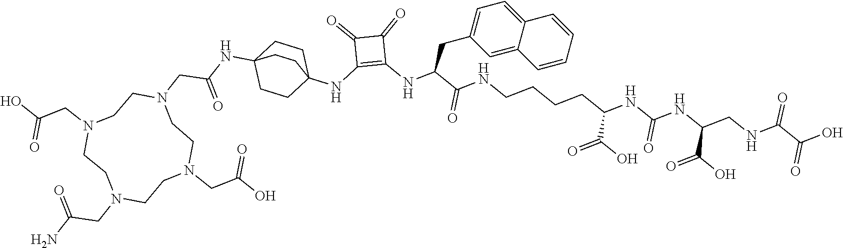 [SMILES: NC(=O)CN1CCN(CC(=O)O)CCN(CC(=O)NC23CCC(Nc4c(N[C@@H](Cc5ccc6ccccc6c5)C(=O)NCCCC[C@H](NC(=O)N[C@@H](CNC(=O)C(=O)O)C(=O)O)C(=O)O)c(=O)c4=O)(CC2)CC3)CCN(CC(=O)O)CC1] | E21 |
| 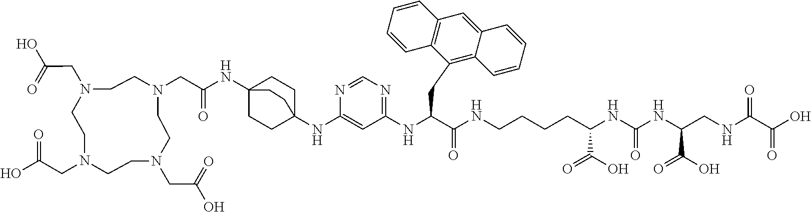 [SMILES: O=C(O)CN1CCN(CC(=O)O)CCN(CC(=O)NC23CCC(Nc4cc(N[C@@H](Cc5c6ccccc6cc6ccccc56)C(=O)NCCCC[C@H](NC(=O)N[C@@H](CNC(=O)C(=O)O)C(=O)O)C(=O)O)ncn4)(CC2)CC3)CCN(CC(=O)O)CC1] | E22 |
| 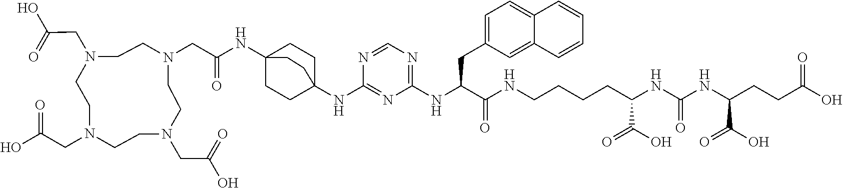 [SMILES: O=C(O)CC[C@H](NC(=O)N[C@@H](CCCCNC(=O)[C@H](Cc1ccc2ccccc2c1)Nc1ncnc(NC23CCC(NC(=O)CN4CCN(CC(=O)O)CCN(CC(=O)O)CCN(CC(=O)O)CC4)(CC2)CC3)n1)C(=O)O)C(=O)O] | E23 |
| 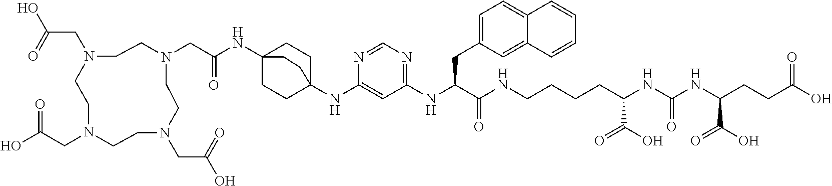 [SMILES: O=C(O)CC[C@H](NC(=O)N[C@@H](CCCCNC(=O)[C@H](Cc1ccc2ccccc2c1)Nc1cc(NC23CCC(NC(=O)CN4CCN(CC(=O)O)CCN(CC(=O)O)CCN(CC(=O)O)CC4)(CC2)CC3)ncn1)C(=O)O)C(=O)O] | E24 |
| 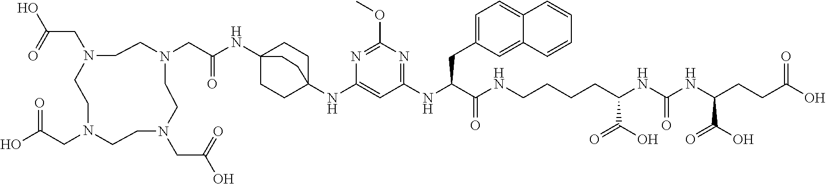 [SMILES: COc1nc(N[C@@H](Cc2ccc3ccccc3c2)C(=O)NCCCC[C@H](NC(=O)N[C@@H](CCC(=O)O)C(=O)O)C(=O)O)cc(NC23CCC(NC(=O)CN4CCN(CC(=O)O)CCN(CC(=O)O)CCN(CC(=O)O)CC4)(CC2)CC3)n1] | E25 |
| 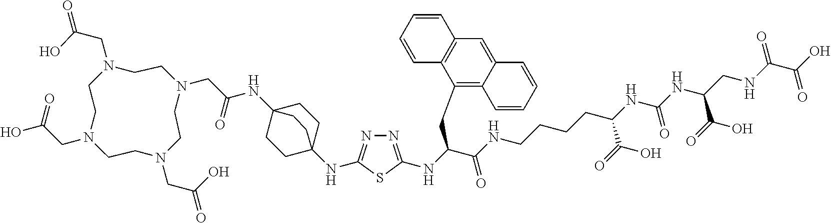 [SMILES: O=C(O)CN1CCN(CC(=O)O)CCN(CC(=O)NC23CCC(Nc4nnc(N[C@@H](Cc5c6ccccc6cc6ccccc56)C(=O)NCCCC[C@H](NC(=O)N[C@@H](CNC(=O)C(=O)O)C(=O)O)C(=O)O)s4)(CC2)CC3)CCN(CC(=O)O)CC1] | E26 |
| 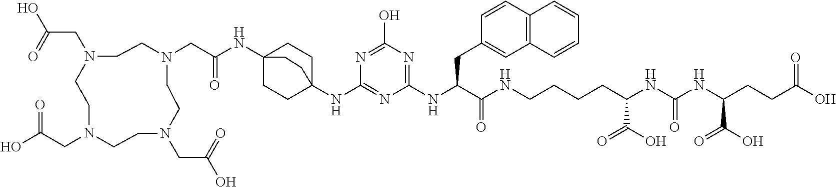 [SMILES: O=C(O)CC[C@H](NC(=O)N[C@@H](CCCCNC(=O)[C@H](Cc1ccc2ccccc2c1)Nc1nc(O)nc(NC23CCC(NC(=O)CN4CCN(CC(=O)O)CCN(CC(=O)O)CCN(CC(=O)O)CC4)(CC2)CC3)n1)C(=O)O)C(=O)O] | E27 |
| 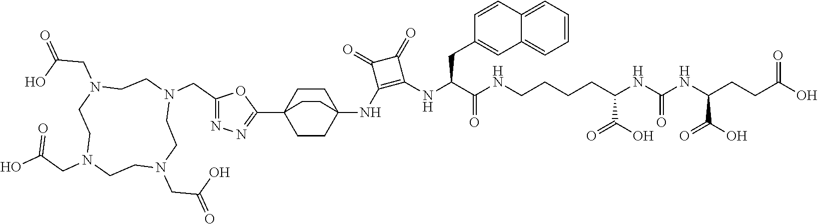 [SMILES: O=C(O)CC[C@H](NC(=O)N[C@@H](CCCCNC(=O)[C@H](Cc1ccc2ccccc2c1)Nc1c(NC23CCC(c4nnc(CN5CCN(CC(=O)O)CCN(CC(=O)O)CCN(CC(=O)O)CC5)o4)(CC2)CC3)c(=O)c1=O)C(=O)O)C(=O)O] | E28 |

TABLE 2A

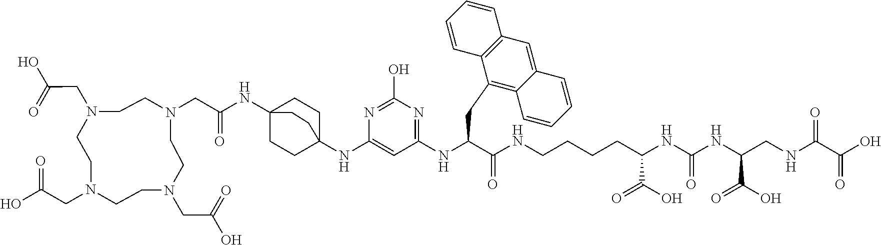 [SMILES: O=C(O)CN1CCN(CC(=O)O)CCN(CC(=O)NC23CCC(Nc4cc(N[C@@H](Cc5c6ccccc6cc6ccccc56)C(=O)NCCCC[C@H](NC(=O)N[C@@H](CNC(=O)C(=O)O)C(=O)O)C(=O)O)nc(O)n4)(CC2)CC3)CCN(CC(=O)O)CC1]

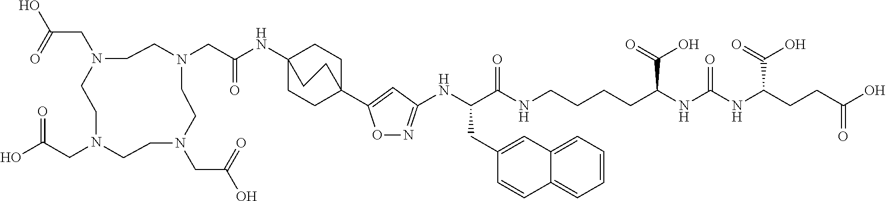 [SMILES: O=C(O)CC[C@H](NC(=O)N[C@@H](CCCCNC(=O)[C@H](Cc1ccc2ccccc2c1)Nc1cc(C23CCC(NC(=O)CN4CCN(CC(=O)O)CCN(CC(=O)O)CCN(CC(=O)O)CC4)(CC2)CC3)on1)C(=O)O)C(=O)O]

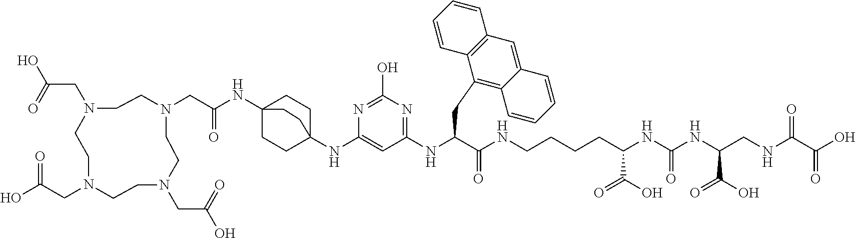 [SMILES: O=C(O)CN1CCN(CC(=O)O)CCN(CC(=O)NC23CCC(Nc4cc(N[C@@H](Cc5c6ccccc6cc6ccccc56)C(=O)NCCCC[C@H](NC(=O)N[C@@H](CNC(=O)C(=O)O)C(=O)O)C(=O)O)nc(O)n4)(CC2)CC3)CCN(CC(=O)O)CC1]

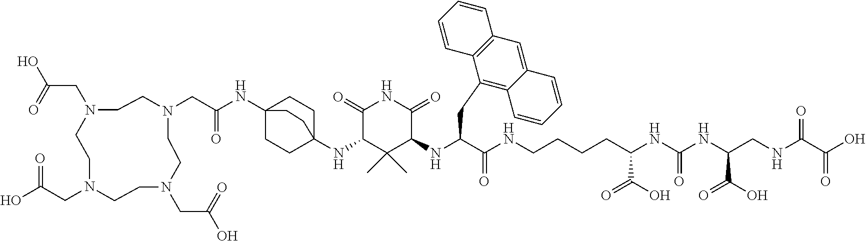 [SMILES: CC1(C)[C@H](N[C@@H](Cc2c3ccccc3cc3ccccc23)C(=O)NCCCC[C@H](NC(=O)N[C@@H](CNC(=O)C(=O)O)C(=O)O)C(=O)O)C(=O)NC(=O)[C@H]1NC12CCC(NC(=O)CN3CCN(CC(=O)O)CCN(CC(=O)O)CCN(CC(=O)O)CC3)(CC1)CC2]

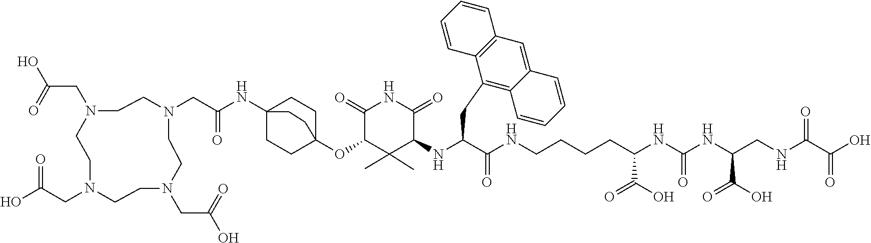 [SMILES: CC1(C)[C@H](N[C@@H](Cc2c3ccccc3cc3ccccc23)C(=O)NCCCC[C@H](NC(=O)N[C@@H](CNC(=O)C(=O)O)C(=O)O)C(=O)O)C(=O)NC(=O)[C@H]1OC12CCC(NC(=O)CN3CCN(CC(=O)O)CCN(CC(=O)O)CCN(CC(=O)O)CC3)(CC1)CC2]

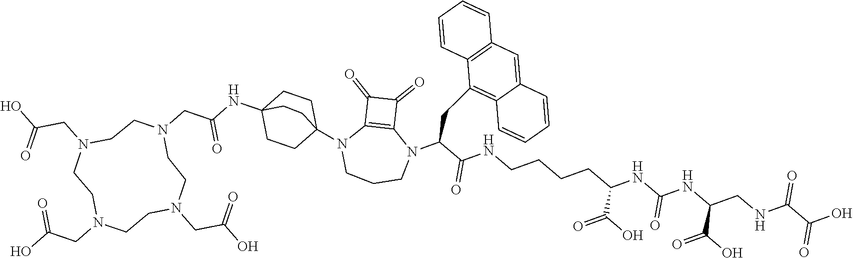 [SMILES: O=C(O)CN1CCN(CC(=O)O)CCN(CC(=O)NC23CCC(N4CCCN([C@@H](Cc5c6ccccc6cc6ccccc56)C(=O)NCCCC[C@H](NC(=O)N[C@@H](CNC(=O)C(=O)O)C(=O)O)C(=O)O)c5c4c(=O)c5=O)(CC2)CC3)CCN(CC(=O)O)CC1]

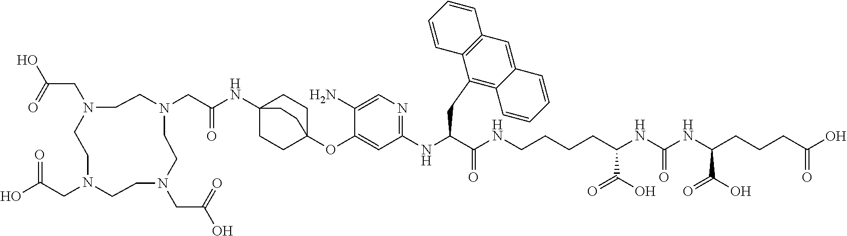 [SMILES: Nc1cnc(N[C@@H](Cc2c3ccccc3cc3ccccc23)C(=O)NCCCC[C@H](NC(=O)N[C@@H](CCCC(=O)O)C(=O)O)C(=O)O)cc1OC12CCC(NC(=O)CN3CCN(CC(=O)O)CCN(CC(=O)O)CCN(CC(=O)O)CC3)(CC1)CC2]

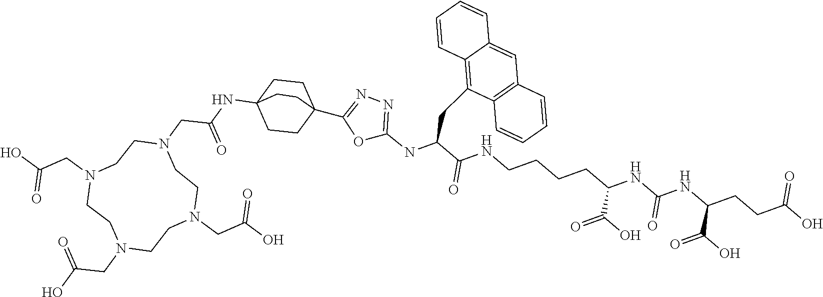 [SMILES: O=C(O)CC[C@H](NC(=O)N[C@@H](CCCCNC(=O)[C@H](Cc1c2ccccc2cc2ccccc12)Nc1nnc(C23CCC(NC(=O)CN4CCN(CC(=O)O)CCN(CC(=O)O)CCN(CC(=O)O)CC4)(CC2)CC3)o1)C(=O)O)C(=O)O]

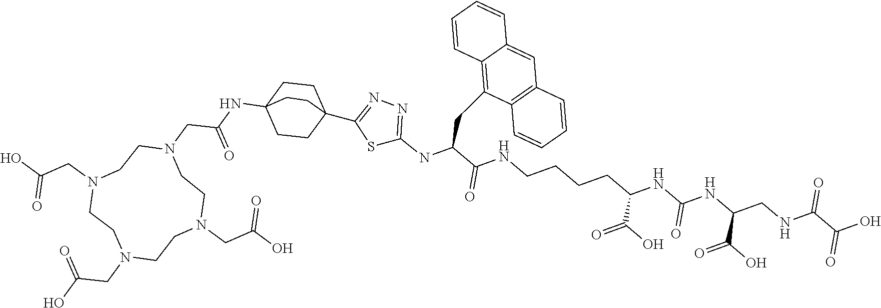 [SMILES: O=C(O)CN1CCN(CC(=O)O)CCN(CC(=O)NC23CCC(c4nnc(N[C@@H](Cc5c6ccccc6cc6ccccc56)C(=O)NCCCC[C@H](NC(=O)N[C@@H](CNC(=O)C(=O)O)C(=O)O)C(=O)O)s4)(CC2)CC3)CCN(CC(=O)O)CC1]

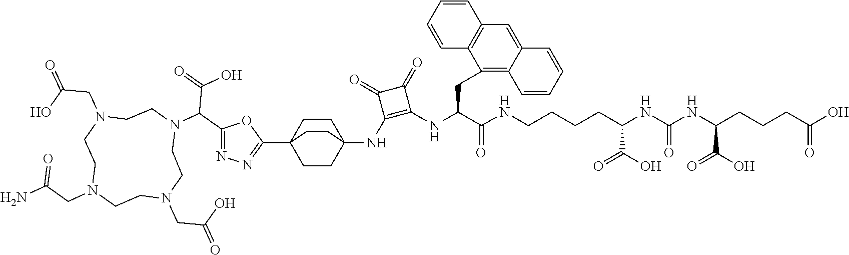 [SMILES: NC(=O)CN1CCN(CC(=O)O)CCN(C(C(=O)O)c2nnc(C34CCC(Nc5c(N[C@@H](Cc6c7ccccc7cc7ccccc67)C(=O)NCCCC[C@H](NC(=O)N[C@@H](CCCC(=O)O)C(=O)O)C(=O)O)c(=O)c5=O)(CC3)CC4)o2)CCN(CC(=O)O)CC1]

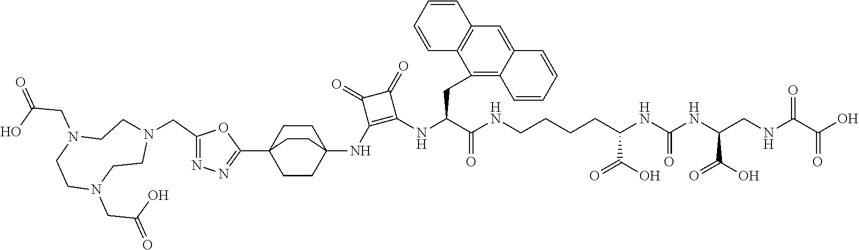 [SMILES: O=C(O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CNC(=O)C(=O)O)C(=O)O)C(=O)O)c(=O)c5=O)(CC3)CC4)o2)CC1]

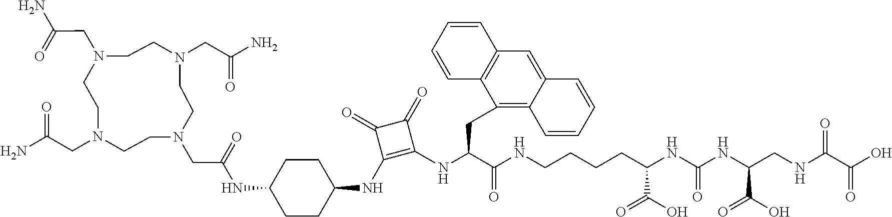 [SMILES: NC(=O)CN1CCN(CC(N)=O)CCN(CC(=O)N[C@H]2CC[C@H](Nc3c(N[C@@H](Cc4c5ccccc5cc5ccccc45)C(=O)NCCCC[C@H](NC(=O)N[C@@H](CNC(=O)C(=O)O)C(=O)O)C(=O)O)c(=O)c3=O)CC2)CCN(CC(N)=O)CC1]

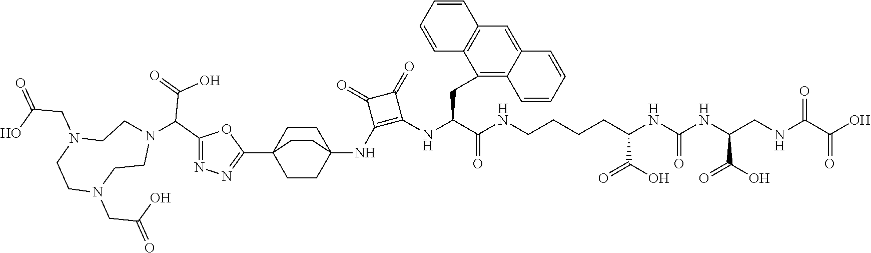 [SMILES: O=C(O)CN1CCN(CC(=O)O)CCN(C(C(=O)O)c2nnc(C34CCC(Nc5c(N[C@@H](Cc6c7ccccc7cc7ccccc67)C(=O)NCCCC[C@H](NC(=O)N[C@@H](CNC(=O)C(=O)O)C(=O)O)C(=O)O)c(=O)c5=O)(CC3)CC4)o2)CC1]

In one embodiment, the compounds provided herein are single diastereoisomers. In one embodiment, the compounds provided herein are mixtures of diastereoisomers. In one embodiment, the compounds provided herein have a diastereomeric excess (de) of at least about 50, at least about 80%, or at least about 90%. In one embodiment, the compounds provided herein have a diastereomeric excess of at least about 95%, at least about 97%, at least about 99%. In one embodiment, the compounds provided herein have a diastereomeric excess of at least about 99.5%. In one embodiment, the compounds provided herein have a diastereomeric excess of at least about 99.9%.

In one embodiment, the compounds provided herein are used as diagnostic agents. In one embodiment, the compounds provided herein are used as therapeutic agents. In one embodiment, the compounds provided herein are used as theranostic agents.

In one embodiment, provided herein is a complex formed by a compound provided herein and a metal cation. In one embodiment, the complex is formed when Z is a chelating agent.

In one embodiment, the metal cation is a divalent or trivalent metal cation. In one embodiment, the metal cation is a cation of Cr, Ga, In, Tc, Re, La, Yb, Sm, Ho, Y, Pm, Dy, Er, Lu, Sc, Pr, Gd, Bi, Ru, Pd, Rh, Sb, Ba, Hg, Eu, Tl, Pb, Cu, Re, Au, Ac, Th, or Ag. In one embodiment, the metal cation is a cation of Ga. In one embodiment, the metal cation is a cation of Lu.

In one embodiment, the metal cation is a cation of ⁵¹Cr, ⁶⁷Ga, ⁶⁸Ga, ⁸⁹Zr, ¹¹¹In, ^{99m}Tc, ¹⁸⁶Re, ¹⁸⁸Re, ¹³⁹La, ⁴⁰La, ¹⁷⁵Yb, ¹⁵³Sm, ¹⁶⁶Ho, ⁸⁸Y, ⁹⁰Y, ¹⁴⁹Pm, ⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁹⁷Ru, ¹⁰⁹Pd, ¹⁰⁵Rh, ^{101m}Rh, ¹¹⁹Sb, ¹²⁸Ba, ⁹⁷Hg, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁹⁸Au, ²²⁵Ac, ²²⁷Th, or ¹⁹⁹Ag. In one embodiment, the metal cation is a cation of ⁶⁸Ga. In one embodiment, the metal cation is a cation of ¹⁷⁷Lu. In one embodiment, the metal cation is ¹⁷⁷Lu³⁺. In one embodiment, the metal cation is ⁶⁸Ga³⁺. In one embodiment, the metal cation is ¹¹¹In³⁺. In one embodiment, the metal cation is ^{99m}Tc⁴⁺. In one embodiment, the metal cation is ⁹⁰Y³⁺. In one embodiment, the metal cation is ²⁰³Pb²⁺. In one embodiment, the metal cation is ²¹²Pb²⁺. In one embodiment, the metal cation is ⁶⁴Cu²⁺. In one embodiment, the metal cation is ²²⁵Ac³⁺.

In one embodiment, any complex formed by a compound in Table 2 or Table 2A and a metal cation provided herein is specifically provided herein. In one embodiment, any complex formed by a compound in Table 2 or Table 2A and ¹⁷⁷Lu³⁺ is specifically provided herein. In one embodiment, any complex formed by a compound in Table 2 or Table 2A and ⁶⁸Ga³⁺ is specifically provided herein. In one embodiment, any complex formed by a compound in Table 2 or Table 2A and ¹¹¹In³⁺ is specifically provided herein. In one embodiment, any complex formed by a compound in Table 2 or Table 2A and ^{99m}Tc⁴⁺ is specifically provided herein. In one embodiment, any complex formed by a compound in Table 2 or Table 2A and ⁹⁰Y³⁺ is specifically provided herein. In one embodiment, any complex formed by a compound in Table 2 or Table 2A and ²⁰³Pb²⁺ is specifically provided herein. In one embodiment, any complex formed by a compound in Table 2 or Table 2A and ²¹²Pb²⁺ is specifically provided herein. In one embodiment, any complex formed by a compound in Table 2 or Table 2A and ⁶⁴Cu²⁺ is specifically provided herein. In one embodiment, any complex formed by a compound in Table 2 or Table 2A and ²²⁵Ac³⁺ is specifically provided herein.

In one embodiment, the complex is a complex in Table 3, or a stereoisomer, a mixture of stereoisomers, a tautomer, or a pharmaceutically acceptable salt thereof.

TABLE 3

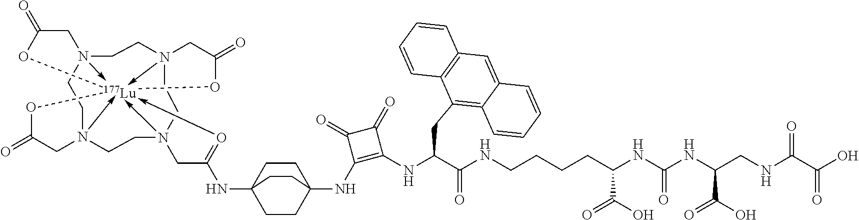

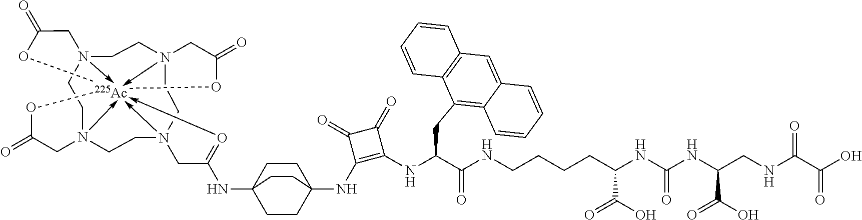

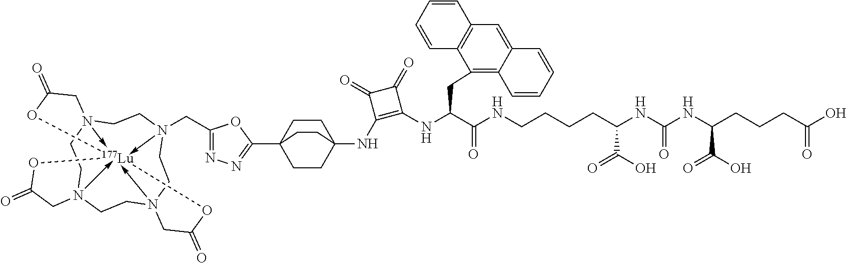

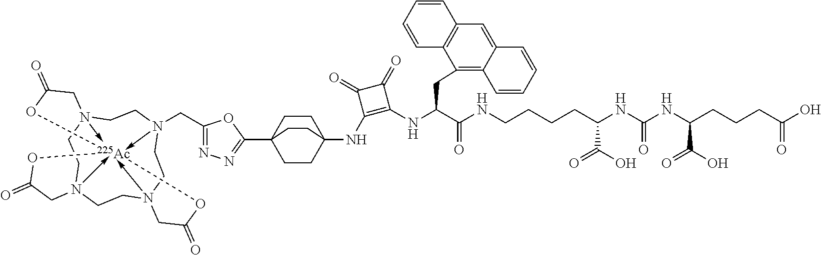

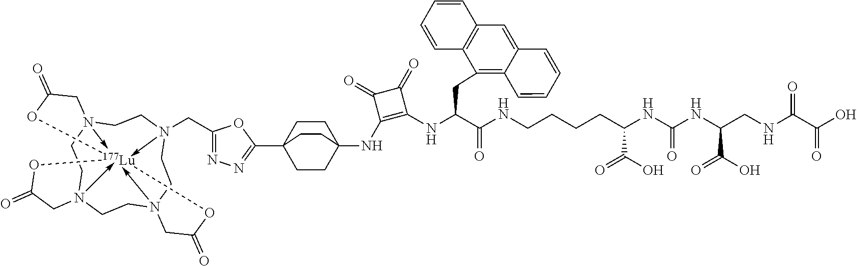

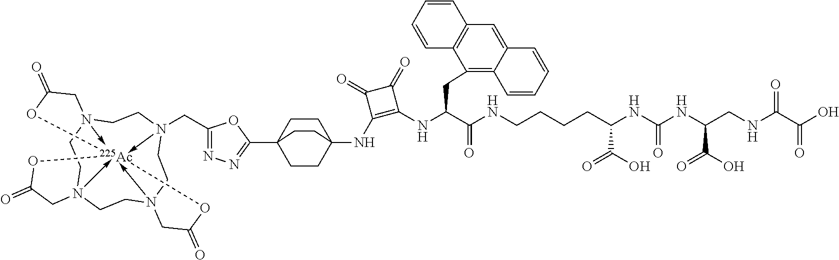

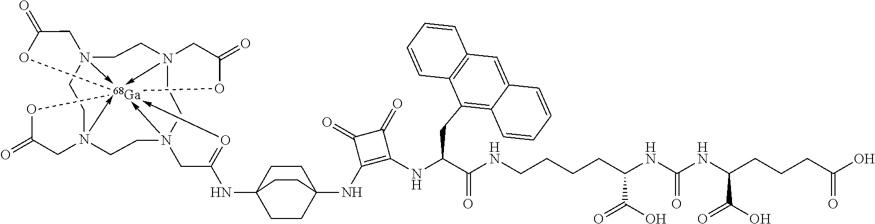

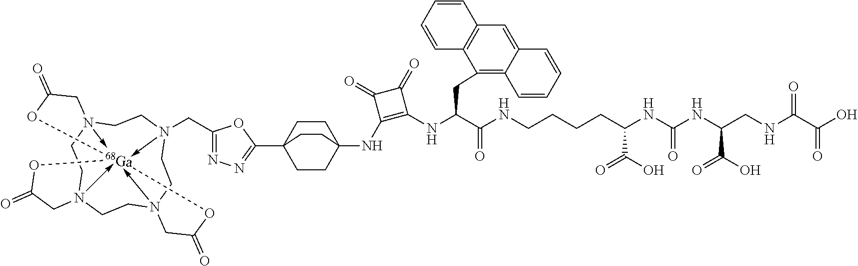

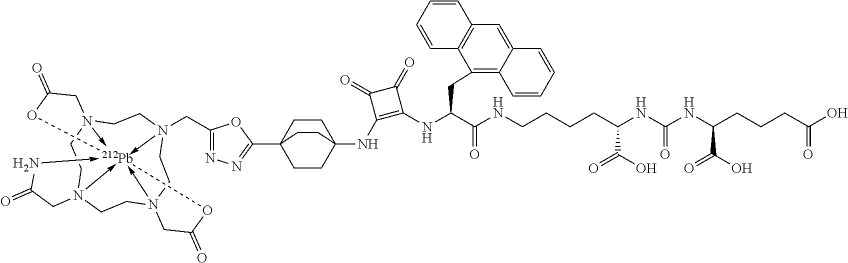

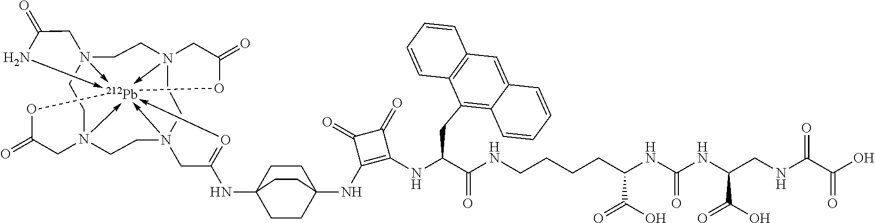

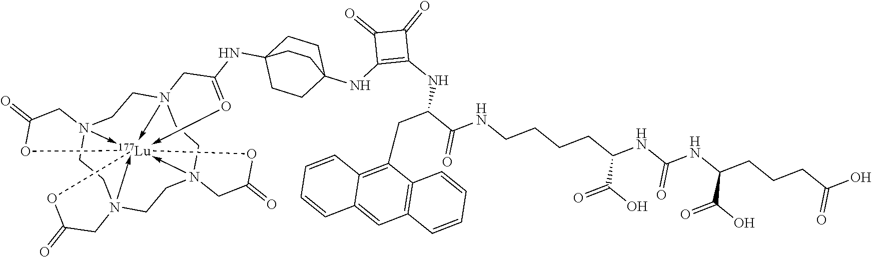

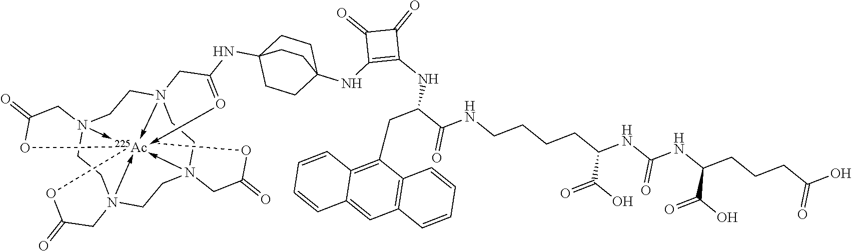

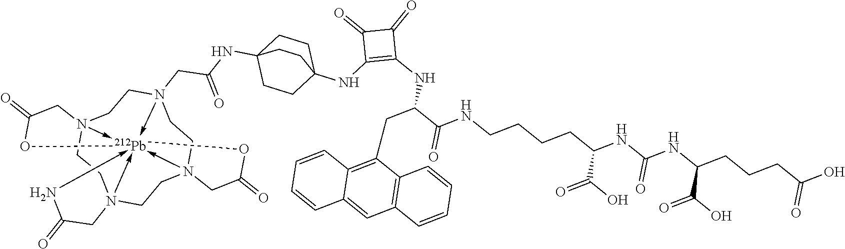

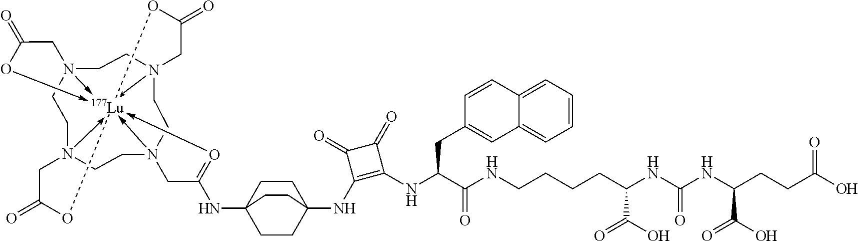

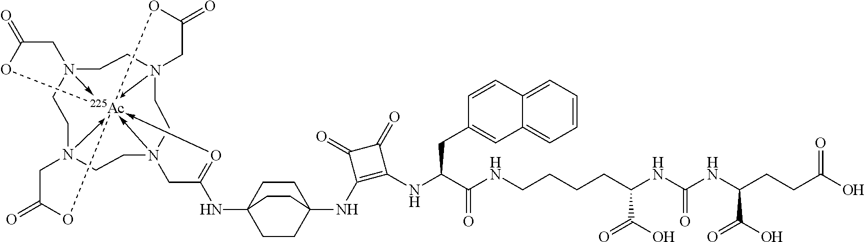

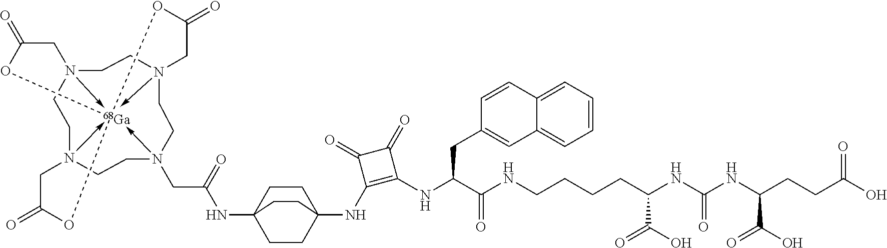

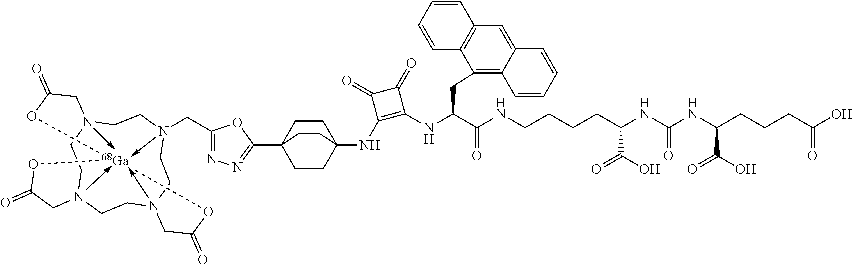

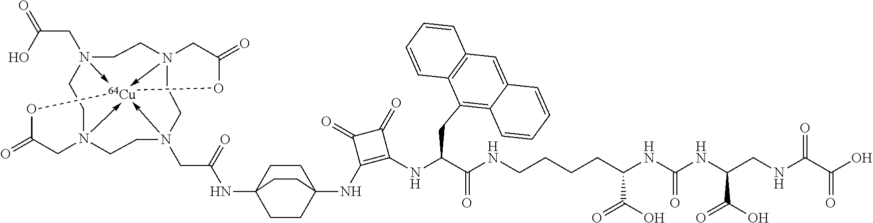

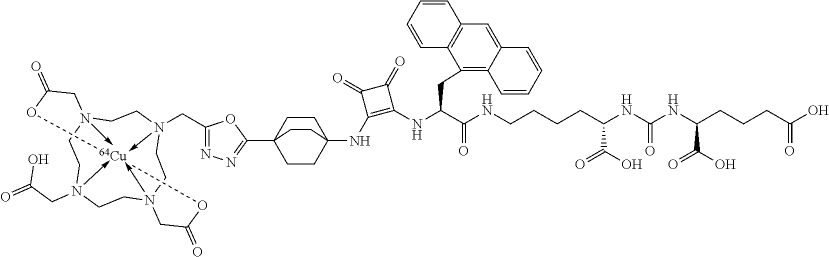

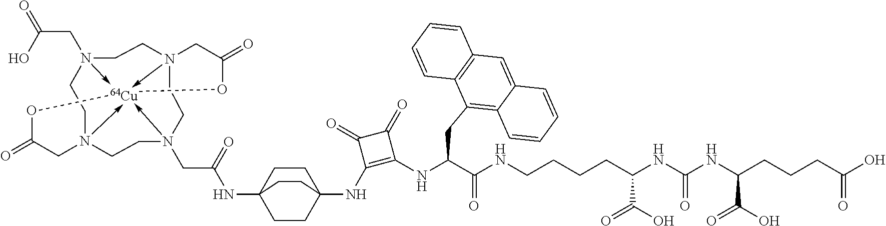

As used herein and unless otherwise specified, the → and ---shown in the structures provided herein are merely for illustration purpose of the possible chelating between the compound and the metal cation. It does not mean chelating necessarily happens as indicated by the → and ---. It does not mean chelating cannot happen between other atoms of the compound and the metal cation.

In one embodiment, without being limited by a particular theory, a compound or complex provided herein exhibit suitable cellular uptake in PSMA transfected cells, as well as tumor uptake in PSMA-positive tumors. In one embodiment, without being limited by a particular theory, a compound or complex provided herein demonstrate potent PSMA enzymatic inhibition, great tumor uptake and/or retention. In one embodiment, without being limited by a particular theory, small animal PET/CT or SPECT/CT delineate the tumor volume with great tumor-to-blood, tumor-to-kidney ratio, and long-term tumor retention, and resulting in completely tumor inhibition in the in vivo efficacy study.

In one embodiment, the complex provided herein are used as diagnostic agents. In one embodiment, the complex provided herein are used as therapeutic agents. In one embodiment, the complex provided herein are used as theranostic agents.

### Pharmaceutical Composition and Method of Use

In one embodiment, provided herein is a pharmaceutical composition comprising a compound provided herein or a complex provided herein and a pharmaceutically acceptable excipient.

In one embodiment, provided herein is a virus-like particle (VLP) comprising a compound provided herein, wherein Z is a viral structural protein. In one embodiment, the virus-like particle is loaded with a substance selected from the group consisting of an agent and a nucleic acid. In one embodiment, the agent that the virus-like particle is loaded with is a cytostatic and/or cytotoxic agent provided herein. In one embodiment, the nucleic acid that the virus-like particle is loaded with is a nucleic acid provided herein.

In one embodiment, provided herein is a pharmaceutical composition comprising a compound provided herein, a liposome provided herein, or a virus-like particle provided herein, and a pharmaceutically acceptable excipient.

As used herein and unless otherwise specified, a PSMA positive disease or disorder (e.g., a PSMA positive cancer) refers to a disease or disorder (e.g., cancer) characterized by an elevated expression (e.g., overexpression) of PSMA. In one embodiment, the elevated expression is compared to, e.g., a healthy subject. In one embodiment, the PSMA positivity is determined by a method provided herein (e.g., using a compound or complex provided herein), or by a method known in the art, including methods approved by U.S. FDA (e.g., IHC staining, PSMA imaging).

In one embodiment, provided herein is a method for the diagnosis of a disease or disorder characterized by overexpression of prostate-specific membrane antigen (PSMA) in a subject, comprising administering to the subject a diagnostically effective amount of a compound provided herein, a complex provided herein, or a pharmaceutical composition provided herein. In one embodiment, a compound provided herein, a complex provided herein, or a pharmaceutical composition provided herein is for use in the diagnosis of a disease or disorder characterized by overexpression of prostate-specific membrane antigen (PSMA) in a subject.

In one embodiment, provided herein is a method for the treatment of a disease or disorder characterized by overexpression of prostate-specific membrane antigen (PSMA) in a subject, comprising administering to the subject a therapeutically effective amount of a compound provided herein or a pharmaceutical composition provided herein. In one embodiment, a compound provided herein or a pharmaceutical composition provided herein is for use in the treatment of a disease or disorder characterized by overexpression of prostate-specific membrane antigen (PSMA) in a subject.

In one embodiment, provided herein is a method of treating or diagnosing cancer. In one embodiment, provided herein is a method of treating a PSMA positive cancer, comprising administering a therapeutically effective amount of a compound described herein, a complex described herein, or a pharmaceutical composition described herein to a subject in need thereof. In one embodiment, provided herein is a method of diagnosing a PSMA positive cancer, comprising administering a therapeutically effective amount of a compound described herein, a complex described herein, or a pharmaceutical composition described herein to a subject in need thereof.

In one embodiment, provided herein is a method for the treatment of a disease or disorder characterized by overexpression of prostate-specific membrane antigen (PSMA) in a subject, comprising administering to the subject a therapeutically effective amount of a compound provided herein, a liposome provided herein, a virus-like particle (VLP) provided herein, or a pharmaceutical composition provided herein. In one embodiment, a compound provided herein, a liposome provided herein, a virus-like particle (VLP) provided herein, or a pharmaceutical composition provided herein is for use in the treatment of a disease or disorder characterized by overexpression of prostate-specific membrane antigen (PSMA) in a subject.

In one embodiment, the administration is intravenous administration, intramuscular administration, intraarterial administration, intrathecal administration, intracapsular administration, intraorbital administration, intracardiac administration, intradermal administration, intraperitoneal administration, transtracheal administration, subcutaneous administration, subcuticular administration, intraarticular administration, subcapsular administration, subarachnoid administration, intraspinal administration, or intrasternal administration. In one embodiment, the administration is intravenous administration.

In one embodiment, the disease is cancer. In one embodiment, the disease cancer is PSMA positive cancer. PSMA expression has been detected in various cancers (e.g. Rowe et al., 2015, Annals of Nuclear Medicine 29:877-882; Sathekge et al., 2015, Eur J Nucl Med Mol Imaging 42: 1482-1483; Verburg et al., 2015, Eur J Nucl Med Mol Imaging 42: 1622-1623; and Pyka et al., J Nucl Med Nov. 19, 2015 jnumed.115.164442), the entirely of each of which are incorporated herein by reference. In one embodiment, the cancer is prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer, adenoid cystic carcinoma, salivary duct carcinoma, or sarcoma. In one embodiment, the cancer is prostate cancer. In one embodiment, the cancer is adenoid cystic carcinoma. In one embodiment, the cancer is salivary duct carcinoma. In one embodiment, the cancer is sarcoma.

In one embodiment, the subject is an animal. In one embodiment, the subject is a mammal. In one embodiment, the subject is a human.

Also provided herein is a method of detecting cells or tissues expressing prostate-specific membrane antigen (PSMA) comprising (i) contacting the PSMA-expressing cells or tissues with a compound or complex described herein and (ii) applying one or more imaging method to detect the cells or tissues. In one embodiment, the imaging method comprises positron emission tomography (PET), single-photon emission computed tomography (SPECT), magnetic resonance imaging (MRI), computed tomography (CT), scintigraphy imaging, luminescence imaging, or fluorescence imaging, or a combination thereof.

In one embodiment, the PSMA-expressing cells or tissues comprise prostate cells or tissues, spleen cells or tissues, or kidney cells or tissues.

In one embodiment, the detecting is performed in vivo. In one embodiment, the detecting is performed ex vivo. In one embodiment, the detecting is performed in vitro.

In one embodiment, provided herein is a kit comprising a compound provided herein, a complex provided herein, or a pharmaceutical composition provided herein and instructions for the diagnosis or treatment of a disease or disorder provided herein. In one embodiment, provided herein is a kit comprising a compound provided herein, a complex provided herein, a liposome provided herein, a virus-like particle (VLP) provided herein, or a pharmaceutical composition provided herein, and instructions for the treatment of a disease or disorder.

It is understood that any embodiment of the compounds provided herein, as set forth above, and any specific substituent and/or variable in the compounds provided herein, as set forth above, may be independently combined with other embodiments and/or substituents and/or variables of the compounds to form embodiments not specifically set forth above. In addition, in the event that a list of substituents and/or variables is listed for any particular group or variable, it is understood that each individual substituent and/or variable may be deleted from the particular embodiment and/or claim and that the remaining list of substituents and/or variables will be considered to be within the scope of embodiments provided herein.

It is understood that in the present description, combinations of substituents and/or variables of the depicted formulae are permissible only if such contributions result in stable compounds.

### Examples

Certain embodiments of the invention are illustrated by the following non-limiting examples.

### Methods of Preparation

### Prep-HPLC Purification Method 1

The compound was purified on Shimadzu LC-20AP and UV detector. The column used was Shim-pack GIS C18 (250*20) mm, 10 μm. Column flow was 15 mL/min. Mobile phase were used (A) 0.10% TFA in Water and (B) Acetonitrile. Purification was carried out employing a linear gradient from 5% to 35% of (B) Acetonitrile for 20 or 30 min. The UV spectra were recorded at 220 nm & 254 nm.

### Prep-HPLC Purification Method 2

The compound was purified on Shimadzu LC-20AP and UV detector. The column used was Shim-pack GIS C18 (250*20) mm, 10 μm. Column flow was 15 mL/min. Mobile phase were used (A) 0.1% NH₃ in Water and (B) Acetonitrile. Purification was carried out employing a linear gradient from 5% to 35% of (B) Acetonitrile for 20 or 30 min. The UV spectra were recorded at 220 nm & 254 nm.

### Prep-HPLC Purification Method 3

The compound was purified on Shimadzu LC-20AP and UV detector. The column used was Shim-pack GIS C18 (250*20) mm, 10 μm. Column flow was 15 mL/min. Mobile phase were used (A) 0.1% NH₄HCO₃ in Water and (B) Acetonitrile. Purification was carried out employing a linear gradient from 2% to 35% of (B) Acetonitrile for 25 min or 30 min. The UV spectra were recorded at 220 nm & 254 nm.

### Methods of Preparation

Compounds provided herein may be prepared using reactions and techniques known in the art and those described herein.

### Preparation of Intermediates

### D1: Tri-tert-butyl 2,2′,2″-(10-(2-hydrazineyl-2-oxoethyl)-1,4,7,10-tetraazacyclododecane-1,4,7-triyl)triacetate

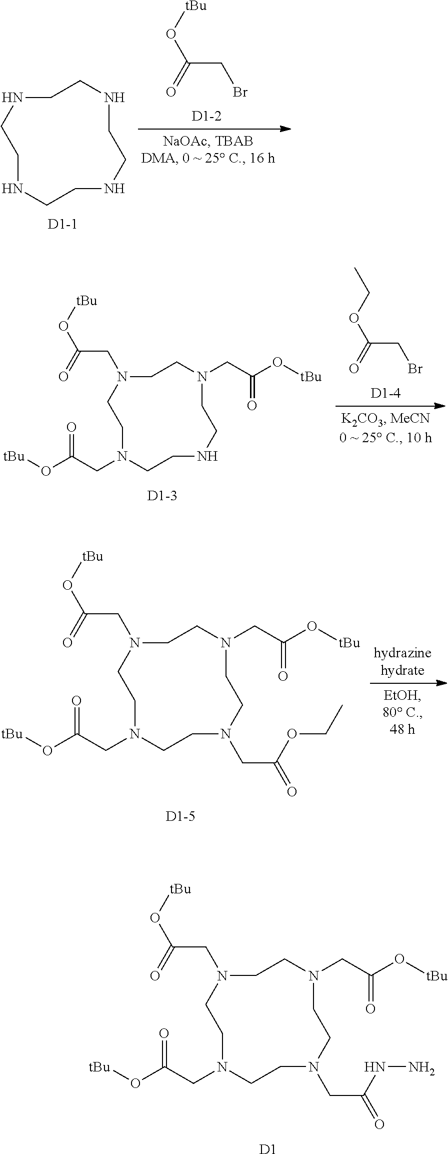

### Step 1

To a solution of D1-1 (10.00 g, 58.00 mmol), sodium acetate (19.05 g, 232.00 mmol) and tetrabutylammonium bromide (430.00 mg, 1.34 mmol) in DMA (N,N-dimethylacetamide) (84 mL) was added D1-2 (33.5 mL, 232.00 mmol) at 0° C. The mixture was stirred at 25° C. under N₂ atmosphere overnight. The reaction was diluted with 330 mL of water, adjusted pH to 8.8-9.0 with sodium carbonate solution, filtered and washed with water (20 mL×2). The filter cake was added to 90 mL of ethanol, heated to 45° C. to give a clear solution, to which was added 270 mL of water, stirred at room temperature for 2 h, and filtered. The filter cake was washed with water (20 mL×2) and dried over to obtain D1-3 (26.80 g, 52.10 mmol, yield: 90.0%) as a white solid. LC-MS (ESI) m/z: 515 [M+H]⁺.

### Step 2

To a solution of D1-3 (25.00 g, 48.60 mmol) in ACN (acetonitrile) (250 mL) were added K₂CO₃ (6.71 g, 48.60 mmol) and D1-4 (5.95 mL, 53.40 mmol) at 0° C. The mixture was stirred at 25° C. under N₂ for 10 h. The reaction mixture was filtered and evaporated to dryness to give the crude product D1-5 (25.00 g, crude) as colorless oil. LC-MS (ESI) m/z: 601 [M+H]⁺.

### Step 3

To a solution of D1-5 (25.00 g, 41.60 mmol) in EtOH (ethanol) (750 mL) was added hydrazine hydrate (239 mL, 4.16 mol). The mixture was stirred at 80° C. under N₂ for 2 days. The resulting mixture was evaporated under reduced pressure to give crude, which was purified by flash chromatography to afford the compound D1 (13.60 g, 23.18 mmol, yield: 47.7% by two steps) as a white solid. LC-MS (ESI) m/z: 587 [M+H]⁺.

### D2: di-tert-butyl (((S)-6-((S)-2-amino-3-(naphthalen-2-yl)propanamido)-1-(tert-butoxy)-1-oxohexan-2-yl)carbamoyl)-L-glutamate

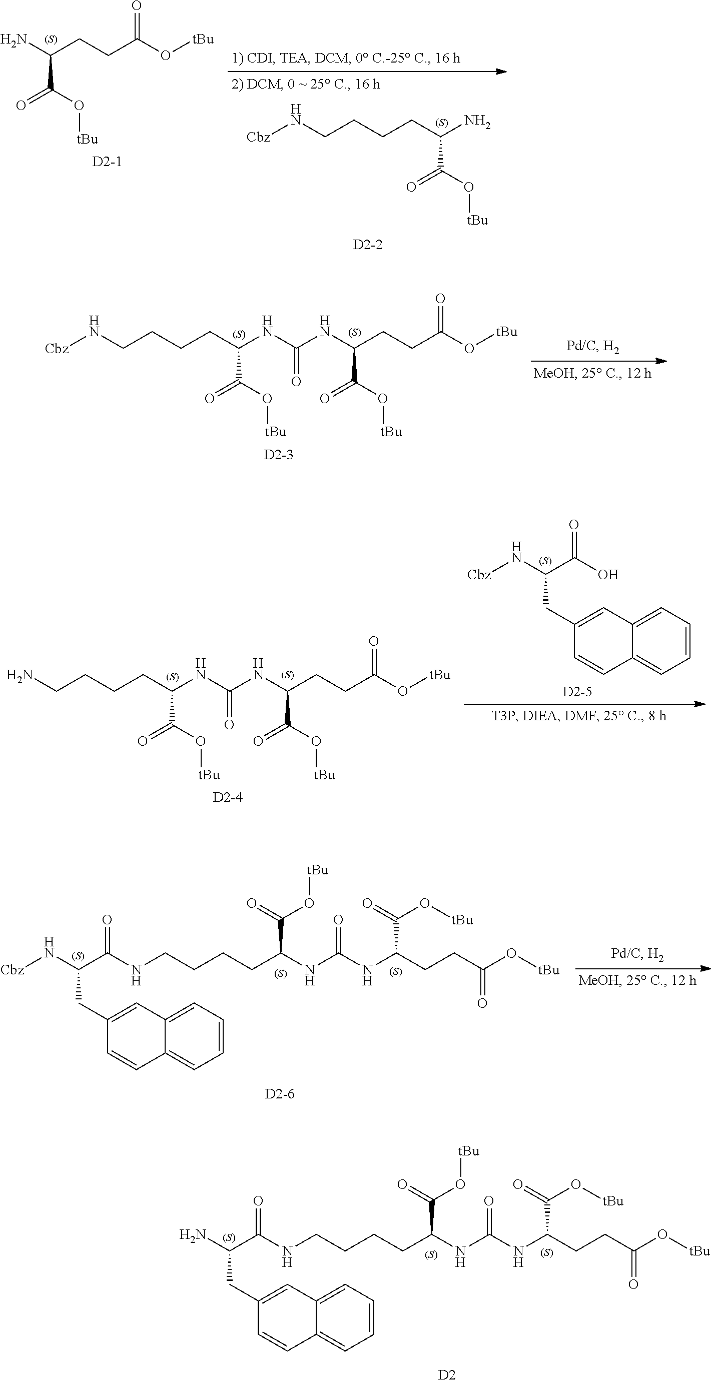

### Step 1

To a solution of D2-1 (25.00 g, 85.00 mmol) in DCM (dichloromethane) (260 mL) were added TEA (triethylamine) (47 mL, 338.00 mmol) and N,N-dimethylpyridin-4-amine (413.00 mg, 3.38 mmol) at 0° C. Then, a solution of di(1H-imidazol-1-yl)methanone (15.07 g, 93.00 mmol) in DCM (85 mL) and was added slowly over a period of 30 min. The mixture was stirred at 25° C. under N₂ for 16 h. The reaction was quenched with Saturated NaHCO₃ (aq.). The aqueous layer was extracted with DCM (250 mL×2). The combined organic layers were washed with brine (500 mL×1), dried over Na₂SO₄, filtered and concentrated to give the crude product, which was dissolved in DCM (410 mL), followed by addition of D2-2 (22.79 g, 61.10 mmol) and DIEA (N,N-diisopropylethylamine) (47.4 mL, 272.00 mmol). The mixture was stirred at 25° C. under N₂ for 16 h. The reaction was quenched with 1.0 mol/L HCl (aq.). The aqueous layer was extracted with DCM (500 mL×2). The combined organic layers were washed with brine (500 mL×1). The organic layer was dried over Na₂SO₄, filtered and concentrated to give the crude product, which was purified by flash chromatography to afford the compound D2-3 (30.00 g, 48.20 mmol, yield: 71.1%) as colorless oil. LC-MS (ESI) m/z: 622 [M+H]⁺.

### Step 2

To a solution of D2-3 (25.00 g, 40.20 mmol) in MeOH (methanol) (150 mL) was added 10% wet Pd/C (2.50 g). The mixture was stirred at 25° C. under H₂ for 12 h. The suspension was filtered through a pad of celite and washed with MeOH (50 mL×2). The combined filtrates were concentrated to dryness to give D2-4 (16.80 g, 34.50 mmol, yield: 86.0%). LC-MS (ESI) m/z: 488 [M+H]⁺.

### Step 3

To a solution of D2-5 (2.00 g, 5.72 mmol) in DMF (N,N-dimethylformamide) (20 mL) were added DIEA (4.00 mL, 22.90 mmol) and D2-4 (2.79 g, 5.72 mmol), T3P (1-propanephosphonic acid cyclic anhydride) (5.88 mL, 11.45 mmol). The mixture was stirred at 25° C. under N₂ for 8 h. The reaction was quenched with H₂O (50 mL). The aqueous layer was extracted with EA (ethyl acetate) (200 mL×2). The combined organic layers were washed with Saturated NaCl (aq.) (150 mL×1), dried over Na₂SO₄, filtered and concentrated to give the crude product. The crude product was purified by silica gel column chromatography to afford D2-6 (1.60 g, 1.95 mmol, yield: 34.1%). LC-MS (ESI) m/z: 819 [M+H]⁺.

### Step 4

To a solution of D2-6 (1.60 g, 1.95 mmol) in MeOH (32 mL) were added 10% wet Pd/C (0.16 g). The mixture was stirred at 25° C. under H₂ for 12 h. The suspension was filtered through a pad of celite and washed with MeOH (20 mL×2). The combined filtrates were concentrated to dryness to afford D2 (1.00 g, 1.46 mmol, yield: 74.7%). LC-MS (ESI) m/z: 685 [M+H]⁺.

### D3: 4-(((tert-butoxycarbonyl)amino)methyl)bicyclo[2.2.2]octane-1-carboxylic acid

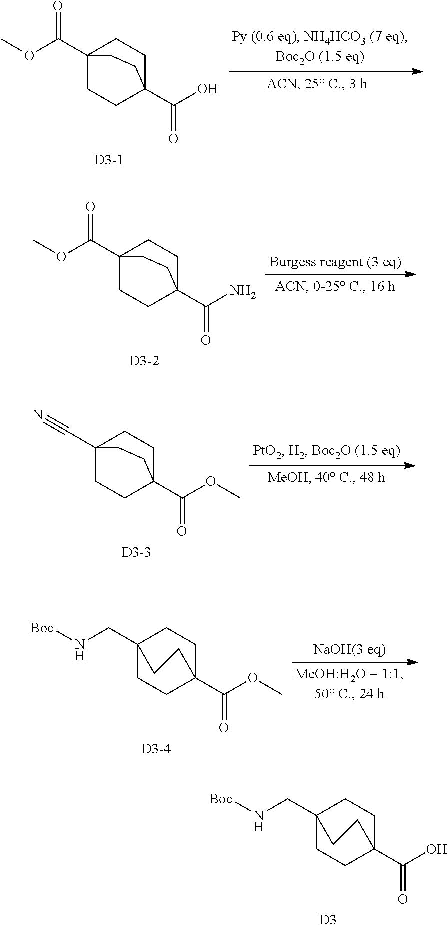

### Step 1

To a solution of D3-1 (20.00 g, 94.00 mmol) in ACN (300 mL) were added Boc₂O (32.80 mL, 141.00 mmol), Pyridine (4.57 mL, 56.50 mmol) and NH₄HCO₃ (52.10 g, 660.00 mmol). The mixture was stirred at 25° C. under N₂ for 3 h. The reaction mixture was filtered, and the filtrate was evaporated to dryness to give the crude product, which was purified by silica gel column chromatography to afford D3-2 (18.00 g, 85.00 mmol, yield: 90.0%) as a white solid. LC-MS (ESI) m/z: 212 [M+H]⁺.

### Step 2

To a solution of D3-2 (25.20 g, 119.00 mmol) in DCM (250 mL) were added Burgess reagent (methyl N-(triethylammonium sulfonyl)carbamate) (85.00 g, 358.00 mmol) at 0° C. The mixture was stirred at 25° C. under N₂ for 16 h. The mixture was then evaporated under reduced pressure to give crude product, which was purified by silica gel column chromatography to afford D3-3 (21.80 g, 113.00 mmol, yield: 95.0%) as a white solid. LC-MS (ESI) m/z: 194 [M+H]⁺.

### Step 3

To a solution of D3-3 (21.80 g, 113.00 mmol) in MeOH (400 mL) were added PtO₂ (2.56 g, 11.28 mmol) and Boc₂O (di-tert-butyl dicarbonate) (39.30 mL, 169.00 mmol). The mixture was stirred at 40° C. under H₂ for 48 h. The reaction mixture was filtered and the filter cake was washed with MeOH (50 mL). The filtrate was then evaporated under reduced pressure to give crude product, which was quenched with water (200 mL) and extracted with EA (250 mL). The combined organic layers were washed with brine (250 mL×3), dried over Na₂SO₄, filtered and concentrated to give the D3-4 (28.80 g, 97.00 mmol, yield: 86.0%) as a white solid. LC-MS (ESI) m/z: 298 [M+H]⁺.

### Step 4

To a solution of D3-4 (32.00 g, 108.00 mmol) in MeOH (150 mL) and H₂O (150 mL) was added NaOH (12.91 g, 323.00 mmol). The mixture was stirred at 50° C. under N₂ for 24 h. The mixture was then evaporated under reduced pressure to give aqueous layer, which was extracted with EA (100 mL×2). The aqueous layer was adjusted pH to 2 with HCl (2N). The resulting mixture was filtered, and the filter cake was evaporated to dryness to give the D3 (28.30 g, 100.00 mmol, yield: 93.0%) as a white solid. LC-MS (ESI) m/z: 284 [M+H]⁺.

### D4: di-tert-butyl (S)-2-(3-((S)-6-((S)-2-amino-3-(anthracen-9-yl)propanamido)-1-(tert-butoxy)-1-oxohexan-2-yl)ureido)hexanedioate

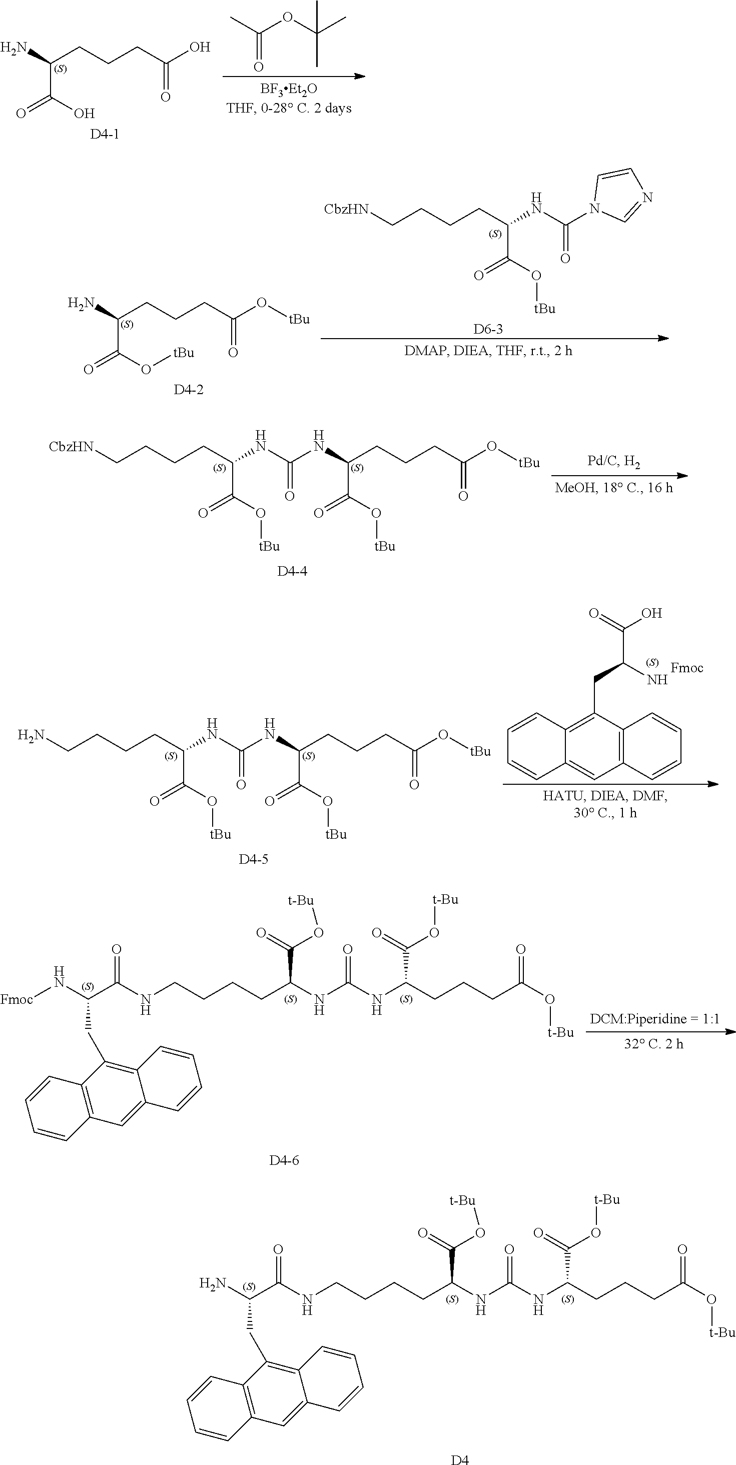

### Step 1

To a solution of D4-1 (2.00 g, 12.41 mmol) in THF (10 mL) at 0° C. were added tert-butyl acetate (6 mL, 12.41 mmol), Boron trifluoride diethyl etherate (6.3 mL, 49.70 mmol) at 0° C. The mixture was stirred at 28° C. under N₂ for 2 days. The reaction was quenched with aq. NaOH (1N, 20 mL). The aqueous layer was extracted with EA (50 mL×3). The combined organic layers were washed with brine (50 mL×2), dried over Na₂SO₄, filtered and concentrated to give the crude product, which was purified by silica gel column chromatography to afford D4-2 (600.00 mg, 12.19 mmol, yield: 17.7%) as yellow oil. LC-MS (ESI) m/z: 274 [M+H]⁺.

### Step 2

To a solution of D4-2 (1.36 g, 4.96 mmol) in THF (15 mL) was added DIEA (1.73 mL, 9.92 mmol), D4-3 (2.56 g, 5.95 mmol) and DMAP (0.06 g, 0.50 mmol). The mixture was stirred at 25° C. under N₂ for 2 h. The reaction was quenched with H₂O (50 mL). The aqueous layer was extracted with EA (50 mL×3). The combined organic layers were washed with brine (50 mL×1), dried over Na₂SO₄, filtered and concentrated to give the crude product, which was purified by silica gel column chromatography to afford D4-4 (2.02 g, 3.18 mmol, yield: 64.1%) as colorless oil. LC-MS (ESI) m/z: 636 [M+H]⁺.

### Step 3

A mixture of D4-4 (1.40 g, 2.20 mmol) and 10% wet Pd/C (0.23 g) in MeOH (20 mL) was degassed under vacuum and purged with H₂ three times. The mixture was stirred at 18° C. under H₂ atmosphere for 16 h. The reaction mixture was filtered and evaporated to dryness to give the crude product, which was purified by silica gel column chromatography to afford D4-5 (791.00 mg, 1.58 mmol, yield: 71.6%) as yellow oil. LC-MS (ESI) m/z: 502 [M+H]⁺.

### Step 4

To a solution of (S)-2-((((9H-fluoren-9-yl)methoxy)carbonyl)amino)-3-(anthracen-9-yl)propanoic acid (2.70 g, 5.54 mmol) in DMF (30 mL) were added HATU (3.16 g, 8.31 mmol), DIEA (3.87 mL, 22.15 mmol) and D4-5 (2.78 g, 5.54 mmol). The mixture was stirred at 30° C. under N₂ for 2 h. The reaction was quenched with Saturated NH₄Cl (50 mL). The aqueous layer was extracted with EA (125 mL×2). The combined organic layers were washed with brine (200 mL×1), dried over Na₂SO₄, filtered, and concentrated in vacuum to give the crude product, which was purified by silica gel column chromatography to afford D4-6 (3.70 g, 3.81 mmol, yield: 68.9%) as yellow oil. LC-MS (ESI) m/z: 971 [M+H]⁺.

### Step 5

To a solution of D4-6 (5.00 g, 5.15 mmol) in DCM (40 mL) was added piperidine (10 mL). The mixture was stirred at 32° C. under N₂ for 2 h. The reaction mixture was concentrated to give a crude product, which was purified by silica gel column chromatography to afford the D4 (2.70 g, 3.60 mmol, yield: 70.0%) as a yellow solid. LC-MS (ESI) m/z: 749 [M+H]⁺.

### D5: tri-tert-butyl (4S,8S,15S)-15-amino-16-(anthracen-9-yl)-1,6,14-trioxo-2,5,7,13-tetraazahexadecane-1,4,8-tricarboxylate

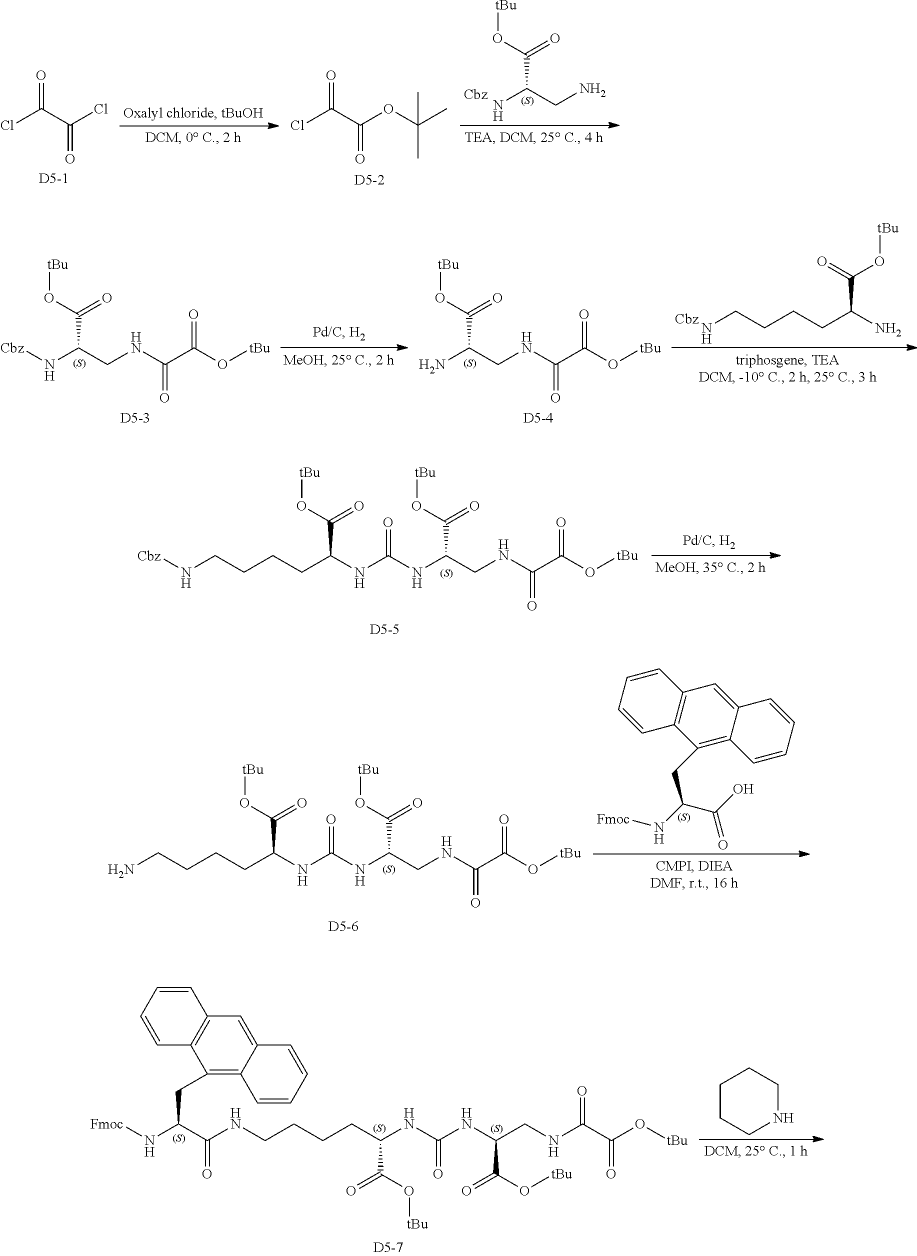

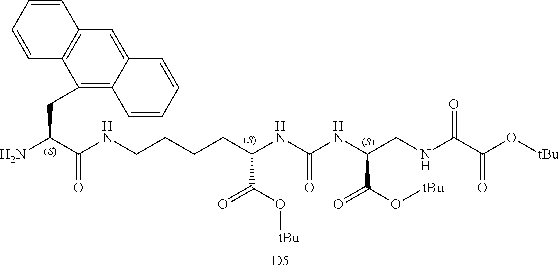

### Step 1

To a solution of D5-1 (5.00 g, 39.40 mmol) in DCM (119 mL) was added t-BuOH (2-methylpropan-2-ol) (2.92 g, 39.40 mmol) at 0° C. The mixture was stirred at 0° C. under N₂ for 2 h. The mixture was then evaporated under reduced pressure to afford D5-2 (5.70 g, crude) as yellow oil, which was used for next step directly without any further purification.

### Step 2

To a solution of D5-2 (5.59 g, 34.00 mmol) in DCM (10 mL) were added tert-butyl (S)-3-amino-2-(((benzyloxy)carbonyl)amino)propanoate (5.00 g, 16.99 mmol) and TEA (9.47 mL, 67.90 mmol). The mixture was stirred at 25° C. under N₂ for 4 h. The resulting mixture was quenched with water (100 mL), extracted with DCM (10 mL×2). The combined organic layers were washed with brine (10 mL×1), dried over Na₂SO₄, filtered and concentrated in vacuum to give the crude product, which was purified by silica gel column chromatography to afford D5-3 (5.07 g, 12.00 mmol, yield: 30.5% with two steps) as yellow oil. LC-MS (ESI) m/z: 423 [M+H]⁺.

### Step 3

To a solution of D5-3 (5.60 g, 13.26 mmol) in MeOH (70 mL) was added 10% Pd/C (1.41 g, 13.26 mmol). The mixture was stirred at 25° C. under H₂ for 12 h. The resulting mixture was filtered and evaporated to dryness to give the D5-4 (3.36 g, crude) as colorless oil. LC-MS (ESI) m/z: 289 [M+H]⁺.

### Step 4

To a solution of tert-butyl N6-((benzyloxy)carbonyl)-L-lysinate (12.27 g, 36.50 mmol) in anhydrous DCM (158 mL) were added Triphosgene (3.80 g, 12.81 mmol) and TEA (20.34 mL, 146.00 mmol) in anhydrous DCM (210 mL) dropwise at −10° C. over 1 h. The reaction mixture was stirred at −10° C. for 2 h, and then a solution of D5-4 (10.52 g, 36.50 mmol) and TEA (10.17 mL, 73.00 mmol) in anhydrous DCM (316 mL) was added over 30 min. The reaction was stirred for another 3 h. The reaction was quenched with water (200 mL) and extracted with DCM (300 mL×2). The combined organic layers were washed brine (800 mL), dried over Na₂SO₄, filtered and concentrated in vacuum to give the crude product, which was purified by reversed phase silica gel column chromatography eluting with ACN in H₂O (0.1% FA) to afford D5-5 (6.37 g, 9.79 mmol, yield: 77.0%) as yellow oil. LC-MS (ESI) m/z: 651 [M+H]⁺.

### Step 5

A mixture of D5-5 (2.53 g, 3.89 mmol) and 10% Pd/C (0.41 g, 0.39 mmol) in MeOH (40 mL) was degassed and purged with H₂ three times. The mixture was stirred at 35° C. under H₂ atmosphere for 2 h. The resulting mixture was filtered and evaporated to dryness to give the D5-6 (2.14 g, crude). LC-MS (ESI) m/z: 517 [M+H]⁺.

### Step 6

To a solution of (S)-2-((((9H-fluoren-9-yl)methoxy)carbonyl)amino)-3-(anthracen-9-yl)propanoic acid (2.00 g, 4.10 mmol) in DMF (10 mL) were added D5-6 (2.23 g, 4.31 mmol), 2-chloro-1-methylpyridinium iodide (1.36 g, 5.33 mmol) and DIEA (2.87 mL, 16.41 mmol) at 25° C. The mixture was stirred at 25° C. under N₂ for 16 h. The resulting mixture was quenched with H₂O (50 mL) and extracted with EA (50 mL×3). The combined organic layers were washed with brine (50 mL×1), dried over Na₂SO₄, filtered and concentrated in vacuum to give a crude product, which was purified by silica gel column chromatography to afford D5-7 (2.10 g, 2.13 mmol, yield: 51.9%) as yellow oil. LC-MS (ESI) m/z: 986 [M+H]⁺.

### Step 7

To a solution of D5-7 (2.00 g, 2.03 mmol) in DCM (16 mL) was added piperidine (4 mL, 40.40 mmol). The mixture was stirred at 25° C. under N₂ for 1 h. The mixture was then evaporated under reduced pressure to give a crude product, which was purified by silica gel column chromatography to afford D5 (602.00 mg, 0.79 mmol, yield: 38.9%) as a yellow solid. LC-MS (ESI) m/z: 764 [M+H]⁺.

### D6: di-tert-butyl (((S)-1-(tert-butoxy)-6-((S)-2-((2-ethoxy-3,4-dioxocyclobut-1-en-1-yl)amino)-3-(naphthalen-2-yl)propanamido)-1-oxohexan-2-yl)carbamoyl)-L-glutamate

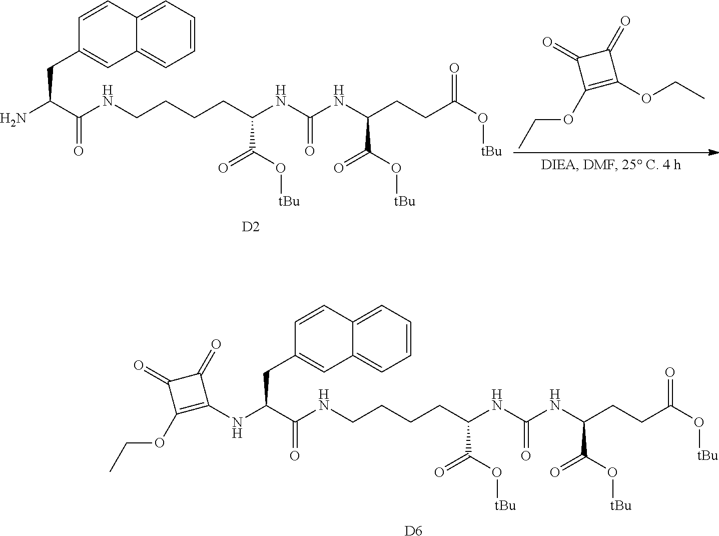

To a solution of D2 (500.00 mg, 0.73 mmol) in DMF (5 mL) were added 3,4-diethoxycyclobut-3-ene-1,2-dione (186.00 mg, 1.10 mmol) and DIEA (0.51 mL, 2.92 mmol). The mixture was stirred at 25° C. under N₂ for 4 h. The reaction was quenched with water (10 mL) and extracted with EA (10 mL×3). The combined organic layers were washed with brine (10 mL×1), dried over Na₂SO₄, filtered and concentrated in vacuum to give the crude product. The crude product was purified by silica gel column chromatography to afford D6 (660.00 mg, crude) as colorless oil. LC-MS (ESI) m/z: 809 [M+H]⁺.

### D7: tert-butyl ((4-aminobicyclo[2.2.2]octan-1-yl)methyl)carbamate

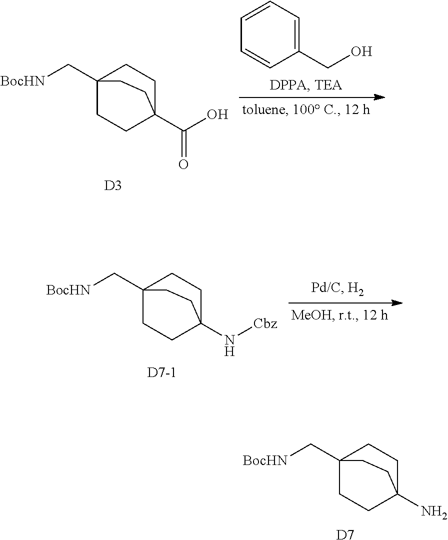

### Step 1

To a solution of D3 (1.00 g, 3.53 mmol) in toluene (10 mL) were added phenylmethanol (0.38 g, 3.53 mmol), diphenylphosphinyl azide (0.77 g, 3.18 mmol) and triethylamine (1.071 g, 10.59 mmol). The mixture was stirred at 100° C. under N₂ for 12 h. The reaction was quenched with water (20 mL) and extracted with EA (10 mL×2). The combined organic layers were washed with brine (10 mL×1), dried over Na₂SO₄, filtered and concentrated in vacuum to give the crude product, which was purified by silica gel column chromatography to afford D7-1 (1.01 g, 2.60 mmol, yield: 73.7%) as yellow oil. LC-MS (ESI) m/z: 389 [M+H]⁺.

### Step 2

To a solution of D7-1 (1.01 g, 2.60 mmol) in MeOH (10 mL) was added 10% wet Pd/C (0.28 g). The suspension was degassed under vacuum and purged with H₂ three times. The mixture was stirred at room temperature under H₂ atmosphere for 12 h. The reaction mixture was filtered and evaporated to dryness to afford D7 (670.00 mg, crude) as colorless oil. LC-MS (ESI) m/z: 255 [M+H]⁺.

### D8: benzyl tert-butyl bicyclo[2.2.2]octane-1,4-diyldicarbamate

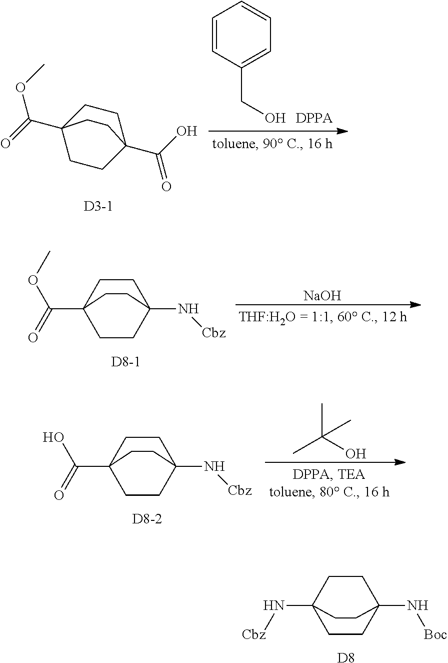

### Step 1

To a solution of D3-1 (8.00 g, 37.70 mmol) in toluene (64 mL) were added phenylmethanol (3.92 mL, 37.70 mmol), diphenyl phosphorazidate (12.18 mL, 56.50 mmol) and TEA (13.13 mL, 94.00 mmol). The mixture was stirred at 90° C. under N₂ for 16 h. The reaction was quenched with H₂O (100 mL) and extracted with EA (100 mL×2). The combined organic layers were washed with brine (100 mL×2), dried over Na₂SO₄, filtered and concentrated in vacuum to give the crude product. The crude product was purified by silica gel column chromatography to afford D8-1 (8.50 g, 26.80 mmol, yield: 71.1%) as a yellow solid. LC-MS (ESI) m/z: 318 [M+H]⁺.

### Step 2

To a solution of D8-1 (8.50 g, 26.8 mmol) in THF (50 mL) and water (50 mL) were added NaOH (3.21 g, 80 mmol). The mixture was stirred at 60° C. under N₂ for 12 h. The mixture was then evaporated under reduced pressure to give crude product, and then quenched with H₂O (50 mL). The aqueous layer was extracted with EA (50 mL×2). The aqueous phase was adjusted pH to 2 with 2N HCl to give a white suspension, which was then filtered. The filter cake was dried in vacuum to give the D8-2 (8.00 g, 26.40 mmol, yield: 98.5%) as a white solid. LC-MS (ESI) m/z: 304 [M+H]⁺.

### Step 3

To a solution of D8-2 (1.00 g, 3.30 mmol) in t-BuOH (15 mL) were added Boc₂O (2.76 mL, 11.87 mmol), TEA (0.55 mL, 3.96 mmol) and diphenylphosphinyl azide (0.96 g, 3.96 mmol). The mixture was stirred at 80° C. under N₂ for 16 h. The reaction was quenched with water (10 mL). The aqueous layer was extracted with EA (10 mL×2). The combined organic layers were washed with brine (10 mL×1), dried over Na₂SO₄, filtered and concentrated in vacuum to give the crude product. The crude product was purified by silica gel column chromatography to afford D8 (1.01 g, 2.70 mmol, yield: 82.1%) as yellow oil. LC-MS (ESI) m/z: 375 [M+H]f.

### D9: tri-tert-butyl 2,2′,2″-(10-((5-(4-aminobicyclo[2.2.2]octan-1-yl)-1,3,4-oxadiazol-2-yl)methyl)-1,4,7,10-tetraazacyclododecane-1,4,7-triyl)triacetate

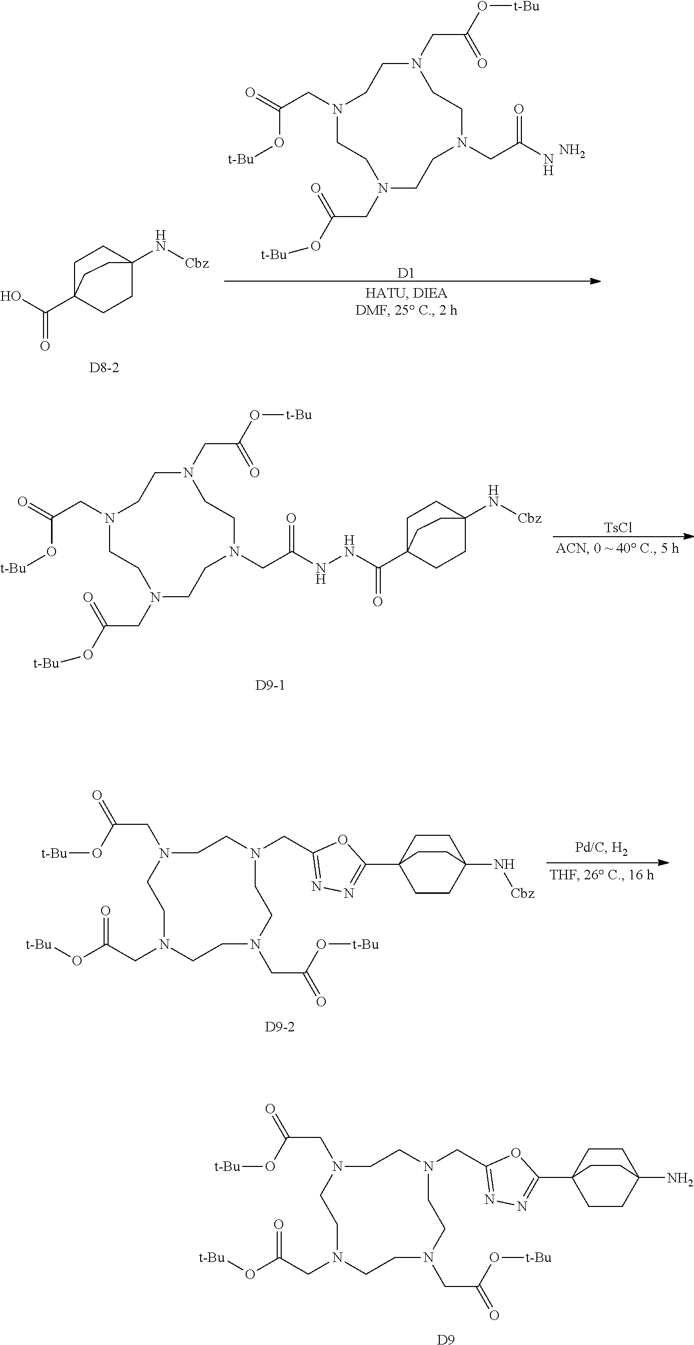

### Step 1

To a solution of D8-2 (0.80 g, 2.64 mmol) in DMF (10 mL) were added HATU (1.50 mg, 3.96 mmol), DIEA (1.38 mL, 7.91 mmol) and D1 (1.70 mg, 2.90 mmol). The mixture was stirred at 25° C. under N₂ for 2 h. The reaction was quenched with H₂O (10 mL) and extracted with EA (100 mL×2). The combined organic layers were washed with brine (150 mL×1), dried over Na₂SO₄, filtered and concentrated in vacuum to give the crude product, which was purified by silica gel column chromatography to afford D9-1 (1.80 g, 2.06 mmol, yield: 78.2%) as yellow oil. LC-MS (ESI) m/z: 872 [M+H]⁺.

### Step 2

To a solution of D9-1 (2.00 g, 2.29 mmol) in MeCN (20 mL) were added TsCl (4-methylbenzenesulfonyl chloride) (1.31 g, 6.88 mmol) and DIEA (2.00 mL, 11.47 mmol) at 0° C. The mixture was stirred at 40° C. under N₂ for 5 h. The resulting mixture was concentrated to give a crude product, which was purified by prep-HPLC (0.10% TFA in water:ACN=2:1) to afford D9-2 (0.33 g, 0.39 mmol, yield: 16.9%) as yellow oil. LC-MS (ESI) m/z: 854 [M+H]⁺.

### Step 3

To a solution of D9-2 (0.33 g, 0.39 mmol) in THF (10 mL) was added 10% Pd/C (41.1 mg). The mixture was stirred at 26° C. under H₂ for 16 h. The resulting mixture was filtered and evaporated to dryness to afford D9 (0.26 g, 0.36 mmol, yield: 93.5%) as yellow oil. LC-MS (ESI) m/z: 720 [M+H]⁺.

### D10: tri-tert-butyl 2,2′,2″-(10-(2-((4-aminobicyclo[2.2.2]octan-1-yl)amino)-2-oxoethyl)-1,4,7,10-tetraazacyclododecane-1,4,7-triyl)triacetate

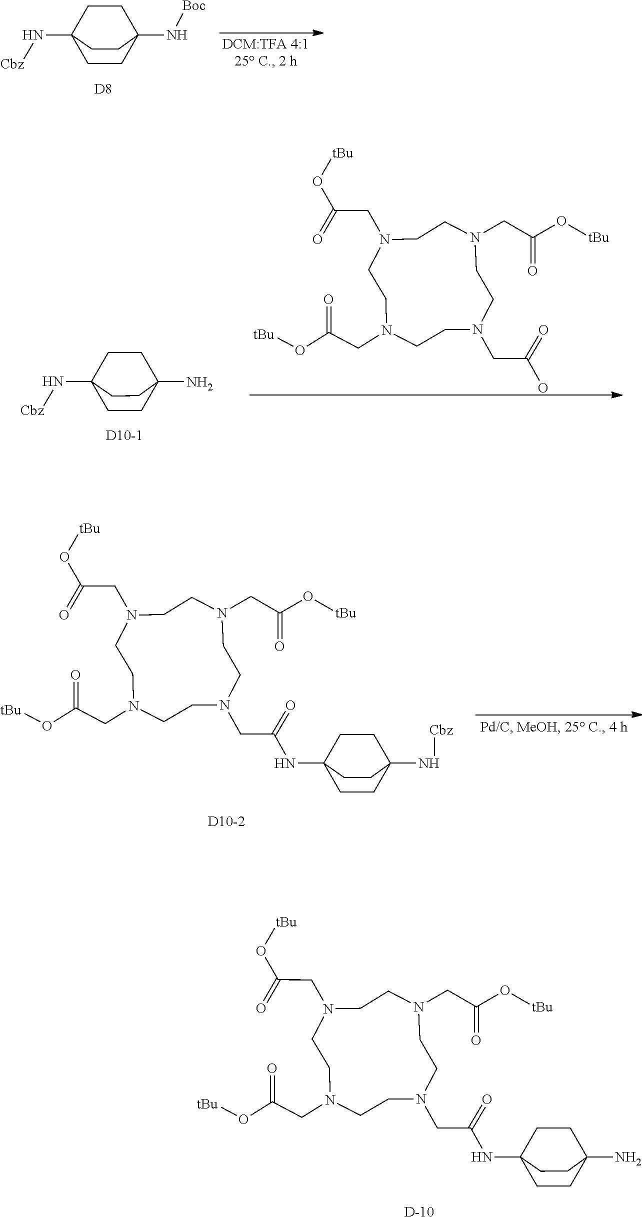

### Step 1

To a solution of D8 (0.40 g, 1.07 mmol) in TFA (1 mL) was added DCM (4.00 mL). The mixture was stirred at 25° C. under N₂ for 2 h. The mixture was then evaporated under reduced pressure to afford crude D10-1 (0.30 g, 1.09 mmol) as a yellow solid. LC-MS (ESI) m/z: 275 [M+H]⁺.

### Step 2

To a solution of D10-1 (0.30 g, 1.09 mmol) in DMF (4.0 mL) were added 2-(4,7,10-tris(2-(tert-butoxy)-2-oxoethyl)-1,4,7,10-tetraazacyclododecan-1-yl)acetic acid (689.00 mg, 1.20 mmol), HATU (624.00 mg, 1.64 mmol) and DIEA (0.76 mL, 4.37 mmol). The mixture was stirred at 25° C. under N₂ for 2 h. The resulting mixture was quenched with water (10 mL) and extracted with EA (10 mL×2). The combined organic layers were washed with brine (10 mL×1), dried over Na₂SO₄, filtered and concentrated in vacuum to give the crude product. The crude product was purified by silica gel column chromatography to afford D10-2 (792.00 mg, 0.96 mmol, yield: 87.0%) as yellow oil. LC-MS (ESI) m/z: 829 [M+H]⁺.

### Step 3

To a solution of D10-2 (790.00 mg, 0.95 mmol) in MeOH (20 mL) was added 10% wet Pd/C (101.00 mg). The mixture was stirred at 25° C. under H₂ for 4 h. The reaction mixture was filtered and concentrated in vacuum to afford D10 (650.00 mg, 0.94 mmol, yield: 98.9%) as colorless oil. LC-MS (ESI) m/z: 695 [M+H]⁺.

### D11 tri-tert-butyl 2,2′,2″-(10-((5-((1r,4r)-4-aminocyclohexyl)-1,3,4-oxadiazol-2-yl)methyl)-1,4,7,10-tetraazacyclododecane-1,4,7-triyl)triacetate

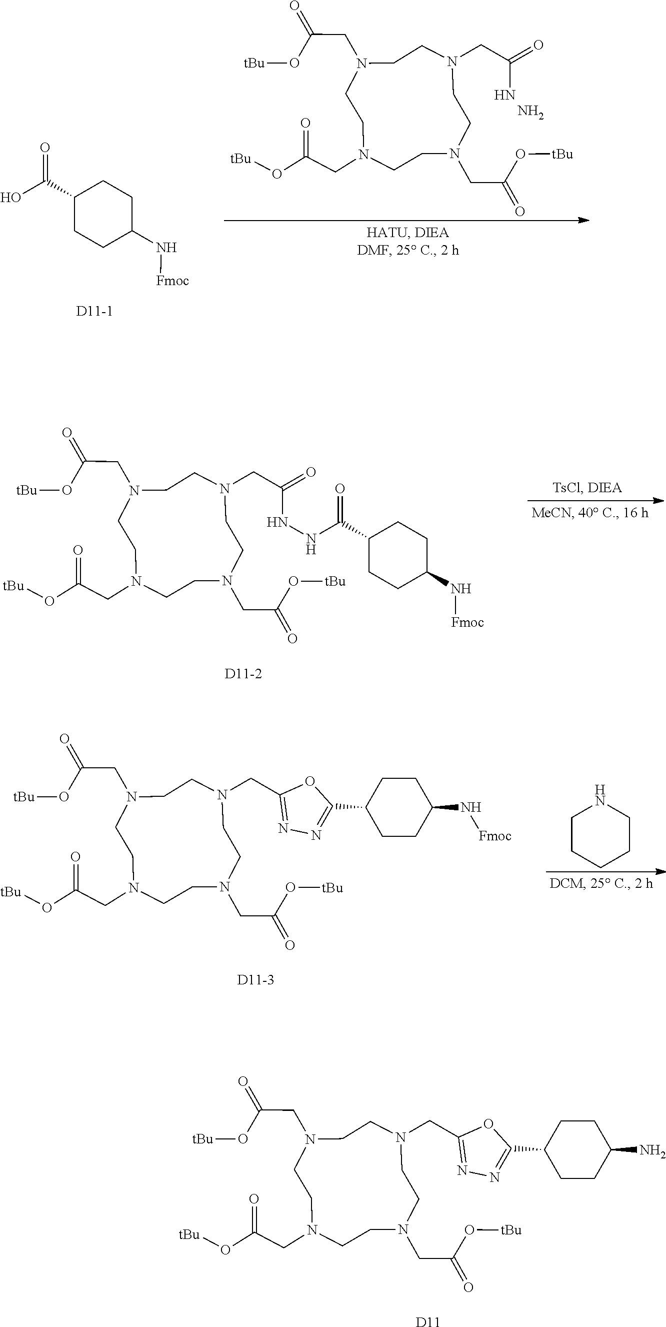

### Step 1

To a solution of D11-1 (700.00 mg, 1.19 mmol) in DMF (14 mL) was added DIEA (0.83 ml, 4.77 mmol), HATU (544.00 mg, 1.43 mmol) and D1 (523.00 mg, 1.43 mmol). The mixture was stirred at 25° C. under N₂ for 2 h. The reaction was quenched with water (10 mL) and extracted with EA (25 mL×2). The combined organic layers were washed with brine (25 mL×2), dried over Na₂SO₄, filtered and concentrated in vacuum to give the crude product. The crude product was purified by silica gel column chromatography to afford D11-2 (500.00 mg, 0.54 mmol, yield: 44.9%) as yellow oil. LC-MS (ESI) m/z: 934 [M+H]⁺.

### Step 2

To a solution of D11-2 (500.00 mg, 0.54 mmol) in MeCN (8 mL) were added TsCl (204.00 mg, 1.07 mmol), DIEA (0.28 ml, 1.61 mmol). The mixture was stirred at 40° C. under N₂ for 16 h The reaction was quenched with water (5 mL) extracted with EA (25 mL×2). The combined organic layers were washed with brine (25 mL×2), dried over Na₂SO₄, filtered and concentrated in vacuum to give the crude product. The crude product was purified by silica gel column chromatography to afford D11-3 (160.00 mg, 0.18 mmol, yield: 32.6%) as a yellow solid. LC-MS (ESI) m/z: 916 [M+H]⁺.

### Step 3

To a solution of D11-3 (140.00 mg, 0.15 mmol) in DCM (1 mL) was added piperidine (0.50 ml, 5.05 mmol). The mixture was stirred at 25° C. under N₂ for 2 h. The mixture was then evaporated under reduced pressure to give a crude product. The crude product was purified by silica gel column chromatography to afford D11 (63.00 mg, 0.09 mmol, yield: 59.4%) as a yellow solid. LC-MS (ESI) m/z: 694 [M+H]⁺.

### D12: di-tert-butyl 2,2′-(4-(2-amino-2-oxoethyl)-10-(2-((4-aminobicyclo[2.2.2]octan-1-yl)amino)-2-oxoethyl)-1,4,7,10-tetraazacyclododecane-1,7-diyl)diacetate

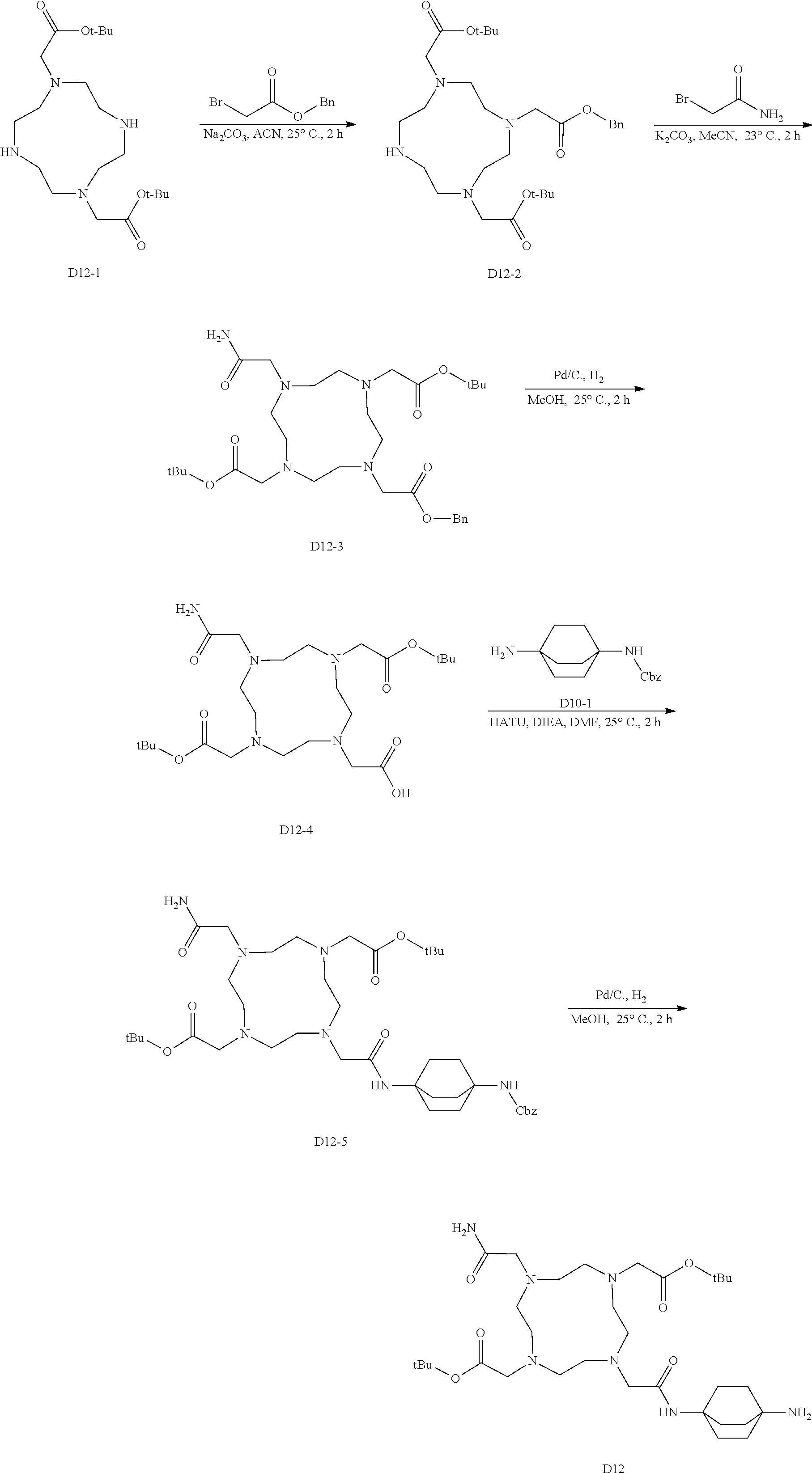

### Step 1

To a solution of D12-1 (500.00 mg, 1.25 mmol) in ACN (5 ml) were added Na₂CO₃ (146.00 mg, 1.37 mmol) and benzyl 2-bromoacetate (0.16 ml, 1.00 mmol) at 0° C. The mixture was stirred at 25° C. for 2 h under N₂ atmosphere. The reaction was filtered, and the filtrate was concentrated and purified to afford D12-2 (246.00 mg, 0.49 mmol, yield: 35.9%) as yellow oil. LC-MS (ESI) m/z: 549 [M+H]⁺.

### Step 2

To a solution of D12-2 (246.00 mg, 0.45 mmol) in ACN (3 ml) were added K₂CO₃ (124.00 mg, 0.90 mmol) and 2-bromoacetamide (93.00 mg, 0.67 mmol) at 23° C. After addition, the mixture was stirred at 23° C. for 2 h under N₂. The reaction was quenched by addition of H₂O (20 mL) and extracted with EA (50 mL×2). The combined organic layers were washed with brine (50 mL), dried over Na₂SO₄, filtered and concentrated to give the crude product D12-3 (306.00 mg, crude). LC-MS (ESI) m/z: 606 [M+H]f.

### Step 3

To a solution of D12-3 (350.00 mg, 0.58 mmol) in MeOH (30 mL) was added 10% wet Pd/C (61.50 mg) at 25° C., and the mixture was stirred at 25° C. under H₂ atmosphere (15 psi) for 2 h. The resulting mixture was filtered and the filtrate was evaporated to dryness to give the crude product D12-4 (289.00 mg, 0.56 mmol, yield: 96.9%) as yellow oil. LC-MS (ESI) m/z: 516 [M+H]⁺.

### Step 4

To a solution of D12-4 (280.00 mg, 0.54 mmol) in DMF (3 mL) were added D10-1 (164.00 mg, 0.60 mmol), HATU (310.00 mg, 0.82 mmol) and DIEA (0.38 mL, 2.17 mmol). The mixture was stirred at 25° C. under N₂ for 2 h. The resulting mixture was quenched with water (10 mL) and extracted with EA (25 mL×2). The combined organic layers were washed with brine (50 mL), dried over Na₂SO₄, filtered and concentrated in vacuum to give the crude product, which was purified by silica gel column chromatography to afford D12-5 (382.00 mg, 0.50 mmol, yield: 92.6%) as colorless oil. LC-MS (ESI) m/z: 772 [M+H]⁺.

### Step 5

To a solution of D12-5 (430.00 mg, 0.56 mmol) in MeOH (10 mL) was added 10% wet Pd/C (43.00 mg) and the mixture was stirred at 25° C. under H₂ atmosphere for 2 h. The reaction mixture was filtered and the filtrate was evaporated to dryness to give the crude product. D12 (289.00 mg, 0.45 mmol, yield: 80.4%) as colorless oil. LC-MS (ESI) m/z: 638 [M+H]⁺.

### D13: di-tert-butyl 2,2′-(4-(2-amino-2-oxoethyl)-1,4,7,10-tetraazacyclododecane-1,7-diyl)diacetate

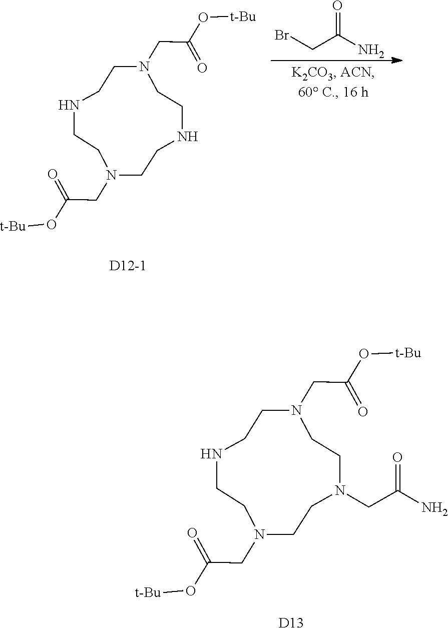

To a solution of D12-1 (5.00 g, 12.48 mmol) in ACN (100 mL) were added K₂CO₃ (6.90 g, 49.9 mmol) and 2-bromoacetamide (2.58 g, 18.72 mmol) in ACN (50 mL) slowly at 60° C. The mixture was stirred at 60° C. under N₂ for 16 h. The mixture was then evaporated under reduced pressure to give crude product, which was purified by reversed phase eluting with ACN in H₂O (0.1% FA) to afford D13 (0.80 g, 1.75 mmol, yield: 14.0%) as an off-white solid. LC-MS (ESI) m/z: 458 [M+H]⁺.

### D14: di-tert-butyl 2,2′-(4-(2-amino-2-oxoethyl)-1,4,7,10-tetraazacyclododecane-1,7-diyl)diacetate

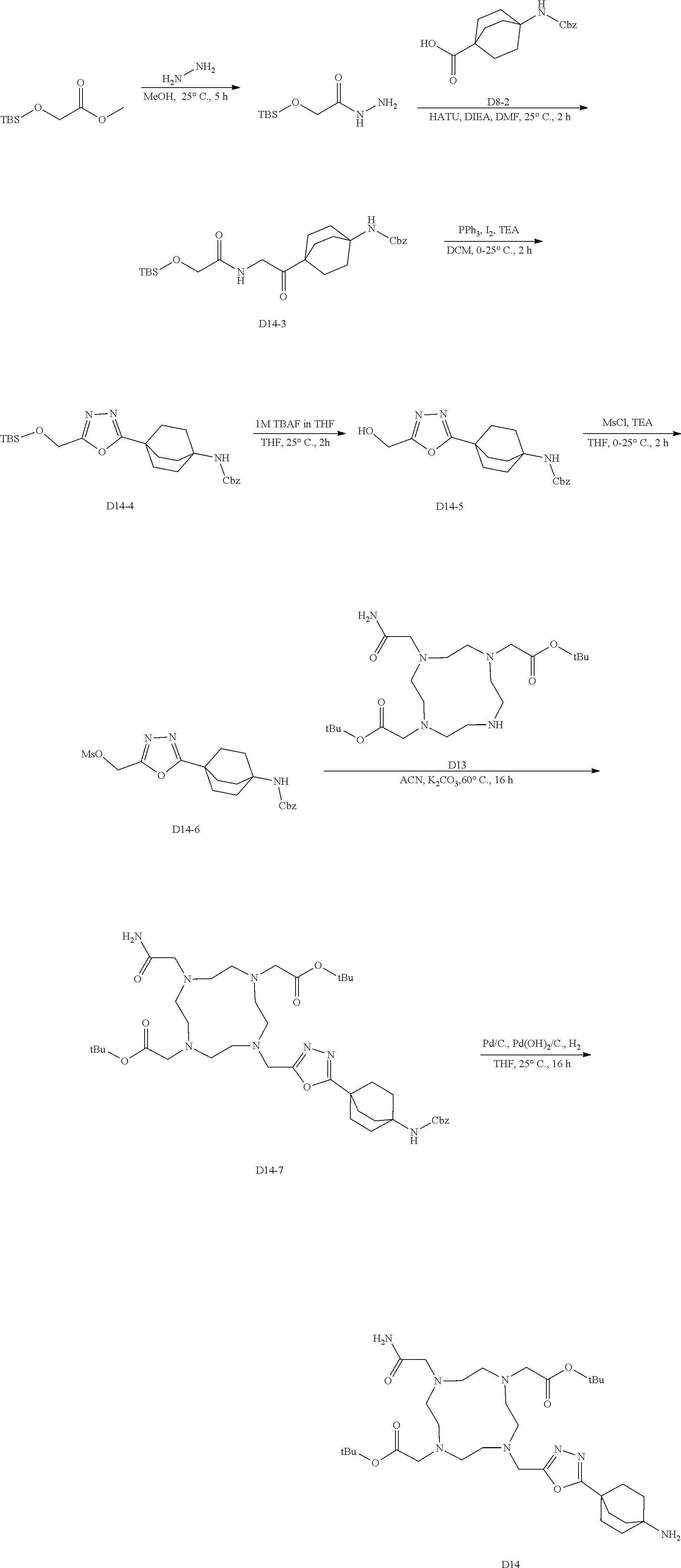

### Step 1:

To a solution of D14-1 (10.00 g, 48.90 mmol) in MeOH (50 mL) was added hydrazinium hydroxide (49.9 mL, 1.03 mol). The mixture was stirred at 25° C. under N₂ for 5 h. The mixture was then evaporated under reduced pressure to give crude product, which was purified by silica gel column chromatography to afford D14-2 (4.30 g, 21.04 mmol, yield: 43.0%) as a white solid. LC-MS (ESI): 205 [M+H]⁺.

### Step 2:

To a solution of D8-2 (2.00 g, 6.59 mmol) in DMF (20 mL) were added DIEA (3.45 mL, 19.78 mmol), HATU (5.01 g, 13.19 mmol) and D14-2 (1.35 g, 6.59 mmol). The mixture was stirred at 25° C. under N₂ for 2 h. The reaction was quenched with H₂O (50 mL) and extracted with EA (150 mL×3). The combined organic layers were washed with brine (100 mL×2), dried over Na₂SO₄, filtered and concentrated in vacuum to give a crude product, which purified by silica gel column chromatography to afford D14-3 (yield: 3 g, 6.13 mmol) as a white solid. LC-MS (ESI): 490 [M+H]⁺.

### Step 3:

To a solution of triphenylphosphine (2.14 g, 8.17 mmol) and 12 (2.07 g, 8.17 mmol) in DCM (100 mL) were added TEA (2.85 mL, 20.42 mmol) and D14-3 (2.00 g, 4.08 mmol) at 0° C. The mixture was stirred at 25° C. under N₂ for 2 h. The mixture was then evaporated under reduced pressure to give a crude product, which was purified by silica gel column chromatography to afford D14-4 (1.4 g, 2.97 mmol, yield: 72.7%) as a white solid. LC-MS (ESI): 472 [M+H]⁺.

### Step 4:

To a solution of D14-4 (1.40 g, 2.97 mmol) in THF (50 mL) was added TBAF (Tetrabutylammonium fluoride) (5.94 mL, 5.94 mmol). The mixture was stirred at 25° C. under N₂ for 2 h. The reaction was quenched with H₂O (50 mL) and extracted with EA (50 mL×2). The combined organic layers were washed with brine (50 mL×2), dried over Na₂SO₄, filtered and concentrated in vacuum to give the crude product, which was purified by silica gel column chromatography to afford D14-5 (0.90 g, 2.52 mmol, yield: 85.0%) as a white solid. LC-MS (ESI): 358 [M+H]⁺.

### Step 5:

To a solution of D14-5 (0.90 g, 2.52 mmol) in THF (10 mL) were added TEA (1.05 mL, 7.55 mmol) and MsCl (0.29 ml, 3.78 mmol) at 0° C. The mixture was stirred at 25° C. under N₂ for 2 h. The reaction was quenched with H₂O (50 mL) and extracted with EA (50 mL×2). The combined organic layers were washed with brine (50 mL×2), dried over Na₂SO₄, filtered and concentrated in vacuum to give the crude product D14-6 (1.10 g, 2.53 mmol, yield: 100%). LC-MS (ESI): 436 [M+H]⁺.

### Step 6:

To a solution of D14-6 (1.10 g, 2.53 mmol) in ACN (20 mL) were added D13 (0.80 g, 1.77 mmol) and K₂CO₃ (1.05 g, 7.58 mmol). The mixture was stirred at 60° C. under N₂ for 16 h. The mixture was then evaporated under reduced pressure to give a crude product, which was purified by silica gel column chromatography to afford D14-7 (1.1 g, 1.38 mmol, yield: 54.6%) as yellow oil. LC-MS (ESI): 797 [M+H]⁺.

### Step 7:

To a solution of D14-7 (1.10 g, 1.38 mmol) in THF (30 mL) were added 10% wet Pd/C (0.18 g, 0.14 mmol) and 10% wet Pd(OH)₂/C (0.19 g, 0.14 mmol). The mixture was stirred at 25° C. under H₂ balloon for 16 h. The resulting mixture was filtered and evaporated under reduced pressure to give crude product D14 (0.91 g, 1.38 mmol, yield: 100%) as yellow oil. LC-MS (ESI): 663 [M+H]f.

### E1: (((S)-1-carboxy-5-((S)-2-((3,4-dioxo-2-((4-((2-(4,7,10-tris(carboxymethyl)-1,4,7,10-tetraazacyclododecan-1-yl)acetamido)methyl)bicyclo[2.2.2]octan-1-yl)amino)cyclobut-1-en-1-yl)amino)-3-(naphthalen-2-yl)propanamido)pentyl)carbamoyl)-L-glutamic acid (Method A)

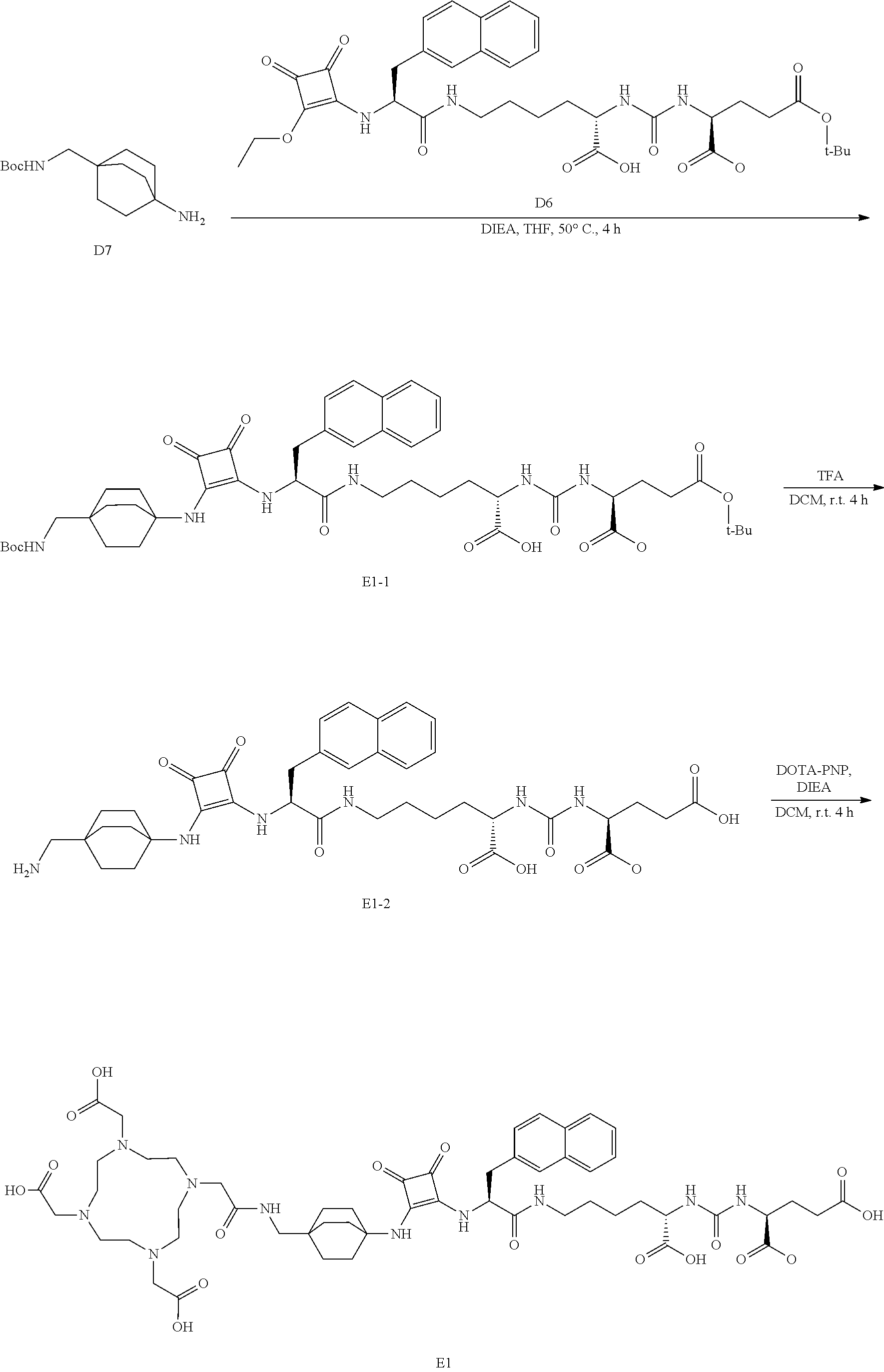

### Step 1

To a solution of D7 (47.2 mg, 0.19 mmol) in THF (0.50 mL) were added D6 (50.00 mg, 0.06 mmol), and DIEA (0.04 mL, 0.25 mmol). The mixture was stirred at 50° C. under N₂ for 4 h. The reaction was quenched with water (10 mL) and extracted with EA (10 mL×2). The combined organic layers were washed with brine (10 mL×1), dried over Na₂SO₄, filtered and concentrated in vacuum to give a crude product. The crude product was purified by silica gel column chromatography to afford E1-1 (14.00 mg, 0.01 mmol, yield: 22.3%) as a white solid. LC-MS (ESI) m/z: 1017 [M+H]⁺.

### Step 2

To a solution of E1-1 (14.00 mg, 0.01 mmol) in DCM (0.50 mL) was added TFA (0.50 mL). The mixture was stirred at 25° C. under N₂ for 4 h. The mixture was then evaporated under reduced pressure to give crude E1-2 (11.00 mg) as a white solid. LC-MS (ESI) m/z: 749 [M+H]⁺.

### Step 3

To a solution of E1-2 (110.00 mg, 0.15 mmol) in DMF (1 mL) were added DOTA-PNP (2,2′,2″-(10-(2-(4-nitrophenoxy)-2-oxoethyl)-1,4,7,10-tetraazacyclododecane-1,4,7-triyl)triacetic acid) (85.00 mg, 0.16 mmol) and DIEA (0.10 mL, 0.59 mmol). The mixture was stirred at room temperature under N₂ for 4 h. The resulting mixture was concentrated to give a crude, which was purified by prep-HPLC (Method 3) to afford the title compound E1 (6.00 mg, 4.87 μmol, yield: 3.3%) as a white solid. LC-MS (ESI) m/z: 1135 [M+H]⁺. ¹H NMR (400 MHz, D₂O) δ 7.81 (td, J=8.7, 6.1 Hz, 3H), 7.64 (s, 1H), 7.48 (ddd, J=7.3, 5.0, 1.8 Hz, 2H), 7.34 (dd, J=8.5, 1.7 Hz, 1H), 4.87 (dd, J=9.4, 5.5 Hz, 1H), 3.99 (dd, J=8.3, 5.0 Hz, 1H), 3.92 (dd, J=8.3, 4.7 Hz, 1H), 3.77 (d, J=8.2 Hz, 3H), 3.66 (p, J=6.6 Hz, 1H), 3.52 (s, 2H), 3.48-3.33 (m, 10H), 3.22-3.01 (m, 12H), 2.89 (s, 2H), 2.29 (dd, J=8.7, 7.1 Hz, 2H), 2.07-1.93 (m, 1H), 1.90-1.75 (m, 1H), 1.67-1.54 (m, 7H), 1.54-1.33 (m, 9H), 1.19 (dt, J=15.4, 6.6 Hz, 2H).

### E2: (S)-2-(3-((S)-5-((S)-3-(anthracen-9-yl)-2-((3,4-dioxo-2-((4-((2-(4,7,10-tris(carboxymethyl)-1,4,7,10-tetraazacyclododecan-1-yl)acetamido)methyl)bicyclo[2.2.2]octan-1-yl)amino)cyclobut-1-en-1-yl)amino)propanamido)-1-carboxypentyl)ureido)hexanedioic acid (Method B)

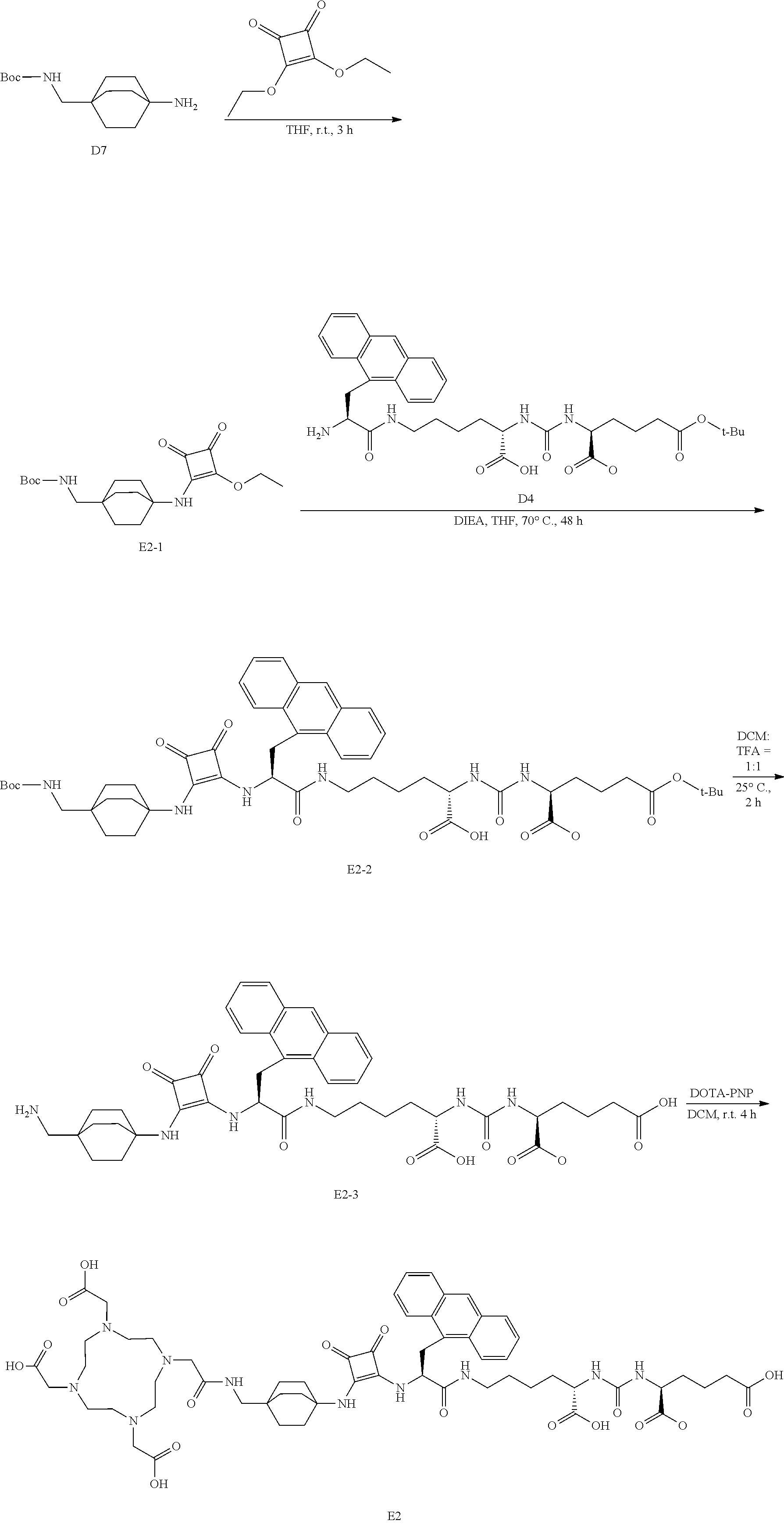

### Step 1

To a solution of 3,4-diethoxycyclobut-3-ene-1,2-dione (0.92 mL, 6.19 mmol) in THF (15 mL) were added TEA (1.036 mL, 7.43 mmol) and D7 (630.00 mg, 2.48 mmol). The mixture was stirred at room temperature under N₂ for 3 h. The resulting mixture was concentrated to give a crude product, which was purified by silica gel column chromatography to afford E2-1 (750.00 mg, 1.98 mmol, yield: 80.0%) as colorless oil. LC-MS (ESI) m/z: 379 [M+H]⁺.

### Step 2

To a solution of d E2-1 (100.00 mg, 0.13 mmol) in THF (0.5 mL) were added DIEA (0.093 mL, 0.534 mmol) and D4 (50.50 mg, 0.13 mmol). The mixture was stirred at 70° C. under N₂ for 48 h. The reaction was quenched with H₂O (10 mL) and extracted with EA (10 mL×2). The combined organic layers were washed with brine (10 mL×1), dried over Na₂SO₄, filtered and concentrated in vacuum to give the crude product, which was purified by silica gel column chromatography to afford E2-2 (68.00 mg, 0.063 mmol, yield: 47.1%) as yellow oil. LC-MS (ESI) m/z: 1081 [M+H]⁺.

### Step 3

A solution of E2-2 (68.00 mg, 0.063 mmol) in DCM (1 mL) and TFA (1 mL) was stirred at 25° C. under N₂ for 2 h. The reaction mixture was concentrated to give E2-3 (48.00 mg, 0.059 mmol, yield: 93.6%) as yellow oil. LC-MS (ESI) m/z: 813 [M+H]⁺.

### Step 4

To a solution of E2-3 (49.00 mg, 0.060 mmol) in DMF (0.5 mL) were added TEA (0.042 mL, 0.31 mmol) and DOTA-PNP (31.70 mg, 0.060 mmol). The mixture was stirred at 25° C. under N₂ for 5 h. The crude product was purified by prep-HPLC (method 3) to afford E2 (9.50 mg, 7.60 μmol, yield: 12.6%) as a white solid. LC-MS (ESI) m/z: 1199 [M+H]⁺. ¹H NMR (400 MHz, D₂O) (8.46 (s, 1H), 8.34-8.23 (m, 2H), 8.09-7.99 (m, 2H), 7.66-7.42 (m, 4H), 4.95-4.83 (m, 1H), 4.27-4.18 (m, 1H), 4.13-4.06 (m, 1H), 4.06-3.96 (m, 2H), 3.89-3.71 (m, 4H), 3.55-3.51 (m, 2H), 3.47-3.33 (m, 11H), 3.26-3.17 (m, 2H), 3.16-3.04 (m, 8H), 2.97-2.89 (m, 2H), 2.27-2.19 (m, 2H), 1.93-1.86 (m, 1H), 1.76-1.67 (m, 2H), 1.62-1.51 (m, 9H), 1.49-1.37 (m, 7H), 1.32-1.22 (m, 2H).

### E3: (4S,8S,15S)-16-(anthracen-9-yl)-15-((3,4-dioxo-2-((4-((2-(4,7,10-tris(carboxymethyl)-1,4,7,10-tetraazacyclododecan-1-yl)acetamido)methyl)bicyclo[2.2.2]octan-1-yl)amino)cyclobut-1-en-1-yl)amino)-1,6,14-trioxo-2,5,7,13-tetraazahexadecane-1,4,8-tricarboxylic acid (Method C)

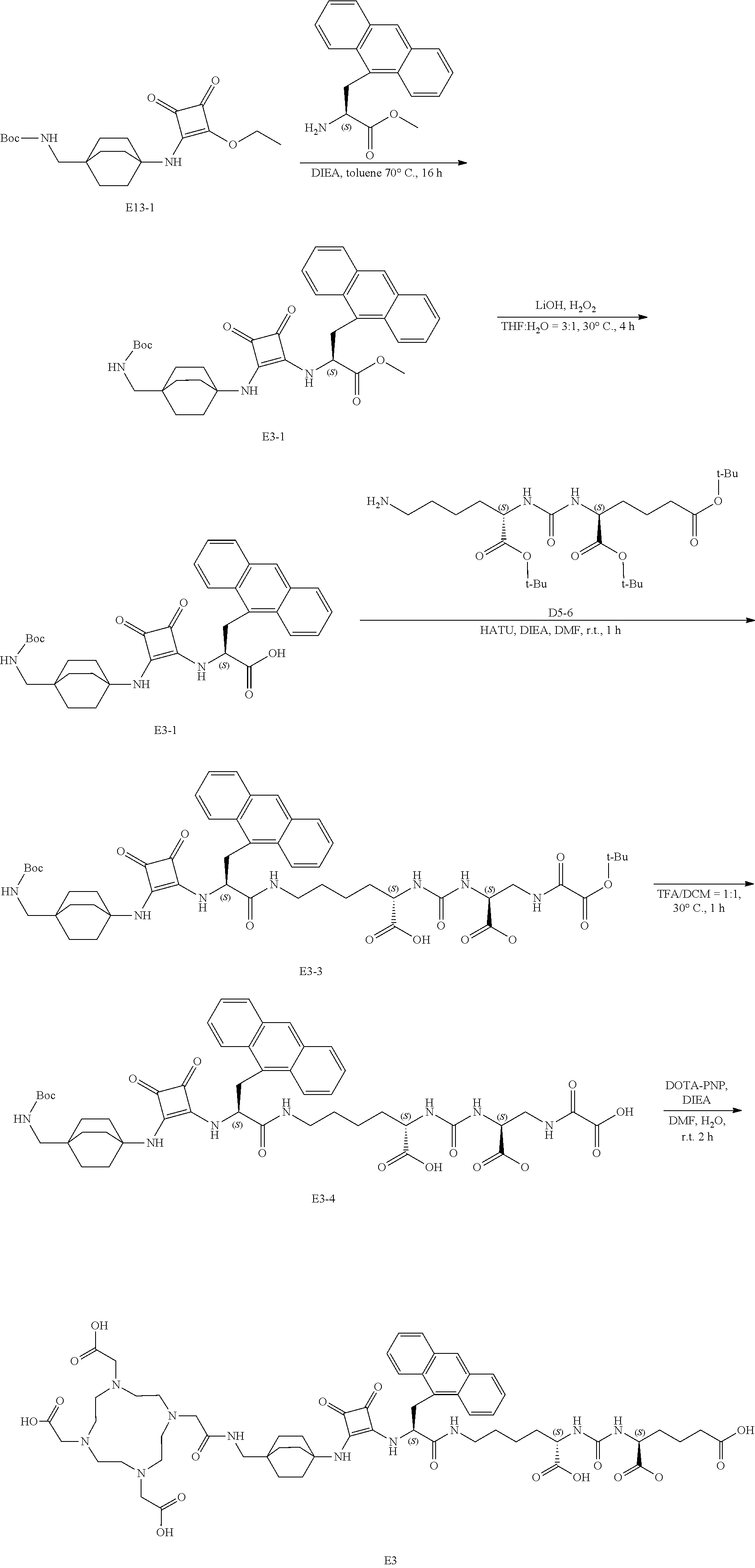

### Step 1

To a solution of E2-1 (407.00 mg, 1.08 mmol) in toluene (5 mL) were added DIEA (0.75 mL, 4.30 mmol) and methyl (S)-2-amino-3-(anthracen-9-yl)propanoate hydrochloride (340.00 mg, 1.08 mmol). The mixture was stirred at 70° C. under N₂ atmosphere for 16 h. The reaction mixture was concentrated to give a crude product, which was purified by silica gel column chromatography to afford E3-1 (500.00 mg, 0.82 mmol, yield: 76.0%) as a white solid. LC-MS (ESI) m/z: 612 [M+H]⁺.

### Step 2

To a solution of E3-1 (250.00 mg, 0.41 mmol) in THF (3 mL) and water (1 mL) were added LiOH (19.57 mg, 0.82 mmol) and H₂O₂ (0.08 mL, 0.82 mmol) at 0° C. The mixture was stirred at 30° C. under N₂ atmosphere for 4 h. The reaction was quenched with aq. Na₂S₂O₃ (5 mL) at 0° C., then adjusted pH to 5 with 1N HCl. The aqueous layer was extracted with EA (10 mL×2). The combined organic layers were washed with brine (10 mL), dried over Na₂SO₄, filtered and concentrated in vacuum to give the crude product E3-2 (240.00 mg, 0.40 mmol) as a yellow solid. LC-MS (ESI) m/z: 598 [M+H]⁺.

### Step 3

To a solution of E3-2 (100.00 mg, 0.17 mmol) in DMF (2 mL) were added D5-6 (104.00 mg, 0.20 mmol), HATU (63.60 mg, 0.17 mmol) and DIEA (0.03 mL, 0.17 mmol). The mixture was stirred at room temperature under N₂ for 1 h. The reaction mixture was quenched with water (10 mL) and exacted with EA (10 mL×2). The combined organic layers were washed with brine (10 mL), dried over Na₂SO₄, filtered and concentrated to give a crude product, which was purified by silica gel column chromatography to afford E3-3 (72.00 mg, 0.07 mmol, yield: 39.3%) as colorless oil. LC-MS (ESI) m/z: 1097 [M+H]⁺.

### Step 4

To a solution of E3-3 (72.00 mg, 0.07 mmol) in DCM (2 mL) was added TFA (1 mL, 12.98 mmol). The mixture was stirred at 30° C. under N₂ for 1 h. The mixture was evaporated under reduced pressure to give crude E3-4 (55.00 mg, 0.07 mmol) as brown oil. LC-MS (ESI) m/z: 828 [M+H]⁺.

### Step 5

To a solution of E3-4 (55.00 mg, 0.07 mmol) in DMF (1 mL) and Water (0.3 mL) were added DOTA-PNP (52.40 mg, 0.10 mmol) and DIEA (0.05 mL, 0.27 mmol). The mixture was stirred at room temperature under N₂ for 2 h. The resulting mixture was concentrated to give a crude, which was purified by prep-HPLC (Method 1) to afford the title compound E3 (27.00 mg, 0.02 mmol, yield: 33.3%) as a white solid. LC-MS (ESI) m/z: 1214 [M+H]⁺. ¹H NMR (400 MHz, DMSO-d6) β 8.72 (t, J=6.0 Hz, 1H), 8.50 (s, 1H), 8.49-8.39 (m, 2H), 8.19-8.11 (m, 2H), 8.07-8.01 (m, 3H), 7.76 (s, 1H), 7.56-7.45 (m, 4H), 6.42 (d, J=8.0 Hz, 1H), 6.31 (d, J=8.1 Hz, 1H), 5.08-4.96 (m, 1H), 4.33-4.20 (m, 1H), 4.06-3.88 (m, 2H), 3.78-3.67 (m, 3H), 3.62 (s, 6H), 3.44-3.38 (m, 2H), 3.21-3.12 (m, 4H), 3.06-2.96 (m, 10H), 2.93-2.89 (m, 2H), 2.85-2.77 (m, 2H), 2.75-2.65 (m, 2H), 1.83-1.70 (m, 6H), 1.51-1.40 (m, 6H), 1.36-1.24 (m, 2H), 0.96-0.77 (m, 4H).

### E4: (S)-2-(3-((S)-5-((S)-3-(anthracen-9-yl)-2-((3,4-dioxo-2-((4-(2-(4,7,10-tris(carboxymethyl)-1,4,7,10-tetraazacyclododecan-1-yl)acetamido)bicyclo[2.2.2]octan-1-yl)amino)cyclobut-1-en-1-yl)amino)propanamido)-1-carboxypentyl)ureido)hexanedioic acid (Method D)

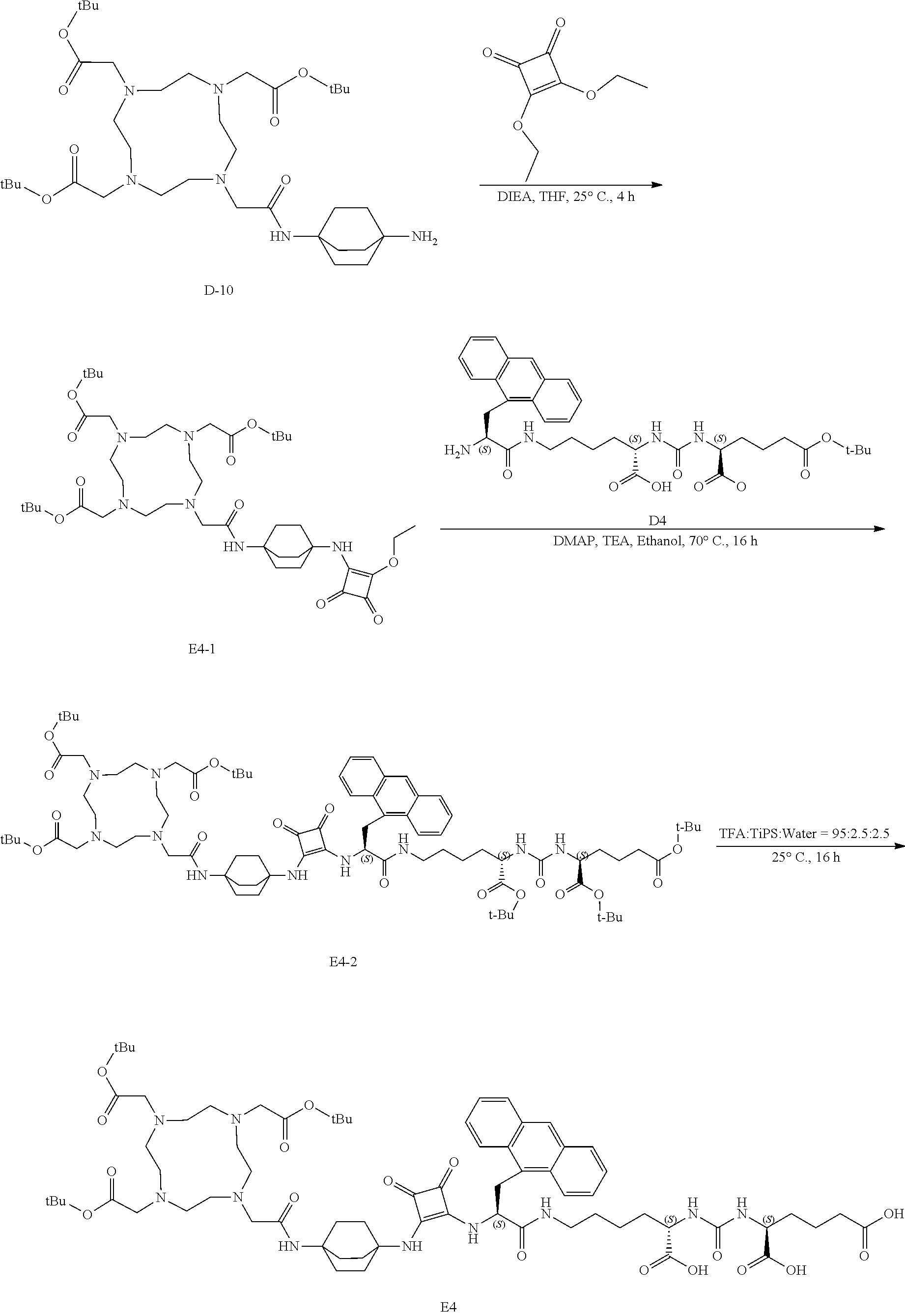

### Step 1

To a solution of D10 (150.00 mg, 0.22 mmol) in THF (1 mL) were added 3,4-diethoxycyclobut-3-ene-1,2-dione (44.10 mg, 0.26 mmol), DIEA (0.15 mL, 0.86 mmol). The mixture was stirred at 25° C. under N₂ for 4 h. The reaction was quenched with water (20 mL) and extracted with EA (20 mL×2). The combined organic layers were washed with brine (10 mL), dried over Na₂SO₄, filtered and concentrated in vacuum to give the crude product, which was purified by silica gel column chromatography to afford E4-1 (149.00 mg, 0.18 mmol, yield: 84.3%) as colorless oil. LC-MS (ESI) m/z: 819 [M+H]⁺.

### Step 2

To a solution of E4-1 (140.00 mg, 0.17 mmol) in EtOH (1.5 mL) was added D4 (90.00 mg, 0.12 mmol), TEA (0.071 mL, 0.51 mmol). The mixture was stirred at 70° C. under N₂ for 16 h. The reaction was quenched with water (20 mL) and extracted with EA (20 mL×3). The combined organic layers were washed with brine, dried over Na₂SO₄, filtered and concentrated in vacuum to give the crude product, which was purified by silica gel column chromatography to afford E4-2 (54.00 mg, 0.035 mmol, yield: 20.8%) as yellow oil. LC-MS (ESI) m/z: 1522 [M+H]⁺.

### Step 3

A solution of E4-2 (54.00 mg, 0.035 mmol) in TFA (0.5 mL), triisopropylsilane (0.013 mL, 0.035 mmol) and water (0.013 mL, 0.72 mmol) was stirred at 25° C. under N₂ for 16 h. The resulting mixture was concentrated to give a crude, which was purified by prep-HPLC (Method 3) to afford the title compound E4 (3.00 mg, 2.44 μmol, yield: 6.9%) as a white solid. LC-MS (ESI) m/z: 1185 [M+H]⁺. ¹H NMR (400 MHz, D₂O) β 8.56 (s, 1H), 8.38 (d, J=8.8 Hz, 2H), 8.14 (d, J=8.3 Hz, 2H), 7.87-7.58 (m, 4H), 4.98-4.95 (m, 1H), 4.29 (d, J=11.7 Hz, 2H), 4.21-4.11 (m, 3H), 4.08-4.04 (m, 1H), 3.87 (s, 1H), 3.71-3.58 (m, 2H), 3.56-3.42 (m, 4H), 3.42-3.31 (m, 2H), 3.19 (d, J=28.4 Hz, 5H), 3.12-3.03 (m, 1H), 2.96-2.65 (m, 5H), 2.46-2.18 (m, 5H), 2.06-1.93 (m, 5H), 1.84-1.67 (m, 7H), 1.64-1.54 (m, 4H), 1.54-1.43 (m, 2H), 1.42-1.24 (m, 5H).

### E6: (((S)-1-carboxy-5-((S)-2-((3,4-dioxo-2-((4-(2-(4,7,10-tris(carboxymethyl)-1,4,7,10-tetraazacyclododecan-1-yl)acetamido)bicyclo[2.2.2]octan-1-yl)amino)cyclobut-1-en-1-yl)amino)-3-(naphthalen-2-yl)propanamido)pentyl)carbamoyl)-L-glutamic acid (Method A)

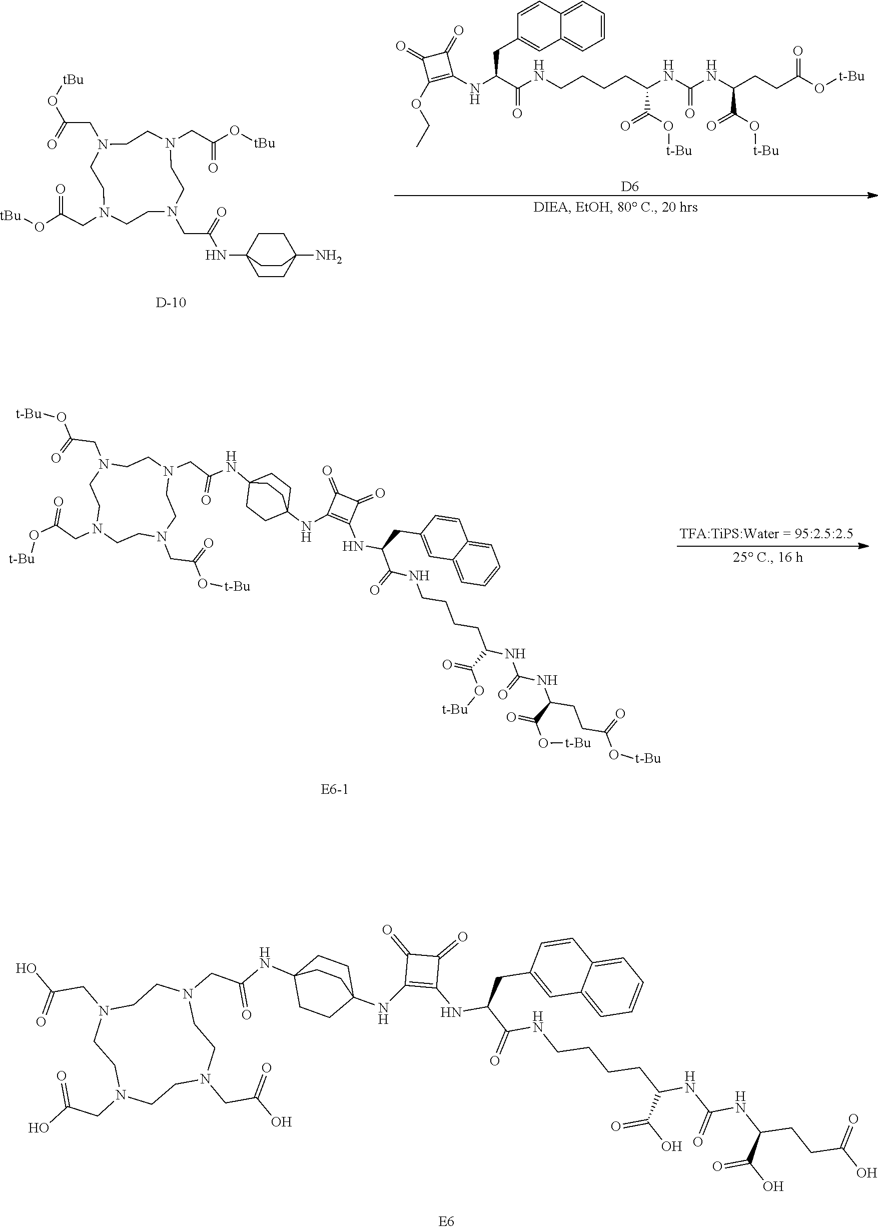

### Step 1:

To a solution of D10 (103.00 mg, 0.15 mmol) in EtOH (0.5 ml) were added DIEA (0.086 ml, 0.49 mmol) and D6 (100.00 mg, 0.12 mmol). The mixture was stirred at 80° C. under N₂ for 20 hrs. The reaction was quenched with H₂O (5 mL). The aqueous layer was extracted with EA (5 mL×2). The combined organic layers were washed with brine (5 mL×2), dried over Na₂SO₄, filtered, and concentrated in vacuum to give a crude product. The crude product was purified by silica gel column chromatography to afford the title compound E6-1 (150.00 mg, 0.10 mol, yield: 83.0%) as yellow oil. LC-MS (ESI) m/z: 1458 [M+H]⁺.

### Step 2:

A solution of E6-1 (100.00 mg, 0.069 mmol) in TFA (0.95 mL), TIPS (0.025 mL) and water (0.025 mL) was stirred at 25° C. under N₂ for 12 hrs. The resulting mixture was concentrated to give a crude, which was purified by Prep-HPLC Purification (Method 1) to afford the title compound E6 (10.00 mg, 8.84 μmol, yield: 12.9%) as a white solid. LC-MS (ESI) m/z: 1121.51 [M+H]⁺. ¹H NMR (400 MHz, D₂O) δ 7.99-7.19 (m, 7H), 5.04-4.88 (m, 1H), 4.34-4.23 (m, 1H), 4.13-4.04 (m, 1H), 4.00-3.59 (m, 8H), 3.56-2.87 (m, 19H), 2.58-2.45 (m, 2H), 2.31-2.08 (m, 2H), 2.09-1.67 (m, 13H), 1.68-1.48 (m, 2H), 1.41-1.03 (m, 4H).

### E7: (S)-2-(3-((S)-5-((S)-3-(anthracen-9-yl)-2-((3,4-dioxo-2-((4-(5-((4,7,10-tris(carboxymethyl)-1,4,7,10-tetraazacyclododecan-1-yl)methyl)-1,3,4-oxadiazol-2-yl)bicyclo[2.2.2]octan-1-yl)amino)cyclobut-1-en-1-yl)amino)propanamido)-1-carboxypentyl)ureido)hexanedioic acid

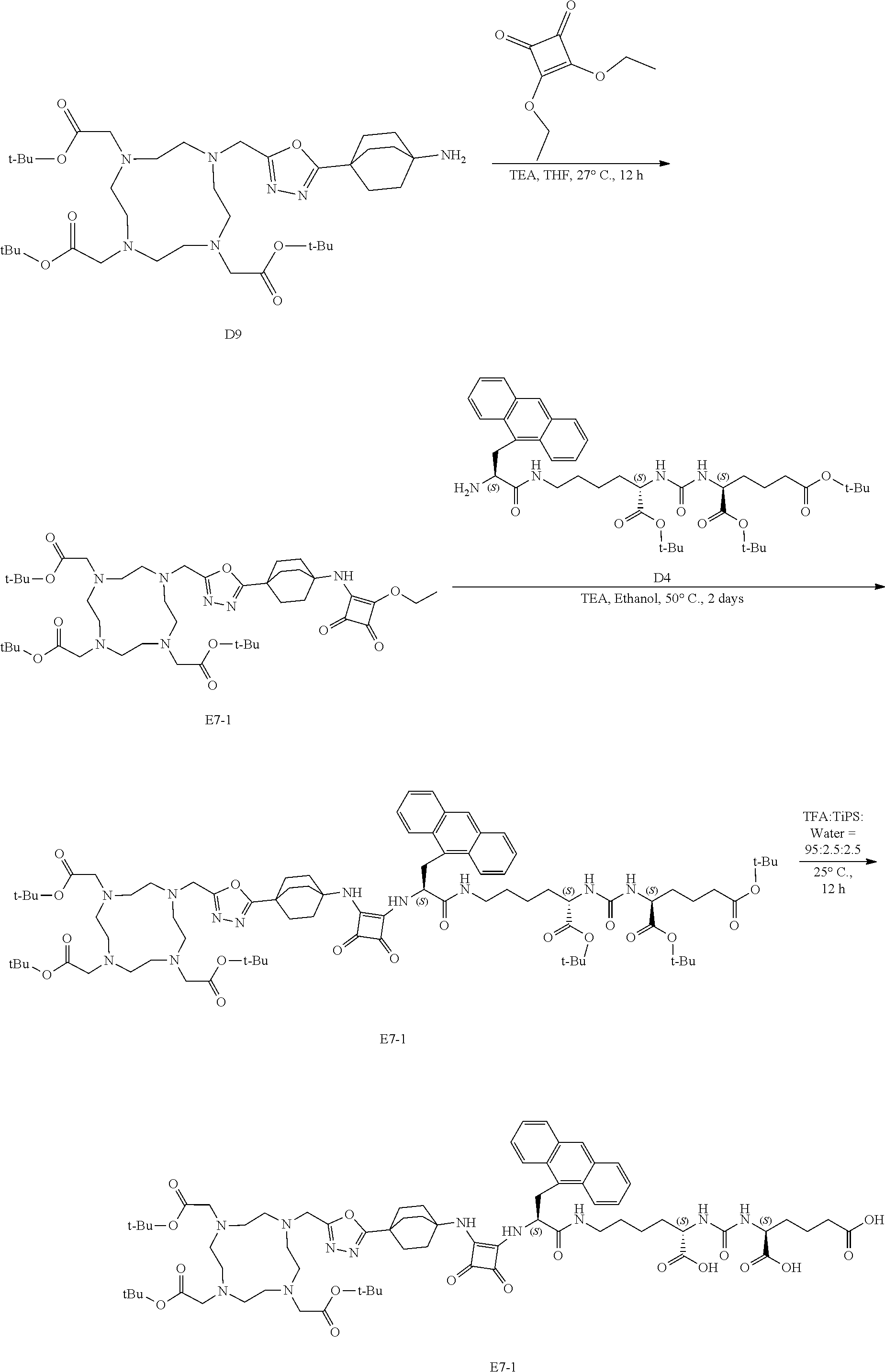

### Step 1:

To a solution of 3,4-diethoxycyclobut-3-ene-1,2-dione (0.134 mL, 0.90 mmol) in THF (8 ml) were added D9 (260.00 mg, 0.36 mmol) and TEA (0.15 mL, 1.08 mmol). The mixture was stirred at 27° C. under N₂ for 12 h. The resulting mixture was concentrated to give a crude product, which was purified by silica gel column chromatography to afford the title compound (280.00 mg, 0.33 mmol, 92% yield) as yellow oil. LC-MS (ESI) m/z: 843.9 [M+H]⁺.

### Step 2:

To a solution of D4 (100.00 mg, 0.13 mmol) in THF (0.5 mL) were added TEA (0.074 mL, 0.534 mmol) and E7-1 (113.00 mg, 0.13 mmol). The mixture was stirred at 50° C. under N₂ for 2 days. The resulting mixture was quenched with H₂O (5 mL). The aqueous layer was extracted with EA (2 mL). The organic layer was washed with brine (1 mL), dried over Na₂SO₄, filtered and concentrated in vacuum to afford the title compound E7-2 (71.00 mg, 0.046 mmol, 34.4% yield). LC-MS (ESI) m/z: 1546.2 [M+H]⁺.

### Step 3:

A solution of E7-2 (71.00 mg, 0.046 mmol) in TFA (0.95 mL), TIPS (0.025 mL) and water (0.025 mL) was stirred at 25° C. under N₂ for 12 h. The resulting mixture was concentrated to give a residue, which was purified by prep-HPLC (method 3) to afford the title compound E7 (23.00 mg, 0.019 mmol, 40.5% yield) as a white solid. LC-MS (ESI) m/z: 1209.9 [M+H]⁺. ¹H NMR (400 MHz, D₂O) δ 8.51 (s, 1H), 8.35 (d, J=8.8 Hz, 2H), 8.11 (d, J=8.8 Hz, 2H), 7.73-7.53 (m, 4H), 4.98-4.87 (m, 1H), 4.36 (s, 2H), 4.25-4.17 (m, 1H), 4.12-4.04 (m, 3H), 3.99-3.85 (m, 4H), 3.65-3.37 (m, 10H), 3.31-3.22 (m, 2H), 3.20-2.94 (m, 8H), 2.31-2.23 (m, 2H), 2.10-1.96 (m, 6H), 1.88-1.56 (m, 12H), 1.48-1.41 (m, 2H), 1.36-1.30 (m, 2H).

The following compounds were prepared using procedures analogous to those described in Examples above. As is appreciated by those skilled in the art, these analogous examples may involve variations in general reaction conditions. The Method column indicates preparatory methods described above used in the preparation of the compounds.

| | |
|---|---|
| 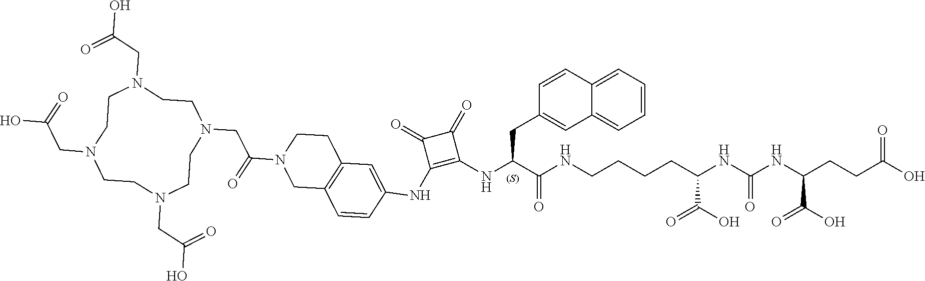 | Synthetic Method: A Purification Method: Method 3 LCMS (ESI) m/z [M + H]⁺: 1129.1 ¹H-NMR (400 MHz, D₂O) δ 7.86 (dd, J = 12.9, 8.3 Hz, 2H), 7.77 (d, J = 8.2 Hz, 1H), 7.70 (s, 1H), 7.56-7.35 (m, 3H), 7.18-7.07 (m, 1H), 6.90 (s, 2H), 5.02 (s, 1H), 4.60 (d, J = 19.4 Hz, 2H), 4.26-4.02 (m, 2H), 3.86 (dd, J = 26.1, 9.3 Hz, 6H), 3.58 (d, J = 21.2 Hz, 3H), 3.44 (d, J = 26.2 Hz, 10H), 3.24 (d, J = 26.6 Hz, 11H), 2.80 (d, J = 28.8 Hz, 2H), 2.39 (t, J = 7.9 Hz, 2H), 2.23-2.04 (m, 1H), 1.93 (dt, J = 14.4, 7.8 Hz, 1H), 1.75 (d, J = 26.5 Hz, 1H), 1.59 (s, 1H), 1.48 (s, 2H), 1.38 (d, J = 6.2 Hz, 2H). |
| E5 | |
| 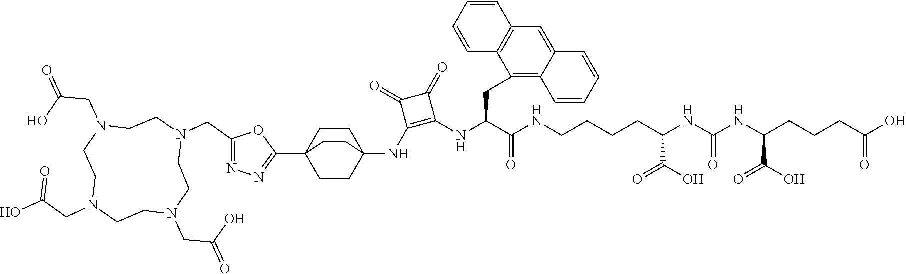 [SMILES: O=C(O)CCC[C@H](NC(=O)N[C@@H](CCCCNC(=O)[C@H](Cc1c2ccccc2cc2ccccc12)Nc1c(NC23CCC(c4nnc(CN5CCN(CC(=O)O)CCN(CC(=O)O)CCN(CC(=O)O)CC5)o4)(CC2)CC3)c(=O)c1=O)C(=O)O)C(=O)O] | Synthetic Method: D Purification Method: Method 3 LCMS (ESI) m/z [M + H]⁺: 1209.9 ¹H-NMR (400 MHz, D₂O) δ 8.51 (s, 1H), 8.35 (d, J = 8.8 Hz, 2H), 8.11 (d, J = 8.8 Hz, 2H), 7.73- 7.53 (m, 4H), 4.98-4.87 (m, 1H), 4.36 (s, 2H), 4.25-4.17 (m, 1H), 4.12-4.04 (m, 3H), 3.99-3.85 (m, 4H), 3.65-3.37 (m, 10H), 3.31-3.22 (m, 2H), 3.20-2.94 (m, 8H), 2.31-2.23 (m, 2H), 2.10-1.96 (m, 6H), 1.88-1.56 (m, 12H), 1.48-1.41 (m, 2H), 1.36-1.30 (m, 2H). |
| E7 | |
| 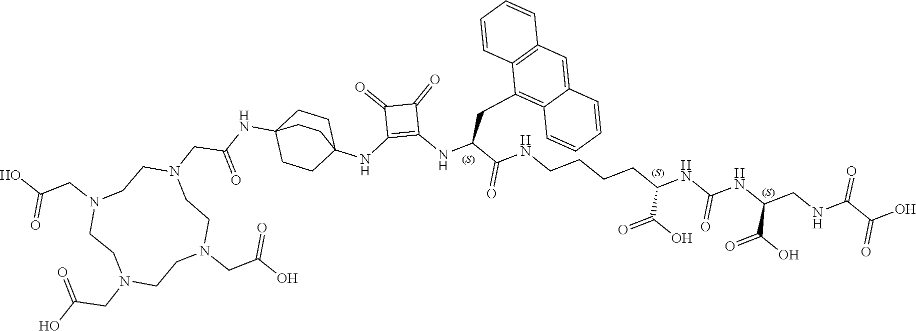 | Synthetic Method: D Purification Method: Method 3 LCMS (ESI) m/z [M + H]⁺: 1200.51 ¹H-NMR (400 MHz, D₂O) δ 8.35 (s, 1H), 8.18 (d, J = 8.8 Hz, 2H), 7.93 (d, J = 8.8 Hz, 2H), 7.52- 7.37 (m, 4H), 4.81-4.71 (m, 1H), 4.15-3.87 (m, 4H), 3.66 (s, 3H), 3.38-3.17 (m, 13H), 3.13-2.89 (m, 10H), 2.82-1.94 (m, 2H), 1.84-1.71 (m, 6H), 1.65-1.43 (m, 8H), 1.33-1.22 (m, 2H), 1.19-1.10 (m, 2H). |
| E8 | |
| 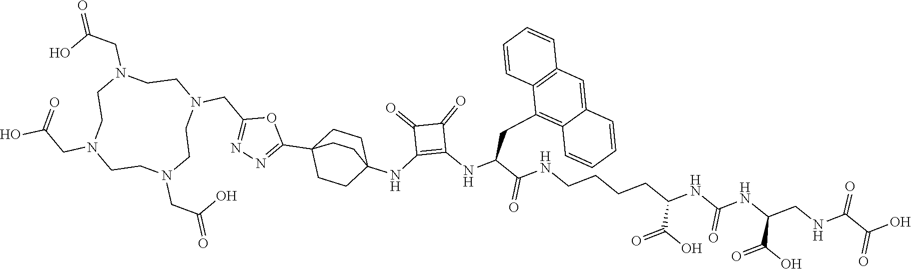 [SMILES: O=C(O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CNC(=O)C(=O)O)C(=O)O)C(=O)O)c(=O)c5=O)(CC3)CC4)o2)CCN(CC(=O)O)CC1] | Synthetic Method: D Purification Method: Method 3 LCMS (ESI) m/z [M + H]⁺: 1225.51 ¹H-NMR (400 MHz, D₂O) δ 8.39 (s, 1H), 8.25 (d, J = 8.5 Hz, 2H), 8.01 (d, J = 10.6 Hz, 2H), 7.70 7.39 (m, 4H), 4.83 (d, J = 2.3 Hz, 1H), 4.41-4.28 (m, 2H), 4.26-4.17 (m, 1H), 4.15-3.75 (m, 7H), 3.63-3.32 (m, 10H), 3.24-2.88 (m, 8H), 2.12- 1.88 (m, 6H), 1.76-1.63 (m, 6H), 1.59-1.46 (m, 2H), 1.46-1.04 (m, 6H), 0.90-0.73 (m, 2H). |
| E9 | |
| 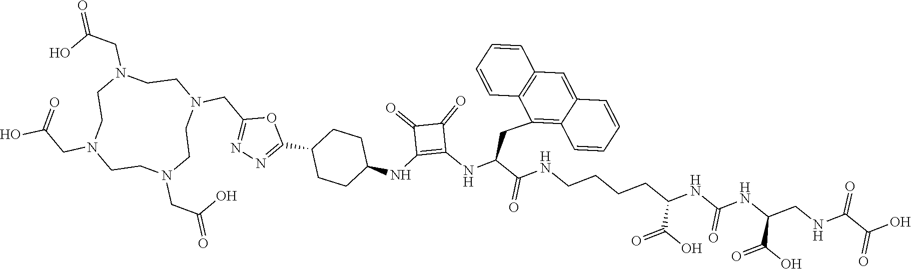 [SMILES: O=C(O)CN1CCN(CC(=O)O)CCN(Cc2nnc([C@H]3CC[C@H](Nc4c(N[C@@H](Cc5c6ccccc6cc6ccccc56)C(=O)NCCCC[C@H](NC(=O)N[C@@H](CNC(=O)C(=O)O)C(=O)O)C(=O)O)c(=O)c4=O)CC3)o2)CCN(CC(=O)O)CC1] | Synthetic Method: D Purification Method: Method 3 LCMS (ESI) m/z [M + H]⁺: 1199.24 ¹H-NMR (400 MHz, D₂O) δ 8.59 (s, 1H), 8.45- 8.36 (m, 2H), 8.16 (d, J = 8.3 Hz, 2H), 7.68 (dd, J = 9.9, 4.5 Hz, 2H), 7.64-7.59 (m, 2H), 5.08-4.94 (m, 1H), 4.43-4.38 (m, 3H), 4.26 (d, J = 1.0 Hz, 1H), 4.21-4.14 (m, 2H), 3.99-3.97 (m, 2H), 3.66 (d, J = 5.9 Hz, 4H), 3.62-3.43 (m, 10H), 3.31- 2.97 (m, 12H), 2.37-2.16 (m, 2H), 2.00-1.92 (m, 1H), 1.84-1.74 (m, 1H), 1.69-1.61 (m, 2H), 1.47- 1.23 (m, 6H), 1.19-1.05 (m, 2H). |
| E10 | |
| 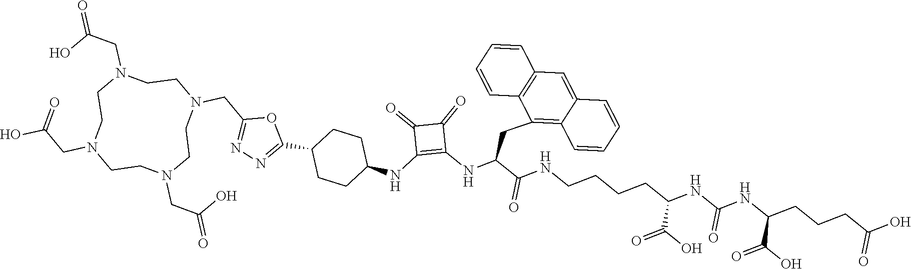 [SMILES: O=C(O)CCC[C@H](NC(=O)N[C@@H](CCCCNC(=O)[C@H](Cc1c2ccccc2cc2ccccc12)Nc1c(N[C@H]2CC[C@H](c3nnc(CN4CCN(CC(=O)O)CCN(CC(=O)O)CCN(CC(=O)O)CC4)o3)CC2)c(=O)c1=O)C(=O)O)C(=O)O] | Synthetic Method: D Purification Method: Method 3 LCMS (ESI) m/z [M + H]⁺: 1184.27 ¹H-NMR (400 MHz, D₂O) δ 8.55 (s, 1H), 8.41 (s, 2H), 8.13 (d, J = 8.4 Hz, 2H), 7.68-7.59 (m, 4H), 4.36 (s, 2H), 4.29 (s, 1H), 4.20 (s, 2H), 4.09 (s, 1H), 4.03 (s, 1H), 3.93 (d, J = 13.1 Hz, 2H), 3.82-3.43 (m, 14H), 3.39 (d, J = 16.8 Hz, 4H), 3.28 (s, 4H), 3.15 (s, 4H), 2.30 (s, 5H), 1.78 (s, 2H), 1.62 (s, 8H), 1.47 (s, 3H). |
| E11 | |
| 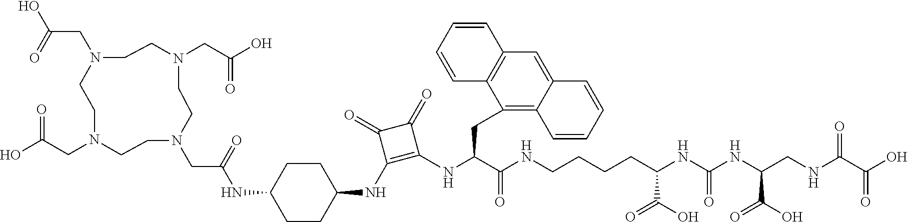 [SMILES: O=C(O)CN1CCN(CC(=O)O)CCN(CC(=O)N[C@H]2CC[C@H](Nc3c(N[C@@H](Cc4c5ccccc5cc5ccccc45)C(=O)NCCCC[C@H](NC(=O)N[C@@H](CNC(=O)C(=O)O)C(=O)O)C(=O)O)c(=O)c3=O)CC2)CCN(CC(=O)O)CC1] | Synthetic Method: B Purification Method: Method 1 LCMS (ESI) m/z [M + H]⁺: 1174.51 ¹H-NMR (400 MHz, D₂O) δ 8.36 (s, 1H), 8.23- 8.12 (m, 2H), 7.99-7.84 (m, 2H), 7.58-7.23 (m, 4H), 4.17-4.08 (m, 1H), 4.04-3.93 (m, 1H), 3.68-3.62 (m, 3H), 3.55 (d, J = 2.6 Hz, 5H), 3.26- 3.02 (m, 22H), 2.10 (t, J = 7.4 Hz, 3H), 1.91 (s, 3H), 1.83-1.75 (m, 2H), 1.75-1.68 (m, 2H), 1.62 (ddd, J = 5.8, 2.9, 1.6 Hz, 3H), 1.47-1.42 (m, 4H). |
| E12 | |
| 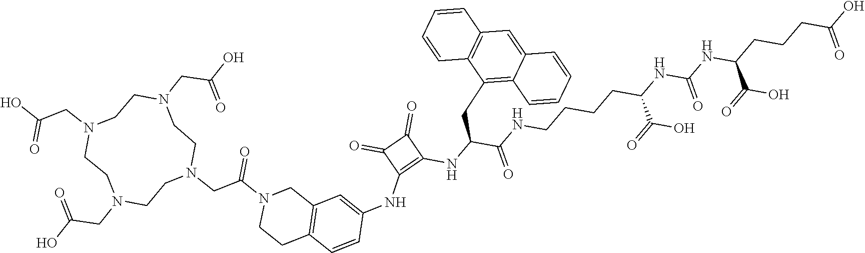 [SMILES: O=C(O)CCC[C@H](NC(=O)N[C@@H](CCCCNC(=O)[C@H](Cc1c2ccccc2cc2ccccc12)Nc1c(Nc2ccc3c(c2)CN(C(=O)CN2CCN(CC(=O)O)CCN(CC(=O)O)CCN(CC(=O)O)CC2)CC3)c(=O)c1=O)C(=O)O)C(=O)O] | Synthetic Method: B Purification Method: Method 3 LCMS (ESI) m/z [M + H]⁺: 1192.51 ¹H-NMR (400 MHz, D₂O) δ 8.41 (s, 1H), 8.34- 8.12 (m, 2H), 8.11-7.76 (m, 2H), 7.65-7.30 (m, 3H), 7.29-6.90 (m, 3H), 6.87-6.73 (m, 1H), 4.28- 4.19 (m, 1H), 4.08-3.98 (m, 2H), 3.94-3.71 (m, 7H), 3.66-3.32 (m, 12H), 3.30-2.99 (m, 10H), 2.96-2.65 (m, 4H), 2.45-2.13 (m, 3H), 1.81-1.54 (m, 5H), 1.54-1.09 (m, 5H). |
| E13 | |
| 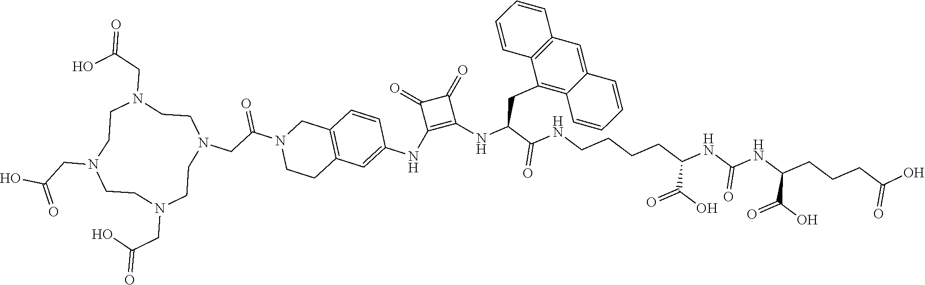 [SMILES: O=C(O)CCC[C@H](NC(=O)N[C@@H](CCCCNC(=O)[C@H](Cc1c2ccccc2cc2ccccc12)Nc1c(Nc2ccc3c(c2)CCN(C(=O)CN2CCN(CC(=O)O)CCN(CC(=O)O)CCN(CC(=O)O)CC2)C3)c(=O)c1=O)C(=O)O)C(=O)O] | Synthetic Method: B Purification Method: Method 3 LCMS (ESI) m/z [M + H]⁺: 1193.51 ¹H-NMR (400 MHz, D₂O) δ 8.33-8.27 (m, 1H), 8.23 (s, 2H), 7.87 (s, 2H), 7.49 (s, 2H), 7.41-7.25 (m, 2H), 7.09-6.99 (m, 1H), 6.83-6.60 (m, 2H), 4.56-4.47 (m, 2H), 4.22-4.12 (m, 1H), 4.03-3.92 (m, 3H), 3.83-3.62 (m, 7H), 3.59-3.49 (m, 2H), 3.47-3.26 (m, 10H), 3.25-2.97 (m, 10H), 2.82- 2.68 (m, 2H), 2.22-2.12 (m, 2H), 1.76-1.64 (m, 2H), 1.61-1.49 (m, 4H), 1.37-1.27 (m, 2H), 1.26- 1.14 (m, 2H). |
| E14 | |
| 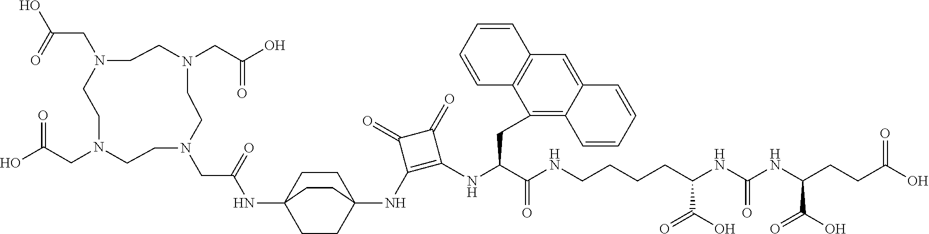 [SMILES: O=C(O)CC[C@H](NC(=O)N[C@@H](CCCCNC(=O)[C@H](Cc1c2ccccc2cc2ccccc12)Nc1c(NC23CCC(NC(=O)CN4CCN(CC(=O)O)CCN(CC(=O)O)CCN(CC(=O)O)CC4)(CC2)CC3)c(=O)c1=O)C(=O)O)C(=O)O] | Synthetic Method: D Purification Method: Method 3 LCMS (ESI) m/z [M + H]⁺: 1171.42 ¹H-NMR (400 MHz, D₂O) δ 8.40 (s, 1H), 8.29- 8.16 (m, 2H), 8.05-7.93 (m, 2H), 7.59-7.43 (m, 4H), 4.17-4.07 (m, 1H), 4.04-3.91 (m, 3H), 3.83- 3.66 (m, 4H), 3.45-3.25 (m, 12H), 3.20-2.99 (m, 10H), 2.28-2.19 (m, 2H), 2.04-1.72 (m, 9H), 1.72-1.50 (m, 8H), 1.39-1.29 (m, 2H), 1.27-1.17 (m, 2H). |
| E15 | |
| 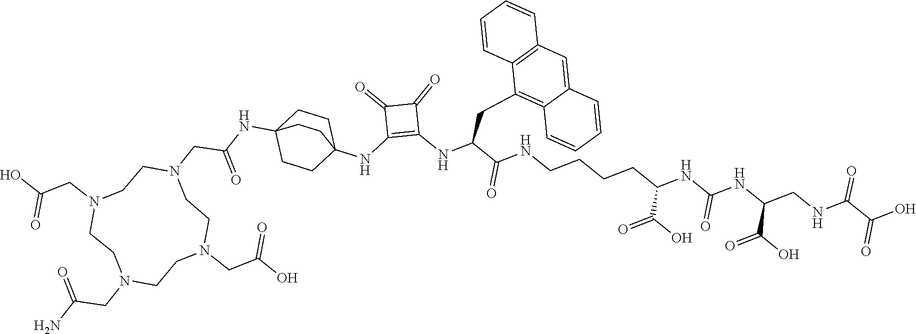 [SMILES: NC(=O)CN1CCN(CC(=O)O)CCN(CC(=O)NC23CCC(Nc4c(N[C@@H](Cc5c6ccccc6cc6ccccc56)C(=O)NCCCC[C@H](NC(=O)N[C@@H](CNC(=O)C(=O)O)C(=O)O)C(=O)O)c(=O)c4=O)(CC2)CC3)CCN(CC(=O)O)CC1] | Synthetic Method: D Purification Method: Method 3 LCMS (ESI) m/z [M + H]⁺: 1199.53 ¹H-NMR (400 MHz, D₂O) δ 8.46 (s, 1H), 8.38 8.19 (m, 2H), 8.12-7.96 (m, 2H), 7.67-7.43 (m, 4H), 4.34 4.32 (m, 1H), 4.19-4.05 (m, 4H), 3.88- 3.56 (m, 9H), 3.53-2.99 (m, 19H), 2.02-1.85 (m, 6H), 1.83-1.54 (m, 8H), 1.40-1.25 (m, 4H). |
| E16 | |
| 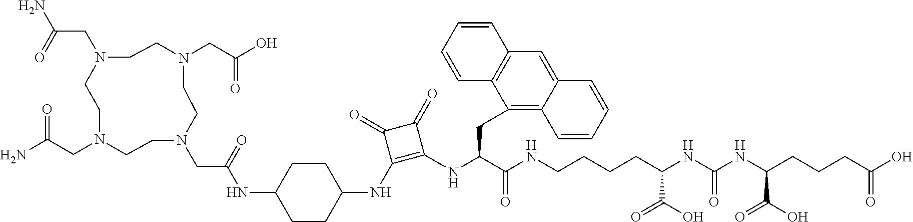 [SMILES: NC(=O)CN1CCN(CC(N)=O)CCN(CC(=O)NC2CCC(Nc3c(N[C@@H](Cc4c5ccccc5cc5ccccc45)C(=O)NCCCC[C@H](NC(=O)N[C@@H](CCCC(=O)O)C(=O)O)C(=O)O)c(=O)c3=O)CC2)CCN(CC(=O)O)CC1] | Synthetic Method: D Purification Method: Method 3 LCMS (ESI) m/z [M + H]⁺: 1184.54 ¹H NMR (400 MHz, D₂O) δ 8.37 (s, 1H), 8.21 (d, J = 8.8 Hz, 2H), 7.97 (d, J = 8.8 Hz, 2H), 7.60- 7.41 (m, 4H), 4.87-4.76 (m, 1H), 4.26-4.19 (m, 2H), 4.15-4.05 (m, 1H), 4.04-3.77 (m, 7H), 3.57- 3.29 (m, 10H), 3.20-2.72 (m, 10H), 2.24-2.11 (m, 2H), 1.98-1.85 (m, 6H), 1.73-1.49 (m, 12H), 1.41-1.29 (m, 2H), 1.27-1.10 (m, 2H). |
| E19 | |
| 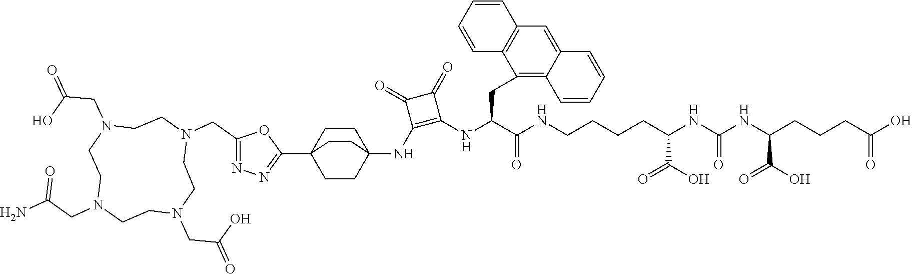 [SMILES: NC(=O)CN1CCN(CC(=O)O)CCN(Cc2nnc(C34CCC(Nc5c(N[C@@H](Cc6c7ccccc7cc7ccccc67)C(=O)NCCCC[C@H](NC(=O)N[C@@H](CCCC(=O)O)C(=O)O)C(=O)O)c(=O)c5=O)(CC3)CC4)o2)CCN(CC(=O)O)CC1] | Synthetic Method: D Purification Method: Method 3 LCMS (ESI) m/z [M + H]⁺: 1209.70 ¹H NMR (400 MHz, D₂O) δ 8.37 (s, 1H), 8.21 (d, J = 8.8 Hz, 2H), 7.97 (d, J = 8.8 Hz, 2H), 7.60- 7.41 (m, 4H), 4.87-4.76 (m, 1H), 4.26-4.19 (m, 2H), 4.15-4.05 (m, 1H), 4.04-3.77 (m, 7H), 3.57- 3.29 (m, 10H), 3.20-2.72 (m, 10H), 2.24-2.11 (m, 2H), 1.98-1.85 (m, 6H), 1.73-1.49 (m, 12H), 1.41-1.29 (m, 2H), 1.27-1.10 (m, 2H). |
| E20 | |

### E17 and E18: (((1S)-1-carboxy-5-(3-(naphthalen-2-yl)-2-((5-(4-((2-(4,7,10-tris(carboxymethyl)-1,4,7,10-tetraazacyclododecan-1-yl)acetamido)methyl)bicyclo[2.2.2]octan-1-yl)-1,3,4-oxadiazol-2-yl)amino)propanamido)pentyl)carbamoyl)-L-glutamic acid (Method E)

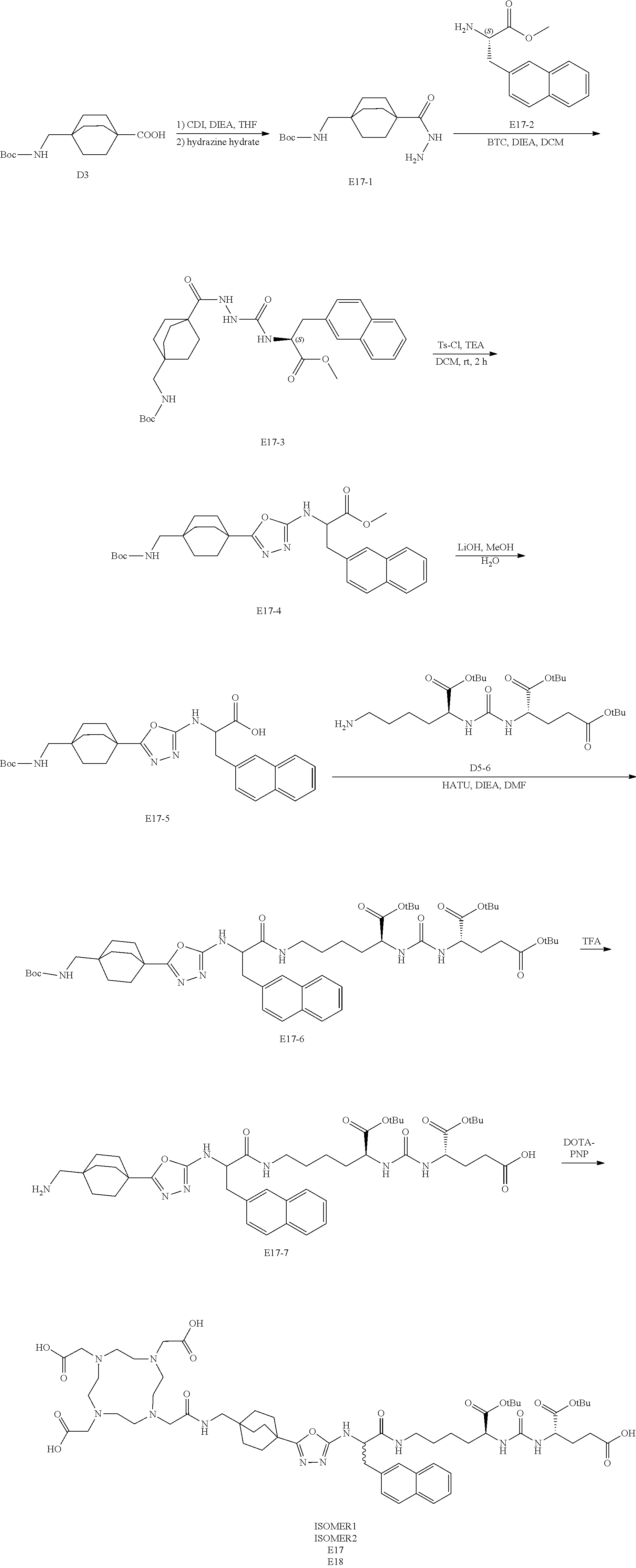

### Step 1

A mixture of D3 (1.00 g, 3.53 mmol) and CDI (1.14 g, 7.06 mmol) in THF (15 ml) was stirred at 25° C. for 1 h. After that the mixture was added into hydrazine H₂O (0.554 ml, 17.64 mmol), the mixture was stirred at 25° C. for another 1 h. The reaction mixture was washed with H₂O (10 mL×3) and extracted with ethyl acetate (10 mL×3). The combined organic layers were concentrated in vacuo to afford E17-1 (1.20 g, 4.03 mmol, 114% yield) as a colorless oil. LCMS (ESI): [M+H]=298.20.

### Step 2

A mixture of triphosgene (0.998 g, 3.36 mmol) in DCM (15 mL) was added E17-2 (0.771 g, 3.36 mmol). The mixture was stirred at 25° C. for 1 h, and then E17-1 (1.00 g, 3.36 mmol) was added into the mixture which was still stirred for another 1 h. The reaction mixture was concentrated to give a crude product, which was purified by silica gel column chromatography to afford E17-3 (900 mg, 1.484 mmol, 44.1% yield) as a white solid. LCMS (ESI): [M+H]=553.20

### Step 3

A mixture of E17-3 (500 mg, 0.905 mmol), TEA (0.252 ml, 1.809 mmol) in DCM (3 ml) was added Ts-C₁ (259 mg, 1.357 mmol). The mixture was stirred at 25° C. for 1 h. The reaction mixture was concentrated to give a crude product, which was purified by silica gel column chromatography to afford E17-4 (300 mg, 0.905 mmol, 47.6% yield) as a white solid. LCMS (ESI): [M+H]⁺=535.10

### Step 4

A solution of E17-4 (200 mg, 0.374 mmol) and LiOH (8.96 mg, 0.374 mmol) in MeOH (2 ml) and Water (1 ml) was stirred at 25° C. for 3 h. The mixture was adjusted pH=5 by HCl (1M) and extracted with EA (10 mL×2). The combined organic layers were washed with brine (10 mL×1), dried over Na₂SO₄, filtered and concentrated in vacuum to give a crude product E17-5 (150 mg, 0.288 mmol, 77% yield). LCMS (ESI) [M+H]⁺=521.20

### Step 5

To a solution of E17-5 (150 mg, 0.288 mmol) in DMF (2 ml) was added HATU (131 mg, 0.346 mmol), DIEA (0.101 ml, 0.576 mmol) and D5-6 (155 mg, 0.317 mmol). The mixture was stirred at 25° C. for 2 h. The reaction mixture was concentrated to give a crude product, which was purified by silica gel column chromatography (Biotage flash) eluting with DCM:MeOH=20/1, collected and concentrated in vacuo to afford E17-6 (170 mg, 0.141 mmol, 48.8% yield) as a colorless oil. LCMS (ESI): [M+H]⁺=990.40

### Step 6

A mixture of E17-6 (170 mg, 0.172 mmol) in TFA (3 ml) was stirred at 25° C. for 5 h. The mixture was concentrated to give a crude E17-7 (100 mg, 0.139 mmol, 81% yield). LCMS (ESI): [M+H]⁺=722.30.

### Step 7

To a solution of E17-7 (100 mg, 0.139 mmol) in DMSO (5 ml) was added DIEA (0.048 ml, 0.278 mmol), DOTA-PNP (72.8 mg, 0.139 mmol). The mixture was stirred at 25° C. for 3 h. The reaction mixture was concentrated to give a crude product, which was purified by prep-HPLC (Method 3) to afford the first elution fraction as isomer 1 E17 (8 mg, 7.08 μmol, 5.11% yield) and second elution fraction as isomer 2 E18 (5 mg, 0.044 mmol, 32.0% yield) as a white solid.

E17: LCMS (ESI): [M+2H]²⁺/2=554.80. ¹H NMR (400 MHz, DMSO-d6) δ ppm 8.05-8.20 (m, 1H) 7.82 (br. s., 4H) 7.46 (br. s., 3H) 7.11-7.26 (m, 1H) 6.60-6.71 (m, 1H) 6.22-6.39 (m, 2H) 4.17-4.34 (m, 2H) 3.98-4.16 (m, 3H) 3.61-3.68 (m, 3H) 2.86-3.06 (m, 14H) 2.71-2.81 (m, 3H) 2.22-2.37 (m, 5H) 1.97-2.08 (m, 4H) 1.68-1.78 (m, 5H) 1.40-1.53 (m, 8H) 1.25 (br. s., 6H) 0.85-0.90 (m, 2H).

E18: LCMS (ESI): [M+2H]²⁺/2=554.80. ¹H NMR (400 MHz, DMSO-d₆) δ ppm 11.23-13.48 (m, 6H) 8.67-8.93 (m, 1H) 8.16 (br. s., 1H) 7.72-7.95 (m, 4H) 7.32-7.59 (m, 3H) 6.34 (d, J=8.33 Hz, 2H) 4.21-4.42 (m, 2H) 3.93-4.20 (m, 3H) 3.61 (br. s., 8H) 3.07-3.22 (m, 7H) 2.76-3.06 (m, 10H) 2.57-2.76 (m, 2H) 2.26 (dd, J=15.31, 7.79 Hz, 6H) 1.81-2.03 (m, 2H) 1.51-1.79 (m, 7H) 1.10-1.51 (m, 9H).

### E21: (4S,8S,15S)-15-((3,4-dioxo-2-((4-(2-(4,7,10-tris(carboxymethyl)-1,4,7,10-tetraazacyclododecan-1-yl)acetamido)bicyclo[2.2.2]octan-1-yl)amino)cyclobut-1-en-1-yl)amino)-16-(naphthalen-2-yl)-1,6,14-trioxo-2,5,7,13-tetraazahexadecane-1,4,8-tricarboxylic acid (Method E)

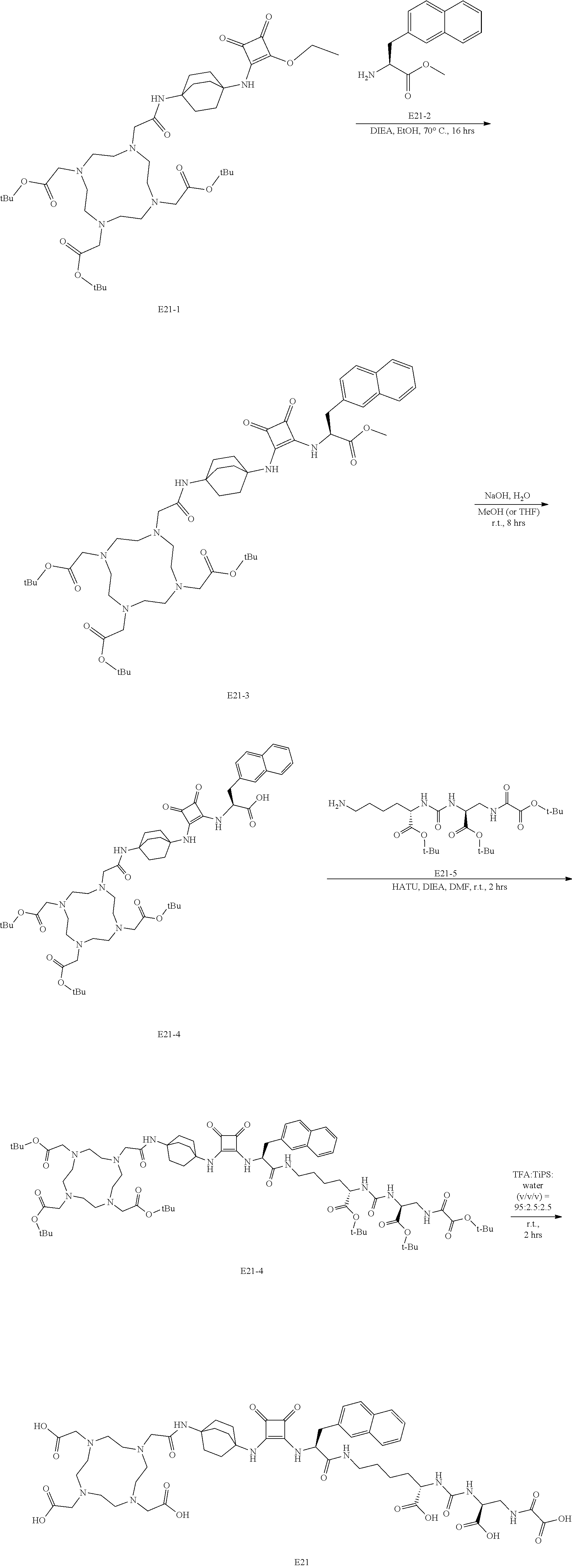

### Step 1:

To a solution of E21-1 (800.00 mg, 0.98 mmol) in EtOH (8 ml) were added DIEA (0.51 ml, 2.93 mmol) and E21-2 (224.00 mg, 0.98 mmol). The mixture was stirred at 70° C. under N₂ for 8 hrs. The resulting mixture was concentrated to give a crude product, which was purified by flash chromatography to afford the title compound E21-3 (527.00 mg, 0.53 mmol, yield: 53.8%) as a yellow solid. LC-MS (ESI) m/z: 1002 [M+H]⁺.

### Step 2:

To a solution of E21-3 (500.00 mg, 0.50 mmol) in THF (5 ml) was added 1N NaOH (1.00 ml, 1.00 mmol) at 0° C. The mixture was stirred at r.t. under N₂ for 4 hrs. The mixture was then evaporated under reduced pressure to afford the title compound E21-4 (520.00 mg, 0.53 mmol, crude) as a yellow solid. LC-MS (ESI) m/z: 988 [M+H]⁺.

### Step 3:

To a solution of E21-4 (350.00 mg, 0.35 mmol) in DMF (3 ml) were added DIEA (0.31 ml, 1.77 mmol), E21-5 (183.00 mg, 0.35 mmol) and HATU (269.00 mg, 0.71 mmol). The mixture was stirred at r.t. under N₂ for 2 hrs. The resulting mixture was quenched with saturated NH₄Cl (aq.). The aqueous layers were extracted with EA (25 mL). The combined organic layers were washed with saturated NaHCO₃ (aq.) (50 mL×1). The combined organic layers were dried over Na₂SO₄, filtered, and concentrated in vacuum to afford the title compound E21-6 (155.00 mg, 0.10 mmol, 29.4% yield). LC-MS (ESI) m/z: 1487 [M+H]⁺.

### Step 4:

A solution of E21-6 (100.00 mg, 0.07 mmol) in TFA (0.95 mL), TIPS (0.025 mL) and water (0.025 mL) was stirred at 25° C. under N₂ for 5 hrs. The resulting mixture was concentrated to give a crude, which was purified by Prep-HPLC Purification (Method 3) to afford the title compound E21 (25.00 mg, 0.02 mmol, yield: 32.0%) as a light yellow solid. LC-MS (ESI) m/z: 1150 [M+H]⁺. ¹H NMR (400 MHz, D₂O) δ 7.77-7.69 (m, 3H), 7.55 (s, 1H), 7.45-7.36 (m, 2H), 7.27-7.23 (m, 1H), 4.82-4.75 (m, 1H), 4.13-4.03 (m, 1H), 3.86-3.64 (m, 5H), 3.52-3.40 (m, 2H), 3.37-3.23 (m, 13H), 3.16-2.95 (m, 11H), 1.87-1.76 (m, 6H), 1.73-1.60 (m, 6H), 1.56-1.48 (m, 1H), 1.44-1.23 (m, 3H), 1.14-1.02 (m, 2H).

### E22: (4S,8S,15S)-16-(anthracen-9-yl)-1,6,14-trioxo-15-((6-((4-(2-(4,7,10-tris(carboxymethyl)-1,4,7,10-tetraazacyclododecan-1-yl)acetamido)bicyclo[2.2.2]octan-1-yl)amino)pyrimidin-4-yl)amino)-2,5,7,13-tetraazahexadecane-1,4,8-tricarboxylic acid (Method F)

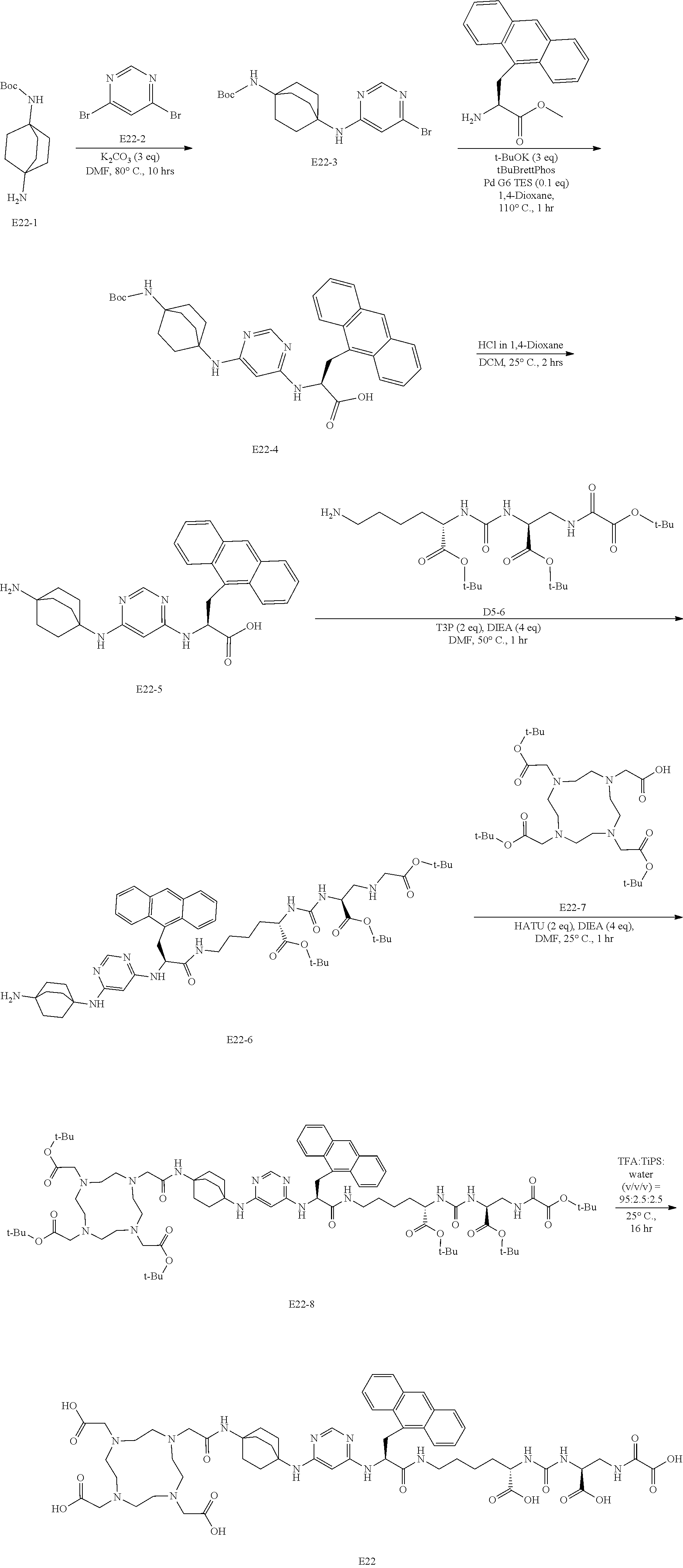

### Step 1:

To a solution of E22-2 (2.00 g, 8.41 mmol) in DMF (20 mL) were added K₂CO₃ (3.49 g, 25.20 mmol) and E22-1 (2.02 g, 8.41 mmol). The mixture was stirred at 80° C. under N₂ for 10 hrs. The resulting mixture was quenched with H₂O (20 mL). The aqueous layer was extracted with EA (100 mL×2). The combined organic layers were washed with brine (200 mL×1), dried over Na₂SO₄, filtered, and concentrated in vacuum to give a crude product, which was purified by silica gel column chromatography to afford the title compound E22-3 (1.10 g, 2.77 mmol, yield: 32.9%) as a white solid. LC-MS (ESI) m/z: 397 [M+H]⁺.

### Step 2:

To a solution of E22-3 (0.50 g, 1.26 mmol) in 1,4-dioxane (5 mL) were added ethyl (S)-2-amino-3-(anthracen-9-yl)propanoate (0.37 g, 1.26 mmol), tBuBrettPhos Pd G6 TES (0.11 g, 0.13 mmol) and potassium tert-butoxide (0.42 g, 3.78 mmol). The mixture was stirred at 110° C. under N₂ for 1 hr. The reaction was quenched with H₂O (10 mL). The aqueous layer was extracted with EA (50 mL×2). The combined organic layers were washed with brine (50 mL×2), dried over Na₂SO₄, filtered, and concentrated in vacuum to give a crude product. The crude product was purified by silica gel column chromatography to afford the title compound E22-4 (210.00 mg, 0.36 mmol, yield: 28.7%) as a yellow solid. LC-MS (ESI) m/z: 582 [M+H]⁺.

### Step 3:

To a solution of E22-4 (100.00 mg, 0.17 mmol) in DCM (1 mL) was added HCl in 1,4-dioxane (1.07 mL, 4.30 mmol). The mixture was stirred at 25° C. under N₂ for 2 hrs. The mixture was then evaporated under reduced pressure to afford the title compound E22-5 (80.00 mg, 0.15 mmol, crude). LC-MS (ESI) m/z: 482 [M+H]⁺.

### Step 3:

To a solution of E22-5 (150.00 mg, 0.31 mmol) in DMF (1 mL) were added DIEA (0.21 ml, 1.25 mmol), D5-6 (241.00 mg, 0.47 mmol) and T3P (0.37 mL, 0.62 mmol). The mixture was stirred at 50° C. under N₂ for 1 hr. The crude product was purified by C₁₈ reversed phase column to afford the title compound E22-6 (110.00 mg, 0.11 mmol, yield: 36.0%) as a yellow solid. LC-MS (ESI) m/z: 980 [M+H]⁺.

### Step 4:

To a solution of E22-7 (61.40 mg, 0.11 mmol) in DMF (0.2 mL) were added DIEA (0.08 mL, 0.43 mmol), HATU (81.00 mg, 0.21 mmol) and E22-6 (105.00 mg, 0.11 mmol). The mixture was stirred at 25° C. under N₂ for 1 hr. The reaction was quenched with H₂O (10 mL). The aqueous layer was extracted with EA (5 mL×4). The combined organic layers were washed with brine (25 mL×3), dried over Na₂SO₄, filtered, and concentrated in vacuum to give a crude product. The crude product was purified by silica gel column chromatography to afford the title compound E22-8 (75.00 mg, 0.05 mmol, yield: 45.6%) as a yellow solid. LC-MS (ESI) m/z: 1535 [M+H]⁺.

### Step 4:

To E22-8 (75.00 mg, 0.05 mmol) were added TFA (0.6 mL), TIPS (0.02 mL) and water (0.02 mL). The mixture was stirred at 25° C. under N₂ for 16 hrs. The mixture was then evaporated under reduced pressure to give crude product. The compound was purified by Prep-HPLC Purification (Method 3) to afford the title compound E22 (6.00 mg, 4.83 μmol, yield: 9.9%) as a white solid. LC-MS (ESI) m/z: 1198 [M+H]⁺. ¹H NMR (400 MHz, D₂O) δ 8.33-8.08 (m, 2H), 7.94-7.68 (m, 3H), 7.61-7.05 (m, 6H), 4.34-4.17 (m, 2H), 4.01-3.68 (m, 5H), 3.64-3.46 (m, 6H), 3.41-3.23 (m, 9H), 3.18-2.90 (m, 10H), 2.04-1.73 (m, 8H), 1.65-1.44 (m, 6H), 1.32-1.09 (m, 3H), 0.94-0.63 (m, 2H).

### E23: (((S)-1-carboxy-5-((S)-3-(naphthalen-2-yl)-2-((4-((4-(2-(4,7,10-tris(carboxymethyl)-1,4,7,10-tetraazacyclododecan-1-yl)acetamido)bicyclo[2.2.2]octan-1-yl)amino)-1,3,5-triazin-2-yl)amino)propanamido)pentyl)carbamoyl)-L-glutamic acid (Method G)

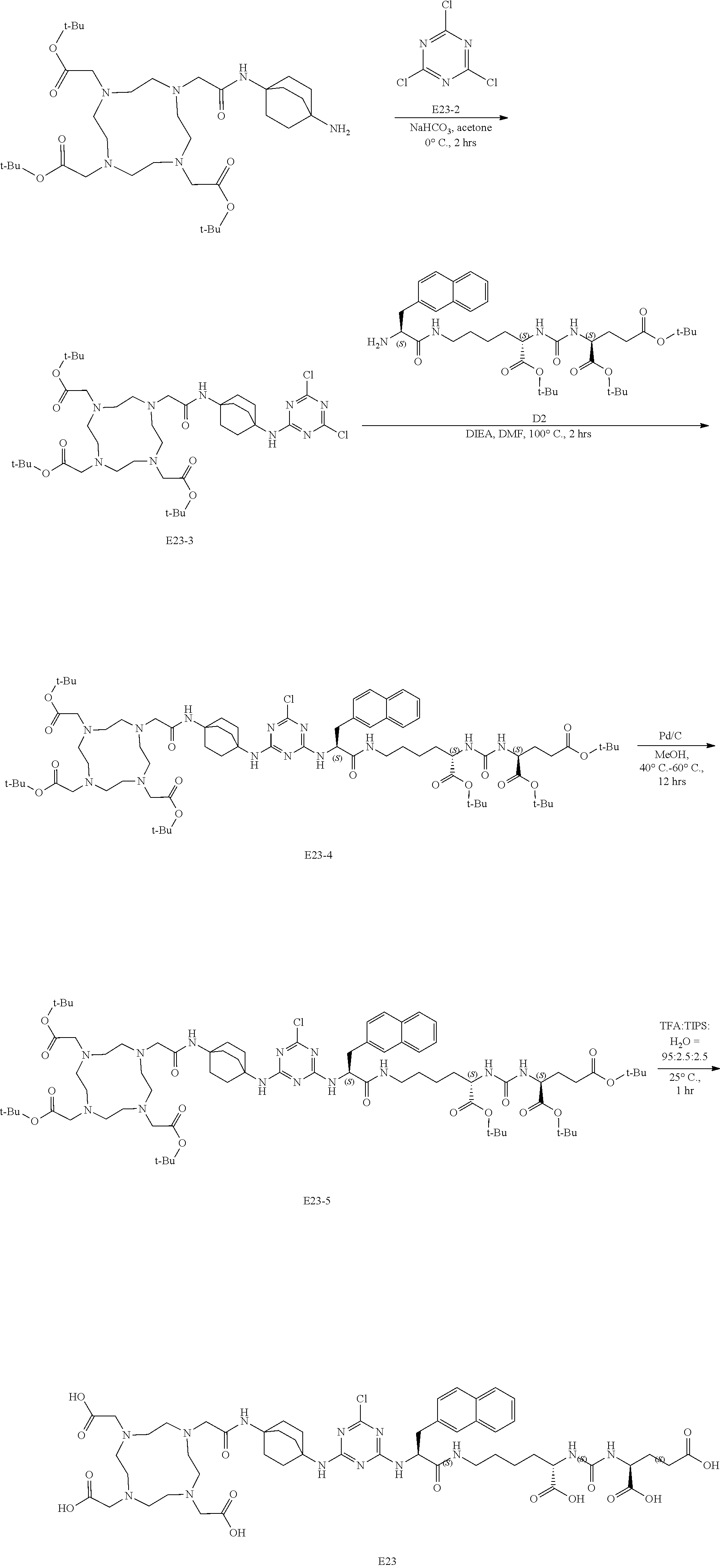

### Step 1:

To a solution of E23-1 (500.00 mg, 0.72 mmol) in ACN (20 mL) were added E23-2 (265.00 mg, 1.44 mmol) and Na₂CO₃ (229.00 mg, 2.16 mmol). The mixture was stirred at 10° C. under N₂ for 12 hrs. The resulting mixture was concentrated to give a crude product, which was purified by flash chromatography to afford the title compound E23-3 (111.00 mg, 0.13 mmol, yield: 18.3%) as a yellow solid. LC-MS (ESI): 842 [M+H]⁺.

### Step 2:

To a solution of E23-3 (100.00 mg, 0.12 mmol) in DMF (1 ml) were added D2 (81.00 mg, 0.12 mmol) and DIEA (0.062 ml, 0.36 mmol). The mixture was stirred at 100° C. under N₂ for 2 hrs. The reaction was quenched with H₂O (3 mL). The aqueous layers were extracted with EA (5 mL×2). The combined organic layers were washed with brine (5 mL×1), dried over Na₂SO₄, filtered, and concentrated in vacuum to give a crude product. The crude product was purified by flash chromatography to afford the title compound E23-4 (51.00 mg, 0.034 mmol, yield: 28.8%) as colorless oil. LC-MS (ESI): 1491 [M+H]⁺.

### Step 3:

To a solution of E23-4 (51.00 mg, 0.034 mmol) in MeOH (2 ml) was added 10% wet Pd/C (3.64 mg, 0.034 mmol). The mixture was stirred at 60° C. under H₂ for 12 hrs. The resulting mixture was filtered and concentrated to afford the title compound E23-5 (12.00 mg, 0.034 mmol, yield: 24.1%) as a white solid. LC-MS (ESI): 1457 [M+H]⁺.

### Step 4:

To a solution of E23-5 (5.00 mg, 3.43 μmol) in TFA (0.95 ml) was added TIPS (0.025 ml) and water (0.025 ml). The mixture was stirred at 25° C. under N₂ for 1 hr. The resulting mixture was then concentrated to give a residue, which was purified by Prep-HPLC (Method 3) to afford the title compound E23 (0.60 mg, 0.48 μmol, yield: 14.1%) as a white solid. LC-MS (ESI): 1120 [M+H]⁺. ¹H NMR (400 MHz, D₂O) δ 7.97-7.70 (m, 5H), 7.50-7.35 (m, 3H), 4.00-3.95 (m, 1H), 3.87-3.81 (m, 1H), 3.73-3.67 (m, 2H), 3.38-3.21 (m, 9H), 3.15-2.92 (m, 12H), 2.76-2.59 (m, 4H), 2.31-2.19 (m, 4H), 1.89-1.81 (m, 12H), 1.55-1.45 (m, 2H), 1.26-1.22 (m, 2H), 1.19-1.17 (m, 2H), 1.04-0.98 (m, 2H).

The following compounds were prepared using procedures analogous to those described in Examples above. As is appreciated by those skilled in the art, these analogous examples may involve variations in general reaction conditions. The Method column indicates preparatory methods described above used in the preparation of the compounds.

| | |
|---|---|
| 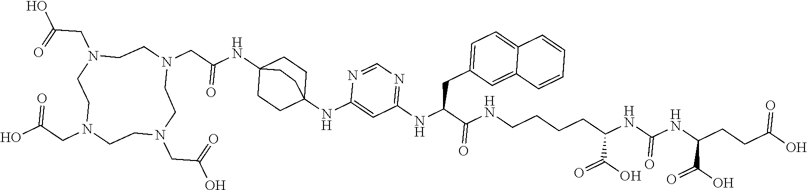 [SMILES: O=C(O)CC[C@H](NC(=O)N[C@@H](CCCCNC(=O)[C@H](Cc1ccc2ccccc2c1)Nc1cc(NC23CCC(NC(=O)CN4CCN(CC(=O)O)CCN(CC(=O)O)CCN(CC(=O)O)CC4)(CC2)CC3)ncn1)C(=O)O)C(=O)O] | Synthetic Method: F Purification Method: Method 3 LCMS (ESI) m/z [M + H]⁺: 1120.2 ¹H-NMR (400 MHz, D₂O) δ 8.04-7.88 (m, 1H), 7.87-7.74 (m, 3H), 7.68 (s, 1H), 7.51-7.42 (m, 2H), 7.42-7.34 (m, 1H), 5.36 (s, 1H), 4.45 (s, 1H), 4.04-3.96 (m, 1H), 3.86-3.74 (m, 1H), 3.58-2.91 (m, 14H), 2.84-2.51 (m, 7H), 2.37-2.08 (m, 8H), 2.03-1.96 (m, 1H), 1.89-1.68 (m, 13H), 1.56- 1.15 (m, 5H), 1.09-0.90 (m, 2H). |
| E24 | |
| 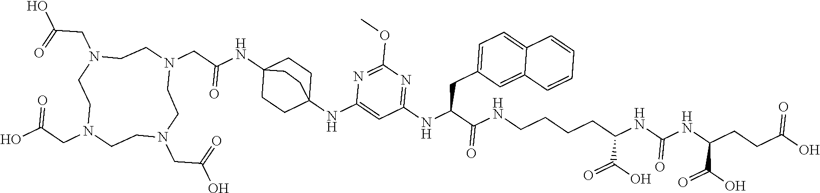 [SMILES: COc1nc(N[C@@H](Cc2ccc3ccccc3c2)C(=O)NCCCC[C@H](NC(=O)N[C@@H](CCC(=O)O)C(=O)O)C(=O)O)cc(NC23CCC(NC(=O)CN4CCN(CC(=O)O)CCN(CC(=O)O)CCN(CC(=O)O)CC4)(CC2)CC3)n1] | Synthetic Method: F Purification Method: Method 3 LCMS (ESI) m/z [M + H]⁺: 1149.3 ¹H-NMR (400 MHz, D₂O) δ 7.92-7.79 (m, 3H), 7.71 (s, 1H), 7.51 (dd, J = 6.7, 3.5 Hz, 2H), 7.45- 7.39 (m, 1H), 4.73 4.62 (m, 1H), 4.54 4.41 (m, 1H), 4.07-3.99 (m, 1H), 3.92-3.72 (m, 8H), 3.53- 2.92 (m, 24H), 2.38-2.25 (m, 2H), 2.11-1.84 (m, 14H), 1.62-1.17 (m, 4H), 1.09-0.91 (m, 2H). |
| E25 | |
| 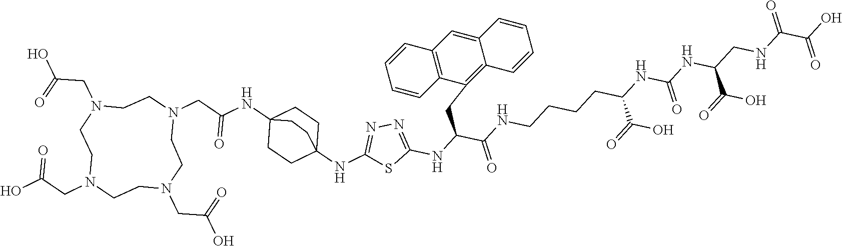 [SMILES: O=C(O)CN1CCN(CC(=O)O)CCN(CC(=O)NC23CCC(Nc4nnc(N[C@@H](Cc5c6ccccc6cc6ccccc56)C(=O)NCCCC[C@H](NC(=O)N[C@@H](CNC(=O)C(=O)O)C(=O)O)C(=O)O)s4)(CC2)CC3)CCN(CC(=O)O)CC1] | Synthetic Method: F Purification Method: Method 3 LCMS (ESI) m/z [1/2M + H]⁺: 603.0 ¹H-NMR (400 MHz, D₂O) δ 8.52-8.39 (m, 1H), 8.24 (d, J = 8.6 Hz, 2H), 8.02 (d, J = 8.4 Hz, 2H), 7.59-7.36 (m, 4H), 4.86-4.74 (m, 1H), 4.55-4.41 (m, 1H), 4.20-4.05 (m, 1H), 3.76-3.68 (m, 2H), 3.53-3.44 (m, 4H), 3.41-3.37 (m, 2H), 3.35-3.26 (m, 6H), 3.14-3.06 (m, 4H), 3.01-2.94 (m, 4H), 2.76-2.62 (m, 4H), 2.28-2.18 (m, 2H), 2.13-2.06 (m, 2H), 1.96-1.83 (m, 6H), 1.78-1.71 (m, 6H), 1.63-1.53 (m, 4H), 1.42-1.35 (m, 2H). |
| E26 | |
| 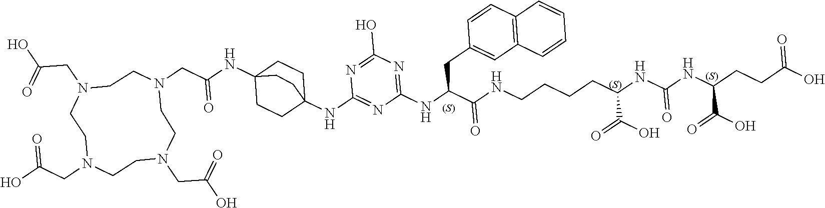 | Synthetic Method: G Purification Method: Method 1 LCMS (ESI) m/z [M + H]⁺: 1136.5 ¹H-NMR (400 MHz, D₂O) δ 7.87-7.80 (m, 3H), 7.72 (s, 1H), 7.50-7.45 (m, 2H), 7.41-7.35 (m, 1H), 4.15-4.09 (m, 1H), 3.99-3.93 (m, 1H), 3.75 3.53 (m, 7H), 3.34-2.99 (m, 20H), 2.42-2.35 (m, 2H), 2.06-1.82 (m, 16H), 1.66-1.53 (m, 1H), 1.52-1.40 (m, 1H), 1.34-1.26 (m, 2H), 1.17-1.03 (m, 2H). |
| E27 | |
| 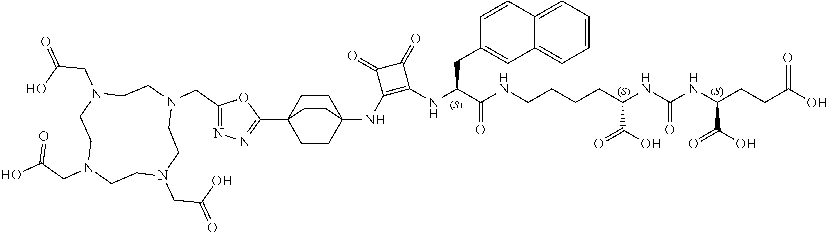 | Synthetic Method: G Purification Method: Method 3 LCMS (ESI) m/z [M + H]⁺: 1146.5 ¹H-NMR (400 MHz, D₂O) δ 7.83-7.75 (m, 3H), 7.62 (s, 1H), 7.50-7.41 (m, 2H), 7.34-7.30 (m, 1H), 4.90-4.82 (m, 1H), 4.80-4.75 (m, 1H), 4.08-3.97 (m, 2H), 3.93-3.85 (m, 2H), 3.42-3.37 (m, 1H), 3.31-3.11 (m, 5H), 3.08- 3.00 (m, 2H), 2.99-2.74 (m, 6H), 2.71-2.47 (m, 6H), 2.42-2.20 (m, 3H), 2.18-2.13 (m, 2H), 2.02-1.86 (m, 8H), 1.83-1.66 (m, 8H), 1.64-1.53 (m, 1H), 1.51-1.40 (m, 1H), 1.40- 1.31 (m, 2H), 1.21-1.11 (m, 2H). |
| E28 | |

### Naaladase Assay

Naaladase activity inhibition was determined using a fluorescence-based assay. Briefly, the solution containing the substrate N-Acetyl-Asp-Glu (Sigma-Aldrich) at 40 μM and the compound at concentrations from 0.15 nM to 1000 nM was mixed at equal volume with recombinant human PSMA (rhPSMA, Sino Biological) at 0.4 μg/mL, in assay buffer (50 mM HEPES, 100 mM NaCl, pH 7.5). The enzymatic reaction was proceeded by incubation at 37° C. for one hour and then stopped by heating at 95° C. for 5 minutes. A solution of 15 mM ortho-phthaldialdehyde (Sigma-Aldrich) was added to all vials and incubated for 10 min at ambient temperature. The reaction solutions were loaded onto a 96-well flat clear bottom black microplates (Corning) and read at excitation and emission wavelengths of 330 and 450 nm, respectively, using a microplate reader (Tecan). The data were analyzed by a one-site total binding regression algorithm of GraphPad Prism (GraphPad Software).

| Ex | IC₅₀(nM) |
|---|---|
| PSMA-617 | 2.4 |
| HTK03149 | 1.6 |
| E1 | 3.3 |
| E2 | 4.0 |
| E3 | 1.2 |
| E4 | 1.4 |
| E5 | 3.0 |
| E6 | 1.5 |
| E7 | 2.1 |
| E8 | 1.6 |
| E9 | 1.0 |
| E10 | 1.7 |
| E11 | 10.0 |
| E12 | 0.9 |
| E13 | 1.7 |
| E14 | 1.7 |
| E15 | 0.8 |
| E16 | 3.5 |
| E17 | 3.5 |
| E18 | 3.7 |
| E20 | 1.8 |
| E21 | 1.5 |
| E22 | 4.9 |
| E24 | 8.6 |
| E25 | 23.1 |
| E26 | 9.5 |
| E27 | 26.6 |
| E28 | 2.1 |

PSMA-617 has the following structure:

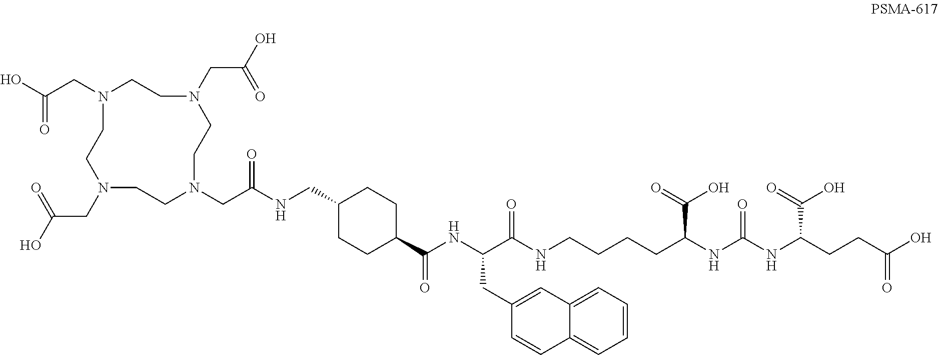 [SMILES: O=C(O)CC[C@H](NC(=O)N[C@@H](CCCCNC(=O)[C@H](Cc1ccc2ccccc2c1)NC(=O)[C@H]1CC[C@H](CNC(=O)CN2CCN(CC(=O)O)CCN(CC(=O)O)CCN(CC(=O)O)CC2)CC1)C(=O)O)C(=O)O]

HTK03149 (described in WO 2020/252598) has the following structure:

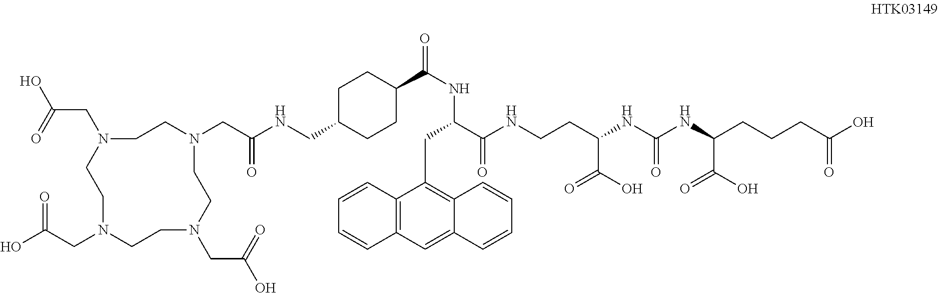 [SMILES: O=C(O)CCC[C@H](NC(=O)N[C@@H](CCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O]

### Radiochemistry

¹⁷⁷Lu labeling: Precursor was mixed with ¹⁷⁷LuCl₃ stock (Isotopia) at a molar ratio of 5:1˜10:1 in the solution (pH 4.76) containing sodium acetate at 0.41 mg/mL and glacial acetic acid at 0.3 mg/mL, or sodium acetate at 4.37 mg/ml and gentisic acid at 1 mg/mL. The mixture was then incubated at 70° C. for 30 min.

⁶⁸Ga labeling: ⁶⁸GaCl₃ solution (1.0 mL, 370 MBq), was eluted from the ⁶⁸Ge/⁶⁸Ga generator (Isotope Technologies Garching) with 0.05 M HCl. ⁶⁸Ga-labelled compound was prepared in sodium acetate buffer (1M, pH 4.2) after incubation of 20 μg of precursor with 20 mCi ⁶⁸GaCl₃ at 95° C. for 20 min. Quality standard: RCP>98% (iTLC), specific activity >0.75 mCi/ug, and activity >1 mCi/ml.

²¹²Pb/²⁰³Pb label: The standard protocol can be found in WO 2021/154921A1, which is incorporated herein by reference.

²²⁵Ac labeling: The standard protocol can be found in WO 2023/191839A2, which is incorporated herein by reference.

Radiochemical purity (RCP) Determination.

RCP was measured by Radio-iTLC. Briefly, ¹⁷⁷Lu-labelled compound was spotted on the iTLC-silica gel chromatography paper (Agilent) and developed in a mobile phase of 0.05 M citric acid/sodium citrate (pH 4.0). The developed strip was scanned with radiometric iTLC scanner (Echert & Ziegler) or cut into 10 equal pieces and proceeded to activity measurement by gamma-counter (Zonkia). RCP acceptance criteria were >90%.

### Cell Uptake Assay

LNCaP (ATCC) cells were cultured in RPMI 1640 medium (Gibco) containing 10% fetal bovine serum (FBS) (Gibco), in 5% carbon dioxide (CO₂), at 37.0° C. in a humidified incubator. 2×10⁵ LNCaP cells/well were seeded in a 24-well plate the day before experiment. 7.4 KBq (200 nCi) of ¹⁷⁷Lu-labeled compound was added into each well, and then incubated in 37° C., 5% CO₂ incubator for varying durations. At each time point, the supernatant, cell membrane and intracellular fractions were collected. The cell membrane fraction was recovered by incubating cells with buffer containing 50 mM glycine and 100 mM NaCl, pH 2.7 for 10 minutes at 37° C., while the intracellular fraction was recovered from cells treated with 1M NaOH. Activity of ¹⁷⁷Lu-labelled compound in every component was detected by gamma counter.

| | *Intracellular (%) | | | **Membrane (%) | | |
|---|---|---|---|---|---|---|
| Ex | 1 hr | 4 hr | 24 hr | 1 hr | 4 hr | 24 hr |
| PSMA-617 | 3.0 | 9.5 | 22.4 | 2.1 | 4.8 | 4.7 |
| HTK03149 | 2.8 | 7.4 | 19.3 | 2.9 | 3.8 | 5.7 |
| E1 | 3.0 | 9.9 | 24.8 | 3.4 | 5.6 | 7.1 |
| E3 | 14.7 | 44.2 | 73.3 | 4.0 | 6.8 | 8.8 |
| E4 | 7.2 | 23.0 | 57.4 | 6.4 | 12.8 | 13.0 |
| E6 | 5.7 | 18.9 | 45.5 | 6.3 | 10.1 | 11.6 |
| E7 | 6.7 | 23.1 | 54.7 | 5.6 | 9.6 | 11.8 |
| E8 | 11.5 | 39.0 | 77.9 | 4.8 | 12.8 | 7.3 |
| E9 | 12.4 | 40.4 | 72.0 | 5.0 | 9.7 | 10.1 |
| E10 | 16.9 | 50.2 | 76.7 | 7.1 | 10.5 | 11.0 |
| E12 | 7.3 | 24.3 | 41.4 | 11.1 | 18.0 | 12.7 |
| E13 | 6.9 | 23.0 | 43.3 | 8.3 | 13.8 | 7.1 |
| E14 | 7.9 | 25.1 | 49.5 | 9.7 | 14.8 | 12.9 |
| E15 | 9.6 | 30.4 | 58.4 | 14.1 | 27.3 | 23.8 |

*The intracellular fraction for each time point is presented
**The cell membrane fraction for each time point is presented

### Biodistribution in LnCAP or 22RV1 CDX Tumor Mouse Models

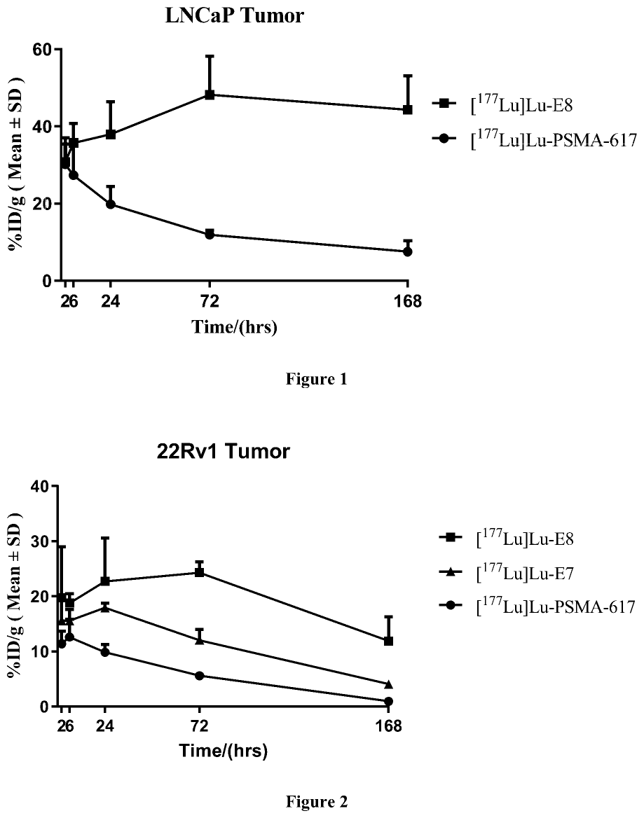

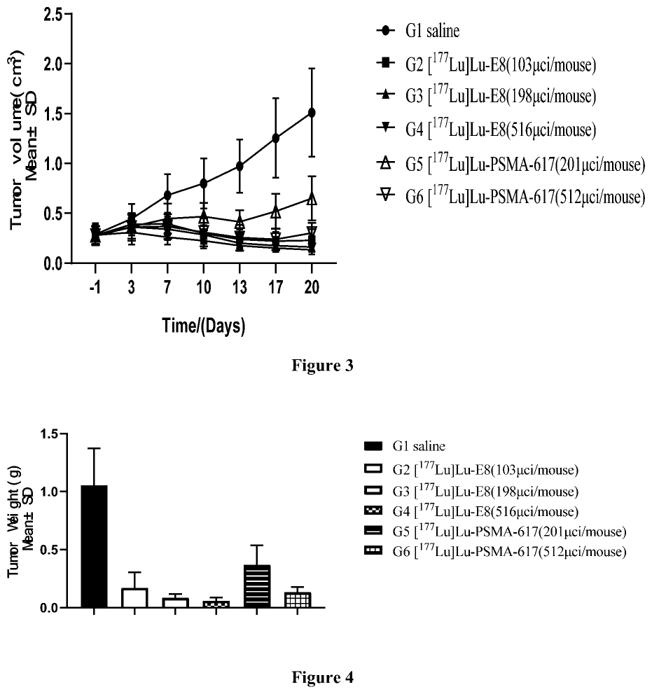

LnCAP and 22RV1 CDX xenograft tumor models were generated by inoculating 5×10⁶ cells/site subcutaneously in male SCID or nude mice (Vital River), respectively. Animals were housed according to IACUC guidance and had accessed to food and water ad libitium. Animals were used for biodistribution study when the tumor dimensions reached around 0.2-0.3 cm³. About 1.10-1.85 MBq of [¹⁷⁷Lu]Lu-E7, [¹⁷⁷Lu]Lu-E8, or [¹⁷⁷Lu]Lu-PSMA-617 was intravenously injected. At each tome point, animals were sacrificed and tissues were collected, weighted, and subjected to radioactivity measurements using gamma counter. Radioactivity in the tissues was presented in % ID/g (percent of injected dose per gram). The data were shown in Tables 4-6, FIG. 1, and FIG. 2.

TABLE 4
Tissue biodistribution of [¹⁷⁷Lu]Lu-E8, [¹⁷⁷Lu]Lu-E7, and [¹⁷⁷Lu]Lu-PSMA-617 in 22Rv1 tumor mouse model.

| | | % ID/g (Mean ± SD) | | | | |
|---|---|---|---|---|---|---|
| Compound | Time (hr) | 2 | 6 | 24 | 72 | 168 |
| [¹⁷⁷Lu]Lu- | Blood | 0.08 ± 0.03 | 0.03 ± 0.01 | 0.01 ± 0.00 | 0.02 ± 0.02 | 0.02 ± 0.00 |
| PSMA-617 | Tumor | 11.38 ± 2.27 | 12.61 ± 3.23 | 9.84 ± 1.45 | 5.57 ± 0.60 | 0.96 ± 0.50 |
| | Heart | 0.10 ± 0.02 | 0.06 ± 0.01 | 0.05 ± 0.01 | 0.05 ± 0.01 | 0.06 ± 0.01 |
| | Liver | 0.09 ± 0.02 | 0.08 ± 0.02 | 0.05 ± 0.01 | 0.04 ± 0.01 | 0.03 ± 0.01 |
| | Spleen | 0.78 ± 0.11 | 0.34 ± 0.10 | 0.14 ± 0.04 | 0.11 ± 0.04 | 0.09 ± 0.03 |
| | Lung | 0.29 ± 0.05 | 0.13 ± 0.03 | 0.06 ± 0.01 | 0.05 ± 0.01 | 0.05 ± 0.00 |
| | Kidney | 53.05 ± 27.39 | 12.27 ± 6.38 | 1.35 ± 0.48 | 0.31 ± 0.01 | 0.08 ± 0.03 |
| | Muscle | 0.09 ± 0.03 | 0.05 ± 0.02 | 0.01 ± 0.00 | 0.02 ± 0.00 | 0.02 ± 0.00 |
| | Bone | 0.26 ± 0.07 | 0.17 ± 0.03 | 0.11 ± 0.03 | 0.18 ± 0.02 | 0.18 ± 0.04 |
| | Salivary | 0.15 ± 0.04 | 0.10 ± 0.02 | 0.06 ± 0.01 | 0.05 ± 0.02 | 0.05 ± 0.01 |
| | Gland | | | | | |
| | Residual | 4.15 ± 0.06 | 1.91 ± 0.02 | 1.21 ± 0.03 | 0.26 ± 0.00 | 0.20 ± 0.00 |
| | Body (% ID) | | | | | |
| | Tumor/ | 151.5 ± 39.40 | 447.29 ± 14.53 | 776.59 ± 235.87 | 353.17 ± 203.83 | 58.51 ± 31.07 |
| | Blood | | | | | |
| | Tumor/ | 0.26 ± 0.15 | 1.17 ± 0.43 | 7.82 ± 2.36 | 17.98 ± 1.41 | 11.77 ± 4.52 |
| | Kidney | | | | | |
| | Tumor/ | 46.97 ± 19.48 | 75.68 ± 27.06 | 92.30 ± 31.06 | 31.97 ± 3.89 | 5.20 ± 2.74 |
| | Bone | | | | | |
| [¹⁷⁷Lu]Lu-E8 | Blood | 0.40 ± 0.14 | 0.08 ± 0.04 | 0.03 ± 0.00 | 0.02 ± 0.00 | 0.02 ± 0.01 |
| | Tumor | 19.71 ± 9.31 | 18.80 ± 1.68 | 22.71 ± 7.83 | 24.33 ± 1.92 | 11.89 ± 4.35 |
| | Heart | 0.57 ± 0.28 | 0.10 ± 0.01 | 0.07 ± 0.00 | 0.07 ± 0.01 | 0.08 ± 0.01 |
| | Liver | 0.34 ± 0.07 | 0.19 ± 0.03 | 0.18 ± 0.05 | 0.22 ± 0.01 | 0.23 ± 0.08 |
| | Spleen | 6.44 ± 3.19 | 0.88 ± 0.26 | 0.28 ± 0.05 | 0.35 ± 0.01 | 0.39 ± 0.11 |
| | Lung | 1.14 ± 0.31 | 0.32 ± 0.11 | 0.12 ± 0.02 | 0.09 ± 0.01 | 0.07 ± 0.02 |
| | Kidney | 135.97 ± 16.72 | 56.50 ± 18.93 | 5.86 ± 1.87 | 0.99 ± 0.04 | 0.26 ± 0.01 |
| | Muscle | 0.23 ± 0.03 | 0.08 ± 0.01 | 0.03 ± 0.00 | 0.02 ± 0.00 | 0.03 ± 0.01 |
| | Bone | 0.47 ± 0.09 | 0.26 ± 0.03 | 0.26 ± 0.04 | 0.34 ± 0.01 | 0.43 ± 0.10 |
| | Salivary | 0.57 ± 0.19 | 0.20 ± 0.07 | 0.08 ± 0.00 | 0.09 ± 0.01 | 0.06 ± 0.01 |
| | Gland | | | | | |
| | Residual | 8.22 ± 0.38 | 5.13 ± 0.24 | 2.13 ± 0.07 | 0.81 ± 0.01 | 1.34 ± 0.01 |
| | Body (% ID) | | | | | |
| | Tumor/ | 62.72 ± 54.35 | 276.92 ± 98.87 | 753.75 ± 165.72 | 1121.44 ± 40.00 | 554.96 ± 352.54 |
| | Blood | | | | | |
| | Tumor/ | 0.15 ± 0.09 | 0.35 ± 0.11 | 3.97 ± 0.93 | 24.54 ± 2.46 | 45.57 ± 16.29 |
| | Kidney | | | | | |
| | Tumor/ | 41.46 ± 19.54 | 73.57 ± 6.68 | 87.22 ± 16.06 | 72.11 ± 4.85 | 27.70 ± 7.20 |
| | Bone | | | | | |
| [¹⁷⁷Lu]Lu-E7 | Blood | 0.23 ± 0.05 | 0.04 ± 0.00 | 0.02 ± 0.00 | 0.02 ± 0.00 | 0.02 ± 0.01 |
| | Tumor | 15.59 ± 4.67 | 15.55 ± 2.01 | 17.91 ± 0.87 | 12.07 ± 1.94 | 4.04 ± 0.66 |
| | Heart | 0.15 ± 0.02 | 0.06 ± 0.01 | 0.05 ± 0.00 | 0.05 ± 0.01 | 0.07 ± 0.01 |
| | Liver | 0.18 ± 0.02 | 0.11 ± 0.01 | 0.11 ± 0.01 | 0.12 ± 0.02 | 0.11 ± 0.02 |
| | Spleen | 0.29 ± 0.03 | 0.12 ± 0.03 | 0.13 ± 0.00 | 0.12 ± 0.01 | 0.15 ± 0.02 |
| | Lung | 0.41 ± 0.08 | 0.12 ± 0.02 | 0.10 ± 0.00 | 0.07 ± 0.01 | 0.06 ± 0.01 |
| | Kidney | 15.24 ± 5.30 | 8.27 ± 2.15 | 6.73 ± 0.66 | 2.88 ± 1.02 | 0.95 ± 0.16 |
| | Muscle | 0.10 ± 0.02 | 0.04 ± 0.02 | 0.02 ± 0.00 | 0.02 ± 0.00 | 0.02 ± 0.00 |
| | Bone | 0.30 ± 0.02 | 0.28 ± 0.05 | 0.26 ± 0.03 | 0.26 ± 0.06 | 0.29 ± 0.10 |
| | Salivary | 0.16 ± 0.01 | 0.07 ± 0.01 | 0.08 ± 0.00 | 0.06 ± 0.01 | 0.06 ± 0.02 |
| | Gland | | | | | |
| | Residual | 5.17 ± 0.12 | 1.71 ± 0.03 | 1.36 ± 0.04 | 1.29 ± 0.06 | 0.23 ± 0.02 |
| | Body (% ID) | | | | | |
| | Tumor/ | 70.15 ± 30.00 | 424.71 ± 27.96 | 847.51 ± 106.60 | 737.10 ± 174.32 | 345.02 ± 219.98 |
| | Blood | | | | | |
| | Tumor/ | 1.05 ± 0.32 | 1.99 ± 0.69 | 2.67 ± 0.15 | 4.48 ± 1.26 | 4.23 ± 0.07 |
| | Kidney | | | | | |
| | Tumor/ | 52.91 ± 18.62 | 57.43 ± 17.95 | 69.95 ± 5.18 | 47.77 ± 16.37 | 15.56 ± 6.16 |
| | Bone | | | | | |

TABLE 5
Tissue biodistribution of [¹⁷⁷Lu]Lu-E8 and [¹⁷⁷Lu]Lu-PSMA-617 in LnCAP tumor mouse model.

| | | % ID/g (Mean ± SD, n = 3) | | | | |
|---|---|---|---|---|---|---|
| Compound | Time (hr) | 2 | 6 | 24 | 72 | 168 |
| [¹⁷⁷Lu]Lu- | Blood | 0.38 ± 0.05 | 0.08 ± 0.04 | 0.03 ± 0.01 | 0.02 ± 0.01 | 0.02 ± 0.00 |
| PSMA-617 | Tumor | 30.16 ± 5.26 | 27.40 ± 7.91 | 19.82 ± 4.66 | 11.92 ± 1.20 | 4.74 ± 1.95 |
| | Heart | 0.27 ± 0.03 | 0.13 ± 0.06 | 0.06 ± 0.02 | 0.06 ± 0.01 | 0.07 ± 0.00 |
| | Liver | 0.21 ± 0.03 | 0.11 ± 0.04 | 0.07 ± 0.02 | 0.06 ± 0.01 | 0.04 ± 0.01 |
| | Spleen | 0.70 ± 0.21 | 0.36 ± 0.04 | 0.16 ± 0.00 | 0.14 ± 0.05 | 0.15 ± 0.08 |
| | Lung | 0.91 ± 0.06 | 0.24 ± 0.06 | 0.09 ± 0.02 | 0.05 ± 0.01 | 0.05 ± 0.01 |
| | Kidney | 66.89 ± 96.14 | 45.58 ± 37.10 | 3.25 ± 1.04 | 0.96 ± 0.33 | 0.26 ± 0.11 |
| | Muscle | 0.15 ± 0.01 | 0.08 ± 0.02 | 0.02 ± 0.00 | 0.02 ± 0.01 | 0.03 ± 0.02 |
| | Bone | 0.34 ± 0.03 | 0.20 ± 0.01 | 0.15 ± 0.01 | 0.19 ± 0.02 | 0.24 ± 0.04 |
| | Gland | 0.56 ± 0.04 | 0.18 ± 0.07 | 0.07 ± 0.00 | 0.06 ± 0.00 | 0.06 ± 0.01 |
| | Residual | 8.53 ± 0.64 | 3.71 ± 1.76 | 4.44 ± 3.53 | 1.03 ± 0.18 | 0.73 ± 0.03 |
| | Body (% ID) | | | | | |
| | Tumor/ | 78.86 ± 4.02 | 388.47 ± 102.96 | 791.68 ± 250.27 | 498.68 ± 84.92 | 209.85 ± 57.90 |
| | Blood | | | | | |
| | Tumor/ | 2.01 ± 1.68 | 1.02 ± 0.92 | 6.21 ± 0.78 | 13.10 ± 3.10 | 18.47 ± 3.57 |
| | Kidney | | | | | |
| | Tumor/ | 88.73 ± 19.34 | 135.69 ± 44.48 | 128.95 ± 33.79 | 63.50 ± 11.13 | 19.59 ± 6.58 |
| | Bone | | | | | |
| [¹⁷⁷Lu]Lu-E8 | Blood | 0.87 ± 0.21 | 0.16 ± 0.04 | 0.03 ± 0.01 | 0.03 ± 0.01 | 0.03 ± 0.01 |
| | Tumor | 30.75 ± 6.34 | 35.70 ± 5.05 | 37.91 ± 8.46 | 48.16 ± 10.04 | 44.31 ± 8.77 |
| | Heart | 1.00 ± 0.40 | 0.26 ± 0.13 | 0.11 ± 0.00 | 0.09 ± 0.00 | 0.11 ± 0.00 |
| | Liver | 0.58 ± 0.17 | 0.33 ± 0.05 | 0.23 ± 0.03 | 0.18 ± 0.02 | 0.15 ± 0.03 |
| | Spleen | 3.87 ± 1.15 | 1.05 ± 0.15 | 0.39 ± 0.14 | 0.31 ± 0.08 | 0.43 ± 0.13 |
| | Lung | 2.97 ± 0.89 | 0.75 ± 0.14 | 0.18 ± 0.03 | 0.08 ± 0.01 | 0.10 ± 0.04 |
| | Kidney | 132.8 ± 12.3 | 121.6 ± 20.9 | 15.74 ± 4.09 | 1.28 ± 0.08 | 0.69 ± 0.32 |
| | Muscle | 0.46 ± 0.09 | 0.14 ± 0.04 | 0.04 ± 0.01 | 0.03 ± 0.00 | 0.04 ± 0.01 |
| | Bone | 0.74 ± 0.16 | 0.33 ± 0.11 | 0.19 ± 0.01 | 0.22 ± 0.02 | 0.26 ± 0.04 |
| | Gland | 1.96 ± 0.74 | 0.51 ± 0.10 | 0.18 ± 0.04 | 0.11 ± 0.01 | 0.10 ± 0.00 |
| | Residual | 16.43 ± 1.14 | 4.69 ± 0.79 | 3.21 ± 2.01 | 0.99 ± 0.20 | 0.91 ± 0.03 |
| | Body (% ID) | | | | | |
| | Tumor/ | 38.44 ± 18.73 | 236.85 ± 57.60 | 1150.30 ± 311.93 | 1689.21 ± 342.70 | 1625.74 ± 208.03 |
| | Blood | | | | | |
| | Tumor/ | 0.24 ± 0.07 | 0.30 ± 0.05 | 2.59 ± 1.05 | 37.54 ± 6.40 | 70.86 ± 22.77 |
| | Kideny | | | | | |
| | Tumor/ | 44.70 ± 20.36 | 116.53 ± 45.73 | 199.66 ± 55.60 | 223.01 ± 43.23 | 170.08 ± 19.34 |
| | Bone | | | | | |

TABLE 6A
Tumor uptake AUC (% ID/g · hr) and T/NT (tumor/non-tumor)
ratios of [¹⁷⁷Lu]Lu-E8 in LnCAP(A) tumor model

| | 2 h to 168 h | | 168 h to infinity | |
|---|---|---|---|---|
| | [¹⁷⁷Lu]Lu- | [¹⁷⁷Lu]Lu- | [¹⁷⁷Lu]Lu- | [¹⁷⁷Lu]Lu- |
| Tissue/Organ | E8 | PSMA-617 | E8 | PSMA-617 |
| Tumor | 5248 | 1708 | 5199.80 | 555.90 |
| Tumor/Blood | 748.97 | 410.87 | 1614.38 | 215.96 |
| Tumor/Kidney | 2.45 | 2.19 | 64.29 | 18.35 |
| Tumor/Bone | 182.92 | 72.16 | 169.77 | 20.00 |
| Tumor/Salivary | 220.13 | 170.12 | 422.38 | 76.59 |
| Gland | | | | |

TABLE 6B
Tumor uptake AUC (% ID/g · hr) and T/NT (tumor/non-tumor)
ratios of [¹⁷⁷Lu]Lu-E8 in 22Rv1 tumor model

| | 2 h~168 h | | 168 h~the infinite | |
|---|---|---|---|---|
| | [¹⁷⁷Lu]Lu- | [¹⁷⁷Lu]Lu- | [¹⁷⁷Lu]Lu- | [¹⁷⁷Lu]Lu- |
| Tissue/Organ | E8 | PSMA-617 | E8 | PSMA-617 |
| Tumor | 2519 | 777.4 | 1395.73 | 112.57 |
| Tumor/Blood | 569.52 | 288.25 | 497.77 | 57.90 |
| Tumor/Kidney | 2.25 | 2.61 | 45.62 | 11.83 |
| Tumor/Bone | 62.98 | 39.56 | 27.62 | 5.19 |
| Tumor/Salivary | 201.04 | 112.83 | 183.55 | 19.15 |
| Gland | | | | |

### Study of Tumor Inhibition Efficacy of [¹¹⁷Lu]Lu-E8 in LnCAP Tumor Model

Method. LnCAP CDX xenograft mouse model was generated as aforementioned. The animals were used for in vivo efficacy study when the average tumor volume reached 300 mm³. Animals were randomized into six groups (G1-G6), 8 animals/group, and dosed with saline as vehicle control, 7.4 and 18.5 MBq [¹⁷⁷Lu]Lu-PSMA-617, or 3.7, 7.4 and 18.5 MBq [¹⁷⁷Lu]Lu-E8, respectively. Tumor volume was measured and recorded twice a week. Animals were euthanized when average tumor volume in the vehicle group reached beyond 1000 mm³ and tumor tissues were collected and weighed. Tumor inhibition efficacy was assessed based on tumor volume or tumor weight at endpoints.

Results. As shown in FIGS. 4 and 5, [¹⁷⁷Lu]Lu-E8 inhibited tumor growth in a dose dependent way, and more strikingly than [¹⁷⁷Lu]Lu-PSMA-617.

### REFERENCE

Deo S V S, Sharma J and Kumar S. GLOBOCAN 2020 Report on Global Cancer Burden: Challenges and Opportunities for Surgical Oncologists. Ann Surg Oncol. 2022; 29(11): 6497-6500.
Dorff T B, Fanti S, Farolfi A, Reiter R E, Sadun T Y and Sartor O. The Evolving Role of Prostate-Specific Membrane Antigen-Based Diagnostics and Therapeutics in Prostate Cancer. Am Soc Clin Oncol Educ Book. 2019 January; 39:321-330.
Heston W D. Characterization and glutamyl preferring carboxypeptidase function of prostate specific membrane antigen: a novel folate hydrolase. Urology. 1997; 49(suppl): 104-112.
Khoshkar Y, Westerberg M, Adolfsson J, Bill-Axelson A, HOlsson H, Eklund M, Akre O, Garmo H and Aly M. Mortality in men with castration-resistant prostate cancer—A long-term follow-up of a population-based real-world cohort. BJUI Compass. 2022 March; 3(2): 173-183.
Kleiburg F, Heijmen L, Gelderblom H, Kielbasa S M, Bovée JV and De Geus-Oei L F. Prostate-specific membrane antigen (PSMA) as a potential target for molecular imaging and treatment in bone and soft tissue sarcomas. Br J Radiol. 2023 Apr. 1; 96(1145):20220886.
O'Keefe D S, Bacich D J, Huang S S and Heston W D. A Perspective on the Evolving Story of PSMA Biology, PSMA-Based Imaging, and Endoradiotherapeutic Strategies. Journal of Nuclear Medicine July 2018, 59 (7) 1007-1013.
Rawla P. Epidemiology of prostate cancer. World J Oncol. 2019, 10(2):63-89.
Sartor O, de Bono J, Chi K N, Fizazi K, Herrmann K, Rahbar K, Tagawa S T, Nordquist L T, Vaishampayan N, El-Haddad G, Park C H, Beer T M, Armour A, Pérez-Contreras W J, DeSilvio M, Kpamegan E, Gericke G, Messmann R A, Morris M J and Krause B J. Lutetium-177-PSMA-617 for Metastatic Castration-Resistant Prostate Cancer. N Engl J Med. 2021 Sep. 16; 385(12):1091-1103.
Terroir M, Lamesa C, Krim M, Vija L, Texier J S, Cassou-Mounat T, Delord J P, Vallot D and Courbon F. RadioLigand Therapy with [¹⁷⁷Lu]Lu-PSMA-617 for Salivary Gland Cancers: Literature Review and First Compassionate Use in France. Pharmaceuticals (Basel). 2023 May 16; 16(5):754-765.
Thurin N H, Rouyer M, Gross-Goupil M, Rebillard X, Soulié M, Haaser T, Roumiguié M, Moulec S L, Capone C, Pierrès M, Lamarque S, Jové J, Bignon E, Droz-Perroteau C, Moore N and Blin P. Epidemiology of metastatic castration-resistant prostate cancer: A first estimate of incidence and prevalence using the French nationwide healthcare database. Cancer Epidemiol. 2020 December; 69:101833.
Wang F, Li Z, Feng X, Yang D and Lin M. Advances in PSMA-targeted therapy for prostate cancer. Prostate Cancer Prostatic Dis. 2022 March; 25(1):11-26.
Wang G, Zhou M, Zang J, Jiang Y, Chen X, Zhu Z and Chen X. A pilot study of ⁶⁸Ga-PSMA-617 PET/CT imaging and ¹⁷⁷Lu-EB-PSMA-617 radioligand therapy in patients with adenoid cystic carcinoma. EJNMMI Res. 2022 Aug. 19; 12(1):52-64.

What is claimed is:

1. A compound of Formula (I): 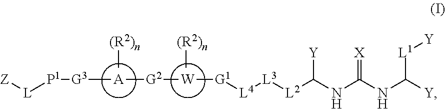
or a stereoisomer, a mixture of stereoisomers, a tautomer, or a pharmaceutically acceptable salt thereof, wherein:
X is O, S, or NH;
each Y is independently —CO₂H, —SO₂H, —SO₃H, —OSO₃H, —PO₂H, —PO₃H₂, —OPO₃H₂, or 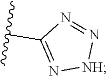 [SMILES: *c1nn[nH]n1]
L¹ is optionally substituted C₁-C₆ alkylene, wherein one or two —CH₂— in the alkylene is independently optionally replaced by a —NHC(═O)—*, —C(═O)NH—*, —O—, —S—, —S(═O)₂—, —S(═O)—, —C(═O)—, —C(═O)O—*, —OC(═O)—*, or —NH—, wherein * refers to the direction toward the Y adjacent to L¹;
L² is optionally substituted C₁-C₆ alkylene, wherein one or two —CH₂— in the alkylene is independently optionally replaced by —O—, —S—, C₃-C₆ cycloalkylene, C₃-C₆ cycloalkenylene, or 3 to 6-membered heterocyclylene;
L³ is —(C═O)—NR′—*, —NR′—(C═O)—*, —C(═O)—, —O—, —S—, —S—S—, —S—CH₂—S—, —S(═O)—, —S(O)₂—, NR′, —NR′—(C═O)—NR′—, —C(═O)—NR′—C(═O)—, —OC(═O)—NR′—*, —NR′C(═O)O—*, —OC(═S)—NR′—*, —NR′C(═S)O—*, 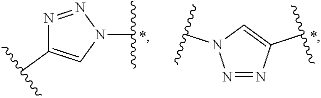 [SMILES: *c1cn(*)nn1 ; *c1cn(*)nn1]
—(C═O)-(3-to 10-membered optionally substituted N-containing ring)-*, or -(3-to 10-membered optionally substituted N-containing ring)-(C═O)—*, wherein * refers to the direction toward L²;
L⁴ is optionally substituted C₁-C₆ alkylene;
G¹ is absent, —O—, —S—, —NR⁴—, —NR⁴—(C═O)—*, —(C═O)—NR⁴—*, or optionally substituted C₂-C₆ alkylene, wherein one or more —CH₂— in the alkylene is independently replaced by a —O—, —S—, —S(═O)₂—, —S(═O)—, —C(═O)—, —C(═O)O—*, —OC(═O)—*, NR⁴, —(C═O)—NR⁴—*, or —NR⁴—(C═O)—*, wherein * refers to the direction toward L⁴;
G² and G³ are each independently absent, —O—, —S—, —NR¹—, —NR¹—(C═O)—*, —(C═O)—NR¹—*, —NR¹—(C═S)—*, or optionally substituted C₁-C₆ alkylene, wherein one or more —CH₂-in the alkylene is independently optionally replaced by a —O—, —S—, —S(═O)₂—, —S(═O)—, —C(═O)—, —C(═O)O—*, —OC(═O)—*, NR¹, —(C═O)—NR¹—*, —NR¹—(C═O)—*, or —NR¹—(C═S)—*, wherein * refers to the direction toward G¹;
R⁴ is H or optionally substituted C₁-C₆ alkyl; or R⁴ and NR¹ of G² together with the intervening atoms form a 5 to 12-membered heterocyclyl ring;
P¹ is absent, NR⁸, C₆-C₁₀ aryl, 5 to 10-membered heteroaryl, C₃-C₁₄ cycloalkyl, or 5 to 14-membered heterocyclyl; wherein the aryl, heteroaryl, cycloalkyl, and heterocyclyl are independently optionally substituted;
R⁸ is independently H or optionally substituted C₁-C₆ alkyl;
Ring W is a 5 to 10-membered heteroaryl, C₃-C₈ cycloalkyl, C₃-C₈ cycloalkenyl, or 5 to 14-membered heterocyclyl;
Ring A is C₆-C₁₀ aryl, 5 to 10-membered heteroaryl, C₃-C₁₄ cycloalkyl, or 5 to 14-membered heterocyclyl;
each R¹ is independently H or optionally substituted C₁-C₆ alkyl;
each R² is independently OH, halogen, oxo, C₁-C₆ alkyl, —O—(C₁-C₆ alkyl), —S—(C₁-C₆ alkyl), —NH₂, —NH—(C₁-C₆ alkyl), or —N(C₁-C₆ alkyl)₂, wherein each alkyl is independently optionally substituted with one or more OH, oxo, or halogen;
n is an integer from 0 to 6 as valency permits;
L is absent or a linker; and
Z is a radioactive moiety, a chelating agent, a fluorescent dye, a contrast agent, a cytostatic or cytotoxic agent, a cytokine, an immunomodulatory molecule, an amphiphilic substance, a nucleic acid, a viral structural protein, a protein, or biotin.

2. The compound of claim 1, wherein G¹ is O.

3. The compound of claim 1, wherein G¹ is NR⁴.

4. The compound of claim 1, which is a compound of Formula (II-A), (II-B), (II-C), or (II-D): 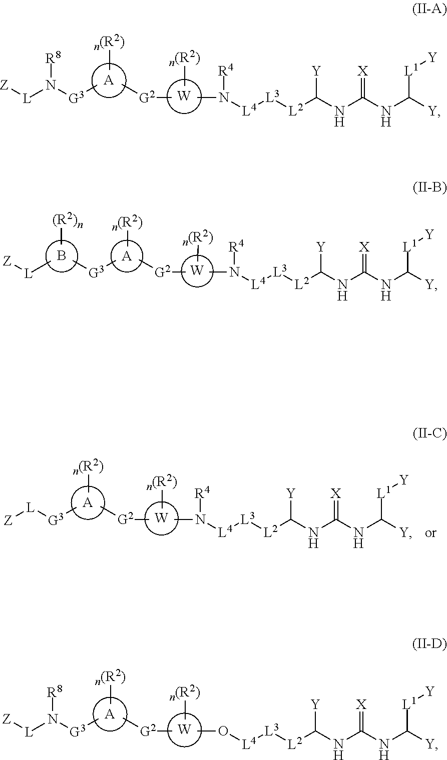
or a stereoisomer, a mixture of stereoisomers, a tautomer, or a pharmaceutically acceptable salt thereof, wherein:
Ring B is C₆-C₁₀ aryl, 5 to 10-membered heteroaryl, C₃-C₁₄ cycloalkyl, or 5 to 14-membered heterocyclyl;
each instance of R² is independently OH, halogen, oxo, C₁-C₆ alkyl, —O—(C₁-C₆ alkyl), —S—(C₁-C₆ alkyl), —NH₂, —NH—(C₁-C₆ alkyl), or —N(C₁-C₆ alkyl)₂, wherein each alkyl is independently optionally substituted with one or more OH, oxo, or halogen; and
each instance of n is an integer from 0 to 6 as valency permits.

5. The compound of any one of claims 1 to 4, wherein L⁴ is —(CH₂)₀₋₃CH(R³)(CH₂)₀₋₃—, or R⁴ is —(CH₂)₁₋₃—R^{3a}, wherein R³ is C₁-C₂₀ alkyl, C₂-C₂₀ alkenyl, C₂-C₂₀ alkynyl, C₃-C₈ cycloalkyl, or —(CH₂)₀₋₃—R^{3a}, wherein each R^{3a} independently is C₆-C₂₀ aryl, C₃-C₁₄ cycloalkyl, 3 to 14-membered heterocyclyl, or 5 to 20-membered heteroaryl, and wherein the alkyl, alkenyl, alkynyl, aryl, cycloalkyl, heterocyclyl, or heteroaryl in R³ or R^{3a} is optionally substituted with one or more halogen, OH, C₁-C₆ alkyl, C₆-C₁₀ aryl, C₆-C₁₀ cycloalkyl, 5- to 10-membered heteroaryl, 3 to 8-membered heterocyclyl, C₆-C₁₀ aryloxy, C₆-C₁₀ cycloalkyloxy, or 5- to 10-membered heteroaryloxy.

6. The compound of claim 5, wherein L⁴ is —(CH₂)₀₋₃CH(CH₂R^{3a})(CH₂)₀₋₃—.

7. The compound of any one of claims 1 to 6, wherein G² is NR¹ or O.

8. The compound of any one of claims 1 to 6, wherein G² is absent or unsubstituted C₁-C₆ alkylene.

9. The compound of any one of claims 1 to 6, wherein G² is C₁-C₆ alkylene, wherein one or two —CH₂— in G² is replaced by a —O—, —S—, —S(═O)₂—, —S(═O)—, —C(═O)—, —C(═O)O—*, —OC(═O)—*, —NH—, —(C═O)—NH—*, —NH—(C═O)—*, or —NR′—(C═S)—*, wherein * refers to the direction toward G¹.

10. The compound of claim 1, which is a compound of Formula (III-A), (III-B), (III-C), (III-D), (III-E), (III-F), or (III-G): 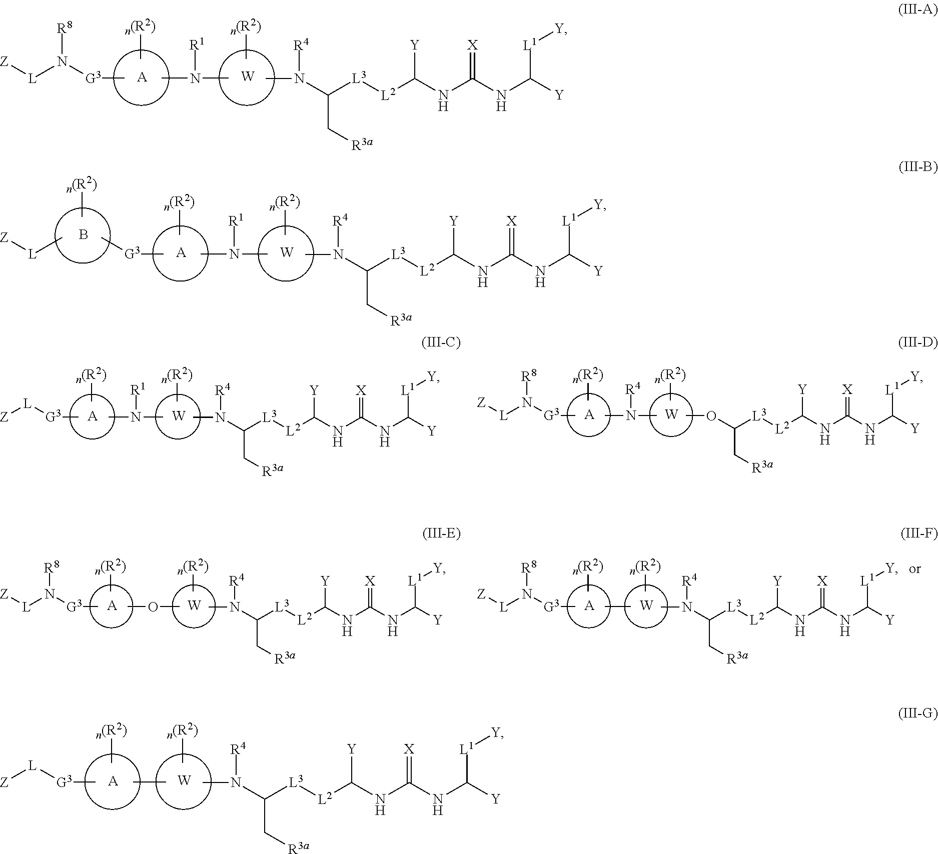
or a stereoisomer, a mixture of stereoisomers, a tautomer, or a pharmaceutically acceptable salt thereof, wherein:
R^{3a} is C₆-C₂₀ aryl, C₃-C₁₄ cycloalkyl, 3 to 14-membered heterocyclyl, or 5 to 20-membered heteroaryl, and wherein the alkyl, alkenyl, alkynyl, aryl, cycloalkyl, heterocyclyl, or heteroaryl in R³ or R^{3a} is optionally substituted with one or more halogen, OH, C₁-C₆ alkyl, C₆-C₁₀ aryl, C₆-C₁₀ cycloalkyl, 5 to 10-membered heteroaryl, 3 to 8-membered heterocyclyl, C₆-C₁₀ aryloxy, C₆-Cia cycloalkyloxy, or 5- to 10-membered heteroaryloxy.

11. The compound of any one of claims 1 to 10, wherein G³ is absent or unsubstituted C₁-C₆ alkylene.

12. The compound of any one of claims 1 to 10, wherein G³ is C₁-C₆ alkylene, wherein one or more —CH₂— in G³ is replaced by a —O—, —S—, —S(═O)₂—, —S(═O)—, —C(═O)—, —C(═O)O—*, —OC(═O)—*, —NH—, —(C═O)—NH—*, —NH—(C═O)—*, or —NR′—(C═S)—*, wherein * refers to the direction toward G¹.

13. The compound of any one of claims 1 to 12, wherein L¹ is unsubstituted C₁-C₆ alkylene, or C₁-C₆ alkylene substituted with one or more halogen.

14. The compound of any one of claims 1 to 12, wherein L¹ is C₁-C₆ alkylene, and wherein one —CH₂— in L¹ is replaced by a —NHC(═O)—*, —C(═O)NH—*, —O—, —S—, —S(═O)₂—, or —S(═O)—, wherein * refers to the direction toward the Y adjacent to L.

15. The compound of any one of claims 1 to 12, wherein L¹ is —CH₂—, —CH₂CH₂—, —CH₂CH₂CH₂—, —CH₂—NHC(═O)—, —CH₂CH₂—NHC(═O)—, —CH₂CH₂CH₂—NHC(═O)—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH₂CH(OH)CH₂—, —CH₂CHFCH₂—, —(CH₂)₂CH(OH)—, —(CH₂)₂CHF—, —CH₂OCH₂—, —CH₂SCH₂—, —CH(OH)CH₂CH₂—, —CH(CH₃)—O—CH₂—, —C(CH₃)₂—O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(═O)NH—CH₂—, —NHC(═O)—CH₂CH₂—, —C(═O)NH—CH(CH₃)—, —NHC(═O)—CH₂CH₂CH₂—, or —C(═O)NH—C(CH₃)₂—.

16. The compound of any one of claims 1 to 15, wherein L² is a straight C₁-C₆ alkylene.

17. The compound of claim 16, wherein L² is —CH₂CH₂CH₂CH₂—.

18. The compound of any one of claims 1 to 17, wherein L³ is —(C═O)—NR′—* or —NR′—(C═O)—*, wherein * refers to the direction toward L².

19. The compound of any one of claims 1 to 18, wherein X is O.

20. The compound of any one of claims 1 to 19, wherein all Y are COOH.

21. The compound of any one of claims 1 to 12, which is a compound of Formula (IV-A1), (IV-A2), (IV-B1), (IV-B2), (IV-C1), or (IV-C2): 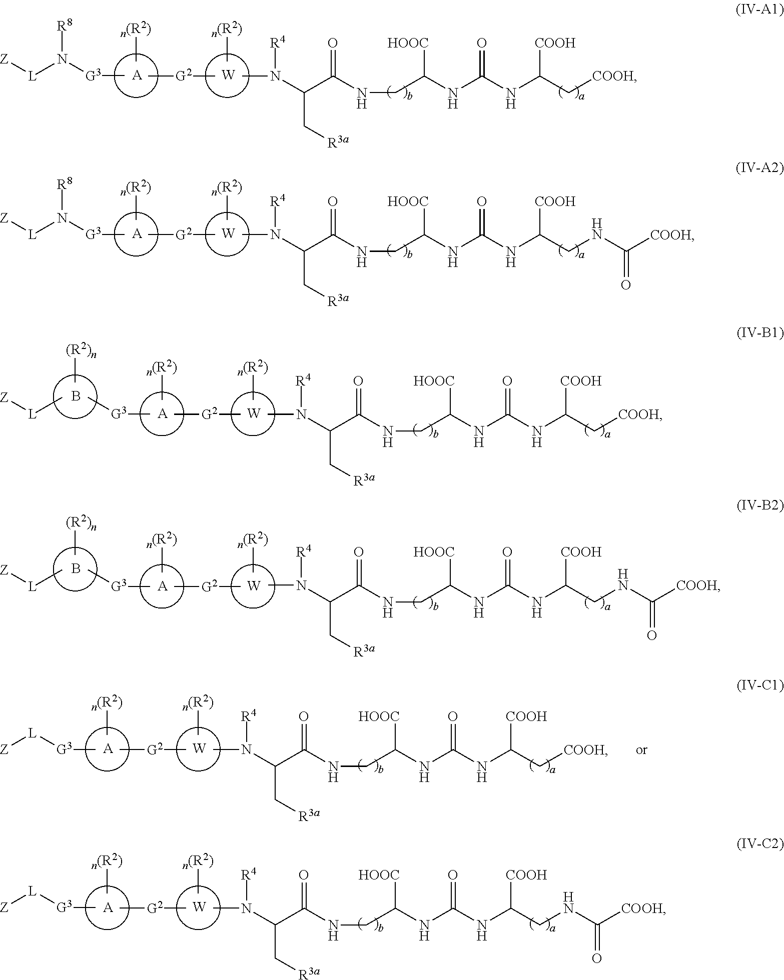
or a stereoisomer, a mixture of stereoisomers, a tautomer, or a pharmaceutically acceptable salt thereof, wherein a and b are each independently an integer from 1 to 5.

22. The compound of claim 21, which is a compound of Formula (V-A1), (V-A2), (V-A3), (V-B1), (V-B2), (V-B3), (V-C1), (V-C2), or (V-C3): 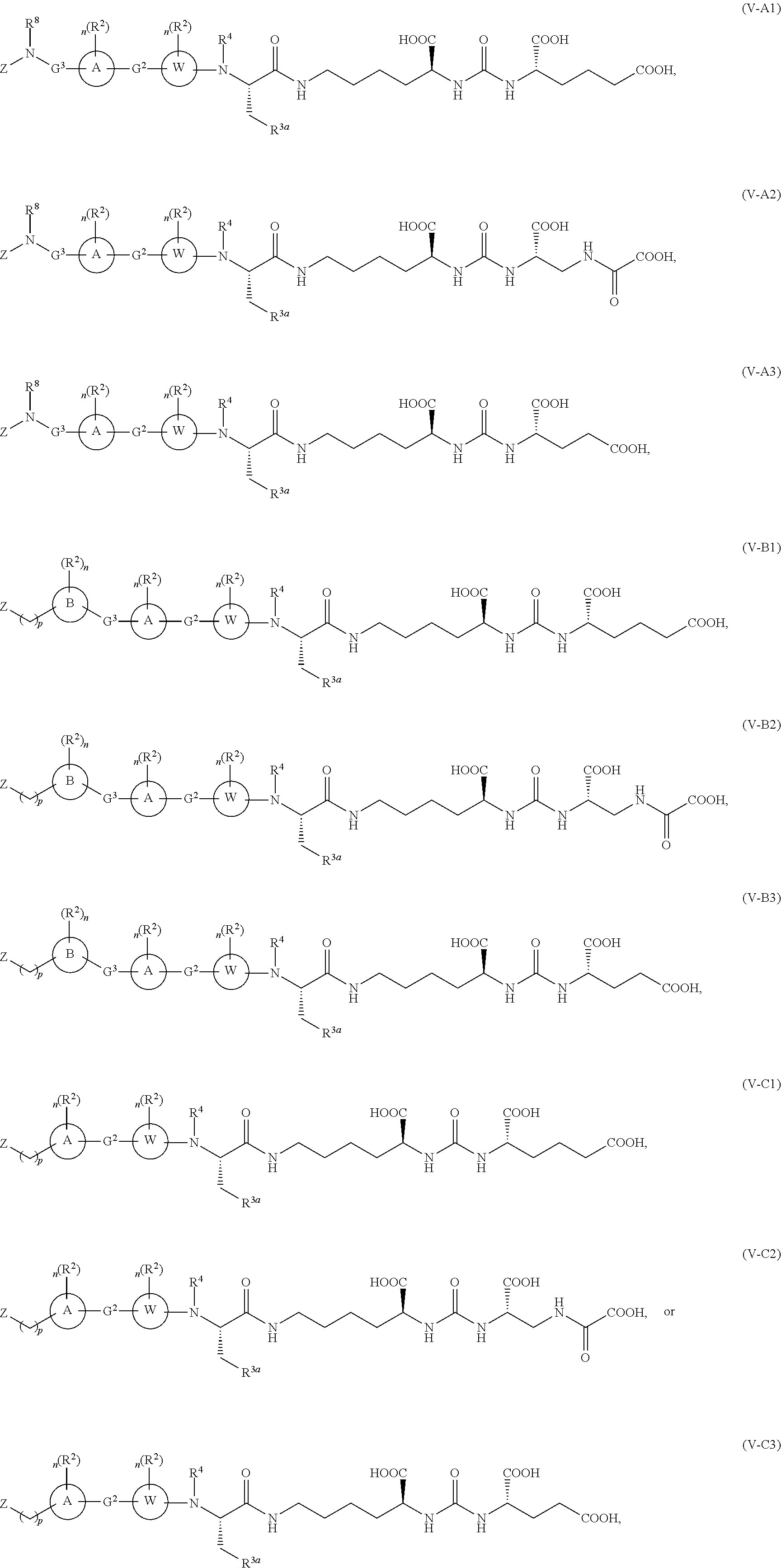
or a stereoisomer, a mixture of stereoisomers, a tautomer, or a pharmaceutically acceptable salt thereof, wherein p is an integer from 0 to 6.

23. The compound of claim 21 or 22, which is a compound of Formula (V-A1′) or (V-B1′): 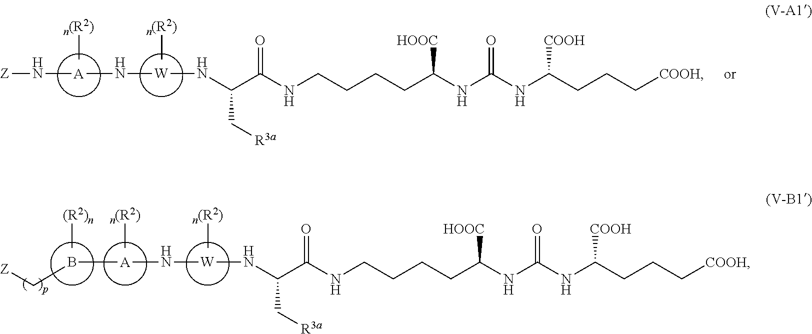
or a stereoisomer, a mixture of stereoisomers, a tautomer, or a pharmaceutically acceptable salt thereof, wherein p is an integer from 0 to 6.

24. The compound of any one of claims 1 to 23, wherein Ring W is a 5 to 10-membered heteroaryl.

25. The compound of claim 24, wherein Ring W is a 5 or 6-membered nitrogen-containing heteroaryl.

26. The compound of any one of claims 1 to 23, wherein Ring W is a C₃-C₈ cycloalkenyl.

27. The compound of claim 26, wherein Ring W is cyclopropenyl, cyclobutenyl, cyclopentenyl, or cyclohexenyl.

28. The compound of any one of claims 1 to 23, wherein Ring W is a 5 to 8-membered heterocyclyl.

29. The compound of claim 28, wherein Ring W is a 5 or 6-membered nitrogen-containing heterocyclyl.

30. The compound of any one of claims 1 to 23, wherein 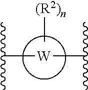
is 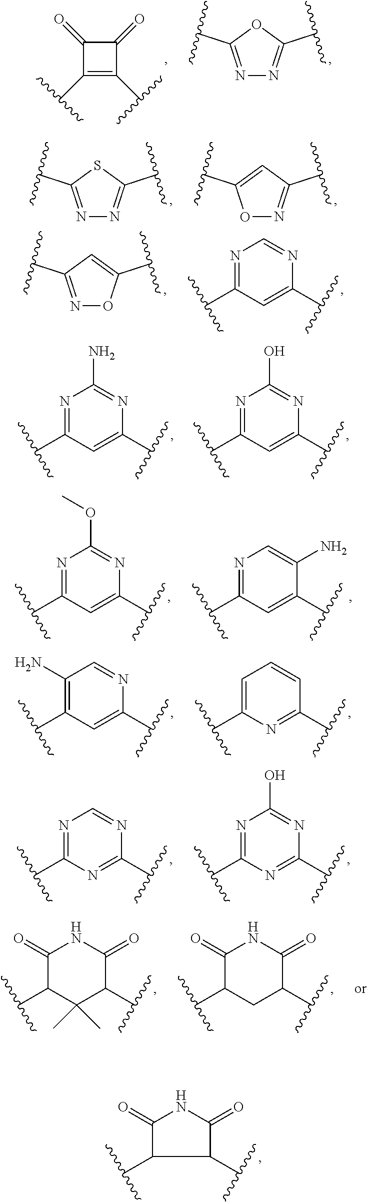
wherein the attachment to the left is to the direction of Z.

31. The compound of any one of claims 4 to 30, wherein Ring B is a 5 or 6-membered heteroaryl.

32. The compound of claim 31, wherein Ring B is a 5 or 6-membered nitrogen-containing heteroaryl.

33. The compound of claim 32, wherein Ring B is 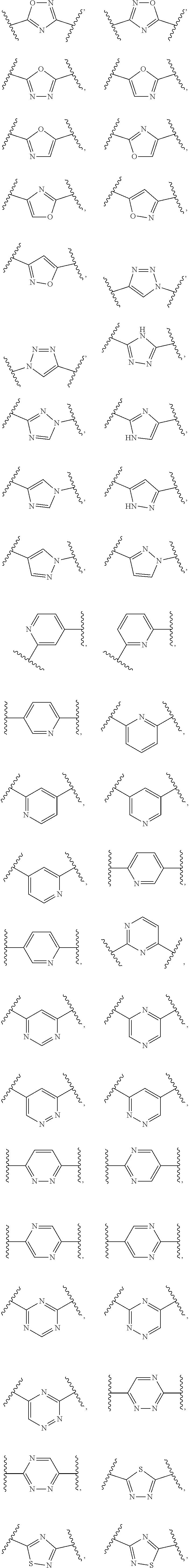 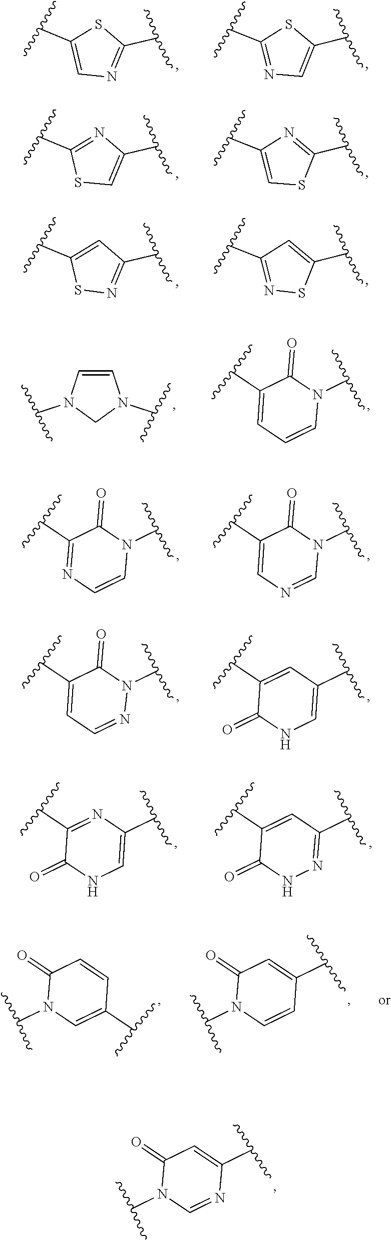
wherein the attachment to the left is to the direction of Z.

34. The compound of any one of claims 1 to 33, wherein Ring A is a fused, bridged or spiro C₅-C₁₂ cycloalkyl, a fused, bridged or spiro 5 to 12-membered heterocyclyl, a fused C₁₀ aryl, or a fused 9 or 10-membered heteroaryl.

35. The compound of claim 34, wherein Ring A is 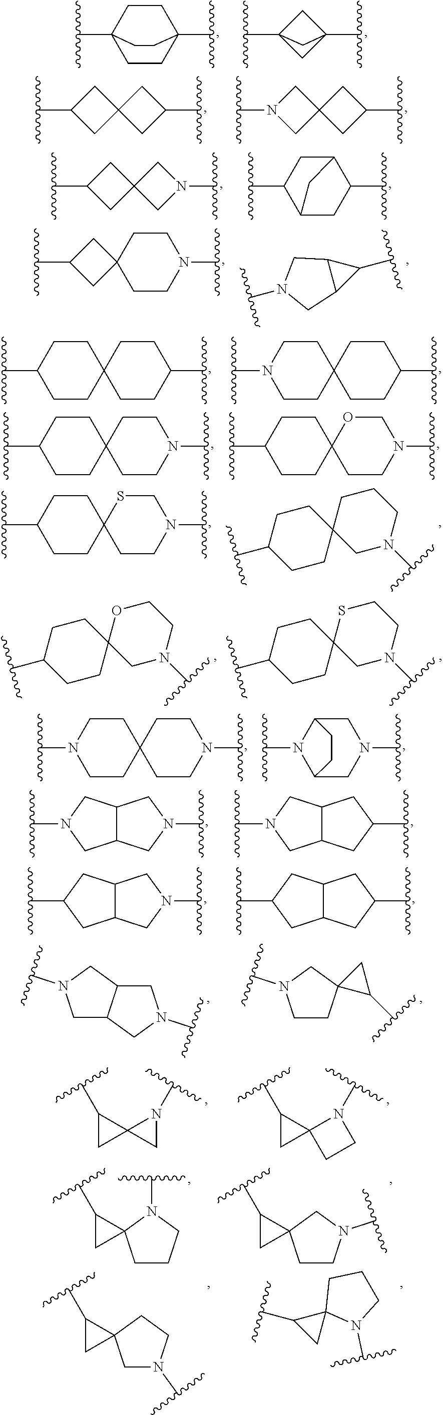 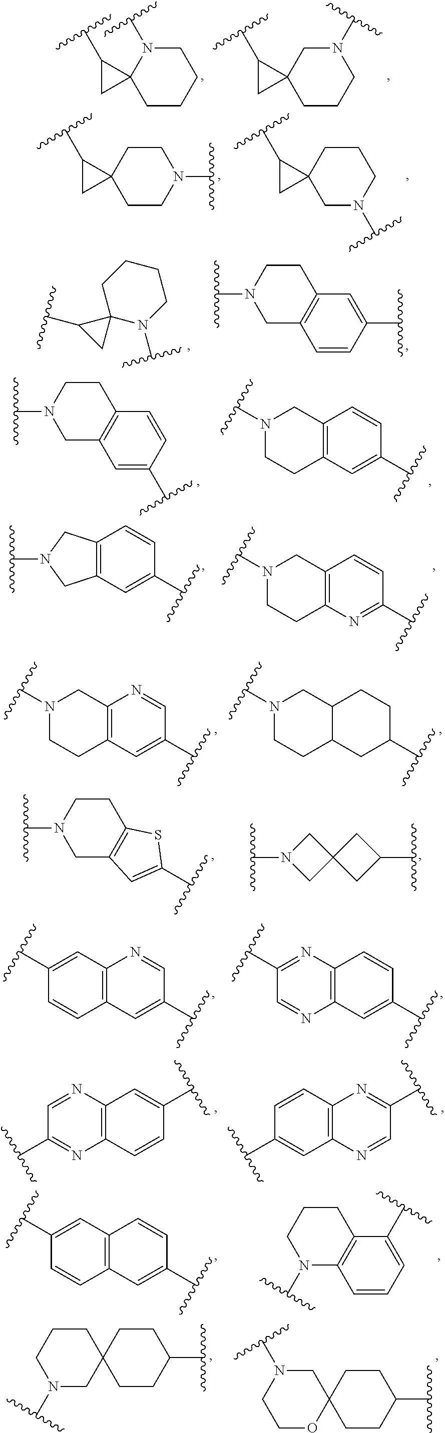 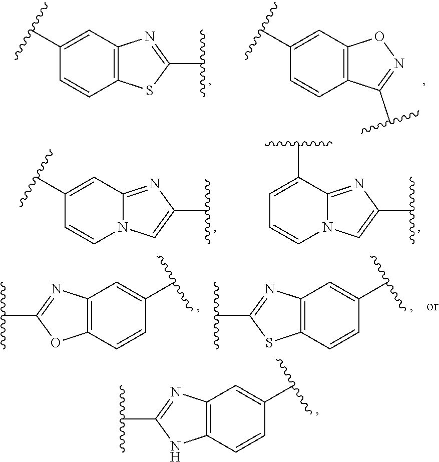
wherein the attachment to the left is to the direction of Z.

36. The compound of any one of claims 1 to 33, wherein Ring A is a monocyclic C₃-C₈ cycloalkyl, or monocyclic 3 to 6-membered heterocyclyl.

37. The compound of any one of claims 5 to 36, wherein R^{3a} is a C₆-C₂₀ aryl, 5 to 20-membered heteroaryl, or 3 to 14-membered heterocyclyl, wherein the aryl, heteroaryl and heterocyclyl are optionally substituted with one or more halogen, OH, C₁-C₆ alkyl, C₆-C₁₀ aryl, C₆-C₁₀ cycloalkyl, C₆-C₁₀ aryloxy, or 3 to 8-membered heterocyclyl.

38. The compound of claim 37, wherein R^{3a} is phenyl, pyridyl, biphenyl, bipyridyl, anthracenyl, acridinyl, acenaphthylenyl, indenyl, phenanthrenyl, phenalenyl, triphenylenyl, naphthalenyl, tetracenyl, chrysenyl, or pyrenyl, wherein R^{3a} is optionally substituted with one or more halogen, OH, or C₁-C₆ alkyl.

39. The compound of claim 37, wherein R^{3a} is

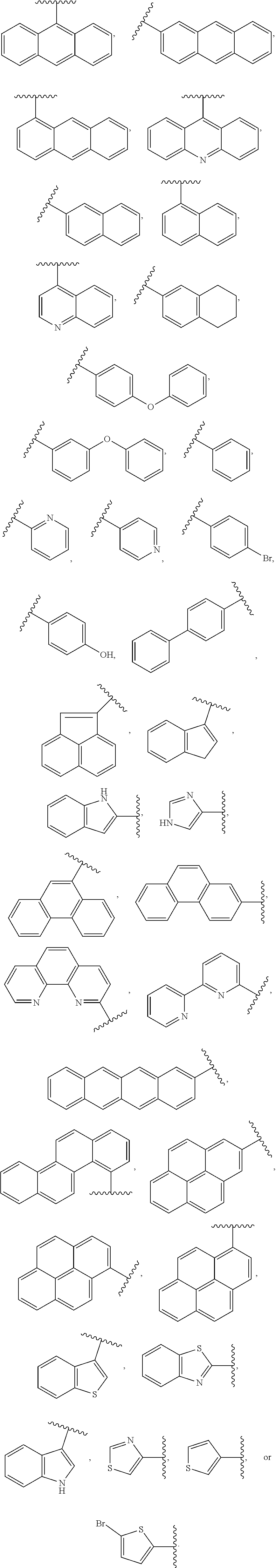

40. The compound of claim 39, wherein R^{3a} is

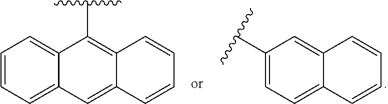 [SMILES: *c1c2ccccc2cc2ccccc12 ; *c1ccc2ccccc2c1]

41. The compound of any one of claims 1 to 40, wherein R is H.

42. The compound of any one of claims 1 to 40, wherein R¹ is C₁-C₆ alkyl optionally substituted with one or more OH, halogen, oxo,

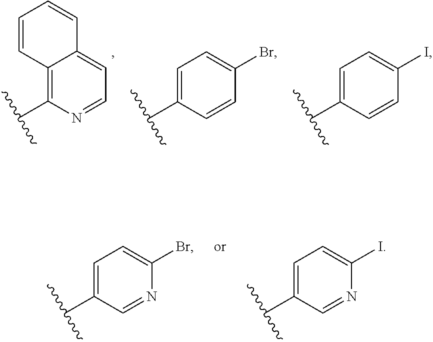

43. The compound of any one of claims 1 to 42, wherein R⁴ is H.

44. The compound of any one of claims 1 to 40, wherein R⁴ and NR¹ of G² together with the intervening atoms form a 5 to 8-membered heterocyclyl ring.

45. The compound of any one of claims 1 to 44, wherein each R² is independently OH, oxo, halogen, NH₂, or C₁-C₆ alkyl.

46. The compound of any one of claims 1 to 45, wherein each n is independently 0, 1, or 2.

47. The compound of any one of claims 1 to 46, wherein L is absent.

48. The compound of any one of claims 1 to 46, wherein L is a linker.

49. The compound of claim 48, wherein L comprises or is an optionally substituted C₁-C₁₂ alkylene, wherein one or more —CH₂— in the alkylene is independently optionally replaced by a —NHC(═O)—*, —C(═O)NH—*, —NHC(═S)—*, —C(═S)NH—*, —O—, —S—, —S(═O)₂—, —S(═O)—, —C(═O)—, —C(═O)O—*, —OC(═O)—*, or —NH—, wherein * refers to the direction toward P¹.

50. The compound of claim 49, wherein the linker is —(CH₂)₁₋₆—.

51. The compound of any one of claims 1 to 50, wherein Z is a radioactive moiety.

52. The compound of claim 51, wherein the radioactive moiety is a fluorescent isotope, a radioisotope, or a radioactive drug.

53. The compound of claim 51, wherein the radioactive moiety is selected from the group consisting of alpha radiation emitting isotopes, beta radiation emitting isotopes, gamma radiation emitting isotopes, Auger electron emitting isotopes, X-ray emitting isotopes, and fluorescence emitting isotopes.

54. The compound of claim 51, wherein the radioactive moiety is a complex formed by a radioisotope of a metal cation and a chelating agent.

55. The compound of claim 51, wherein the radioactive moiety is a complex formed by a cation of ¹⁷⁷Lu, Al¹⁸F, ²⁰³Pb, ²¹²Pb, ⁵¹Cr, ⁶⁷Ga, ⁶⁸Ga, ⁸⁹Zr, ¹¹¹In, ^{99m}Tc, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ⁴⁷Se, ¹⁴²Pr, ¹⁵⁹Gd, ²¹²Bi, ²¹³Bi, ⁹⁷Ru, ¹⁰⁹Pd, ¹⁰⁵Rh, ^{101m}Rh, ¹¹⁹Sb, ¹²⁸Ba, ⁹⁷Hg, ¹⁵¹Eu, ¹⁵³Eu, ¹⁶⁹Eu, ²⁰¹Tl, ⁶⁴Cu, ⁶⁷Cu, ¹⁸⁸Re, ¹⁸⁶Re, ¹⁹⁸Au, ²²⁵Ac, ²²⁷Th, or ¹⁹⁹Ag and a chelating agent in Table 1.

56. The compound of claim 51, wherein the radioactive moiety is a metal-chelating moiety of: ¹⁷⁷Lu-DOTA, ¹⁷⁷Lu-DOTAGA, ⁶⁸Ga-DOTA, ⁹⁰Y-DOTA, Al¹⁸F-NOTA, ²⁰³Pb-TCMC, ²¹²Pb—PSC, ²⁰³Pb—PSC, ²¹²Pb-TCMC, ⁶⁴Cu-DOTA, or ²²⁵Ac-DOTA.

57. The compound of claim 51, wherein the radioactive moiety comprises ¹¹C, ¹⁸F, ⁷²As, ⁷²Se, ¹²³I, ¹²⁴I, ¹³¹I, or ²¹¹At.

58. The compound of any one of claims 1 to 50, wherein Z is a fluorescent dye.

59. The compound of claim 58, wherein the fluorescent dye is an Xanthene, an Acridine, an Oxazine, an Cyanine, a Styryl dye, a Coumarin, a Porphine, a Metal-Ligand-Complex, a Fluorescent protein, a Nanocrystals, a Perylene, a Boron-dipyrromethene, or a Phthalocyanine, or a conjugate or combination thereof.

60. The compound of any one of claims 1 to 50, wherein Z is a chelating agent.

61. The compound of claim 60, wherein the chelating agent is a tetradentate chelating agent, a hexadentate chelating agent, or an octadentate chelating agent.

62. The compound of claim 60 or 61, wherein the chelating agent comprises an optionally substituted 8 to 20-membered nitrogen-containing heterocyclyl.

63. The compound of claim 60, wherein the chelating agent is a chelating moiety of: 1,4,7,10-tetraazacyclododecane-N,N′,N,N′-tetra acetic acid (DOTA), ethylenediaminetetraacetic acid (EDTA), 1,4,7-triazacyclononane-1,4,7-triacetic acid (NOTA), 1,4,7,10-tetraazacyclododecane-1-(glutaric acid)-4,7,10-triacetic acid (DOTAGA), 2-[4,7,10-tris(2-amino-2-oxoethyl)-1,4,7,10-tetrazacyclododec-1-yl]acetamide (TCMC), triethylenetetramine (TETA), iminodiacetic acid, diethylenetriamine-N,N,N′,N′,N″-penta acetic acid (DTPA), bis-(carboxymethyl imidazole)glycine, or 6-hydrazinopyridine-3-carboxylic acid (HYNIC).

64. The compound of claim 60, wherein the chelating agent is a chelating agent in Table 1.

65. The compound of any one of claims 1 to 50, wherein Z is a contrast agent.

66. The compound of claim 65, wherein the contrast agent comprises a paramagnetic agent.

67. A compound in Table 2 or Table 2A, or a stereoisomer, a mixture of stereoisomers, a tautomer, or a pharmaceutically acceptable salt thereof.

68. A complex formed by a compound of any one of claims 1 to 50, 60 to 64, or 67 and a metal cation.

69. The complex of claim 68, wherein the metal cation is a cation of Cr, Ga, In, Tc, Re, La, Yb, Sm, Ho, Y, Pm, Dy, Er, Lu, Sc, Pr, Gd, Bi, Ru, Pd, Rh, Sb, Ba, Hg, Eu, Ti, Pb, Cu, Re, Au, Ac, Th, or Ag.

70. The complex of claim 68, wherein the metal cation is a cation of ⁵¹Cr, ⁶⁷Ga, ⁶⁸Ga, ⁸⁹Zr, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁸Y, ⁹⁰Y, ¹⁴⁹Pm, ¹⁶⁵Dy, ¹⁶⁹Er, ¹⁷⁷Lu, ⁴⁷Sc, ¹⁴²Pr, ⁵⁹Gd, ²¹²Bi, ²¹³Bi, ⁹⁷Ru, ¹⁰⁹Pd, ¹⁰⁵Rh, ^{101m}Rh, ¹¹⁹Sb, ¹²⁸Ba, ¹⁹⁷Hg, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁹⁸Au, ²²⁵Ac, ²²⁷Th, or ¹⁹⁹Ag.

71. The complex of claim 70, wherein the metal cation is ¹⁷⁷Lu³⁺, ⁶⁸Ga³⁺, ¹¹¹In³⁺, ^{99m}Tc⁴⁺, ⁹⁰Y³⁺, ²⁰³Pb²⁺, ²¹²Pb²⁺, ⁶⁴Cu²⁺, or ²²⁵Ac³⁺.

72. A complex in Table 3, or a stereoisomer, a mixture of stereoisomers, a tautomer, or a pharmaceutically acceptable salt thereof.

73. A pharmaceutical composition comprising a compound of any one of claims 1 to 67 or a complex of any one of claims 68 to 72, and a pharmaceutically acceptable excipient.

74. A method of treating or diagnosing a prostate-specific membrane antigen (PSMA) positive cancer, comprising administering a therapeutically effective amount of a compound of any one of claims 1 to 67, a complex of any one of claims 68 to 72, or a pharmaceutical composition of claim 73 to a subject in need thereof.

75. The method of claim 74, wherein the cancer is prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer, adenoid cystic carcinoma, salivary duct carcinoma, or sarcoma.

76. The method of claim 75, wherein the cancer is prostate cancer.

77. A method of detecting cells or tissues expressing prostate-specific membrane antigen (PSMA) comprising (i) contacting the PSMA-expressing cells or tissues with a compound of any one of claims 1 to 67, a complex of any one of claims 68 to 72, or a pharmaceutical composition of claim 73 and (ii) applying one or more imaging method to detect the cells or tissues.

78. The method of claim 77, wherein the imaging method comprises positron emission tomography (PET), single-photon emission computed tomography (SPECT), magnetic resonance imaging (MRI), computed tomography (CT), scintigraphy imaging, luminescence imaging, or fluorescence imaging, or a combination thereof.

79. The method of claim 77 or 78, wherein the PSMA-expressing cells or tissues comprise prostate cells or tissues, spleen cells or tissues, or kidney cells or tissues.

80. The method of any one of claims 77 to 79, wherein the detecting is performed in vivo, ex vivo, or in vitro.
